(19) United States
(12) Patent Application Publication
(10) Pub. No.: US 2026/0273238 A1
(43) Pub. Date: Sep. 17, 2026

(54) CATHETERS AND SYSTEM FOR ISOLATING AN INTERIOR OF A MAMMAL HOLLOW ORGAN

(71) Applicant: PANDICA LTD, London (GB)

(72) Inventors: Aleksei Arievich KASHINTSEV, Saint Petersburg (RU); Vitali PROUTSKI, Dubai (AE)

(21) Appl. No.: 19/668,342

(22) Filed: May 5, 2026

Related U.S. Application Data
(63) Continuation-in-part of application No. 17/790,320, filed on Dec. 28, 2020, now Pat. No. 12,653,660.

(30) Foreign Application Priority Data
Dec. 31, 2019 (RU) 2020100187
Dec. 9, 2020 (RU) 2020140524

(51) Int. Cl. A61M 25/10; A61M 25/01

(52) U.S. Cl. CPC A61M 25/1011; A61M 25/013; A61M 25/10185; A61M 2025/1052; A61M 2025/1061

(57) ABSTRACT

Catheters for isolating an interior of a mammal hollow organ, each comprising: (i) an elongate catheter body designed to be inserted into a lumen of the mammal hollow organ; (ii) two balloons, individually and separately disposed along the elongate catheter body and designed to be inflated to isolate the interior of the mammal hollow organ therebetween; and (iii) a functional channel extending in the elongate catheter body and comprising a functional opening provided in the elongate catheter body between the balloons, wherein the functional channel is designed to allow a negative pressure to be produced in the isolated interior to take a gaseous medium, a liquid medium or a fluid medium therefrom via the functional opening.

### CROSS-REFERENCE TO RELATED APPLICATIONS AND PRIORITY

This patent application is a Continuation-in-Part of U.S. patent application Ser. No. 17/790,320 filed Jun. 30, 2022, which claims priority from PCT Application No. PCT/RU2020/050399 filed Dec. 28, 2020, which claims priority from Russian Patent Application Nos. 2020140524 filed Dec. 9, 2020 and 2020100187 filed Dec. 31, 2019. Each of these patent applications are herein incorporated by reference in its/their entirety.

### FIELD OF THE INVENTION

The present invention relates to medicine, and more particularly to medical equipment used to diagnose, monitor and/or treat inflammatory, autoimmune, infectious, benign and/or malignant diseases, that occur in hollow organs and connected with them organs of a mammal, in particular diseases of pancreas, bile ducts, liver, gastrointestinal tract, and also defects and injuries of hollow organs of a mammal, fistulas, strictures, aneurismal and diverticular dilatations, and other diseases of the hollow organs. In particular, the present invention relates to catheters and a system for isolating an interior of a mammal hollow organ that have an improved safety.

### BACKGROUND

Known in the art are various devices and appliances used to diagnose and/or treat inflammatory, autoimmune, infectious, benign and/or malignant diseases that occur in hollow organs of a mammal, in particular diseases of pancreas, bile duct diseases, liver diseases, and also gastrointestinal wall defects, injuries of the wall of a hollow organ, aneurismal and diverticular dilatation, strictures of hollow organs, bleeding of hollow organs and some other diseases of hollow organs, and diseases of organs connected to hollow organs.

A similar catheter for isolating an interior of a mammal hollow organ is disclosed in U.S. Pat. No. 9,526,874 published on 30 Jun. 2015. The catheter disclosed in U.S. Pat. No. 9,526,874 comprises an elongate catheter body designed to be inserted into a lumen of a mammal hollow organ and two isolating balloons designed to be inflated to isolate the interior of the mammal hollow organ therebetween; and a functional channel extending in the catheter body and comprising a functional opening provided in the elongate catheter body between the isolating balloons, wherein the functional channel is designed to allow: a negative pressure to be produced in the isolated interior to take therefrom a biological fluid being specific to the hollow organ or a liquid or gaseous medium to be supplied into the isolated interior.

A disadvantage of the catheter disclosed in U.S. Pat. No. 9,526,874 is in that it cannot be inserted into a lumen of a mammal hollow organ for a long time due to the lack of physiological connectivity between hollow organ sections adjoining the hollow organ interior isolated by the inflated isolating balloons and being outside the isolated interior of the mammal hollow organ.

Other similar catheters for isolating an interior of a mammal hollow organ are disclosed in U.S. Pat. No. 4,781,677 published on 1 Nov. 1988, U.S. Pat. No. 5,460,610 published on 24 Oct. 1995, international publication No. 2018106788 published on 14 Jun. 2018, U.S. Pat. No. 5,951,514 published on 14 Sep. 1999 and US Patent No. U.S. Pat. No. 5,222,941 published on 29 Jun. 1993.

A main disadvantage of the catheter as disclosed in U.S. Pat. No. 478,167, U.S. Pat. No. 5,460,610, WO 2018106788 or U.S. Pat. No. 5,222,941 is in that the functional opening of the catheter body may be quickly blocked or occluded with a tissue of the mammal hollow organ and/or a material related to the catheter body or catheter structural components and, thus, the catheter cannot be used in the mammal hollow organ for a long time, and the mammal hollow organ in turn may be damaged.

The closest prior art to the present invention is a catheter disclosed in U.S. Pat. No. 5,951,514 (IPC: A61M 29/00; publication date: 14 Sep. 1999). The catheter disclosed in U.S. Pat. No. 5,951,514 has the same disadvantage as the above-mentioned catheters disclosed in U.S. Pat. No. 478,167, U.S. Pat. No. 5,460,610, WO 2018106788 and U.S. Pat. No. 5,222,941.

Therefore, catheters and a system for isolating an interior of a mammal hollow organ are to be further developed, in particular to prevent the functional opening of the catheter body from being blocked or occluded with a hollow organ tissue of the mammal hollow organ and, therefore, to prevent the mammal hollow organ from being damaged when using the catheter within the mammal hollow organ.

Consequently, a main technical problem to be solved by the present invention is to develop catheters and a system for isolating an interior of a mammal hollow organ that would at least partly eliminate the above disadvantage of the prior art, i.e. to eliminate the problem of blockage or occlusion of the functional opening with a hollow organ tissue that may cause damage to the mammal hollow organ during use of the catheter.

### SUMMARY OF INVENTION

An objective of the present invention is to develop catheters and a system for isolating an interior of a mammal hollow organ that would solve at least the above-mentioned main technical problem.

To achieve the objective of the present invention, as embodied and broadly described herein, in a first aspect of the present invention, there is provided a catheter for isolating an interior of a mammal hollow organ, the catheter comprising: (i) an elongate catheter body designed to be inserted into a lumen of the mammal hollow organ; (ii) two balloons, individually and separately disposed along the elongate catheter body and designed to be inflated to isolate the interior of the mammal hollow organ therebetween; and (iii) a functional channel extending in the elongate catheter body and comprising a functional opening provided in the elongate catheter body between the balloons, wherein the functional channel is designed to allow a negative pressure to be produced in the isolated interior to take a gaseous medium, a liquid medium or a fluid medium therefrom via the functional opening, and wherein a spiral-shaped projection divided by a gap into two curved sections is further provided on a catheter body part defined by the two balloons such that the gap comprises the functional opening, and the two curved sections of the spiral-shaped projection enclose the functional opening, wherein the spiral-shaped projection is designed such that the spiral-shaped projection allows a distance between a mucosal tissue of the mammal hollow organ, the mucosal tissue being between the inflated balloons, and the functional opening.

A main technical effect provided by the catheter according to the first aspect of the present invention is improved safety of the catheter due to prevented suction of a hollow organ tissue of the mammal hollow organ (e.g. a mucosal tissue of the mammal hollow organ) into the functional opening of the catheter body that would otherwise cause damage to the hollow organ tissue. In particular, in the present invention according to the first aspect of the present invention, suction of the hollow organ tissue into the functional opening is further prevented due to the fact that the functional opening provided in the elongate catheter body is located in the gap between the two curved sections of the spiral-shaped projection that enclose the functional opening. In other words, in the present invention according to the first aspect, the two curved sections of the spiral-shaped projection enclosing the functional opening do not allow the hollow organ tissue to contact the functional opening directly or approach to the functional opening for a distance appropriate for suction of the hollow organ tissue into the functional opening when the negative pressure is produced in the isolated interior between the inflated balloons.

The catheter according to the first aspect of the present invention provides a further technical effect which is an increased reliability of the catheter due to prevented partial or complete blockage or occlusion of the functional opening that may be caused by suction of a tissue of the mammal hollow organ into the functional opening (i.e. suction of the hollow organ tissue into the functional opening is further prevented due to the fact that the two curved sections of the spiral-shaped projection enclose the functional opening provided in the elongate catheter body, and the functional opening is located in the gap between the two curved sections of the spiral-shaped projection), thereby preventing the inserted catheter from being retrieved from the mammal hollow organ and, then, changing the retrieved catheter to a new catheter or removing the hollow organ tissue sucked into the functional opening from the functional opening of the retrieved catheter.

The catheter according to the first aspect of the present invention provides an additional technical effect which is an increased efficiency of the catheter due to an improved communication between different areas of the isolated interior of the mammal hollow organ that is provided by the spiral-shaped projection provided on the catheter body part defined by the two balloons. In particular, the spiral-shaped projection allows the gaseous medium, the liquid medium or the fluid medium from all the isolated interior of the mammal hollow organ to be taken via the functional opening when producing the negative pressure in the isolated interior of the mammal hollow organ, especially when the mucosal tissue of the mammal hollow organ is closely glued or adhered to the spiral-shaped projection, thereby positively influencing the reliability or the representativity of laboratory-instrumental analysis results obtained for the taken medium.

In an embodiment of the present invention, the spiral-shaped projection is further provided with two additional gaps, wherein the two additional gaps are spaced from the gap such that the gap is positioned between the two additional gaps. The two additional gaps spaced from the gap positioned therebetween further contribute to the above additional technical effect, i.e. further increase the efficiency of the catheter since they further improve the communication between different areas of the isolated interior of the mammal hollow organ, thereby further allowing the gaseous medium, the liquid medium or the fluid medium from all the isolated interior of the mammal hollow organ to be taken via the functional opening when producing the negative pressure in the isolated interior of the mammal hollow organ.

In another embodiment of the present invention according to the first aspect, the gap and the two additional gaps as provided on the catheter body part defined by the two balloons are further circumferentially displaced in relation to each other or aligned with each other along the catheter body part defined by the balloons. Displacement of the gap and the two additional gaps in relation to each other on the circumference of the catheter body part defined by the two balloons or alignment of the gap and the two additional gaps in relation to each other along the length of the catheter body part defined by the two balloons further contributes to the above additional technical effect, i.e. further increases the efficiency of the catheter since the displacement of the gap and the two additional gaps further improves the communication between different areas of the isolated interior of the mammal hollow organ, thereby further allowing the gaseous medium, the liquid medium or the fluid medium from all the isolated interior of the mammal hollow organ to be taken via the functional opening when producing the negative pressure in the isolated interior of the mammal hollow organ.

In one of embodiments of the present invention according to the first aspect, the catheter may further comprise an inflation channel extending in the elongate catheter body to deliver a liquid or gaseous medium to the balloons to provide inflation thereof, wherein the functional channel and the inflation channel are hermetically isolated from each other. Delivery of the liquid or gaseous medium to the balloons through the inflation channel for inflating the balloons provides another technical effect which is a combination of simplified maintenance of balloon sizes as required to isolate the interior of the mammal hollow organ and simplified regulation of sizes or inflation degrees of the balloons. Moreover, the hermetically isolated functional and inflation channels in the catheter according to the first aspect of the present invention provide a further technical effect which is in that the liquid or gaseous medium used to inflate the balloons is prevented from entering the functional channel and, therefore, the gaseous medium, the liquid medium or the fluid medium (in particular, biological contents) taken via the functional opening is prevented from mixing with said inflation medium that would otherwise negatively influence the reliability or representativity of laboratory-instrumental analysis results obtained for the biological liquid medium or biological gaseous medium.

In another embodiment of the present invention according to the first aspect, the catheter comprising the inflation channel may further comprise a main channel extending in the elongate catheter body, wherein the main channel is provided at opposite ends thereof with an inlet and an outlet provided both in the elongate catheter body outside the catheter body part defined by the two balloons, and wherein the main channel is hermetically isolated from the functional channel and the inflation channel, and wherein the main channel is further designed to supply a gaseous medium, a liquid medium or a fluid medium thereinto and further comprises an additional outlet provided at a distal end of the elongate catheter body, the distal end being used for inserting the elongate catheter body into the lumen of the mammal hollow organ.

A further technical effect provided by the main channel of the elongate catheter body is the provision of liquid medical products or the provision of an enteral nutrition mixture, the enteral nutrition mixture providing a mammal organism with basic nutrients, energy, vitamins, macronutrients, micronutrients and/or etc., to an interior of the mammal hollow organ (particularly, to a distal section of the mammal hollow organ) via the additional outlet while sampling a biological liquid medium or biological gaseous medium from the isolated interior of the mammal hollow organ via the functional opening of the functional channel.

Another technical effect provided by the main channel of the elongate catheter body is prevented or excluded formation of congestive and/or inflammatory processes which would occur in the mammal hollow organ due to the accumulation of mucus and other biological contents being specific to the mammal hollow organ outside the catheter body part defined by the balloons. In particular, the formation of congestive and/or inflammatory processes in the mammal hollow organ is prevented or excluded due to the fact that mucus and other biological contents being specific to the mammal hollow organ, when accumulated in the mammal hollow organ, may enter the inlet provided in the elongate catheter body outside the catheter body part defined by the balloons, and may leave through the outlet provided in the elongate catheter body outside the catheter body part defined by the balloons.

Moreover, the above-described positive effects provided by the additional outlet provided at the distal end of the elongate catheter body in combination with the above-described positive effects provided by the inlet and the outlet provided both in the elongate catheter body allow the catheter according to the first aspect of the present invention to be inserted into the lumen of the mammal hollow organ for a long time.

Furthermore, the hermetical isolation of the main channel from the functional channel and the inflation channel in the catheter according to the first aspect of the present invention provides a further technical effect which is in that the liquid supplied to the main channel and/or the mucus and other biological contents entering the inlet of the main channel are prevented from entering the functional channel and the inflation channel, and vice versa. Thus, a biological liquid medium or biological gaseous medium taken via the functional opening of the functional channel is prevented from mixing with said liquid supplied to the main channel and/or with the mucus and other biological contents entering the inlet of the main channel that would otherwise negatively influence the reliability or representativity of laboratory-instrumental analysis results obtained for the biological liquid medium or biological gaseous medium. Also, the liquid medium or inflation medium used to inflate the balloons is prevented from mixing with said liquid supplied to the main channel and/or with the mucus and other biological contents entering the inlet of the main channel that would otherwise negatively influence the inflation degree of the balloons and, therefore, impair the isolation of the mammal hollow organ.

In some embodiments of the present invention according to the first aspect, the catheter according to the first aspect of the present invention further comprises a medium supply channel extending in the elongate catheter body, wherein the medium supply channel is hermetically isolated from the functional channel and the inflation channel, and wherein the medium supply channel is designed to supply a gaseous medium, a liquid medium or a fluid medium thereinto and comprises an end-face opening provided at a distal end of the elongate catheter body, the distal end being used for inserting the elongate catheter body into the lumen of the mammal hollow organ.

A further technical effect provided by the medium supply channel of the elongate catheter body is the provision of liquid medical products or the provision of an enteral nutrition mixture, the enteral nutrition mixture providing a mammal organism with basic nutrients, energy, vitamins, macronutrients, micronutrients and/or etc., to an interior of the mammal hollow organ (in particular, to a distal section of the mammal hollow organ) via the end-face opening while sampling a biological liquid medium or biological gaseous medium from the isolated interior of the mammal hollow organ via the functional opening of the functional channel.

Furthermore, the medium supply channel of the elongate catheter body in the catheter according to the first aspect of the present invention and the end-face opening provided in the medium supply channel allow the catheter according to the first aspect of the present invention to be inserted into the lumen of the mammal hollow organ for a long time.

Also, the hermetical isolation of the medium supply channel from the functional channel and the inflation channel in the catheter according to the first aspect of the present invention provides a further technical effect which is in that the medical products (e.g. the enteral or parenteral nutrition mixture) supplied to the medium supply channel are prevented from entering any of the functional channel and the inflation channel, and vice versa. Thus, the biological liquid medium or the biological gaseous medium taken via the functional opening of the functional channel are prevented from mixing with the medical products (e.g. the enteral or parenteral nutrition mixture) supplied to the medium supply channel that would otherwise negatively influence the reliability or representativity of laboratory-instrumental analysis results obtained for the biological liquid medium or biological gaseous medium. Also, the liquid medium or inflation medium used to inflate the balloons is prevented from mixing with the medical products (e.g. the enteral or parenteral nutrition mixture) supplied to the medium supply channel that would otherwise negatively influence the inflation degree of the balloons and, therefore, impair the isolation of the mammal hollow organ.

In various embodiments of the present invention according to the first aspect, the catheter according to the first aspect of the present invention further comprises a shut-off valve installed in a proximal terminus of the medium supply channel.

The shut-off valve installed in a proximal terminus of the medium supply channel blocks the leakage or the backflow of the biological contents (e.g. bile) and/or the medical products (e.g. the enteral or parenteral nutrition mixture) previously supplied to the interior of the mammal hollow organ (particularly, the distal section of the mammal hollow organ) entering the medium supply channel via the end-face opening of the medium supply channel under an excessively increased pressure (e.g. an excessively increased intra-abdominal pressure), in particular from the medium supply channel in the direction to a proximal opening of the medium supply channel, the proximal opening being opposite to the end-face opening of the medium supply channel, i.e. prevents the leakage or the backflow of the biological contents and/or the medical products available in the medium supply channel that would otherwise be sprayed to the user via the proximal opening of the medium supply channel.

In other embodiments of the present invention according to the first aspect, the catheter according to the first aspect of the present invention further comprises a medium drainage channel extending in the elongate catheter body, wherein the medium drainage channel is provided at opposite ends thereof with an inlet and an outlet provided both in the elongate catheter body outside the catheter body part defined by the two balloons, and wherein the medium drainage channel is hermetically isolated from the functional channel, the inflation channel and the medium supply channel. The inlet and the outlet of the medium drainage channel provide one more technical effect which is prevented or excluded formation of congestive and/or inflammatory processes which would occur in the mammal hollow organ due to the accumulation of mucus and other biological contents being specific to the mammal hollow organ outside the catheter body part defined by the balloons. In particular, the formation of congestive and/or inflammatory processes in the mammal hollow organ is prevented or excluded due to the fact that mucus and other biological contents being specific to the mammal hollow organ, when accumulated in the mammal hollow organ, may enter the inlet provided in the elongate catheter body outside the catheter body part defined by the balloons, and may leave through the outlet provided in the elongate catheter body outside the catheter body part defined by the balloons. Furthermore, both the inlet and the outlet of the medium drainage channel of the elongate catheter body allow the catheter according to the first aspect of the present invention to be inserted into the lumen of the mammal hollow organ for a long time.

Also, the hermetical isolation of the medium drainage channel from the functional channel, the inflation channel and the medium supply channel in the catheter according to the first aspect of the present invention provides a further technical effect which is in that biological contents (e.g. a mucus and other biological contents) transferred through the medium drainage channel via the inlet and the outlet are prevented from entering any of the functional channel, the inflation channel and the medium supply channel, and vice versa. Thus, the biological liquid medium or the biological gaseous medium taken via the functional opening of the functional channel are prevented from mixing with the biological contents transferred through the medium drainage channel via the inlet and the outlet that would otherwise negatively influence the reliability or representativity of laboratory-instrumental analysis results obtained for the biological liquid medium or biological gaseous medium. Also, the liquid medium or inflation medium used to inflate the balloons is prevented from mixing with the biological contents transferred through the medium drainage channel via the inlet and the outlet that would otherwise negatively influence the inflation degree of the balloons and, therefore, impair the isolation of the mammal hollow organ. Also, the medical products (e.g. the enteral or parenteral nutrition mixture) supplied to the medium supply channel are prevented from mixing with the biological contents transferred through the medium drainage channel via the inlet and the outlet that would otherwise negatively influence the reliability or safety of the catheter.

In some other embodiments of the present invention according to the first aspect, the medium drainage channel provided in the elongate catheter body of the catheter according to the first aspect further comprises an end-face opening provided at the distal end of the elongate catheter body, wherein the end-face opening of the medium drainage channel is in fluid communication with the inlet of the medium drainage channel and with the outlet of the medium drainage channel.

The end-face opening of the medium drainage channel in combination with the inlet and the outlet of the medium drainage channel allows a flow comprised of biological liquid media, biological gaseous media or biological fluid media (i.e. any combination of a biological liquid media and a biological gaseous media), the flow being specific to the mammal hollow organ (i.e. a flow of biological contents being specific to the mammal hollow organ), to be passed bidirectionally through the medium drainage channel under a natural pressure produced by the mammal hollow organ, i.e. passed through the medium drainage channel from the proximal section of the mammal hollow organ to the distal section of the mammal hollow organ or vice versa so that the mammal hollow organ may not be blocked or occluded by excessively accumulated biological liquid media, biological gaseous media or biological fluid media (i.e. excessively accumulated biological contents being specific to the mammal hollow organ), thereby preventing the mammal hollow organ from being damaged (for example, due to an initiated clotting and thrombosis conditioned by the accumulated biological liquid media, biological gaseous media or biological fluid media).

In different embodiments of the present invention according to the first aspect, the catheter further comprises a shut-off valve installed in a proximal terminus of the medium drainage channel extending in the elongate catheter body. The shut-off valve installed in a proximal terminus of the medium drainage channel blocks the leakage or the backflow of the biological contents (e.g. bile) available in the medium drainage channel under an excessively increased pressure (e.g. an excessively increased intra-abdominal pressure) from the medium drainage channel in the direction to a proximal opening of the elongate catheter body, i.e. prevents the backflow of the biological contents available in the medium drainage channel that would otherwise be sprayed to the user via the proximal opening of the elongate catheter body.

In various embodiments of the present invention according to the first aspect, the elongate catheter body is further provided with a loop or a thread designed to be gripped with an endoscope for guiding the gripped catheter body within the mammal hollow organ. The loop or thread provided on the elongate catheter body simplifies the advancement or the movement of the gripped catheter body within the mammal hollow organ to a desired placement (target) site in the lumen of the mammal hollow organ.

In different embodiments of the present invention according to the first aspect, the elongate catheter body is further provided with a net or enclosure being permeable to a liquid medium and/or a gaseous medium, wherein the net or enclosure at least partly encloses a catheter body part defined by the balloons so as to cover the functional opening, and wherein the net or enclosure is attached to the balloons or encloses them such that the net or enclosure becomes strained when the balloons are inflated.

Use of the net or enclosure being permeable to a liquid medium and/or gaseous medium for covering the functional opening in the catheter according to the first aspect of the present invention provides an additional technical effect which is improved safety and increased reliability of the catheter, in particular due to prevented suction of the hollow organ tissue into the functional opening. The attachment of the net or enclosure to the two balloons, the net or enclosure becoming strained between the balloons when inflating the balloons in the catheter according to the first aspect of the present invention, contributes to the above additional technical effect which is the improved safety and increased reliability of the catheter, in particular due to prevented suction of the tissue of the hollow organ into the functional opening of the catheter. The enclosure of the two balloons with the net or enclosure, the net or enclosure becoming strained between the balloons when inflating the balloons in the catheter according to the first aspect of the present invention, also contributes to the above additional technical effect which is the improved safety and increased reliability of the catheter, in particular due to prevented suction of the tissue of the hollow organ into the functional opening of the catheter.

To achieve the objective of the present invention, as embodied and broadly described herein, in a second aspect of the present invention, there is provided a catheter for isolating an interior of a mammal hollow organ, the catheter comprising: (i) an elongate catheter body designed to be inserted into a lumen of the mammal hollow organ; (ii) two balloons, individually and separately disposed along the elongate catheter body and designed to be inflated to isolate the interior of the mammal hollow organ therebetween; and (iii) a functional channel extending in the elongate catheter body and comprising a functional opening provided in the elongate catheter body between the balloons, wherein the functional channel is designed to allow a negative pressure to be produced in the isolated interior to take a gaseous medium, a liquid medium or a fluid medium therefrom via the functional opening, and wherein two spiral-shaped projections divided by a gap into two pairs of curved sections are further provided on a catheter body part defined by the two balloons such that the gap comprises the functional opening, and the two pairs of curved sections enclose the functional opening, wherein the two spiral-shaped projections are designed such that both the spiral-shaped projections allow a distance between a mucosal tissue of the mammal hollow organ, the mucosal tissue being between the inflated balloons, and the functional opening.

The catheter according to the second aspect of the present invention also provides the above-mentioned main technical effect which is the improved safety of the catheter due to prevented suction of a hollow organ tissue of the mammal hollow organ (e.g. a mucosal tissue of the mammal hollow organ) into the functional opening of the catheter body that would otherwise cause damage to the hollow organ tissue. In particular, in the present invention according to the second aspect of the present invention, suction of the hollow organ tissue into the functional opening is further prevented due to the fact that the functional opening provided in the elongate catheter body is located in the gap between the two pairs of curved sections that enclose the functional opening. In other words, in the present invention according to the second aspect, the two pairs of curved sections that enclose the functional opening do not allow the hollow organ tissue to contact the functional opening directly or approach to the functional opening for a distance appropriate for suction of the hollow organ tissue into the functional opening when the negative pressure is produced in the isolated interior between the inflated balloons.

The catheter according to the second aspect of the present invention also provides the above-mentioned further technical effect which is the increased reliability of the catheter due to prevented partial or complete blockage or occlusion of the functional opening that may be caused by suction of a tissue of the mammal hollow organ into the functional opening (i.e. suction of the hollow organ tissue into the functional opening is further prevented due to the fact that the two pairs of curved sections enclose the functional opening provided in the elongate catheter body, and the functional opening is located in the gap between the two pairs of curved sections), thereby preventing the inserted catheter from being retrieved from the mammal hollow organ and, then, changing the retrieved catheter to a new catheter or removing the hollow organ tissue sucked into the functional opening from the functional opening of the retrieved catheter.

The catheter according to the second aspect of the present invention also provides the above-mentioned additional technical effect which is the increased efficiency of the catheter due to an improved communication between different areas of the isolated interior of the mammal hollow organ that is provided by the two spiral-shaped projections provided on the catheter body part defined by the two balloons. In particular, both the spiral-shaped projections allow the gaseous medium, the liquid medium or the fluid medium from all the isolated interior of the mammal hollow organ to be taken via the functional opening when producing the negative pressure in the isolated interior of the mammal hollow organ, especially when the mucosal tissue of the mammal hollow organ is closely glued or adhered to both the spiral-shaped projections, thereby positively influencing the reliability or the representativity of laboratory-instrumental analysis results obtained for the taken medium.

In an embodiment of the present invention according to the second aspect, the two spiral-shaped projections are further provided with two additional gaps, wherein the two additional gaps are spaced from the gap such that the gap is positioned between the two additional gaps. The two additional gaps spaced from the gap positioned therebetween further contribute to the above additional technical effect, i.e. further increase the efficiency of the catheter since they further improve the communication between different areas of the isolated interior of the mammal hollow organ, thereby further allowing the gaseous medium, the liquid medium or the fluid medium from all the isolated interior of the mammal hollow organ to be taken via the functional opening when producing the negative pressure in the isolated interior of the mammal hollow organ.

In another embodiment of the present invention according to the second aspect, the gap and the two additional gaps as provided on the catheter body part defined by the two balloons are further circumferentially displaced in relation to each other or aligned with each other along the catheter body part defined by the two balloons. Displacement of the gap and the two additional gaps in relation to each other on the circumference of the catheter body part defined by the two balloons or alignment of the gap and the two additional gaps in relation to each other along the catheter body part defined by the two balloons further contributes to the above additional technical effect, i.e. further increases the efficiency of the catheter since the displacement of the gap and the two additional gaps further improves the communication between different areas of the isolated interior of the mammal hollow organ, thereby further allowing the gaseous medium, the liquid medium or the fluid medium from all the isolated interior of the mammal hollow organ to be taken via the functional opening when producing the negative pressure in the isolated interior of the mammal hollow organ.

In still another embodiment of the present invention according to the second aspect, a spiral-shaped channel is further defined between the two spiral-shaped projections, and the functional opening is within the spiral-shaped channel. The spiral-shaped channel further defined between the two spiral-shaped projections and comprising the functional opening provided in the gap between the two pairs of curved sections further contributes to the above additional technical effect, i.e. further increases the efficiency of the catheter since the spiral-shaped channel further improves the communication between different areas of the isolated interior of the mammal hollow organ, thereby further allowing the gaseous medium, the liquid medium or the fluid medium from all the isolated interior of the mammal hollow organ to be taken via the functional opening when producing the negative pressure in the isolated interior of the mammal hollow organ.

In one of embodiments of the present invention according to the second aspect, the catheter may further comprise an inflation channel extending in the elongate catheter body to deliver a liquid or gaseous medium to the balloons to provide inflation thereof, wherein the functional channel and the inflation channel are hermetically isolated from each other. Delivery of the liquid or gaseous medium to the balloons through the inflation channel for inflating the balloons provides another technical effect which is a combination of simplified maintenance of balloon sizes as required to isolate the interior of the mammal hollow organ and simplified regulation of sizes or inflation degrees of the balloons. Moreover, the hermetically isolated functional and inflation channels in the catheter according to the second aspect of the present invention provide a further technical effect which is in that the liquid or gaseous medium used to inflate the balloons is prevented from entering the functional channel and, therefore, the gaseous medium, the liquid medium or the fluid medium (in particular, biological contents) taken via the functional opening is prevented from mixing with said inflation medium that would otherwise negatively influence the reliability or representativity of laboratory-instrumental analysis results obtained for the biological liquid medium or biological gaseous medium.

In another embodiment of the present invention according to the second aspect, the catheter comprising the inflation channel may further comprise a main channel extending in the elongate catheter body, wherein the main channel is provided at opposite ends thereof with an inlet and an outlet provided both in the elongate catheter body outside the catheter body part defined by the two balloons, and wherein the main channel is hermetically isolated from the functional channel and the inflation channel, and wherein the main channel is further designed to supply a gaseous medium, a liquid medium or a fluid medium thereinto and further comprises an additional outlet provided at a distal end of the elongate catheter body, the distal end being used for inserting the elongate catheter body into the lumen of the mammal hollow organ.

A further technical effect provided by the main channel of the elongate catheter body is the provision of liquid medical products or the provision of an enteral nutrition mixture, the enteral nutrition mixture providing a mammal organism with basic nutrients, energy, vitamins, macronutrients, micronutrients and/or etc., to an interior of the mammal hollow organ (particularly, to a distal section of the mammal hollow organ) via the additional outlet while sampling a biological liquid medium or biological gaseous medium from the isolated interior of the mammal hollow organ via the functional opening of the functional channel.

Another technical effect provided by the main channel of the elongate catheter body is prevented or excluded formation of congestive and/or inflammatory processes which would occur in the mammal hollow organ due to the accumulation of mucus and other biological contents being specific to the mammal hollow organ outside the catheter body part defined by the balloons. In particular, the formation of congestive and/or inflammatory processes in the mammal hollow organ is prevented or excluded due to the fact that mucus and other biological contents being specific to the mammal hollow organ, when accumulated in the mammal hollow organ, may enter the inlet provided in the elongate catheter body outside the catheter body part defined by the balloons, and may leave through the outlet provided in the elongate catheter body outside the catheter body part defined by the balloons.

Moreover, the above-described positive effects provided by the additional outlet provided at the distal end of the elongate catheter body in combination with the above-described positive effects provided by the inlet and the outlet provided both in the elongate catheter body allow the catheter according to the second aspect of the present invention to be inserted into the lumen of the mammal hollow organ for a long time.

Furthermore, the hermetical isolation of the main channel from the functional channel and the inflation channel in the catheter according to the second aspect of the present invention provides a further technical effect which is in that the liquid supplied to the main channel and/or the mucus and other biological contents entering the inlet of the main channel are prevented from entering the functional channel and the inflation channel, and vice versa. Thus, a biological liquid medium or biological gaseous medium taken via the functional opening of the functional channel is prevented from mixing with said liquid supplied to the main channel and/or with the mucus and other biological contents entering the inlet of the main channel that would otherwise negatively influence the reliability or representativity of laboratory-instrumental analysis results obtained for the biological liquid medium or biological gaseous medium. Also, the liquid medium or inflation medium used to inflate the balloons is prevented from mixing with said liquid supplied to the main channel and/or with the mucus and other biological contents entering the inlet of the main channel that would otherwise negatively influence the inflation degree of the balloons and, therefore, impair the isolation of the mammal hollow organ.

In some embodiments of the present invention according to the second aspect, the catheter according to the second aspect of the present invention further comprises a medium supply channel extending in the elongate catheter body, wherein the medium supply channel is hermetically isolated from the functional channel and the inflation channel, and wherein the medium supply channel is designed to supply a gaseous medium, a liquid medium or a fluid medium thereinto and comprises an end-face opening provided at a distal end of the elongate catheter body, the distal end being used for inserting the elongate catheter body into the lumen of the mammal hollow organ.

A further technical effect provided by the medium supply channel of the elongate catheter body is the provision of liquid medical products or the provision of an enteral nutrition mixture, the enteral nutrition mixture providing a mammal organism with basic nutrients, energy, vitamins, macronutrients, micronutrients and/or etc., to an interior of the mammal hollow organ (in particular, to a distal section of the mammal hollow organ) via the end-face opening while sampling a biological liquid medium or biological gaseous medium from the isolated interior of the mammal hollow organ via the functional opening of the functional channel.

Furthermore, the medium supply channel of the elongate catheter body in the catheter according to the second aspect of the present invention and the end-face opening provided in the medium supply channel allow the catheter according to the second aspect of the present invention to be inserted into the lumen of the mammal hollow organ for a long time.

Also, the hermetical isolation of the medium supply channel from the functional channel and the inflation channel in the catheter according to the second aspect of the present invention provides a further technical effect which is in that the medical products (e.g. the enteral or parenteral nutrition mixture) supplied to the medium supply channel are prevented from entering any of the functional channel and the inflation channel, and vice versa. Thus, the biological liquid medium or the biological gaseous medium taken via the functional opening of the functional channel are prevented from mixing with the medical products (e.g. the enteral or parenteral nutrition mixture) supplied to the medium supply channel that would otherwise negatively influence the reliability or representativity of laboratory-instrumental analysis results obtained for the biological liquid medium or biological gaseous medium. Also, the liquid medium or inflation medium used to inflate the balloons is prevented from mixing with the medical products (e.g. the enteral or parenteral nutrition mixture) supplied to the medium supply channel that would otherwise negatively influence the inflation degree of the balloons and, therefore, impair the isolation of the mammal hollow organ.

In various embodiments of the present invention according to the second aspect, the catheter according to the second aspect of the present invention further comprises a shut-off valve installed in a proximal terminus of the medium supply channel.

The shut-off valve installed in a proximal terminus of the medium supply channel blocks the leakage or the backflow of the biological contents (e.g. bile) and/or the medical products (e.g. the enteral or parenteral nutrition mixture) previously supplied to the interior of the mammal hollow organ (particularly, the distal section of the mammal hollow organ) entering the medium supply channel via the end-face opening of the medium supply channel under an excessively increased pressure (e.g. an excessively increased intra-abdominal pressure), in particular from the medium supply channel in the direction to a proximal opening of the medium supply channel, the proximal opening being opposite to the end-face opening of the medium supply channel, i.e. prevents the leakage or the backflow of the biological contents and/or the medical products available in the medium supply channel that would otherwise be sprayed to the user via the proximal opening of the medium supply channel.

In other embodiments of the present invention according to the second aspect, the catheter according to the second aspect of the present invention further comprises a medium drainage channel extending in the elongate catheter body, wherein the medium drainage channel is provided at opposite ends thereof with an inlet and an outlet provided both in the elongate catheter body outside the catheter body part defined by the two balloons, and wherein the medium drainage channel is hermetically isolated from the functional channel, the inflation channel and the medium supply channel. The inlet and the outlet of the medium drainage channel provide one more technical effect which is prevented or excluded formation of congestive and/or inflammatory processes which would occur in the mammal hollow organ due to the accumulation of mucus and other biological contents being specific to the mammal hollow organ outside the catheter body part defined by the balloons. In particular, the formation of congestive and/or inflammatory processes in the mammal hollow organ is prevented or excluded due to the fact that mucus and other biological contents being specific to the mammal hollow organ, when accumulated in the mammal hollow organ, may enter the inlet provided in the elongate catheter body outside the catheter body part defined by the balloons, and may leave through the outlet provided in the el ongate catheter body outside the catheter body part defined by the balloons. Furthermore, both the inlet and the outlet of the medium drainage channel of the elongate catheter body allow the catheter according to the second aspect of the present invention to be inserted into the lumen of the mammal hollow organ for a long time.

Also, the hermetical isolation of the medium drainage channel from the functional channel, the inflation channel and the medium supply channel in the catheter according to the second aspect of the present invention provides a further technical effect which is in that biological contents (e.g. a mucus and other biological contents) transferred through the medium drainage channel via the inlet and the outlet are prevented from entering any of the functional channel, the inflation channel and the medium supply channel, and vice versa. Thus, the biological liquid medium or the biological gaseous medium taken via the functional opening of the functional channel are prevented from mixing with the biological contents transferred through the medium drainage channel via the inlet and the outlet that would otherwise negatively influence the reliability or representativity of laboratory-instrumental analysis results obtained for the biological liquid medium or biological gaseous medium. Also, the liquid medium or inflation medium used to inflate the balloons is prevented from mixing with the biological contents transferred through the medium drainage channel via the inlet and the outlet that would otherwise negatively influence the inflation degree of the balloons and, therefore, impair the isolation of the mammal hollow organ. Also, the medical products (e.g. the enteral or parenteral nutrition mixture) supplied to the medium supply channel are prevented from mixing with the biological contents transferred through the medium drainage channel via the inlet and the outlet that would otherwise negatively influence the reliability or safety of the catheter.

In some other embodiments of the present invention according to the second aspect, the medium drainage channel provided in the elongate catheter body of the catheter according to the second aspect further comprises an end-face opening provided at the distal end of the elongate catheter body, wherein the end-face opening of the medium drainage channel is in fluid communication with the inlet of the medium drainage channel and with the outlet of the medium drainage channel.

The end-face opening of the medium drainage channel in combination with the inlet and the outlet of the medium drainage channel allows a flow comprised of biological liquid media, biological gaseous media or biological fluid media (i.e. any combination of a biological liquid media and a biological gaseous media), the flow being specific to the mammal hollow organ (i.e. a flow of biological contents being specific to the mammal hollow organ), to be passed bidirectionally through the medium drainage channel under a natural pressure produced by the mammal hollow organ, i.e. passed through the medium drainage channel from the proximal section of the mammal hollow organ to the distal section of the mammal hollow organ or vice versa so that the mammal hollow organ may not be blocked or occluded by excessively accumulated biological liquid media, biological gaseous media or biological fluid media (i.e. excessively accumulated biological contents being specific to the mammal hollow organ), thereby preventing the mammal hollow organ from being damaged (for example, due to an initiated clotting and thrombosis conditioned by the accumulated biological liquid media, biological gaseous media or biological fluid media).

In different embodiments of the present invention according to the second aspect, the catheter further comprises a shut-off valve installed in a proximal terminus of the medium drainage channel extending in the elongate catheter body. The shut-off valve installed in a proximal terminus of the medium drainage channel blocks the leakage or the backflow of the biological contents (e.g. bile) available in the medium drainage channel under an excessively increased pressure (e.g. an excessively increased intra-abdominal pressure) from the medium drainage channel in the direction to a proximal opening of the elongate catheter body, i.e. prevents the backflow of the biological contents available in the medium drainage channel that would otherwise be sprayed to the user via the proximal opening of the elongate catheter body.

In various embodiments of the present invention according to the second aspect, the elongate catheter body is further provided with a loop or a thread designed to be gripped with an endoscope for guiding the gripped catheter body within the mammal hollow organ. The loop or thread provided on the elongate catheter body simplifies the advancement or the movement of the gripped catheter body within the mammal hollow organ to a desired placement (target) site in the lumen of the mammal hollow organ.

In different embodiments of the present invention according to the second aspect, the elongate catheter body is further provided with a net or enclosure being permeable to a liquid medium and/or a gaseous medium, wherein the net or enclosure at least partly encloses a catheter body part defined by the balloons so as to cover the functional opening, and wherein the net or enclosure is attached to the balloons or encloses them such that the net or enclosure becomes strained when the balloons are inflated.

Use of the net or enclosure being permeable to a liquid medium and/or gaseous medium for covering the functional opening in the catheter according to the second aspect of the present invention provides an additional technical effect which is improved safety and increased reliability of the catheter, in particular due to prevented suction of the hollow organ tissue into the functional opening. The attachment of the net or enclosure to the two balloons, the net or enclosure becoming strained between the balloons when inflating the balloons in the catheter according to the second aspect of the present invention, contributes to the above additional technical effect which is the improved safety and increased reliability of the catheter, in particular due to prevented suction of the tissue of the hollow organ into the functional opening of the catheter. The enclosure of the two balloons with the net or enclosure, the net or enclosure becoming strained between the balloons when inflating the balloons in the catheter according to the second aspect of the present invention, also contributes to the above additional technical effect which is the improved safety and increased reliability of the catheter, in particular due to prevented suction of the tissue of the hollow organ into the functional opening of the catheter.

To achieve the objective of the present invention, as embodied and broadly described herein, in a third aspect of the present invention, there is provided a catheter for isolating an interior of a mammal hollow organ, the catheter comprising: (i) an elongate catheter body designed to be inserted into a lumen of the mammal hollow organ; (ii) two balloons, individually and separately disposed along the elongate catheter body and designed to be inflated to isolate the interior of the mammal hollow organ therebetween; and (iii) a functional channel extending in the elongate catheter body and comprising a functional opening provided in the elongate catheter body between the balloons, wherein the functional channel is designed to allow a negative pressure to be produced in the isolated interior to take a gaseous medium, a liquid medium or a fluid medium therefrom via the functional opening, and wherein two ring-shaped projections enclosing the functional opening are further provided on the elongate catheter body between the balloons such that the functional opening is between the two ring-shaped projections, and wherein the two ring-shaped projections are designed such that they allow a distance between a mucosal tissue of the mammal hollow organ, the mucosal tissue being between the inflated balloons, and the functional opening, and wherein each of the two ring-shaped projections is provided with a traverse groove or cut, and wherein the traverse groove or cut is designed such that the traverse groove or cut allows the gaseous medium, the liquid medium or the fluid medium to be taken via the functional opening to pass therethrough.

The catheter according to the third aspect of the present invention also provides the above-mentioned main technical effect, in particular improves safety of the catheter due to prevented suction of a tissue of the mammal hollow organ into the functional opening of the catheter body that would otherwise cause damage to the hollow organ tissue. In particular, in the present invention according to the third aspect of the present invention, suction of the hollow organ tissue into the functional opening is further prevented due to the fact that the functional opening provided in the elongate catheter body is between the ring-shaped projections enclosing the functional opening. In other words, in the present invention according to the third aspect, the ring-shaped projections enclosing the functional opening do not allow the hollow organ tissue to contact the functional opening directly or approach to the functional opening for a distance appropriate for suction of the hollow organ tissue into the functional opening when the negative pressure is produced in the isolated interior between the inflated balloons.

The catheter according to the third aspect of the present invention provides the further technical effect mentioned above for the catheter according to the first aspect of the present invention, in particular increases reliability of the catheter due to prevented partial or complete blockage or occlusion of the functional opening that may be caused by suction of a tissue of the mammal hollow organ into the functional opening (i.e. suction of the hollow organ tissue into the functional opening is further prevented due to the fact that the ring-shaped projections enclose the functional opening provided in the elongate catheter body, and the functional opening is between the ring-shaped projections), thereby preventing the inserted catheter from being retrieved from the mammal hollow organ and, then, changing the retrieved catheter to a new catheter or removing the hollow organ tissue sucked into the functional opening from the functional opening of the retrieved catheter.

Furthermore, the catheter according to the third aspect of the present invention provides a first new technical effect which is extended or increased volume of the isolated interior of the mammal hollow organ, the isolated interior being used for producing the negative pressure therein. In particular, the extended or increased volume of the isolated interior of the mammal hollow organ is provided by the catheter according to the third aspect of the present invention due to the fact that the projections enclosing the functional opening are ring-shaped. The extended or increased volume of the isolated interior of the mammal hollow organ allows a controlled negative pressure to be produced in the isolated interior, the negative pressure having a level required to provide the suction of a liquid or gaseous medium from the isolated interior and being specific to the mammal hollow organ having different physiological features, thereby preventing an excessive negative pressure from being produced in the isolated interior that would otherwise result in suction of a tissue of the mammal hollow organ into the functional opening and, therefore, would result in damage to the hollow organ tissue. Besides, the controlled negative pressure having a level required to provide the suction of the liquid or gaseous medium from the isolated interior and being specific to the mammal hollow organ further contributes to the above mentioned further technical effect, namely increased reliability of the catheter.

Furthermore, the catheter according to the third aspect of the present invention provides a second new technical effect which is improved utility of the catheter. The second new technical effect is further provided by the catheter according to the third aspect of the present invention due to the fact that the projections enclosing the functional opening positioned therebetween are provided between the balloons, and the enclosing projections are ring-shaped. In particular, the improved utility of the catheter is provided due to the fact that possibilities of production of the controlled negative pressure in the isolated interior of the mammal hollow organ, the negative pressure having a level required to provide the suction, are essentially expanded, wherein such expanded possibilities are conditioned by an increased volume of the isolated interior of the mammal hollow organ, the isolated interior being used for producing the controlled negative pressure therein.

Furthermore, the catheter according to the third aspect of the present invention provides a third new technical effect which is the appropriate centering of the functional opening in relation to a target zone of the mammal hollow organ, the target zone being used for sucking a liquid or gaseous medium therefrom. In particular, the third new technical effect is further provided by the catheter according to the third aspect of the present invention due to the fact that the projections enclosing the functional opening positioned therebetween are provided between the balloons, and the enclosing projections are ring-shaped.

Traverse grooves or cuts provided in the two ring-shaped projections also provide the above-mentioned additional technical effect which is the increased efficiency of the catheter due to an improved communication between different areas of the isolated interior of the mammal hollow organ that is provided by said traverse grooves or cuts. In particular, the traverse grooves or cuts provided in the two ring-shaped projections allow the gaseous medium, the liquid medium or the fluid medium from all the isolated interior of the mammal hollow organ to be taken via the functional opening when producing the negative pressure in the isolated interior of the mammal hollow organ, especially when the mucosal tissue of the mammal hollow organ is closely glued or adhered to the spiral-shaped projection, thereby positively influencing the reliability or the representativity of laboratory-instrumental analysis results obtained for the taken medium.

In an embodiment of the present invention according to the third aspect, each of the two ring-shaped projections is further provided with one or more though-holes, each being designed to allow the gaseous medium, the liquid medium or the fluid medium to be taken via the functional opening to pass therethrough. The though-holes further provided in the ring-shaped projections further contribute to the above additional technical effect, i.e. further increase the efficiency of the catheter since they further improve the communication between different areas of the isolated interior of the mammal hollow organ, thereby further allowing the gaseous medium, the liquid medium or the fluid medium from all the isolated interior of the mammal hollow organ to be taken via the functional opening when producing the negative pressure in the isolated interior of the mammal hollow organ.

In one of embodiments of the present invention according to the third aspect, the catheter may further comprise an inflation channel extending in the elongate catheter body to deliver a liquid or gaseous medium to the balloons to provide inflation thereof, wherein the functional channel and the inflation channel are hermetically isolated from each other. Delivery of the liquid or gaseous medium to the balloons through the inflation channel for inflating the balloons provides another technical effect which is a combination of simplified maintenance of balloon sizes as required to isolate the interior of the mammal hollow organ and simplified regulation of sizes or inflation degrees of the balloons. Moreover, the hermetically isolated functional and inflation channels in the catheter according to the third aspect of the present invention provide a further technical effect which is in that the liquid or gaseous medium used to inflate the balloons is prevented from entering the functional channel and, therefore, the gaseous medium, the liquid medium or the fluid medium (in particular, biological contents) taken via the functional opening is prevented from mixing with said inflation medium that would otherwise negatively influence the reliability or representativity of laboratory-instrumental analysis results obtained for the biological liquid medium or biological gaseous medium.

In another embodiment of the present invention according to the third aspect, the catheter comprising the inflation channel may further comprise a main channel extending in the elongate catheter body, wherein the main channel is provided at opposite ends thereof with an inlet and an outlet provided both in the elongate catheter body outside the catheter body part defined by the two balloons, and wherein the main channel is hermetically isolated from the functional channel and the inflation channel, and wherein the main channel is further designed to supply a gaseous medium, a liquid medium or a fluid medium thereinto and further comprises an additional outlet provided at a distal end of the elongate catheter body, the distal end being used for inserting the elongate catheter body into the lumen of the mammal hollow organ.

A further technical effect provided by the main channel of the elongate catheter body is the provision of liquid medical products or the provision of an enteral nutrition mixture, the enteral nutrition mixture providing a mammal organism with basic nutrients, energy, vitamins, macronutrients, micronutrients and/or etc., to an interior of the mammal hollow organ (particularly, to a distal section of the mammal hollow organ) via the additional outlet while sampling a biological liquid medium or biological gaseous medium from the isolated interior of the mammal hollow organ via the functional opening of the functional channel.

Another technical effect provided by the main channel of the elongate catheter body is prevented or excluded formation of congestive and/or inflammatory processes which would occur in the mammal hollow organ due to the accumulation of mucus and other biological contents being specific to the mammal hollow organ outside the catheter body part defined by the balloons. In particular, the formation of congestive and/or inflammatory processes in the mammal hollow organ is prevented or excluded due to the fact that mucus and other biological contents being specific to the mammal hollow organ, when accumulated in the mammal hollow organ, may enter the inlet provided in the elongate catheter body outside the catheter body part defined by the balloons, and may leave through the outlet provided in the elongate catheter body outside the catheter body part defined by the balloons.

Moreover, the above-described positive effects provided by the additional outlet provided at the distal end of the elongate catheter body in combination with the above-described positive effects provided by the inlet and the outlet provided both in the elongate catheter body allow the catheter according to the third aspect of the present invention to be inserted into the lumen of the mammal hollow organ for a long time.

Furthermore, the hermetical isolation of the main channel from the functional channel and the inflation channel in the catheter according to the third aspect of the present invention provides a further technical effect which is in that the liquid supplied to the main channel and/or the mucus and other biological contents entering the inlet of the main channel are prevented from entering the functional channel and the inflation channel, and vice versa. Thus, a biological liquid medium or biological gaseous medium taken via the functional opening of the functional channel is prevented from mixing with said liquid supplied to the main channel and/or with the mucus and other biological contents entering the inlet of the main channel that would otherwise negatively influence the reliability or representativity of laboratory-instrumental analysis results obtained for the biological liquid medium or biological gaseous medium. Also, the liquid medium or inflation medium used to inflate the balloons is prevented from mixing with said liquid supplied to the main channel and/or with the mucus and other biological contents entering the inlet of the main channel that would otherwise negatively influence the inflation degree of the balloons and, therefore, impair the isolation of the mammal hollow organ.

In various embodiments of the present invention, at least one or each of the inlet and the outlet of the main channel provided in the elongate catheter body is a cone-shaped hole tapering towards an interior of the main channel.

In the catheter according to the third aspect of the present invention, the cone-shaped inlet of the main channel and/or the cone-shaped outlet of the main channel in the catheter according to the third aspect of the present invention further eliminate or reduce the leakage of the gaseous medium, the liquid medium or the fluid medium (e.g. medical products such as the enteral or parenteral nutrition mixture) supplied to the main channel from the inside of the main channel into the distal and proximal sections of the mammal hollow organ through the outlet and/or the inlet, while simultaneously improving the drainage of the biological contents being specific to the mammal hollow organ from the distal and proximal sections of the mammal hollow organ into the inside of the main channel through the outlet and/or inlet. Therefore, in such various embodiments of the present invention, conical shapes of the outlet and inlet of the main channel allow the excessive accumulation of the biological contents being specific to the mammal hollow organ to be minimized in the distal section of the mammal hollow organ, the distal section being defined by the inflated balloon positioned closer or closest to the distal end of the elongate catheter body, and/or in the proximal section of the mammal hollow organ, the proximal section being located outside the isolated interior of the mammal hollow organ and being defined by the inflated balloon positioned farther or farthest from the distal end of the elongate catheter body, thereby minimizing the formation of congestive processes and/or inflammatory processes in the distal section of the mammal hollow organ and/or in the proximal section of the mammal hollow organ that could be caused by the accumulated biological contents, respectively.

In some embodiments of the present invention according to the third aspect, the catheter according to the third aspect of the present invention further comprises a medium supply channel extending in the elongate catheter body, wherein the medium supply channel is hermetically isolated from the functional channel and the inflation channel, and wherein the medium supply channel is designed to supply a gaseous medium, a liquid medium or a fluid medium thereinto and comprises an end-face opening provided at a distal end of the elongate catheter body, the distal end being used for inserting the elongate catheter body into the lumen of the mammal hollow organ.

A further technical effect provided by the medium supply channel of the elongate catheter body is the provision of liquid medical products or the provision of an enteral nutrition mixture, the enteral nutrition mixture providing a mammal organism with basic nutrients, energy, vitamins, macronutrients, micronutrients and/or etc., to an interior of the mammal hollow organ (in particular, to a distal section of the mammal hollow organ) via the end-face opening while sampling a biological liquid medium or biological gaseous medium from the isolated interior of the mammal hollow organ via the functional opening of the functional channel.

Furthermore, the medium supply channel of the elongate catheter body in the catheter according to the third aspect of the present invention and the end-face opening provided in the medium supply channel allow the catheter according to the third aspect of the present invention to be inserted into the lumen of the mammal hollow organ for a long time.

Also, the hermetical isolation of the medium supply channel from the functional channel and the inflation channel in the catheter according to the third aspect of the present invention provides a further technical effect which is in that the medical products (e.g. the enteral or parenteral nutrition mixture) supplied to the medium supply channel are prevented from entering any of the functional channel and the inflation channel, and vice versa. Thus, the biological liquid medium or the biological gaseous medium taken via the functional opening of the functional channel are prevented from mixing with the medical products (e.g. the enteral or parenteral nutrition mixture) supplied to the medium supply channel that would otherwise negatively influence the reliability or representativity of laboratory-instrumental analysis results obtained for the biological liquid medium or biological gaseous medium. Also, the liquid medium or inflation medium used to inflate the balloons is prevented from mixing with the medical products (e.g. the enteral or parenteral nutrition mixture) supplied to the medium supply channel that would otherwise negatively influence the inflation degree of the balloons and, therefore, impair the isolation of the mammal hollow organ.

In various embodiments of the present invention according to the third aspect, the catheter according to the third aspect of the present invention further comprises a shut-off valve installed in a proximal terminus of the medium supply channel.

The shut-off valve installed in a proximal terminus of the medium supply channel blocks the leakage or the backflow of the biological contents (e.g. bile) and/or the medical products (e.g. the enteral or parenteral nutrition mixture) previously supplied to the interior of the mammal hollow organ (particularly, the distal section of the mammal hollow organ) entering the medium supply channel via the end-face opening of the medium supply channel under an excessively increased pressure (e.g. an excessively increased intra-abdominal pressure), in particular from the medium supply channel in the direction to a proximal opening of the medium supply channel, the proximal opening being opposite to the end-face opening of the medium supply channel, i.e. prevents the leakage or the backflow of the biological contents and/or the medical products available in the medium supply channel that would otherwise be sprayed to the user via the proximal opening of the medium supply channel.

In other embodiments of the present invention according to the third aspect, the catheter according to the third aspect of the present invention further comprises a medium drainage channel extending in the elongate catheter body, wherein the medium drainage channel is provided at opposite ends thereof with an inlet and an outlet provided both in the elongate catheter body outside the catheter body part defined by the two balloons, and wherein the medium drainage channel is hermetically isolated from the functional channel, the inflation channel and the medium supply channel. The inlet and the outlet of the medium drainage channel provide one more technical effect which is prevented or excluded formation of congestive and/or inflammatory processes which would occur in the mammal hollow organ due to the accumulation of mucus and other biological contents being specific to the mammal hollow organ outside the catheter body part defined by the balloons. In particular, the formation of congestive and/or inflammatory processes in the mammal hollow organ is prevented or excluded due to the fact that mucus and other biological contents being specific to the mammal hollow organ, when accumulated in the mammal hollow organ, may enter the inlet provided in the elongate catheter body outside the catheter body part defined by the balloons, and may leave through the outlet provided in the elongate catheter body outside the catheter body part defined by the balloons. Furthermore, both the inlet and the outlet of the medium drainage channel of the elongate catheter body allow the catheter according to the third aspect of the present invention to be inserted into the lumen of the mammal hollow organ for a long time.

Also, the hermetical isolation of the medium drainage channel from the functional channel, the inflation channel and the medium supply channel in the catheter according to the third aspect of the present invention provides a further technical effect which is in that biological contents (e.g. a mucus and other biological contents) transferred through the medium drainage channel via the inlet and the outlet are prevented from entering any of the functional channel, the inflation channel and the medium supply channel, and vice versa. Thus, the biological liquid medium or the biological gaseous medium taken via the functional opening of the functional channel are prevented from mixing with the biological contents transferred through the medium drainage channel via the inlet and the outlet that would otherwise negatively influence the reliability or representativity of laboratory-instrumental analysis results obtained for the biological liquid medium or biological gaseous medium. Also, the liquid medium or inflation medium used to inflate the balloons is prevented from mixing with the biological contents transferred through the medium drainage channel via the inlet and the outlet that would otherwise negatively influence the inflation degree of the balloons and, therefore, impair the isolation of the mammal hollow organ. Also, the medical products (e.g. the enteral or parenteral nutrition mixture) supplied to the medium supply channel are prevented from mixing with the biological contents transferred through the medium drainage channel via the inlet and the outlet that would otherwise negatively influence the reliability or safety of the catheter.

In some other embodiments of the present invention according to the third aspect, the medium drainage channel provided in the elongate catheter body of the catheter according to the third aspect further comprises an end-face opening provided at the distal end of the elongate catheter body, wherein the end-face opening of the medium drainage channel is in fluid communication with the inlet of the medium drainage channel and with the outlet of the medium drainage channel.

The end-face opening of the medium drainage channel in combination with the inlet and the outlet of the medium drainage channel allows a flow comprised of biological liquid media, biological gaseous media or biological fluid media (i.e. any combination of a biological liquid media and a biological gaseous media), the flow being specific to the mammal hollow organ (i.e. a flow of biological contents being specific to the mammal hollow organ), to be passed bidirectionally through the medium drainage channel under a natural pressure produced by the mammal hollow organ, i.e. passed through the medium drainage channel from the proximal section of the mammal hollow organ to the distal section of the mammal hollow organ or vice versa so that the mammal hollow organ may not be blocked or occluded by excessively accumulated biological liquid media, biological gaseous media or biological fluid media (i.e. excessively accumulated biological contents being specific to the mammal hollow organ), thereby preventing the mammal hollow organ from being damaged (for example, due to an initiated clotting and thrombosis conditioned by the accumulated biological liquid media, biological gaseous media or biological fluid media).

In different embodiments of the present invention according to the third aspect, the catheter further comprises a shut-off valve installed in a proximal terminus of the medium drainage channel extending in the elongate catheter body. The shut-off valve installed in a proximal terminus of the medium drainage channel blocks the leakage or the backflow of the biological contents (e.g. bile) available in the medium drainage channel under an excessively increased pressure (e.g. an excessively increased intra-abdominal pressure) from the medium drainage channel in the direction to a proximal opening of the elongate catheter body, i.e. prevents the backflow of the biological contents available in the medium drainage channel that would otherwise be sprayed to the user via the proximal opening of the elongate catheter body.

In various embodiments of the present invention according to the third aspect, the elongate catheter body is further provided with a loop or a thread designed to be gripped with an endoscope for guiding the gripped catheter body within the mammal hollow organ. The loop or thread provided on the elongate catheter body simplifies the advancement or the movement of the gripped catheter body within the mammal hollow organ to a desired placement (target) site in the lumen of the mammal hollow organ.

In other various embodiments of the present invention according to the third aspect, at least one or each of the inlet and the outlet of the medium drainage channel is a cone-shaped hole tapering towards an interior of the medium drainage channel.

In the catheter according to the third aspect of the present invention, the cone-shaped inlet and/or the cone-shaped outlet of the medium drainage channel provided in the elongate catheter body eliminate or reduce the leakage of the biological contents being specific to the mammal hollow organ from the inside of the medium drainage channel into the proximal section of the mammal hollow organ through the inlet when the excessively increased pressure within the mammal hollow organ is provided. In particular, in such various embodiments of the present invention, the excessively increased pressure provided within the mammal hollow organ causes the passing of the biological contents being specific to the mammal hollow organ in a backwards direction (i.e. from the distal section of the mammal hollow organ back to the proximal section of the mammal hollow organ), so that the biological contents under the excessively increased pressure enter the medium drainage channel through both the outlet of the medium drainage channel and the end-face opening of the medium drainage channel and then transferred via the medium drainage channel towards the proximal end of the elongate catheter body, while the conical shape of the inlet prevents the transferred biological contents from entering the proximal section of the mammal hollow organ via the inlet and, therefore, allow the transferred biological contents to be substantially completely drained via a proximal opening of the medium drainage channel, proximal opening being provided at a proximal end of the elongate catheter body.

In different embodiments of the present invention according to the third aspect, the elongate catheter body is further provided with a net or enclosure being permeable to a liquid medium and/or a gaseous medium, wherein the net or enclosure at least partly encloses a catheter body part defined by the balloons so as to cover the functional opening, and wherein the net or enclosure is attached to the balloons or encloses them such that the net or enclosure becomes strained when the balloons are inflated.

Use of the net or enclosure being permeable to a liquid medium and/or gaseous medium for covering the functional opening in the catheter according to the third aspect of the present invention provides an additional technical effect which is improved safety and increased reliability of the catheter, in particular due to prevented suction of the hollow organ tissue into the functional opening. The attachment of the net or enclosure to the two balloons, the net or enclosure becoming strained between the balloons when inflating the balloons in the catheter according to the third aspect of the present invention, contributes to the above additional technical effect which is the improved safety and increased reliability of the catheter, in particular due to prevented suction of the tissue of the hollow organ into the functional opening of the catheter. The enclosure of the two balloons with the net or enclosure, the net or enclosure becoming strained between the balloons when inflating the balloons in the catheter according to the third aspect of the present invention, also contributes to the above additional technical effect which is the improved safety and increased reliability of the catheter, in particular due to prevented suction of the tissue of the hollow organ into the functional opening of the catheter.

To achieve the objective of the present invention, as embodied and broadly described herein, in a fourth aspect of the present invention, there is provided a system for isolating an interior of a mammal hollow organ, the system comprising: the catheter according to any of the above-described embodiments of the first, second or third aspect of the present invention; and a functional device connected to the functional channel to allow the negative pressure to be produced in the isolated interior of the mammal hollow organ to take the gaseous medium, the liquid medium or the fluid medium therefrom via the functional opening.

The system according to the fourth aspect of the present invention provides all the technical effects mentioned above for the catheter according to the first, second or third aspect of the present invention.

### BRIEF DESCRIPTION OF THE DRAWINGS

While the specification concludes with claims particularly pointing out and distinctly claiming the present invention, it is believed the same will be better understood from the following description taken in conjunction with the accompanying drawings, which illustrate, in a non-limiting fashion, the best mode presently contemplated for carrying out the present invention, and in which like reference numerals designate like parts throughout the drawings, wherein:

FIG. 1.1 illustrates a general view of a first catheter according to the present invention, the first catheter having deflated isolating balloons;

FIG. 1.2 illustrates a general view of the first catheter according to the present invention, the first catheter having inflated isolating balloons and being provided with a connector provided on a proximal end of an elongate catheter body of the first catheter;

FIG. 1.3 illustrates another general view of the first catheter according to the present invention, the first catheter having the inflated isolating balloons and being provided with the connector;

FIGS. 1.4-1.5 illustrate other general views of the first catheter according to the present invention, the first catheter having the inflated isolating balloons;

FIGS. 1.6-1.7 illustrate different longitudinal cross-sections of the first catheter shown in FIGS. 1.4-1.5;

FIG. 1.8 illustrates a general view of a connector to be mounted on the proximal end of the elongate catheter body of the first catheter;

FIGS. 1.9-1.10 illustrate longitudinal cross-sections of the connector shown in FIG. 1.8;

FIG. 1.11 illustrates an enlarged view of two ring-shaped projections provided on the elongate catheter body of the first catheter between the two isolating balloons;

FIGS. 1.12 illustrates a partial view of a distal balloon provided on the elongate catheter body of the first catheter shown in FIGS. 1.4-1.5;

FIG. 1.13 illustrates a longitudinal cross-section of the distal balloon shown in FIG. 1.12;

FIG. 1.14 illustrates an enlarged view of the distal end of the elongate catheter body of the first catheter shown in FIGS. 1.4-1.5;

FIG. 1.15 illustrates a general view of the first catheter according to the present invention, the first catheter having a net or enclosure provided on the deflated isolating balloons;

FIG. 1.16 illustrates a longitudinal cross-section of the first catheter shown in FIG. 1.15;

FIG. 1.17 illustrates a general view of the first catheter according to the present invention, the first catheter having a net or enclosure provided on the inflated isolating balloons;

FIGS. 1.18-1.19 illustrate longitudinal cross-sections of the first catheter shown in FIG. 1.17;

FIG. 2.1 illustrates a general view of a second catheter according to the present invention, the second catheter having deflated isolating balloons;

FIG. 2.2 illustrates a longitudinal cross-section of the second catheter shown in FIG. 2.1;

FIG. 2.3 illustrates another longitudinal cross-section of the second catheter shown in FIG. 2.1;

FIG. 2.4 illustrates a general view of the second catheter according to the present invention, the second catheter having inflated isolating balloons;

FIG. 2.5 illustrates a partial view of the second catheter shown in FIG. 2.4;

FIGS. 2.6-2.11 illustrate different longitudinal cross-sections of the second catheter shown in FIG. 2.5;

FIG. 2.12 illustrates a general view of the second catheter according to the present invention, the second catheter having a net or enclosure provided on deflated isolating balloons;

FIG. 2.13 illustrates a longitudinal cross-section of the second catheter shown in FIG. 2.12;

FIG. 2.14 illustrates a general view of the second catheter according to the present invention, the second catheter having a net or enclosure provided on inflated isolating balloons;

FIG. 2.15 illustrates a longitudinal cross-section of the second catheter shown in FIG. 2.14;

FIG. 2.16 illustrates another longitudinal cross-section of the second catheter shown in FIG. 2.14;

FIG. 2.17 illustrates a distal isolating balloon provided at a distal end of an elongate catheter body of the second catheter;

FIG. 2.18 illustrates an enlarged view of the distal isolating balloon shown in FIG. 2.17;

FIG. 2.19 illustrates a general view of a connector to be mounted on a proximal end of the elongate catheter body of the second catheter;

FIGS. 2.20-2.23 illustrate different longitudinal cross-sections of the connector shown in FIG. 2.19;

FIG. 2.24 illustrates a traverse groove, slot or cut provided in two ring-shaped projections provided on the elongate catheter body of the second catheter between two isolating balloons;

FIG. 2.25 illustrates an alternative embodiment of the traverse groove, slot or cut provided in the two ring-shaped projections provided on the elongate catheter body of the second catheter between two isolating balloons;

FIG. 2.26 illustrates traverse grooves, slots or cuts provided in the two ring-shaped projections provided on the elongate catheter body of the second catheter between two isolating balloons;

FIG. 3.1 illustrates a general view of a third catheter according to the present invention, the third catheter having a helical or spiral-shaped projection externally provided on a catheter body part defined by isolating balloons provided on the elongate catheter body of the third catheter;

FIG. 3.2 illustrates another general view of a third catheter according to the present invention, the third catheter having a helical or spiral-shaped projection externally provided on a catheter body part defined by isolating balloons provided on the elongate catheter body of the third catheter;

FIG. 3.3 illustrates an enlarged view of the catheter body part defined by isolating balloons provided on the elongate catheter body of the third catheter shown in FIG. 3.1 or FIG. 3.2;

FIG. 4.1 illustrates a general view of a fourth catheter according to the present invention, the fourth catheter having two helical or spiral-shaped projections externally provided on a catheter body part defined by isolating balloons provided on the elongate catheter body of the fourth catheter;

FIG. 4.2 illustrates an enlarged view of the catheter body part defined by isolating balloons provided on the elongate catheter body of the fourth catheter shown in FIG. 4.1;

FIG. 4.3 illustrates a general view of an alternative fourth catheter according to the present invention, the alternative fourth catheter having two helical or spiral-shaped projections externally provided on a catheter body part defined by isolating balloons provided on the elongate catheter body of the fourth catheter;

FIG. 4.4 illustrates an enlarged view of the catheter body part defined by isolating balloons provided on the elongate catheter body of the alternative fourth catheter shown in FIG. 4.3;

FIG. 5.1 illustrates a general view of a fifth catheter according to the present invention, the fifth catheter having two helical or spiral-shaped projections externally provided on a catheter body part defined by isolating balloons provided on the elongate catheter body of the fifth catheter;

FIG. 5.2 illustrates an enlarged view of the catheter body part defined by isolating balloons provided on the elongate catheter body of the fifth catheter shown in FIG. 5.1;

FIG. 5.3 illustrates a general view of an alternative fifth catheter according to the present invention, the alternative fifth catheter having two helical or spiral-shaped projections externally provided on a catheter body part defined by isolating balloons provided on the elongate catheter body of the fifth catheter;

FIG. 5.4 illustrates an enlarged view of the catheter body part defined by isolating balloons provided on the elongate catheter body of the alternative fifth catheter shown in FIG. 5.3.

### DETAILED DESCRIPTION

In the context of the present document, unless explicitly stated otherwise, the term “patient” means first of all a potentially sick person (a member of the mammalian class) seeking medical advice or remaining under medical observation to have a disease diagnosed and/or treated, wherein the term “patient” also means potentially sick mammalian animals remaining under medical observation to diagnose and/or treat their disease.

Furthermore, in the context of the present document, unless expressly stated otherwise, the term “mammal” means a human or an animal, in particular anthropoid and non-human primates, dogs, cats, horses, camels, donkeys, cows, sheep, pigs, and other well-known mammals.

Furthermore, in the context of this document, unless expressly stated otherwise, the term “user” means any suitably skilled health care professional authorized to insert the catheter according to the present invention into a mammal hollow organ (in particular, a human hollow organ), remove the catheter according to the present invention from a mammal hollow organ and/or manipulate the catheter according to the present invention inserted into a mammal hollow organ, wherein the healthcare professional may be, for example, surgeon, oncologist, endoscopist, thoracic surgeon, angiosurgeon, urologist, veterinarian, etc.

Nowadays, the most advanced method of diagnosing and/or treating inflammatory diseases, autoimmune diseases, infectious diseases, benign diseases and/or malignant diseases, the diseases occurring in mammal hollow organs or occurring in other mammal organs connected to the mammal hollow organs (in particular, diseases of pancreas, bile ducts, liver, gastrointestinal tract, and also defects and injuries of mammal hollow organs, fistulas, strictures, aneurismal and diverticular dilatations and/or etc.), is a liquid biopsy based on the determination of disease-specific features in biological fluids of a mammal organism, the biological fluids being specific to a mammal hollow organ or other mammal organs connected to the mammal hollow organ. In particular, the liquid biopsy used to diagnose and/or treat pancreatic cancer of a mammal at an initial stage and precancerous transformation is based on the detection of circulating pathologic cells, circulating tumor DNA, RNA, proteins, peptides, metabolites, as well as circulating tumor exosomes in the biological liquids of the mammal organism (e.g. in blood or pancreatic juice).

One critical aspect of the liquid biopsy is an amount of a sampled material, so that it is required to have a sufficient amount of the sampled material for diagnosing a disease. Another critical aspect of the liquid biopsy is localization of a disease in a mammalian organism, in particular tumor localization, since pathologic genetic or other diagnostic material isolated from a sample may be typical of cancers of different mammal hollow organs or other mammal organs connected to the mammal hollow organs.

In particular, in view of the above reason, to diagnose a cancer, mucinous and intraepithelial neoplasms of the pancreas, pancreatic juice is the most appropriate diagnostic liquid to be used for detecting circulating trumor cells, DNA, RNA, proteins, peptides, metabolites, exosomes therein. However, sampling of pancreatic juice is the most difficult problem which is effectively solved by the catheter 100 according to any one of the below-described embodiments of the present invention. Structural and design features and functionalities of the catheter 100 according to the present invention are described in details below in context of solving an illustrative task of sampling/collecting pancreatic juice from duodenal papillas, however the scope of the present invention is not limited by them.

In particular, the catheter 100 according to the present invention is also affectively used for sampling/collecting different biological liquid media or biological gaseous media specific to different mammal hollow organs to diagnose different diseases specific to different mammal hollow organs, monitor the progression of different diseases specific to different mammal hollow organs, treat different diseases specific to different mammal hollow organs, prognosticate different diseases specific to different mammal hollow organs, assess the severity of different diseases specific to different mammal hollow organs, determine a risk group for the mammal, determine conditions of different mammal hollow organs, determine stages of development of different diseases specific to different mammal hollow organs, determine different anomalies or malfunctions of different mammal hollow organs, etc.

It is to note that the minor duodenal papilla and the major duodenal papilla (also referred to in the art as Santorini's papilla and Fateri's papilla, respectively) represent each an anatomical structure in the form of a hemispherical, conical or flattened elevation located at the end of the longitudinal fold of the mucous membrane in the middle of the descending part of the duodenum, in particular about 12-14 cm below the pylorus, wherein in most cases one opening common for the bile and pancreatic ducts is exposed to the duodenum lumen, and in other cases the pancreatic duct is exposed 2-4 cm above the duodenal papilla. A hepato-pancreatic ampoule is located in the duodenal papilla, the ampoule being used for receiving bile and digestive juices of the pancreas and contains the Oddi sphincter regulating the flow of bile or pancreatic juice into the duodenum and preventing the intestinal contents from entering the bile and pancreatic ducts. Therefore, the major duodenal papilla in the duodenum of a mammal is usually 12-14 cm below the pylorus, and the minor duodenal papilla is 2-4 cm above the major duodenal papilla.

In particular, in order to provide the flow of pancreatic juice into the lumen of mammal duodenum, it is necessary to create a physiological negative pressure around the Fateri's papilla or major duodenal papilla at a level of 40-100 mmH2O, wherein the physiological negative pressure is normally created in the mammal duodenum by peristalsis of the mammal duodenum. Another criterion is the need for isolated sampling of pancreatic juice with inactive digestive enzymes, i.e. without gastric and duodenal juice and contents therein, since presence of gastric and duodenal juice and contents in the sampled pancreatic juice with inactive digestive enzymes would otherwise result in activation of the enzymes and digestion of cells, DNA, RNA, proteins, peptides, metabolites, exosomes necessary for performing diagnostics.

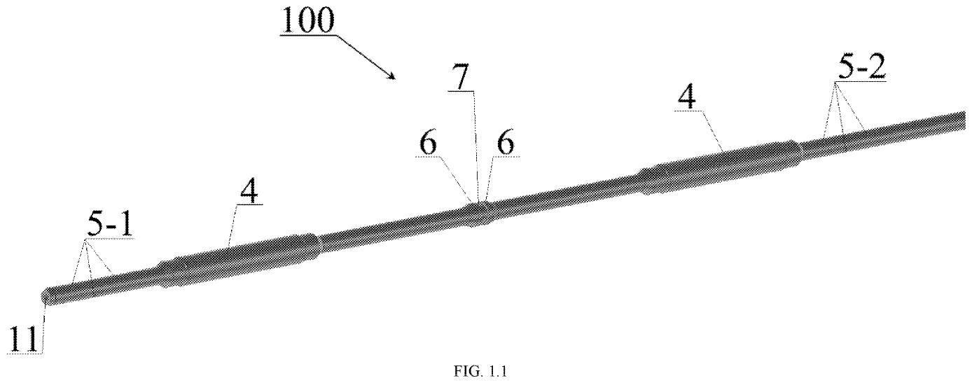

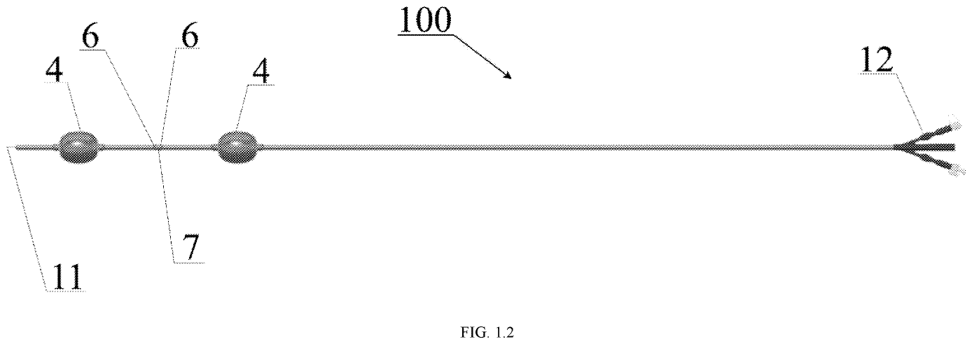

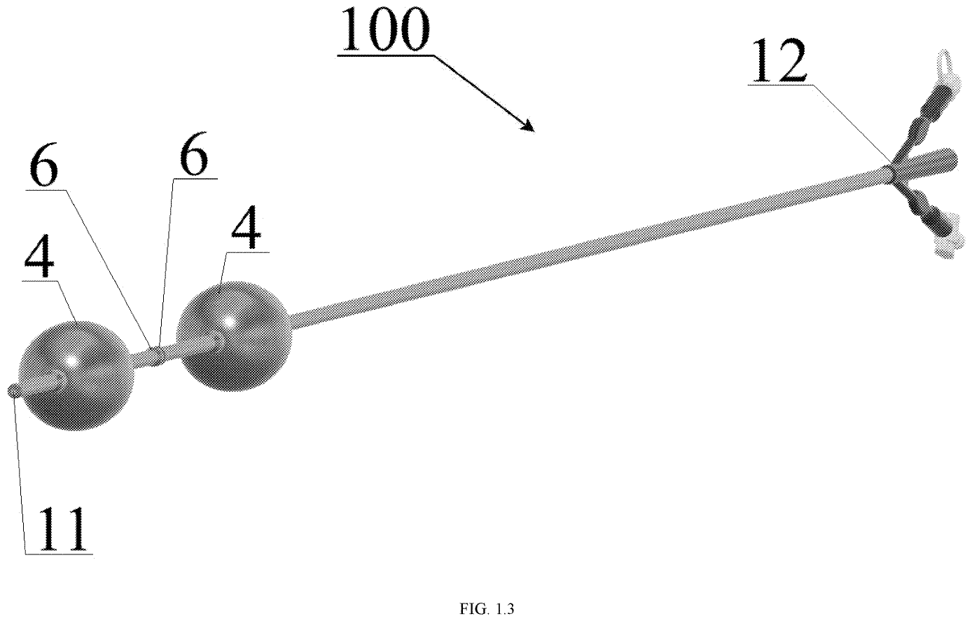

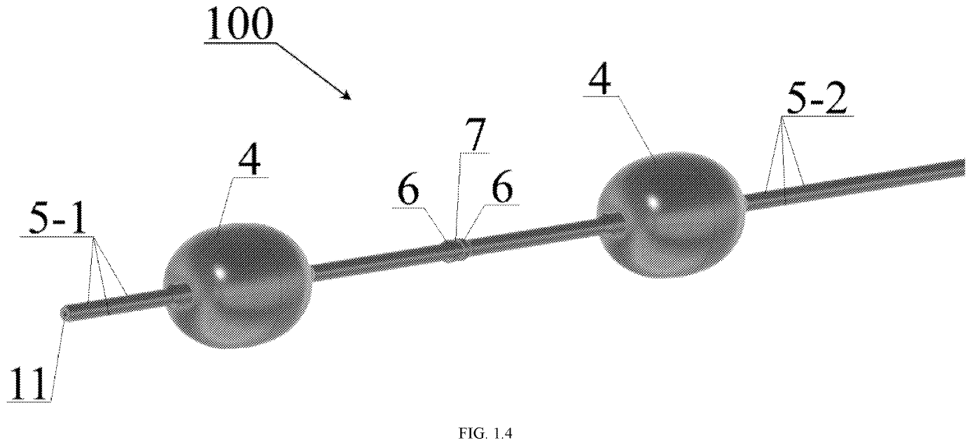

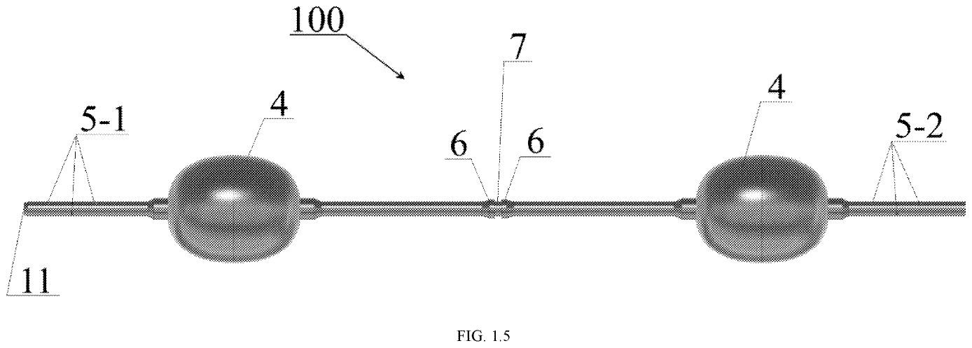

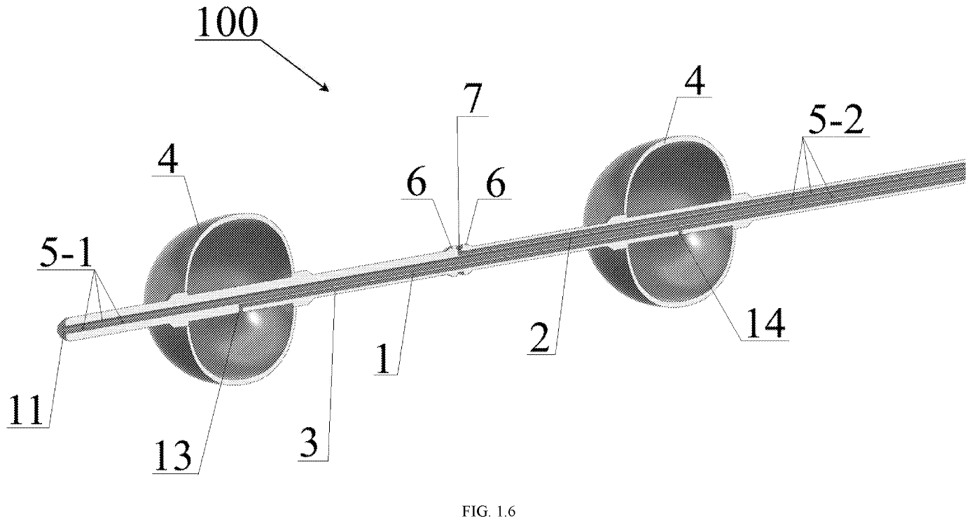

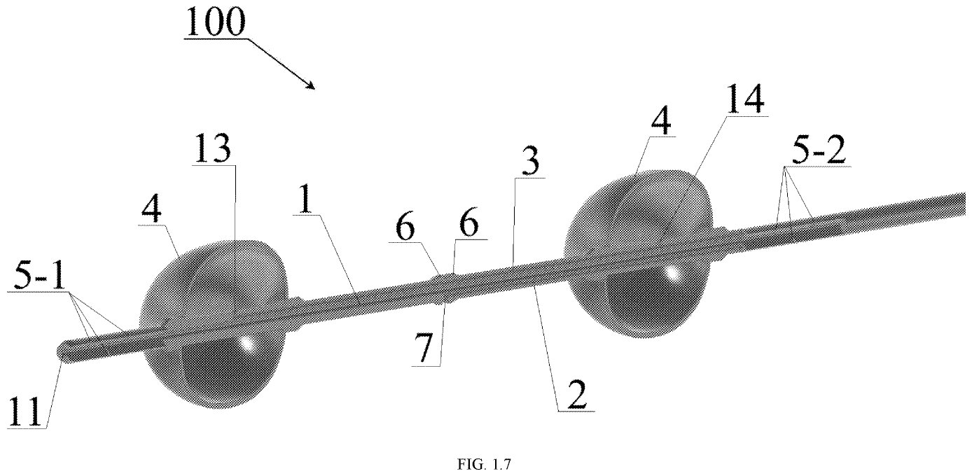

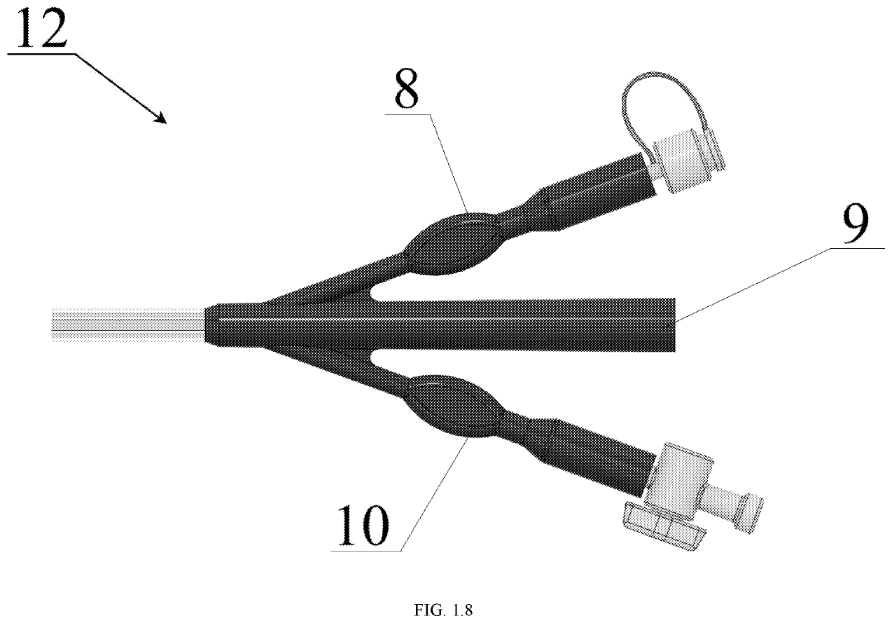

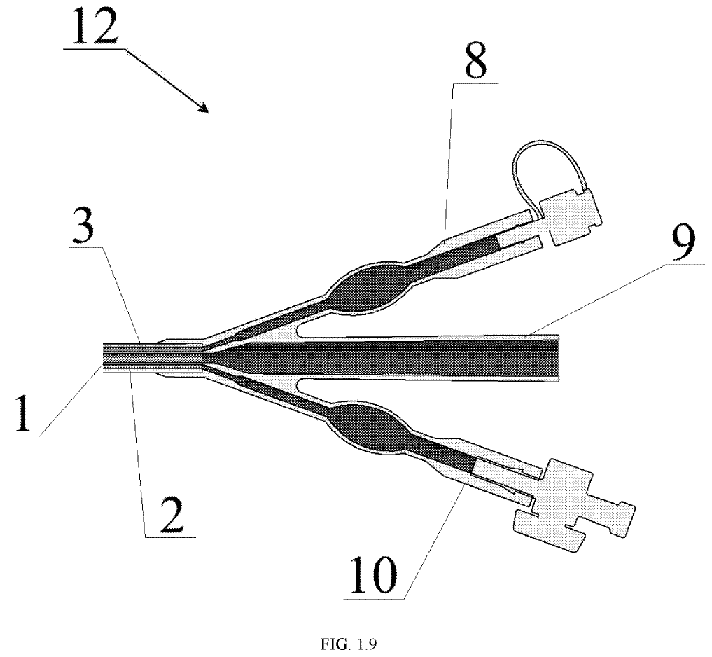

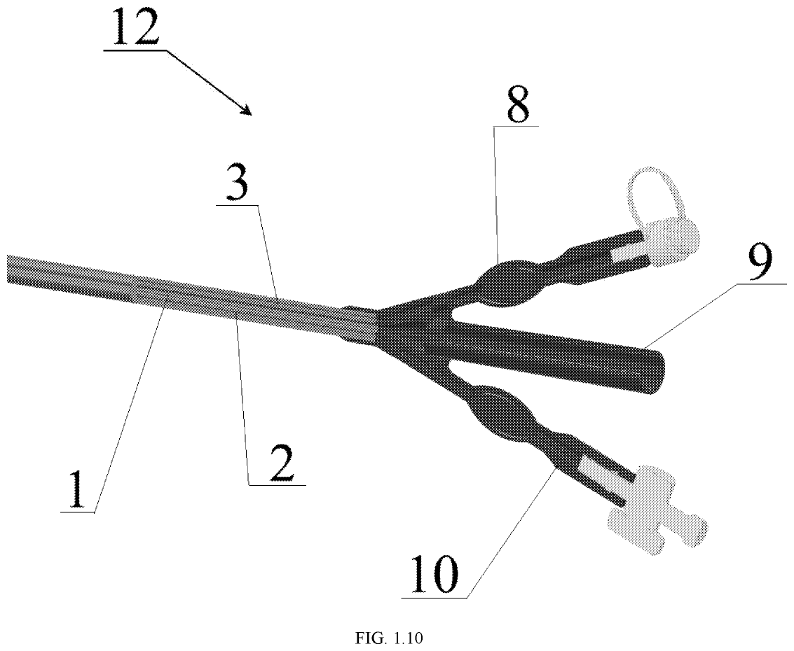

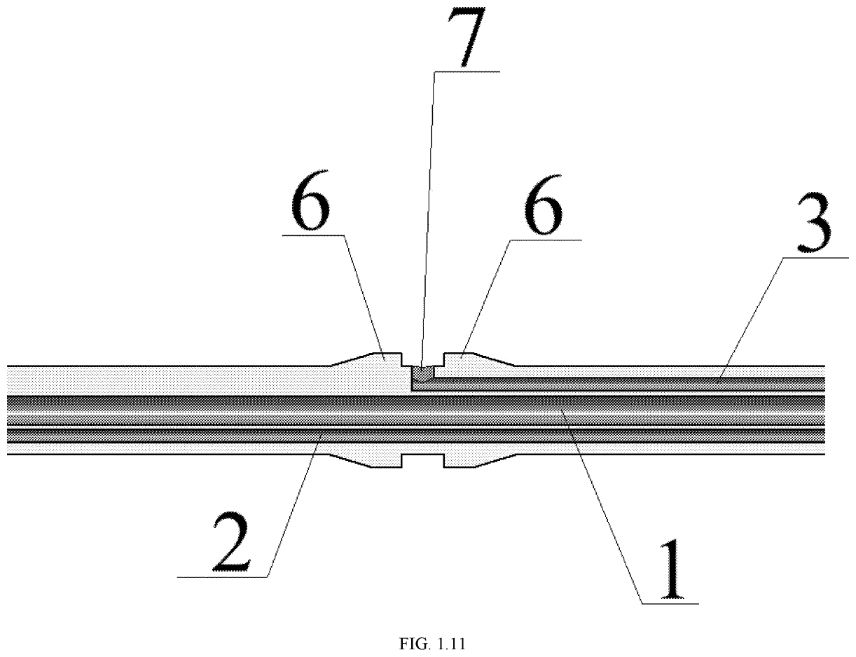

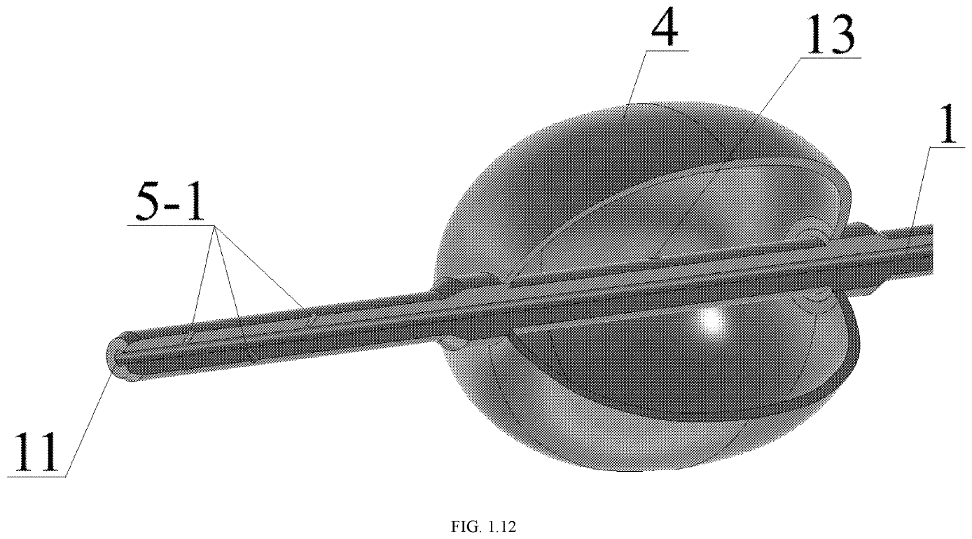

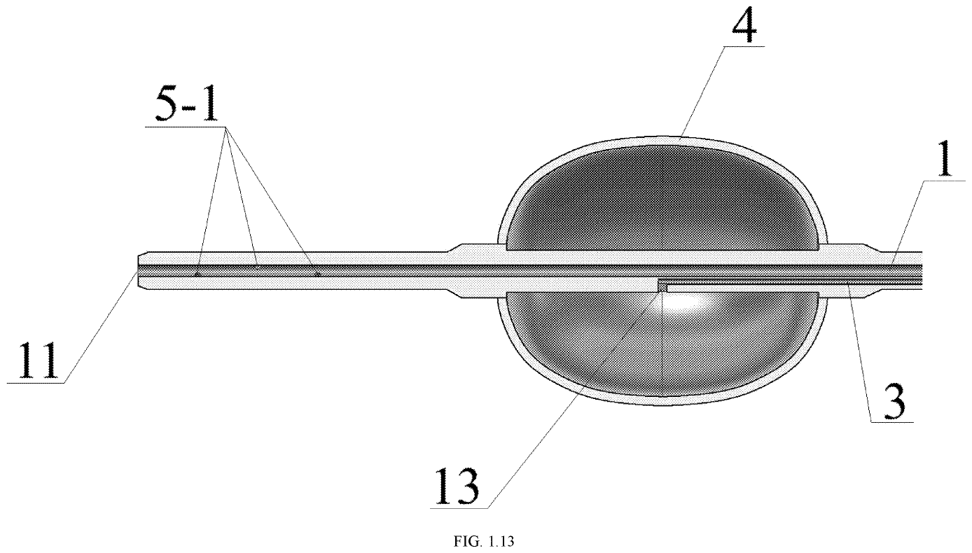

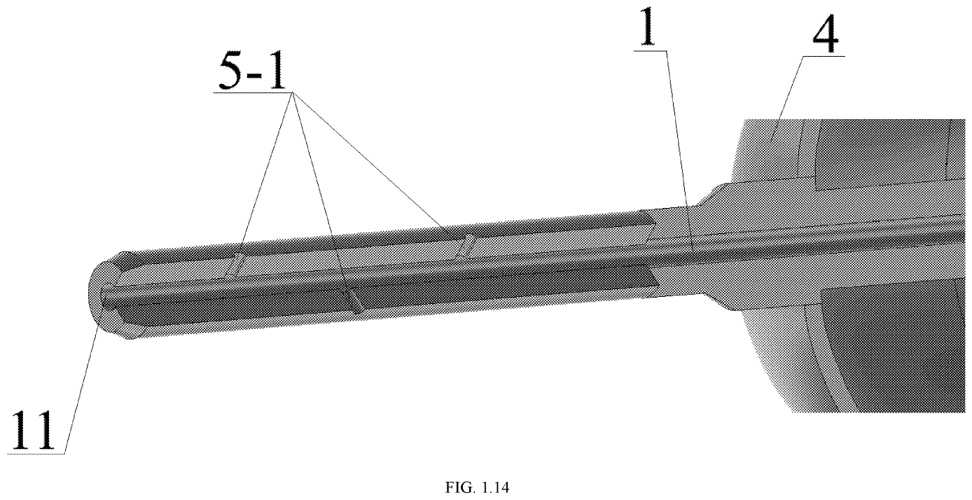

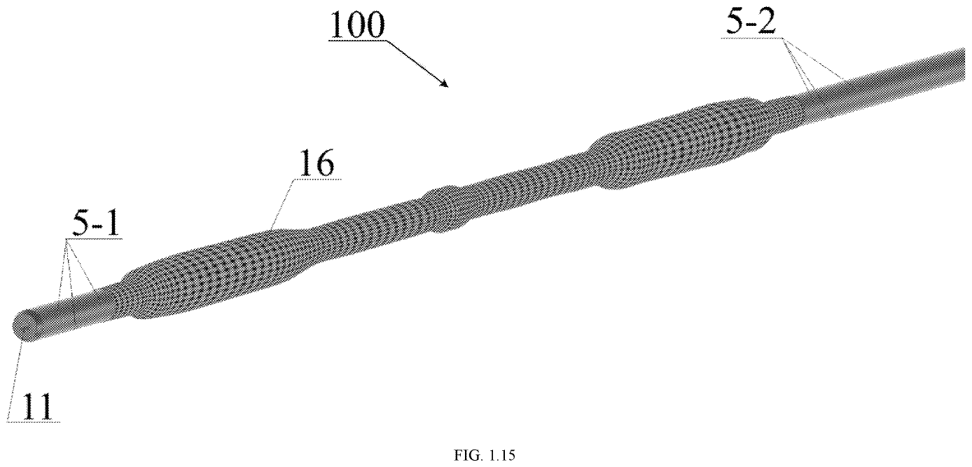

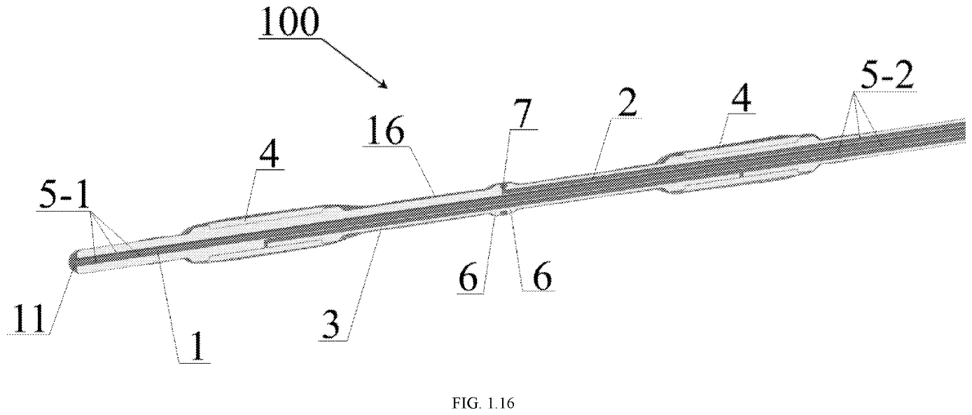

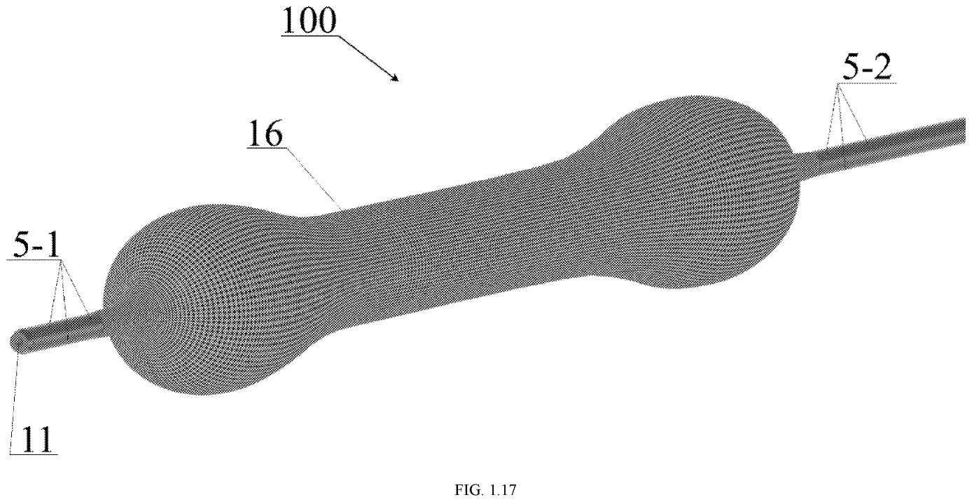

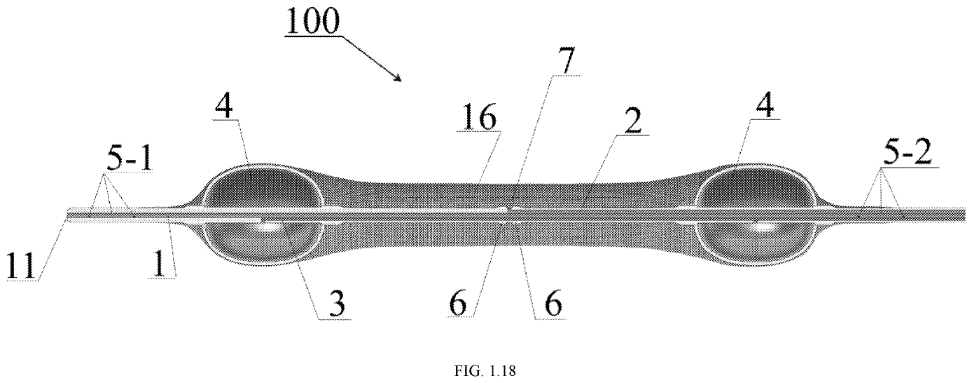

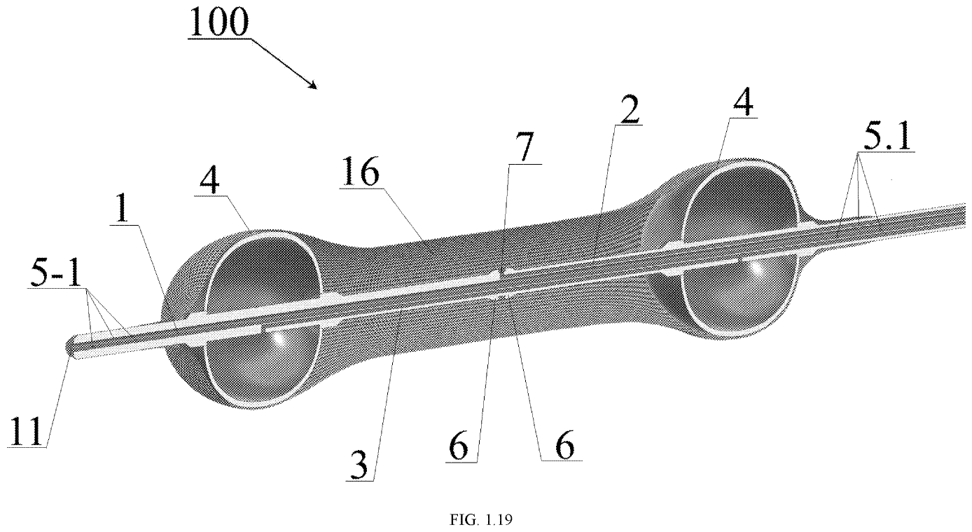

FIGS. 1.1-1.7 schematically illustrate a catheter 100 for isolating an interior of a mammal hollow organ according to the present invention, wherein the catheter 100 is formed as a flexible hollow tube. The catheter 100 has an elongate catheter body designed to be inserted by a user into a lumen of the mammal hollow organ, advanced by the user within the lumen of the mammal hollow organ towards a placement site and removed by the user from the lumen of the mammal hollow organ. In particular, the elongate catheter body has dimensions (in particular, a length and a thickness) and flexibility that are both suitable for performing the user-assisted insertion of the catheter 100 into the mammal hollow organ, the user-assisted advancement of the catheter 100 within the mammal hollow organ towards the placement site and the user-assisted removal of the catheter 100 from the mammal hollow organ. It is to note that the appropriate flexibility of the elongate catheter body simplifies the advancement of the catheter 100 within relatively curved sections of the mammal hollow organ when inserting the catheter 100 into the lumen of the mammal hollow organ or when removing the catheter 100 from the mammal hollow organ. It is to further note that the appropriate flexibility of the elongate catheter body also simplifies the placement of the catheter 100 within a relatively curved section of the mammal hollow organ.

The catheter 100 shown in FIGS. 1.1-2.7 may be used for any patient, in particular any human or animal (i.e. any mammal).

The user manipulating the catheter 100 for inserting the catheter 100 into the lumen of the mammal hollow organ, advancing the catheter 100 within the mammal hollow organ to the placement site in the mammal hollow organ and/or removing the catheter 100 from the mammal hollow organ may be an appropriately skilled healthcare professional, such as, for example, surgeon, oncologist, endoscopist, thoracic surgeon, angiosurgeon, urologist, veterinarian, etc.

The elongate catheter body in the catheter 100 has a distal end and a proximal end opposite to the distal end, wherein the distal end of the elongate catheter body is used for inserting the catheter 100 into the lumen of the mammal hollow organ followed by advancing the catheter 100 within the lumen of the mammal hollow organ to the placement site, in particular a lumen of any mammal hollow organ related to a mammal digestive system (e.g. any organs of gastrointestinal tract such as mouth or oral cavity, pharynx, esophagus, stomach, small intestine including duodenum, jejunum, ileum, large intestine including cecum, appendix, colon, rectum, and anal canal), other mammal accessory digestive organs (e.g. salivary glands, liver, gallbladder, pancreas, bile ducts, etc.), a mammal respiratory system (e.g. nasal cavity, pharynx, larynx, trachea, bronchi, bronchioles), a mammal urinary system (e.g. minor and major renal calyces, renal pelvis, ureters, urinary bladder, urethra), a mammal reproductive system (e.g. vas deferens or Ductus deferens, urethra, ejaculatory ducts, uterine tubes or Fallopian tubes, uterus including the cervix, vagina), mammal cardiovascular system (e.g. heart, blood vessels such as arteries, veins or capillaries) or a mammal lymphatic system (e.g. lymphatic vessels or nodes).

In an example, when the catheter 100 is used for isolating an interior of a mammal duodenum, the catheter 100 is inserted into a nasal passage of a mammal body followed by advancing the inserted catheter 100 along the mammal duodenum to a placement site in the mammal duodenum, the placement site corresponding to the interior to be isolated.

As shown in FIGS. 1.2-1.3, the distal end of the elongate catheter body in the catheter 100 is provided with an axial opening 11, and the proximal end of the elongate catheter body in the catheter 100, the proximal end being opposite to the distal end of the elongate catheter body, is equipped with a connector 12 hermetically connected to the proximal end of the elongate catheter body, wherein the connector 12 is a three-way connector. It is to note that the distal end of the elongate catheter body is within an interior of a distal section of the mammal hollow organ, the distal section being located outside the isolated interior of the mammal hollow organ, and the proximal end of the elongate catheter body is located outside a mammal body (e.g. outside the nasal passage of the mammal body when the catheter 100 is used for isolating the interior of the mammal duodenum). When the elongate catheter body of the catheter 100 is inserted into the lumen of the mammal hollow organ, the axial opening 11 is opened to the interior of the distal section of the mammal hollow organ and in fluid communication with the interior of the distal section of the mammal hollow organ.

In an embodiment of the present invention, the connector 12 may be releasably attached to the proximal end of the elongate catheter body. In another embodiment of the present invention, the connector 12 may be integral with the proximal end of the elongate catheter body.

In particular, as shown in FIGS. 1.8-1.10, the connector 12 in the catheter 100 may be a pipe or a tube provided with three separate branches or terminals: a central terminal 9, and two side terminals 8 and 10 hermetically isolated from the central terminal 9, wherein each of the terminals 8, 9, 10 has an opening provided at a free end of said terminal. The side terminal 8 has an inner channel 8-1 provided inside the side terminal 8, wherein the opening provided at the free end of the side terminal 8 is in fluid communication with the inner channel 8-1. The central terminal 9 has an inner channel 9-1 provided inside the central terminal 9, wherein the opening provided at the free end of the central terminal 9 is in fluid communication with the inner channel 9-1. The side terminal 10 has an inner channel 10-1 provided inside the side terminal 10, wherein the opening provided at the free end of the side terminal 10 is in fluid communication with the inner channel 10-1.

It is to note that all the terminals 8, 9, 10 of the connector 12 are located outside the mammal body when the elongate catheter body of the catheter 100 is inserted into the lumen of the mammal hollow organ.

The terminals 8, 9, 10 in the connector 12 are each designed to have an appropriate functional appliance or functional device (not shown) operatively connected thereto.

In particular, the central terminal 9 positioned between the side terminals 8, 10 in the connector 12 is designed to connect a first functional device (not shown) thereto. It is to note that the inner channel of the central terminal 9 initially communicates with the ambient atmosphere or environment via the opening of the central terminal 9 when the elongate catheter body is inserted into the lumen of the mammal hollow organ, and the first functional device is not connected to the central terminal 9.

The first functional device, when connected to the central terminal 9, is used or actuated for supplying a gaseous medium, a liquid medium or a fluid medium (i.e. a combination of a liquid medium and a gaseous medium), for example an enteral or parenteral nutrition mixture, under a pressure to the inner channel of the central terminal 9 via the opening of the central terminal 9. It is to note that the pressure generated by the first functional device to supply the gaseous medium, the liquid medium or the fluid medium into the central terminal 9 may be set/controlled/regulated/adjusted automatically or manually by a skilled person (e.g. the user).

Thus, the first functional device used for supplying the gaseous medium, the liquid medium or the fluid medium to the central terminal 9 may be implemented as an injection syringe or a medical dropper filled with the gaseous medium, the liquid medium or the fluid medium to be supplied to the central terminal 9. In particular, the first functional device may be implemented as an injection syringe or a medical dropper filled with an enteral or parenteral nutrition mixture so as to provide the mammal organism with basic nutrients, energy, vitamins, macronutrients, micronutrients and/or etc. Alternatively, the first functional device may be implemented as an automated or semi-automated device suitable for connecting the central terminal 9 thereto to supply the gaseous medium, the liquid medium or the fluid medium, for example the enteral or parenteral nutrition mixture, to the central terminal 9. In an illustrative example, the first functional device connected to the central terminal 9 for supplying the gaseous medium, the liquid medium or the fluid medium to the central terminal 9 may be used to provide the nutrition support or clinical nutrition to the mammal when the catheter 100 is inserted into the lumen of duodenum or another target part of gastrointestinal tract of the mammal. Furthermore, the first functional device, when connected to the central terminal 9 for supplying the gaseous medium, the liquid medium or the fluid medium thereto, may be used, for example, to sanitize the mammal stomach and duodenum.

The side terminal 8 of the connector 12 is designed to connect a second functional device (not shown) thereto. It is to note that the inner channel of the side terminal 8 initially communicates with the ambient atmosphere or environment via the opening of the side terminal 8 when the elongate catheter body is inserted into the lumen of the mammal hollow organ, and the second functional device is not connected to the side terminal 8.

The second functional device, when connected to the side terminal 8, is used for evacuating or aspirating an air medium or air under a pressure from the inner channel of the side terminal 8 via the opening of the side terminal 8. It is to note that the pressure generated by the second functional device to aspirate or evacuate the air from the side terminal 8 may be set/controlled/regulated/adjusted automatically or manually by a skilled person (e.g. the user) so that to have the generated pressure corresponding to a standard or natural pressure specific to the mammal hollow organ, thereby preventing the mammal hollow organ from being damaged when actuating the second functional device. In particular, to adjust the pressure to be generated by the second functional device for aspirating or evacuating the air, information disclosed in prior art documents, for example, in U.S. Pat. No. 6,712,798, may be used by the skilled person.

Thus, the second functional device used for aspirating or evacuating the air medium or air under the pressure from the side terminal 8 may be implemented as a medical suction apparatus, an aspiration device or an aspirator (not shown), each comprising (i) a storage reservoir or a storage container (not shown) for collecting a gaseous medium, a liquid medium or a fluid medium specific to the mammal hollow organ and (ii) an air compressor (not shown) for aspirating or evacuating the air under the pressure. In other words, the storage container of the second functional device may be in fluid communication with the opening of the side terminal 8. In an embodiment of the present invention, the storage container may be operatively connected to the second functional device, so that the storage container may be in fluid communication with the opening of the side terminal 8 via the second functional device.

The second functional device, when connected to the side terminal 8, may be alternatively used for supplying a liquid medium, a gaseous medium or a fluid medium (i.e. a combination of a liquid medium and a gaseous medium) under a pressure into the inner channel of the side terminal 8 via the opening of the side terminal 8. It is to note that the pressure generated by the second functional device to supply the liquid medium, the gaseous medium or the fluid medium to the side terminal 8 may be adjusted by the skilled person (e.g. the user) so that to have the generated pressure corresponding to a standard or natural pressure specific to the mammal hollow organ, thereby preventing the mammal hollow organ from being damaged when actuating the second functional device.

Thus, the second functional device used for supplying the liquid medium, the gaseous medium or the fluid medium into the side terminal 8 may be a special pumping/supplying device for pumping/supplying a gaseous medium (e.g. a medical air, a gaseous analgesic, a gaseous anesthetic, medical aerosols in a gaseous phase and other medical gases specific to a particular disease related to the mammal hollow organ), a liquid medium (e.g. water, a liquid contrast agent, an irrigating liquid solution, a sterile saline, a liquid anesthetic, a liquid analgesic, liquid medications/medicine/drugs, a medical suspension, a medical emulsion and other medical liquids specific to a particular disease related to the mammal hollow organ) or a fluid medium (e.g. medical aerosols in a liquid phase and other medical fluids specific to a particular disease related to the mammal hollow organ) under a particular pressure into a particular mammal hollow organ, the pressure being specific to the particular mammal hollow organ. For example, the second functional device may be any appropriate medium supply device for supplying a liquid medium, a gaseous medium or a fluid medium (i.e. a combination of a liquid medium and a gaseous medium) under a pressure, particularly may be implemented as an injection syringe filled with a liquid to be supplied, a medical dropper filled with a liquid to be supplied or other automated or semi-automated appliances or devices which are suitable to be connected to the side terminal 8 and used for supplying the gaseous medium, the liquid medium or the fluid medium to the side terminal 8 under the pressure.

In an embodiment of the present invention the same second functional device may be used for aspirating or evacuating the air medium or the air under the pressure from the side terminal 8 or supplying the liquid medium, the gaseous medium or the fluid medium into the side terminal 8. In particular, in the present embodiment of the present invention, the second functional device may be configured to operate in an air-evacuating mode or a medium-supplying mode, so that the second functional device may be activated by the user in the air-evacuating mode for evacuating the air medium or the air under the pressure from the side terminal 8 or may be activated by the user in the medium-supplying mode for supplying the liquid medium, the gaseous medium or the fluid medium into the side terminal 8.

The side terminal 10 in the connector 12 is designed to connect a third functional device (not shown) thereto. It is to note that the inner channel of the side terminal 10 initially communicates with the ambient atmosphere or environment via the opening of the side terminal 10 when the elongate catheter body is inserted into the lumen of the mammal hollow organ, and the third functional device is not connected to the side terminal 10.

The third functional device, when connected to the side terminal 10, is used or actuated for supplying a gaseous medium (e.g. air, another appropriate gas or another appropriate gas mixture) or a liquid medium (e.g. water, another appropriate liquid substance or another appropriate liquid solution) under a pressure into the inner channel of the side terminal 10 via the opening of the side terminal 10. It is to note that the pressure generated by the third functional device to supply the gaseous medium or the liquid medium to the side terminal 10 may be set/controlled/regulated/adjusted automatically or manually by a skilled person (e.g. the user).

Thus, the third functional device for supplying the gaseous medium or the liquid medium under the pressure to the side terminal 10 may be implemented as an injection syringe filled with the gaseous medium (e.g. air, another appropriate gas or another appropriate gas mixture) or the liquid medium (e.g. water, another appropriate liquid substance or another appropriate liquid solution) to be supplied to the side terminal 10, a medical dropper filled the gaseous medium (e.g. air, another appropriate gas or another appropriate gas mixture) or the liquid medium (e.g. water, another appropriate liquid substance or another appropriate liquid solution) to be supplied or an automated or semi-automated supplying device suitable for connecting the side terminal 10 thereto to supply the gaseous medium (e.g. air, another appropriate gas or another appropriate gas mixture) or the liquid medium (e.g. water, another appropriate liquid substance or another appropriate liquid solution) to the side terminal 10.

In an embodiment of the present invention, the third functional device may be alternatively implemented as an automated, semi-automated or manual medical pump for pumping the gaseous medium (e.g. air, another appropriate gas or another appropriate gas mixture) or the liquid medium (e.g. water, another appropriate liquid substance or another appropriate liquid solution) under a controlled pressure to the side terminal 10, wherein the pressure may be preliminary set/pre-determined/adjusted by a skilled person (e.g. the user), may be automatically controlled/regulated/adjusted during the operation of the medical pump or may correspond to an operation mode of the medical pump, the operation mode being selected/set by the skilled person (e.g. the user), and wherein a volume of the gaseous medium or liquid medium to be supplied by the medical pump may be preliminary set/pre-determined/adjusted by the skilled person (e.g. the user), may be automatically controlled/regulated/adjusted during the operation of the medical pump or may correspond to an operation mode of the medical pump, the operation mode being selected/set by the skilled person (e.g. the user).

It is to note that the catheter 100 according to any of the embodiments described in the present document in combination with (i) the above-described first functional device designed to be connected to the central terminal 9 of the connector 12 for supplying the gaseous medium, the liquid medium or the fluid medium (i.e. the combination of the liquid medium and the gaseous medium), for example for supplying an enteral or parenteral nutrition mixture, into the central terminal 9, (ii) the above-described second functional device designed to be connected to the side terminal 8 of the connector 12 for evacuating or aspirating the air medium or the air under the pressure from the side terminal 8 or for supplying the liquid medium, the gaseous medium or the fluid medium (i.e. the combination of the liquid medium and the gaseous medium) under the pressure into the side terminal 8 and/or (iii) the third functional device designed to be connected to the side terminal 10 of the connector 12 for supplying the gaseous medium or the liquid medium under the pressure into the inner channel of the side terminal 10 may form a system (not shown) for isolating an interior of a mammal hollow organ or may constitute corresponding functional components of the system, wherein the system may be used for sampling or taking a biological gaseous medium, a biological liquid medium or a biological fluid medium (i.e. any appropriate combination of a biological liquid medium and a biological gaseous medium) being specific to the mammal hollow organ, i.e. sampling or taking a biological contents. In an example, the system comprised of the catheter 100 in combination with the first functional device, the second functional device and/or the third functional device may be used for isolating an interior of a mammal duodenum so as to sample a pancreatic juice and/or bile, so that different diseases specific to the mammal duodenum may be diagnosed, monitored, treated, prognosticated by analyzing the sampled pancreatic juice and/or the sampled bile in a special laboratory, thereby allowing the severity of different diseases specific to the mammal duodenum to be assessed by analyzing the sampled pancreatic juice and/or the sampled bile in a special laboratory, allowing the determination of a mammal risk group related to the mammal duodenum by analyzing the sampled pancreatic juice and/or the sampled bile in a special laboratory, allowing the determination of a condition of the mammal duodenum by analyzing the sampled pancreatic juice and/or the sampled bile in a special laboratory, allowing the determination of a development stage of different diseases specific to the mammal duodenum by analyzing the sampled pancreatic juice and/or the sampled bile in a special laboratory or allowing the determination of different anomalies or malfunctions of the mammal duodenum by analyzing the sampled pancreatic juice and/or the sampled bile in a special laboratory.

Furthermore, as shown in FIGS. 1.1-1.7, the catheter body of the catheter 100 is externally provided with two isolating balloons 4, each being formed as an expanding or inflating soft reservoir, wherein the isolating balloons 4 are spaced at a predetermined distance from each other and from the distal end of the elongate catheter body. Thus, the two isolating balloons 4 are individually and separately disposed along the elongate catheter body of the catheter 100 and designed to be inflated to isolate the interior of the mammal hollow organ therebetween.

When the catheter 100 is inserted into the mammal hollow organ, one of the isolating balloons 4, namely the isolating balloon 4 being the farthest from the distal end of the catheter 100, becomes positioned farther than an area of interest in the mammal hollow organ, and the other isolating balloon 4 becomes isolated up to the area of interest in the mammal hollow organ, wherein the functional opening 7 is opposite to the area of interest or next thereto. In an illustrative example, wherein the catheter 100 is inserted into the mammal duodenum, the area of interest is opposite or adjacent to one of the minor and major duodenal papillas or located between the minor duodenal papilla and the major duodenal papilla. In another example, the area of interest may be opposite or adjacent to an intestinal fistula, opposite or adjacent to a wall injury, opposite or adjacent to a tumor, etc.

Furthermore, the elongate catheter body of the catheter 100 comprises three separate functional channels provided in the elongate catheter body of the catheter 100: (1) a main channel 1 extending within the elongate catheter body of the catheter 100, (2) a supply or inflation channel 2 extending within the elongate catheter body of the catheter 100 and used for delivering/supplying a gaseous medium (e.g. air, another appropriate gas or another appropriate gas mixture) or a liquid medium (e.g. water, another appropriate liquid substance or another appropriate liquid solution) to the two isolating balloons 4 to expand or inflate both the isolating balloons 4 or to provide expansion or inflation of the two isolating balloons 4, wherein the inflation channel 2 is hermetically isolated from the main channel 1 and has one or more holes, each hole being provided in the elongate catheter body and being opened to an interior of a corresponding one of the two isolating balloons 4, and (3) a functional channel 3 extending within the elongate catheter body of the catheter 100 and comprising a functional opening (7), the functional opening (7) being provided in the elongate catheter body of the catheter 100 between the isolating balloons 4 and being in fluid communication with the functional channel (3), wherein the functional channel 3 is hermetically isolated from the inflation channel 2 and hermetically isolated from the main channel 1. Thus, the main channel 1 is hermetically isolated from the inflation channel 2 and from the functional channel 3 within the elongate catheter body of the catheter 100, the inflation channel 2 is hermetically isolated from the main channel 1 and from the functional channel 3 within the elongate catheter body of the catheter 100, and the functional channel 3 is hermetically isolated from the main channel 1 and from the inflation channel 2 within the elongate catheter body of the catheter 100. In other words, the main channel 1, the inflation channel 2 and the functional channel 3 are hermetically isolated from each other within the elongate catheter body of the catheter 100.

The inflation channel 2 of the elongate catheter body and the functional channel 3 of the elongate catheter body extend both within a part of the main channel 1.

The main channel 1 of the elongate catheter body is in fluid communication with the central terminal 9 of the connector 12 and extends substantially along all the length of the elongate catheter body, wherein the main channel 1 of the elongate catheter body is designed to be in fluid communication with the interior of the mammal hollow organ via the axial opening 11. In particular, the main channel 1 has an inlet hole or an inlet at the proximal end of the elongate catheter body, the inlet being opened to the inner channel of the central terminal 9 to allow the main channel 1 of the elongate catheter body to be in fluid communication with the inner channel of the central terminal 9, and also has an outlet hole or an outlet corresponding to the axial opening 11 of the elongate catheter body to allow the main channel 1 of the elongate catheter body to be in fluid communication with the interior of the mammal hollow organ.

When the elongate catheter body of the catheter 100, the elongate catheter body having the connector 12 hermetically connected to the proximal end of the elongate catheter body, is initially inserted into the lumen of the mammal hollow organ (i.e. when both the isolating balloons 4 are not inflated), the main channel 1 comprising the axial opening 11 still communicates with the ambient atmosphere or environment via the inlet of the main channel 1, via the inner channel of the central terminal 9 and the opening of the central terminal 9, while the axial opening 11 of the main channel 1 is in fluid communication with the interior of the mammal hollow organ (e.g., esophagus, stomach and duodenum, small intestine or large intestine, and also respiratory tracts, biliary tract, urinary tracts, vessels, etc.).

When both the isolating balloons 4 are then inflated by using the third functional device (not shown) connected to the side terminal 10 of the connector 12, the main channel 1 comprising the axial opening 11 remain to be in communication with the ambient atmosphere or environment via the inlet of the main channel 1, via the inner channel of the central terminal 9 and the opening of the central terminal 9, while the axial opening 11 of the main channel 1 is in fluid communication only with the interior of the distal section of the mammal hollow organ.

When the first functional device (not shown) is then connected to the central terminal 9 of the connector 12, the gaseous medium, the liquid medium or the fluid medium may be firstly supplied under the pressure from the first functional device to the inner channel of the central terminal 9 via the opening of the central terminal 9, then supplied from the inner channel of the central terminal 9 to the main channel 1 via the inlet of the main channel 1, and then finally supplied from the main channel 1 to the interior of the distal section of the mammal hollow organ via the axial opening 11. For example, the above-described first functional device, when connected to the central terminal 9 of the connector 12, may be used or actuated for supplying medical products, in particular the enteral or parenteral nutrition mixture, to the interior of the distal section of the mammal hollow organ via the inner channel of the central terminal 9 and the main channel 1 of the elongate catheter body.

As shown in FIGS. 1.6-1.7, the inflation channel 2 of the elongate catheter body is in fluid communication with the side terminal 10 of the connector 12 and extends substantially along a part of the length of the catheter 100, wherein the inflation channel 2 of the elongate catheter body is also in fluid communication with each of the two isolating balloons 4 via a corresponding one of two outlets 13, 14 provided in the elongate catheter body of the catheter 100. In other words, the inflation channel 2 of the elongate catheter body is in communication with both the isolating balloons 4 via the outlets 13, 14. In particular, the inflation channel 2 has an inlet hole or an inlet, the inlet being opened to the inner channel of the side terminal 10 to allow the inflation channel 2 of the elongate catheter body to be in fluid communication with the inner channel of the side terminal 10 via the inlet of the inflation channel 2 and via the opening of the side terminal 10. Besides, the inflation channel 2 has two outlet holes or two outlets, each corresponding to one of the outlets 13, 14 of the elongate catheter body, to allow the inflation channel 2 of the elongate catheter body to be in fluid communication with the interiors of the two isolating balloons 4. When the elongate catheter body of the catheter 100, the elongate catheter body having the connector 12 hermetically connected to the proximal end of the elongate catheter body, is initially inserted into the lumen of the mammal hollow organ, the inlet of the inflation channel 2 communicates with the ambient atmosphere or environment via the inner channel of the side terminal 10 and via the opening of the side terminal 10, and the outlets of the inflation channel 2 are in fluid communication with corresponding interiors of the two isolating balloons 4. When the third functional device (not shown) is then connected to the side terminal 10 of the connector 12, the gaseous medium (e.g. air, another appropriate gas or another appropriate gas mixture) or the liquid medium (e.g. water, another appropriate liquid substance or another appropriate liquid solution) having a pre-determined volume corresponding to a target expansion/inflation degree may be firstly supplied under the pressure from the third functional device to the inner channel of the side terminal 10 via the opening of the side terminal 10, then supplied from inner channel of the side terminal 10 to the inflation channel 2 via the inlet of the inflation channel 2, and then finally supplied from the inflation channel 2 to the interiors of the two isolating balloons 4 via the outlets 13, 14. Thus, the third functional device, when connected to the side terminal 10 of the connector 12, is used for simultaneously filling both the isolating balloons 4 with the gaseous medium (e.g. air, another appropriate gas or another appropriate gas mixture) or the liquid medium (e.g. water, another appropriate liquid substance or another appropriate liquid solution) via the inflation channel 2 of the elongate catheter body so as to have two expanded or inflated isolating balloons 4 each having an increased size or volume or to provide the expansion or inflation of the two isolating balloons 4 to the increased size or volume. It is to note that use of the inflation channel 2 for simultaneously expanding or inflating the two isolating balloons 4 by means of the third functional device connected to the side terminal 10 of the connector 12 allows sizes of the two isolating balloons 4 to be easily maintained and/or regulated to have inflation degrees of the two isolating balloons 4 that are required to isolate the interior of the mammal hollow organ, thereby further increasing/improving the safety of the catheter 100.

When the elongate catheter body of the catheter 100 is inserted into a lumen of any particular mammal hollow organ, a pressure to be used for simultaneously filling, by means of the third functional device, the isolating balloons 4 with the gaseous medium or the liquid medium may be selected by the skilled person (e.g. the user) on the basis of information disclosed in prior art documents, for example, U.S. Pat. No. 7,722,568.

It is to note that the increased size or the increased volume of the two isolating balloons 4 each having the expanded or inflated state allows the inflated isolating balloons 4 to closely adjoin to a mucosal tissue of the mammal hollow organ and, therefore, can result in bilateral obturation or blockage of the lumen of the mammal hollow organ (for example, the lumen of the mammal duodenum) having the catheter 100 inserted thereinto, thereby allowing a part of the elongate catheter body of the catheter 100, the catheter body part being provided with the functional opening 7, to be isolated between the inflated isolating balloons 4 within the mammal hollow organ. Therefore, the inflated isolating balloons 4 allow a target section of the mammal hollow organ (for example, a target section of the mammal hollow organ that contains major and minor duodenal papillas of the duodenum, aneurysm, a wall defect of the mammal hollow organ, etc.) to be isolated from proximal and distal sections of the mammal hollow organ, thereby excluding or preventing a target biological gaseous medium, a target biological liquid medium or a target biological fluid medium (i.e. a target biological contents) available in the isolated target section of the mammal hollow organ from mixing with other biological gaseous media, biological liquid media or biological fluid media available in the proximal and distal sections of the mammal hollow organ, wherein the inflated isolating balloons 4 allow both the elongate catheter body of the catheter 100 to be secured or fixed within the lumen of the mammal hollow organ (for example, within the lumen of the mammal duodenum) due to close adjoining of the isolating balloons 4 by their external sides to an inner wall surface of the mammal hollow organ. For example, when the elongate catheter body of the catheter 100 is inserted into the lumen of mammal duodenum for isolating the interior of the mammal duodenum, the inflated isolating balloons 4 allow the minor duodenal papilla and/or the major duodenal papilla of the mammal duodenum to be isolated from proximal and distal sections of the gastrointestinal tract, thereby preventing a target biological liquid, the target biological liquid being a mixture of pancreatic juice and bile available in the isolated interior of the mammal duodenum, from mixing with other biological liquids (e.g. gastric juice and/or other duodenal contents) specific to the proximal and distal sections of the mammal duodenum, resulting in that digestive enzymes contained in the pancreatic juice remain inactive.

It is to note that the isolating balloons 4 are in fluid communication with each other via the inflation channel 2, thereby allowing the gaseous medium or the liquid medium used to expand or inflate the two isolating balloons 4 to be uniformly redistributed between interiors of the two isolating balloons 4 via the inflation channel 2, for example during the passage of a peristaltic wave of the intestine or esophagus.

In an embodiment of the present invention, the isolating balloons 4 may be formed as two separate self-inflating balloons. In another embodiment of the present invention, the isolating balloons 4 may be formed both as balloons designed to be expanded or inflated with air supplied by the third functional device to the inflation channel 2 of the elongate catheter body via the opening of the side terminal 10 of the connector 12. In still another embodiment of the present invention, the isolating balloons 4 may be formed both as balloons designed to be expanded or inflated in any other way known in the art.

In other embodiments of the present invention, two inflation channels each corresponding to the above-described inflation channel 2 may be separately provided within the elongate catheter body of the catheter 100, each inflation channel being used for expanding or inflating a corresponding one of the isolating balloons 4 by simultaneously filling said isolating balloon 4 with a gaseous medium (e.g. air, another appropriate gas or another appropriate gas mixture) or a liquid medium (e.g. water, another appropriate liquid substance or another appropriate liquid solution) under a pressure via the inflation channel, wherein the two inflation channels are hermetically isolated from each other within the elongate catheter body of the catheter 100. In such embodiments of the present invention, the above-described side terminal 10 of the connector 12 may be formed as two separate inflation terminals, each being in fluid communication with a corresponding one of the two inflation channels of the elongate catheter body of the catheter 100 and being used to connect an inflation device thereto (e.g. the above-described third functional device) to deliver/supply the gaseous medium or the liquid medium under the pressure to said inflation terminal for expanding or inflating said inflation terminal. In other words, in such embodiments of the present invention, the connector 12 may be provided with four terminals (i.e. the connector 12 may be a four-way connector): a supplying terminal corresponding to the above-described central terminal 9, a functional terminal corresponding to the above-described side terminal 8 and two inflation terminals each corresponding to the above-described side terminal 10, wherein the supplying terminal, the functional terminal and the two inflation terminals are hermetically isolated from each other. In a variant of such embodiments of the present invention, each of the two inflation terminals may be provided with the below-described shut-off valve.

In one embodiment of the present invention, the side terminal 10 in the connector 12 may be optionally equipped with an isolation or shut-off valve (not shown) operated by the user or automatically operated to prevent the backflow or escape of the gaseous medium (e.g. air, another appropriate gas or another appropriate gas mixture) or the liquid medium (e.g. water, another appropriate liquid substance or another appropriate liquid solution) from the isolating balloons 4, the inflation channel 2 and the side terminal 10. It is to further note that the shut-off valve may be used to regulate or adjust the inflation degree of the isolating balloons 4, wherein the user may visually monitor or control the inflation degree by using an endoscope. In particular, the user may reduce the sizes or volumes of the inflated isolating balloons 4 by deflecting or venting the gaseous medium or the liquid medium from the inflation channel 2 by partly or completely opening the shut-off valve.

The functional channel 3 extends substantially along a part of the length of the elongate catheter body of the catheter 100 and is in fluid communication with the side terminal 8 of the connector 12, wherein the functional channel 3 has two ports provided at opposite ends of the functional channel 3, wherein one of the ports corresponds to the functional opening 7, and the other port of the functional channel 3 is provided at the proximal end of the elongate catheter body of the catheter 100 and is opened to the inner channel of the side terminal 8 of the connector 12 to allow the functional channel 3 of the elongate catheter body to be in fluid communication with the inner channel of the side terminal 8, wherein the functional opening 7 is in fluid communication with the opening of the side terminal 8 via the functional channel 3 and via the inner channel of the side terminal 8.

When the elongate catheter body of the catheter 100, the elongate catheter body having the connector 12 hermetically connected to the proximal end of the elongate catheter body, is initially inserted into the lumen of the mammal hollow organ, the functional opening 7 of the functional channel 3 is opened to the interior of the mammal hollow organ, and the other port of the functional channel 3 communicates with the ambient atmosphere or environment via the inner channel of the side terminal 8 and via the opening of the side terminal 8.

When the two isolating balloons 4 are expanded or inflated by means of the third functional device (not shown) connected to the side terminal 10 of the connector 12 so as to isolate the target section of the mammal hollow organ between the inflated isolating balloons 4 (i.e. to isolate an interior of the mammal hollow organ, the interior corresponding to the target section of the mammal hollow organ and being positioned between the inflated isolating balloons 4), the functional opening 7 of the functional channel 3 is opened to the interior of the isolated target section of the mammal hollow organ, the other port of the functional channel 3 remains in communication with the ambient atmosphere or environment via the inner channel of the side terminal 8 and via the opening of the side terminal 8.

When the second functional device (not shown) is then connected to the side terminal 8 of the connector 12 and actuated by the user, air medium or air available in the interior of the isolated target section of the mammal hollow organ is removed therefrom under the pressure via the functional opening 7 opened to the interior of the isolated target section of the mammal hollow organ, the functional channel 3 and the inner channel of the side terminal 8 so as to generate or produce a negative pressure (also referred to in the art as a technical vacuum) in the interior of the isolated target section of the mammal hollow organ (i.e. in the isolated interior of the mammal hollow organ) between the inflated isolating balloons 4, thereby promoting the aspiration or the evacuation of the biological gaseous medium (e.g. an intestinal gas, an alveolar air, a pathological gas, etc.), biological liquid medium (e.g. bile and pancreatic juice, pancreatic secretion or pus, blood or bronchial secretion) or biological fluid medium (i.e. any appropriate combination of a biological liquid medium and a biological gaseous medium) being specific to the mammal hollow organ, i.e. the biological contents being specific to the mammal hollow organ. The biological gaseous medium, biological liquid medium or biological fluid medium (i.e. the biological contents) to be sampled or taken by the second functional device from the isolated interior of the mammal hollow organ is firstly aspirated/evacuated from the isolated interior of the mammal hollow organ to the functional channel 3 via the functional opening 7, then aspirated/evacuated from the functional channel 3 to the inner channel of the side terminal 8 via the port of the functional channel 3 and finally aspirated/evacuated from inner channel of the side terminal 8 to the storage container (not shown) via the opening of the side terminal 8 so as to collect the evacuated biological gaseous medium, biological liquid medium or biological fluid medium in the storage container (i.e. the evacuated biological contents). Thus, the functional channel 3 of the elongate catheter body is designed to allow the negative pressure (i.e. the technical vacuum) to be generated or produced in the interior of the isolated target section of the mammal hollow organ to sample or take the biological gaseous medium, biological liquid medium or biological fluid medium (i.e. the biological contents) therefrom via the functional opening 7, the functional opening 7 being provided in the elongate catheter body of the catheter 100 between the isolating balloons 4 or being provided in the catheter body part defined by the isolating balloons 4. It is to note that the second functional device (not shown), when used for sampling or taking the biological gaseous medium, biological liquid medium or biological fluid medium (i.e. the biological contents) from the interior of the isolated target section of the mammal hollow organ, operates as the aspirator, the functional channel 3 substantially serves as an aspiration channel, and the functional opening 7 substantially serves as an aspiration opening.

It is to further note that the second functional device connected to the side terminal 8 and actuated by the user for generating or producing the negative pressure in the interior of the isolated target section of the mammal hollow organ additionally allows any gaseous medium (e.g. a medical air, a gaseous analgesic, a gaseous anesthetic, medical aerosols in a gaseous phase and other medical gases specific to a particular disease related to the mammal hollow organ), any liquid medium (e.g. water, a liquid contrast agent, an irrigating liquid solution, a sterile saline, a liquid anesthetic, a liquid analgesic, liquid medications/medicine/drugs, a medical suspension, a medical emulsion and other medical liquids specific to a particular disease related to the mammal hollow organ) or any fluid medium, the medium being previously supplied into said interior of the isolated target section of the mammal hollow organ by means of the second functional device or any another appropriate functional device, to be at least partly or completely sampled/taken from said interior of the isolated target section of the mammal hollow organ and then finally collected in the above-described storage container.

Alternatively, the second functional device (not shown), when connected to the side terminal 8 of the connector 12, may be actuated by the user for supplying the gaseous medium (e.g. a medical air, a gaseous analgesic, a gaseous anesthetic, medical aerosols in a gaseous phase and other medical gases specific to a particular disease related to the mammal hollow organ), the liquid medium (e.g. water, a liquid contrast agent, an irrigating liquid solution, a sterile saline, a liquid anesthetic, a liquid analgesic, liquid medications/medicine/drugs, a medical suspension, a medical emulsion and other medical liquids specific to a particular disease related to the mammal hollow organ) or the fluid medium (e.g. medical aerosols in a liquid phase and other medical fluids specific to a particular disease related to the mammal hollow organ) under the pressure into the interior of the isolated target section of the mammal hollow organ (i.e. the isolated interior of the mammal hollow organ) between the inflated isolating balloons 4. Thus, the functional channel 3 of the elongate catheter body is alternatively designed to allow the gaseous medium, the liquid medium or the fluid medium to be supplied under the pressure into the interior of the isolated target section of the mammal hollow organ via the functional opening 7 provided in the elongate catheter body of the catheter 100 between the isolating balloons 4. For example, the gaseous medium, the liquid medium or the fluid medium supplied by the second functional device (not shown) into the interior of the isolated target section of the mammal hollow organ via the functional opening 7 may be used for sanitization of said isolated interior of the mammal hollow organ. It is to note that the second functional device (not shown), when used for supplying the gaseous medium, the liquid medium or the fluid medium under the pressure into the isolated interior of the mammal hollow organ, operates as the supplying device, the functional channel 3 substantially serves as an medium supply channel, and the functional opening 7 substantially serves as a medium supply opening.

In an embodiment of the present invention, the same second functional device may be firstly configured to supply the gaseous medium, the liquid medium or the fluid medium into the interior of the isolated target section of the mammal hollow organ via the functional opening 7 of the elongate catheter body (e.g. activated by the user to operate in the medium-supplying mode) and then reconfigured to produce the negative pressure in said isolated interior (i.e. activated or switched by the user to operate in the in the air-evacuating mode) to at least partly or completely take the previously supplied gaseous medium, the previously supplied liquid medium or the previously supplied fluid medium from said isolated interior of the mammal hollow organ.

In an embodiment of the present invention, the second functional device connected to the side terminal 8 of the connector 12 is the aspirator comprising the storage container and the air compressor, so that the aspirator, when activated by the user, activates the air compressor. In the present embodiment of the present invention, when the elongate catheter body of the catheter 100 is inserted into the lumen of the mammal duodenum, and when the isolating balloons 4 are expanded or inflated by the third functional device (not shown) to isolate the interior of the target section of the mammal duodenum, the target section being positioned between the inflated isolating balloons 4, the activated air compressor of the aspirator allows air available in the interior of the isolated target section of the mammal duodenum to be evacuated or aspirated under the pressure from said interior of the isolated target section of the mammal duodenum via the functional opening 7 and allows air available in the storage container of the aspirator to be evacuated or aspirated under the pressure from the storage container, thereby producing the negative pressure in the interior of the isolated target section of the mammal duodenum. Besides, in the present embodiment of the present invention, the negative pressure produced by the air compressor of the aspirator in the interior of the isolated target section of the mammal duodenum allows the biological gaseous medium, the biological liquid medium or the biological fluid medium (i.e. a biological contents) to be taken from said interior of the isolated target section of the mammal duodenum via the functional opening 7 and also allows the taken biological contents to be supplied into the storage container of the aspirator via the functional channel 3 and via the inner channel of the side terminal 8 for storing or collecting the supplied biological contents in the storage container of the aspirator for a predetermined time period. Alternatively, in the present embodiment of the present invention, the side terminal 8 of the connector 12 may be used to connect the second functional device thereto for administering or supplying the gaseous medium, the liquid medium or the fluid medium (i.e. the medium) into the inner channel of the side terminal 8, transferring the supplied medium through the functional channel 3 and allowing the transferred medium to enter the interior of the isolated target section of the mammal duodenum via the functional opening 7. For example, the medium may be a drug or a coloring agent, so that the medium, when supplied into the interior of the isolated target section of the mammal duodenum, may affect a mucosal tissue of an inner wall of the isolated target section of the mammal duodenum to have a diagnostic effect and/or therapeutic effect on the mucosal tissue.

In another embodiment of the present invention, the elongate catheter body of the catheter 100 has the following two channels provided therein: (1) the above-described inflation channel 2 designed to allow the isolating balloons 4 to be expanded or inflated, and (2) the above-described functional channel 3 designed to allow the negative pressure to be produced in the interior of the isolated target section of the mammal hollow organ to sample or take the gaseous medium, the liquid medium or the fluid medium therefrom via the functional opening 7 or to allow the gaseous medium, the liquid medium or the fluid medium to be supplied into the interior of the isolated target section of the mammal hollow organ via the functional opening 7.

Furthermore, as shown in FIG. 1.11, the elongate catheter body of the catheter 100 is externally provided with two ring-shaped projections 6, wherein the ring-shaped projections 6 have identical sizes (e.g. radius/diameter, thickness, etc.) and identical shapes (i.e. the two ring-shaped projections 6 are identically designed). Each of the two ring-shaped projections 6 has a radius which is more than that of the elongate catheter body of the catheter 100 and less than that of any one of the inflated isolating balloons 4. The two ring-shaped projections 6 are spaced along the length of the elongate catheter body of the catheter 100 and located between the isolating balloons 4, wherein the functional opening 7 is positioned between the two ring-shaped projections 6. It is to note that the functional opening 7 is adjacent to both the ring-shaped projections 6, wherein the two ring-shaped projections 6 are equally spaced from the functional opening 7 along a length of the catheter body part defined between the isolating balloons 4 (i.e. a distance between one of the two ring-shaped projections 6 and the functional opening 7 is equal to a distance between the other ring-shaped projection 6 and the functional opening 7). In other words, the two ring-shaped projections 6 are equally positioned on different sides of the functional opening 7 along the length of the elongate catheter body of the catheter 100 and each located next or close to the functional opening 7. Therefore, the two ring-shaped projections 6 enclosing the functional opening 7 are further provided on the elongate catheter body between the isolating balloons 4 such that the functional opening 7 is between said two ring-shaped projections 6.

It is to note that the two ring-shaped projections 6 enclosing the functional opening 7 are both designed such that the two ring-shaped projections 6 allow a distance between the functional opening 7 and a mucosal tissue of an inner wall of the mammal hollow organ, the mucosal tissue corresponding to the isolated target section of the mammal hollow organ and being between the inflated isolating balloons 4, to be provided when the negative pressure is produced by the second functional device in the interior of the isolated target section of the mammal hollow organ. In other words, the two ring-shaped projections 6 enclosing the functional opening 7 prevent the mucosal tissue of the inner wall of the mammal hollow organ, the mucosal tissue being between the inflated isolating balloons 4 and corresponding to the isolated target section of the mammal hollow organ, from contacting with the functional opening 7 or from approaching the functional opening 7 for a distance appropriate for suction of the mucosal tissue into the functional opening 7 when the negative pressure is produced by the second functional device in the isolated target section of the mammal hollow organ, thereby preventing the mucosal tissue of the inner wall of the isolated target section of the mammal hollow organ from being damaged and preventing the functional opening 7 from being blocked or occluded by the mucosal tissue of the inner wall of the isolated target section of the mammal hollow organ.

In other words, the two ring-shaped projections 6 prevent the suction of the mucosal tissue of the inner wall of the isolated target section of the mammal hollow organ into the functional opening 7, while allowing the gaseous medium, the liquid medium or the fluid medium available in the interior of the isolated target section of the mammal hollow organ to be aspirated or evacuated by the second functional device (not shown) from the interior of the isolated target section of the mammal hollow organ. Therefore, the two ring-shaped projections 6 enclosing the functional opening 7 improve a safety of the catheter 100.

In an embodiment of the present invention, the elongate catheter body of the catheter 100 may be further externally provided with two annular bulges or annular protrusions (not shown), wherein the two protrusions are provided on the elongate catheter body outside the catheter body part defined by the isolating balloons 4 and each equally spaced from a corresponding one of the two isolating balloons 4.

Furthermore, as shown in details in FIGS. 1.1, 1.4-1.7 and 1.12-1.14, three auxiliary holes 5-1 and three auxiliary holes 5-2 are provided in the elongate catheter body outside of the catheter body part defined by the isolating balloons 4. In particular, the three auxiliary holes 5-1 are spaced at a predetermined distance from each other along the length of the elongate catheter body and spaced all at a predetermined distance from the distal end of the elongate catheter body, and the three auxiliary holes 5-2 are spaced at a predetermined distance from each other along the length of the elongate catheter body and spaced all at a predetermined distance from the proximal end of the elongate catheter body, wherein all the three auxiliary holes 5-1 are also spaced at a predetermined distance from a corresponding one of the two isolating balloons 4, and wherein all the three auxiliary holes 5-2 are also spaced at a predetermined distance from the other isolating balloon 4. In other words, the three auxiliary holes 5-2 and the three auxiliary holes 5-1 are separated from each other by means of the catheter body part defined by the isolating balloons 4.

It is to note that the three auxiliary holes 5-1 and three auxiliary holes 5-2 are all in fluid communication with the main channel 1 of the elongate catheter body of the catheter 100, so that the three auxiliary holes 5-1 are all in fluid communication with all the three auxiliary holes 5-2 via the main channel 1.

In an embodiment of the present invention, the elongate catheter body is provided with at least one auxiliary hole 5-1 (e.g. one, two, three, four, five, six, seven, eight, nine, ten or more auxiliary holes 5-1) and at least one auxiliary hole 5-2 (e.g. one, two, three, four, five, six, seven, eight, nine, ten or more auxiliary holes 5-2).

When the distal end of the elongate catheter body is inserted into the lumen of the mammal hollow organ for inserting the catheter 100 into the mammal hollow organ, the three auxiliary holes 5-1 positioned closer to the distal end of the elongate catheter body serve as outlets and are located in the distal section of the mammal hollow organ, and the three auxiliary holes 5-2 positioned further from the distal end of the elongate catheter body serve as inlets and are located in the proximal section of the mammal hollow organ.

It is to note that the auxiliary holes 5-1, 5-2 allow contents being specific to the mammal hollow organ (e.g. air, urine, blood, mucus, gastric or intestinal contents with inactive digestive ferments, other contents in the mammal hollow organ etc.), including the biological contents being specific to the mammal hollow organ, to flow or pass to the distal section of the mammal hollow organ without entering the interior isolated between the inflated isolating balloons 4 of the catheter 100, thereby eliminating or preventing the excessive accumulation of said contents in a proximal section of the mammal hollow organ, the proximal section being located outside the isolated interior of the mammal hollow organ and being defined by the inflated isolating balloon 4 positioned farther or farthest from the distal end of the elongate catheter body of the catheter 100, and, therefore, eliminating or preventing the formation of congestive processes and/or inflammatory processes in the proximal section of the mammal hollow organ that could be caused by the accumulated contents.

Alternatively, when the distal end of the elongate catheter body is inserted into the lumen of the mammal hollow organ for inserting the catheter 100 into the mammal hollow organ, the three auxiliary holes 5-1 positioned closer to the distal end of the elongate catheter body serve as inlets and are located in the distal section of the mammal hollow organ, and the three auxiliary holes 5-2 positioned further from the distal end of the elongate catheter body serve as outlets and are located in the proximal section of the mammal hollow organ. It is to further note that the auxiliary holes 5-1, 5-2 alternatively allow contents being specific to the mammal hollow organ (e.g. air, urine, blood, mucus, gastric or intestinal contents with inactive digestive ferments, other contents in the mammal hollow organ etc.), including the biological contents being specific to the mammal hollow organ, to flow or pass to the proximal section of the mammal hollow organ without entering the interior isolated between the inflated isolating balloons 4 of the catheter 100, thereby eliminating or preventing the excessive accumulation of said contents in the distal section of the mammal hollow organ, the distal section being located outside the isolated interior of the mammal hollow organ and being defined by the inflated isolating balloon 4 positioned closer or closest to the distal end of the elongate catheter body of the catheter 100, and, therefore, eliminating or preventing the formation of congestive processes and/or inflammatory processes in the distal section of the mammal hollow organ that could be caused by the accumulated contents.

Thus, as described above, depending on a particular mammal hollow organ having the elongate catheter body of the catheter 100 inserted into the lumen thereof, the three auxiliary holes 5-1 and three auxiliary holes 5-2 may be used as outlets and inlets, respectively, or vice versa (i.e. as inlets and outlets, respectively). In particular, the functional role of the auxiliary holes 5-1, 5-2 depends on a direction of a biological flow under a natural pressure, the biological flow being specific to the particular mammal hollow organ having the elongate catheter body of the catheter 100 inserted into the lumen thereof.

In other words, the auxiliary holes 5-1, 5-2 allow a flow comprised of biological liquid media, biological gaseous media or biological fluid media (i.e. any combination of a biological liquid media and a biological gaseous media), the flow being specific to the mammal hollow organ (i.e. a flow of biological contents being specific to the mammal hollow organ), to be passed bidirectionally through the main channel 1 of the elongate catheter body under a natural pressure produced by the mammal hollow organ, i.e. passed through the main channel 1 of the elongate catheter body from the proximal section of the mammal hollow organ to the distal section of the mammal hollow organ (wherein the flow enters the main channel 1 though the three auxiliary holes 5-2 operating as inlets and leaves the main channel 1 through the three auxiliary holes 5-1 operating as outlets) or vice versa (wherein the flow enters the main channel 1 though the three auxiliary holes 5-1 operating as inlets and exits the main channel 1 through the three auxiliary holes 5-2 operating as outlets), so that the mammal hollow organ may not be blocked or occluded by excessively accumulated biological liquid media, biological gaseous media or biological fluid media (i.e. excessively accumulated biological contents being specific to the mammal hollow organ), thereby preventing the mammal hollow organ from being damaged (for example, due to an initiated clotting and thrombosis conditioned by the accumulated biological liquid media, biological gaseous media or biological fluid media). Therefore, in the catheter 100, the formation of congestive processes and/or inflammatory processes in the mammal hollow organ due to the biological liquid media, biological gaseous media or biological fluid media (i.e. the biological contents) accumulated outside the interior isolated between the inflated balloons 4 is prevented or excluded due to the provided bidirectional passage through the main channel 1 of the elongate catheter body, so that said biological liquid media, biological gaseous media or biological fluid media (i.e. the biological contents), when accumulated in the proximal mammal hollow organ, may enter the main channel 1 through the three auxiliary holes 5-1 and may leave the main channel 1 through the three auxiliary holes 5-2 or vice versa (i.e. said biological liquid media, biological gaseous media or biological fluid media, when accumulated in the distal mammal hollow organ, may enter the main channel 1 through the three auxiliary holes 5-2 and may leave the main channel 1 through the three auxiliary holes 5-1). In some embodiments of the present invention, in the catheter 100, a proximal terminus of the main channel 1 (in particular, a section of the main channel 1 that is between the three auxiliary holes 5-2 and the inner channel of the central terminal 9) may be further provided with a shut-off valve (not shown) installed inside the proximal terminus of the main channel 1, wherein the shut-off valve is designed to be operated by the user or automatically operated to prevent the backflow of the biological contents available in the main channel 1 (especially, under an excessively increased pressure such as an excessively increased intra-abdominal pressure) from the main channel 1 to the central terminal 9. Thus, the shut-off valve installed within the proximal terminus of the main channel 1 prevents any biological contents (e.g. bile) from being uncontrollably passed from the main channel 1 to the central terminal 9 and, therefore, prevents the passed biological contents from being sprayed to the user via the opening of the central terminal 9. In other words, the shut-off valve installed within the proximal terminus of the main channel 1 is designed to block the leakage of the biological contents available in the main channel 1 under an excessively increased pressure from the main channel 1 in the direction to the opening of the central terminal 9. It is to note that the shut-off valve installed within the proximal terminus of the main channel 1 allows the fourth functional device, when connected to the central terminal 9 and actuated by the user, to provide the controlled discharge of the biological contents from the main channel 1 and from the distal and proximal sections of the mammal hollow organ (i.e. sections of the mammal hollow organ that are outside the isolated interior of the mammal hollow organ) via the opening of the central terminal 9.

In other embodiments of the present invention, in the catheter 100, the central terminal 9 may be further provided with a shut-off valve (not shown) installed inside the inner channel of the central terminal 9, wherein the shut-off valve is designed to be operated by the user or automatically operated to prevent the backflow of the biological contents available in the main channel 1 (especially, under an excessively increased pressure such as an excessively increased intra-abdominal pressure) from the main channel 1 to the opening of the central terminal 9. Thus, the shut-off valve installed within the inner channel of the central terminal 9 prevents any biological contents (e.g. bile) from being uncontrollably passed from the main channel 1 to the opening of the central terminal 9 and, therefore, prevents the passed biological contents from being sprayed to the user via the opening of the central terminal 9. In other words, the shut-off valve installed within the inner channel of the central terminal 9 is designed to block the leakage of the biological contents available in the main channel 1 under an excessively increased pressure from the main channel 1 in the direction to the opening of the central terminal 9. It is to note that the shut-off valve installed within inner channel of the central terminal 9 allows the fourth functional device, when connected to the central terminal 9 and actuated by the user, to provide the controlled discharge of the biological contents from the main channel 1 and from the distal and proximal sections of the mammal hollow organ (i.e. sections of the mammal hollow organ that are outside the isolated interior of the mammal hollow organ) via the opening of the central terminal 9.

In various embodiments of the present invention, in the catheter 100, at least one or each of the auxiliary holes 5-1 of the main channel 1 and/or at least one or each of the auxiliary holes 5-2 of the main channel 1 may be cone-shaped holes, i.e. may have each a geometric shape tapering towards the interior of the main channel 1. In other words, in such various embodiments of the present invention, at least one of the auxiliary holes 5-1 of the main channel 1 and/or at least one of the auxiliary holes 5-2 of the main channel 1 may be tapered holes or conical holes tapering towards the interior of the main channel 1, thereby reducing leakage of the gaseous medium, the liquid medium or the fluid medium (e.g. medical products such as the enteral or parenteral nutrition mixture) supplied to the main channel 1 by means of the first functional device from the inside of the main channel 1 into the distal and proximal sections of the mammal hollow organ through the auxiliary holes 5-1 and/or the auxiliary holes 5-2 and simultaneously improving the drainage of the biological contents being specific to the mammal hollow organ from the distal and proximal sections of the mammal hollow organ into the inside of the main channel 1 through the auxiliary holes 5-1 and/or the auxiliary holes 5-2. Therefore, in such various embodiments of the present invention, conical shapes of the auxiliary holes 5-1, 5-2 of the main channel 1 allow the excessive accumulation of the biological contents being specific to the mammal hollow organ to be minimized in the distal section of the mammal hollow organ, the distal section being defined by the inflated isolating balloon 4 positioned closer or closest to the distal end of the elongate catheter body of the catheter 100, and/or the proximal section of the mammal hollow organ, the proximal section being located outside the isolated interior of the mammal hollow organ and being defined by the inflated isolating balloon 4 positioned farther or farthest from the distal end of the elongate catheter body of the catheter 100, thereby minimizing the formation of congestive processes and/or inflammatory processes in the distal section of the mammal hollow organ and/or the proximal section of the mammal hollow organ that could be caused by the accumulated biological contents, respectively.

In an embodiment of the present invention, the main channel 1 provided within the elongate catheter body of the catheter 100 comprises one or more auxiliary holes 5-1 (e.g. one, two, three, four, five, six, seven, eight, nine, ten or more auxiliary holes 5-1) and one or more auxiliary holes 5-2 (e.g. one, two, three, four, five, six, seven, eight, nine, ten or more auxiliary holes 5-2).

In another embodiment of the present invention, the main channel 1 provided within the elongate catheter body of the catheter 100 only comprises one or more auxiliary holes 5-2 (e.g. one, two, three, four, five, six, seven, eight, nine, ten or more auxiliary holes 5-2), wherein auxiliary holes 5-1 are not provided in the elongate catheter body of the catheter 100.

In an embodiment of the present invention, the elongate catheter body of the catheter 100 is provided only with one or more auxiliary holes 5-1 (e.g. one, two, three, four, five, six, seven, eight, nine, ten or more auxiliary holes 5-1) and one or more auxiliary holes 5-2 (e.g. one, two, three, four, five, six, seven, eight, nine, ten or more auxiliary holes 5-2) and not provided with the axial opening 11, i.e. the main channel 1 does not comprise the axial opening 11 and only comprises one or more auxiliary holes 5-1 and one or more auxiliary holes 5-2, wherein the auxiliary holes 5-1 and the auxiliary holes 5-2 are in fluid communication with each other via the main channel 1.

In another embodiment of the present invention, the elongate catheter body of the catheter 100 is provided only with the axial opening 11 and one or more auxiliary holes 5-2 (e.g. one, two, three, four, five, six, seven, eight, nine, ten or more auxiliary holes 5-2) and not provided with the auxiliary holes 5-1, i.e. the main channel 1 does not comprise the auxiliary holes 5-1 and only comprises the axial opening 11 and one or more auxiliary holes 5-2, wherein the axial opening 11 and the auxiliary holes 5-2 are in fluid communication with each other via the main channel 1.

Furthermore, as shown in FIGS. 1.15-1.19, the elongate catheter body of the catheter 100 is provided with a net or enclosure 16 connected or attached to both the annular protrusions 15 such that the net or enclosure 16 completely or at least partly envelopes/encloses the two isolating balloons 4 and the catheter body part defined by the isolating balloons 4, thereby completely or at least partly enveloping or covering the functional opening 7, wherein the net or enclosure 16 is elastic, tensile or stretchable. In particular, FIGS. 1.15-1.16 illustrate the net or enclosure 16 having a nonstretched or nonstrained state, i.e. a state when both the insolating balloons 4 are not inflated by the third functional device (not shown).

Furthermore, the net or enclosure 16 is made of a permeable material or a porous material permeable to a gaseous medium, a liquid medium and/or a fluid medium (i.e. a gas, a liquid or a fluid) or has a cellular or grid structure permeable to a gaseous medium, a liquid medium and/or a fluid medium (i.e. a gas, a liquid or a fluid). In other words, the net or enclosure 16 is designed to allow the gaseous medium, the liquid medium and/or the fluid medium (as any appropriate combination of the gaseous medium and the liquid medium), i.e. the gaseous medium and/or the liquid medium, to flow or pass therethrough.

In one embodiment of the present invention, the enclosure 16 provided on the elongate catheter body of the catheter 100 is a grid enclosure or a grid envelope designed to be permeable to a gaseous medium, a liquid medium or a fluid medium (i.e. permeable to a gas, a liquid or a fluid).

It is to note that the net or enclosure 16 attached to both the annular protrusions 15 envelopes/encloses both the isolating balloons 4 such that the net or enclosure 16 becomes at least partly stretched or strained when both the insolating balloons 4 are inflated by the third functional device (not shown), while the two insolating balloons 4 become tightly enclosed by the strained net or enclosure 16 as shown in FIGS. 1.17-1.19.

In an alternative embodiment of the present invention, the net or enclosure 16 is mounted or pulled only on both the insolating balloons 4 (i.e. the annular protrusions 15 are not provided on the elongate catheter body of the catheter 100) such that the net or enclosure 16 becomes at least partly stretched or strained when both the insolating balloons 4 are inflated by the third functional device (not shown), while the two insolating balloons 4 become tightly enclosed by the strained net or enclosure 16.

In another alternative embodiment of the present invention, the net or enclosure 16 is engaged only on both the insolating balloons 4 (i.e. the annular protrusions 15 are not provided on the elongate catheter body of the catheter 100) such that the net or enclosure 16 becomes at least partly stretched or strained when both the insolating balloons 4 are inflated by the third functional device (not shown), while the two insolating balloons 4 become tightly enclosed by the strained net or enclosure 16.

In still another alternative embodiment of the present invention, the net or enclosure 16 is connected or attached to both the insolating balloons 4 (i.e. the annular protrusions 15 are not provided on the elongate catheter body of the catheter 100) such that the net or enclosure 16 becomes at least partly stretched or strained when both the insolating balloons 4 are inflated by the third functional device (not shown), while the two insolating balloons 4 become tightly enclosed by the strained net or enclosure 16.

In other embodiments of the present invention, the net or enclosure 16 provided on the elongate catheter body of the catheter 100 encloses both the insolating balloons 4 (i.e. the annular protrusions 15 are not provided on the elongate catheter body of the catheter 100) such that the net or enclosure 16 becomes at least partly stretched or strained when both the insolating balloons 4 are inflated by the third functional device (not shown), while the two insolating balloons 4 remain completely or partly tightly enclosed by the strained net or enclosure 16.

The strained net or enclosure 16 shown in FIGS. 1.17-1.19 forms an outer casing or an outer frame which operates as an elastic protective cover for completely or at least partly enclosing both the catheter body part defined by the two isolating balloons 4 and the functional opening 7 provided in the catheter body part between the two isolating balloons 4, thereby allowing the mucosal tissue of the inner wall of the mammal hollow organ, the mucosal tissue being in contact with the strained net or enclosure 16 in the isolated target section of the mammal hollow organ, to be moved or displaced for a predetermined distance away from the functional opening 7, wherein said distance depends at least on a tension degree or a strain level of the strained net or enclosure 16, and the tension degree or the strain level of the strained net or enclosure 16 depends in turn at least on inflation degrees of the isolating balloons.

It is to further note that the outer casing or an outer frame formed by the strained net or enclosure 16 generally has a cylindrical shape, an ovoid shape or an ellipsoid shape, wherein the outer casing or the outer frame is easily penetrable by the gaseous medium, the liquid medium or the fluid medium (i.e. the gas, the liquid or the fluid) available in the interior of the isolated target section of the mammal hollow organ due to its cellular structure, thereby allowing the gaseous medium, the liquid medium or the fluid medium to flow or pass therethrough when the negative pressure is produced by the second functional device (not shown) in the interior of the isolated target section of the mammal hollow organ.

In particular, the strained net or enclosure 16 shown in FIGS. 1.17-1.19 allows the biological gaseous medium (e.g. an intestinal gas, an alveolar air, a pathological gas, etc.), biological liquid medium (e.g. bile and pancreatic juice, pancreatic secretion or pus, blood or bronchial secretion) or biological fluid medium (i.e. any appropriate combination of a biological liquid medium and a biological gaseous medium) being specific to the mammal hollow organ (i.e. the biological contents being specific to the mammal hollow organ) to penetrate, flow or pass through the net or enclosure 16 and also allows said passed medium to be evacuated from the interior of the isolated target section of the mammal hollow organ by using the second functional device (not shown).

The strained net or enclosure 16 shown in FIGS. 1.17-1.19 also allows the gaseous medium (e.g. a medical air, a gaseous analgesic, a gaseous anesthetic, medical aerosols in a gaseous phase and other medical gases specific to a particular disease related to the mammal hollow organ), the liquid medium (e.g. water, a liquid contrast agent, an irrigating liquid solution, a sterile saline, a liquid anesthetic, a liquid analgesic, liquid medications/medicine/drugs, a medical suspension, a medical emulsion and other medical liquids specific to a particular disease related to the mammal hollow organ) or the fluid medium previously supplied to the interior of the isolated target section of the mammal hollow organ to penetrate, flow or pass through the net or enclosure 16 and also allows said passed medium to be evacuated, by means of the second functional device (not shown), from the interior of the isolated target section of the mammal hollow organ to the functional channel 3 via the functional opening 7.

Moreover, the strained net or enclosure 16 shown in FIGS. 1.17-1.19 prevents the mucosal tissue of the inner wall of the isolated target section of the mammal hollow organ from glueing or adhering to the functional opening 7, thereby constantly allowing the aspiration or the evacuation of the gaseous medium, the liquid medium or the fluid medium (i.e. the gas, the liquid or the fluid) from the interior of the isolated target section of the mammal hollow organ without causing damage to the mucosal tissue of the inner wall of the isolated target section of the mammal hollow organ and without blocking or occluding the functional opening 7 with the mucosal tissue of the inner wall of the isolated target section of the mammal hollow organ. In other words, the strained net or enclosure 16 prevents suction of the mucosal tissue of the inner wall of the isolated target section of the mammal hollow organ into the functional opening 7, while allowing the gaseous medium, the liquid medium or the fluid medium available in the interior of the isolated target section of the mammal hollow organ to be aspirated or evacuated by the second functional device (not shown) from the interior of the isolated target section of the mammal hollow organ. Therefore, the net or enclosure 16 further improves the safety of the catheter 100.

It is to note that the net or enclosure 16 and the ring-shaped projections 6 are intended separately or in combination to improve the safety of the catheter 100.

It is to further note that the net or enclosure 16 in the strained state shown in FIGS. 1.17-1.19 redistributes the pressure applied by the inflated isolating balloons 4 to the inner wall of the mammal hollow organ for isolating the interior of the target section of the mammal hollow organ between the inflated isolating balloons 4 and, therefore, prevents lesions of the inner wall of the mammal hollow organ from being caused by the pressure applied by the inflated isolating balloons 4 or prevents damages to the inner wall of the mammal hollow organ from being caused by the pressure applied by the inflated isolating balloons 4, thereby further improving the safety of the catheter 100. Additionally, the net or enclosure 16 in the strained state pulverizes or sprays the gaseous medium, the liquid medium or the fluid medium (i.e. the gas, the liquid or the fluid) supplied by the second functional device (not shown) into the interior of the isolated target section of the mammal hollow organ and, therefore, prevents the mucosal tissue of the inner wall of the isolated target section of the mammal hollow organ from being intensively effected by a flow of the supplied gaseous medium, the supplied liquid medium or the supplied fluid medium that would otherwise damage the effected mucosal tissue, thereby further improving the safety of the catheter 100.

In an alternative embodiment of the present invention, the net or enclosure 16 having a strained state or at least partly strained state is preliminary engaged or secured on both the ring-shaped projections 6 (i.e. the annular protrusions 15 are not provided on the elongate catheter body of the catheter 100, and both the isolating balloons 4 are not enclosed by the net or enclosure 16) such that the net or enclosure 16 completely or at least partly encloses a catheter body part defined by the ring-shaped projections 6 (i.e. a catheter body part located between the ring-shaped projections 6 and comprising the functional opening 7 provided in said catheter body part), thereby completely or at least partly enveloping or covering the functional opening 7. In the present alternative embodiment of the present invention, the strained net or enclosure 16 engaged or secured on both the ring-shaped projections 6 allows a predetermined distance between the mucosal tissue of the inner wall of the mammal hollow organ, the mucosal tissue being in contact with the strained net or enclosure 16 in the isolated target section of the mammal hollow organ, and the functional opening 7 to be defined or set, wherein the predetermined distance depends at least on a tension degree or a strain level of the strained net or enclosure 16 and the radius of the ring-shaped projections 6.

It is to further note that the net or enclosure 16 and the ring-shaped projections 6 are intended separately or in combination to increase the reliability of the catheter 100. In particular, the net or enclosure 16 and/or the ring-shaped projections 6 prevent the functional opening 7 from being partially or completely blocked/occluded by the mucosal tissue sucked into the functional opening 7, thereby (i) preventing the inserted catheter 100 from being retrieved from the lumen of the mammal hollow organ and preventing a new catheter 100 from being subsequently inserted back into the lumen of the mammal hollow organ (i.e. in replacement of the retrieved catheter 100) or (ii) preventing the retrieved catheter 100 from being cleaned by removing the sucked mucosal tissue from the functional opening 7 of the retrieved catheter 100 and preventing the cleaned catheter 100 from being reinserted back into the lumen of the mammal hollow organ.

In some embodiments of the present invention, the elongate catheter body of the catheter 100 is provided only with the inflation channel 2 and the functional channel 3, wherein the main channel 1 is not provided within the elongate catheter body of the catheter 100.

According to an embodiment of the present invention, each of the two ring-shaped projections 6 used to enclose the functional opening 7 and provided on the elongate catheter body of the catheter 100 is further provided with a traverse groove or cut provided in each of the two ring-shaped projections 6 having a square form. Alternatively, in such embodiment of the present invention, the traverse groove or cut provided in each of the two ring-shaped projections 6 provided on the elongate catheter body of the catheter 100 has a rectangular form, circle form, triangle form, oval form or any another appropriate geometrical form known in the art. It is to note that the each of the two traverse grooves or cuts of the two ring-shaped projections 6 provided on the elongate catheter body of the catheter 100 in such embodiment of the present invention is designed such that the traverse groove or cut through the entire width of a corresponding one of the two ring-shaped projections 6, wherein the width is defined between an inner side of the ring-shaped projection 6, the inner side being faced to the functional opening 7, and an outer side of the ring-shaped projection 6, the outer side being faced to a corresponding one of the two isolating balloons 4. In such embodiment of the present invention, in case when the negative pressure is produced in the isolated interior of the mammal hollow organ by using the second functional device, the mucosal tissue of the inner wall of the isolated target section of the mammal hollow organ may be closely glued or adhered to both the ring-shaped projections 6 provided on the elongate catheter body of the catheter 100 around all the circumference of the two ring-shaped projections 6 or a part of the circumference of the two ring-shaped projections 6, thereby splitting or dividing the isolated interior of the mammal hollow organ into two separate spaces: (i) an inner space defined within the isolated interior of the mammal hollow organ by (i-a) the inner sides of the two ring-shaped projections 6, (i-b) the part of the elongate catheter body of the catheter 100 between the inner sides of the two ring-shaped projections 6 and (i-c) a portion of the mucosal tissue of the inner wall of the isolated target section of the mammal hollow organ, the portion of the mucosal tissue being closely glued or adhered to both the ring-shaped projections 6 around all the circumference of the two ring-shaped projections 6 or a part of the circumference of the two ring-shaped projections 6 and being positioned between the inner sides of the two ring-shaped projections 6, and (ii) an outer space defined within the isolated interior of the mammal hollow organ by (ii-a) the outer sides of the two ring-shaped projections 6, (ii-b) parts of the elongate catheter body of the catheter 100 between the outer sides of the two ring-shaped projections 6 and the two isolating balloons 4 and (ii-c) portions of the mucosal tissue of the inner wall of the isolated target section of the mammal hollow organ that are positioned between the outer sides of the two ring-shaped projections 6 and the two isolating balloons 4, wherein the inner space is generally surrounded by the outer space or within the outer space, and wherein both the inner space and the outer space in combination form the isolated interior of the mammal hollow organ. In other words, in such embodiment of the present invention, when the negative pressure is produced in the isolated interior of the mammal hollow organ by using the second functional device, the inner space and the outer space surrounding the inner space may be defined within isolated interior of the mammal hollow organ and separated from each other by the mucosal tissue being closely glued or adhered to both the ring-shaped projections 6 provided on the elongate catheter body of the catheter 100 around all the circumference of the two ring-shaped projections 6 or a part of the circumference of the two ring-shaped projections 6. Meanwhile, in such embodiment of the present invention, each of the two traverse grooves or cuts as provided in a corresponding one of the two ring-shaped projections 6 provided on the elongate catheter body of the catheter 100 is designed such that said traverse groove or cut defines a through passage or through channel which allows the gaseous media, liquid media or fluid media (including the biological contents) to be aspirated or evacuated through the defined through passage/channel from the outer space of the isolated interior of the mammal hollow organ. In other words, in such embodiment of the present invention, both the through passages or channels provided by the traverse grooves or cuts of the two ring-shaped projections 6 provided on the elongate catheter body of the catheter 100 separately or in combination allow the gaseous media, liquid media or fluid media (including the biological contents) to be to aspirated or evacuated not only from the inner space of the isolated interior of the mammal hollow organ, the inner space being generally isolated within the isolated interior of the mammal hollow organ between the two ring-shaped projections 6 when the mucosal tissue of the inner wall of the isolated target section of the mammal hollow organ is closely glued or adhered to both the ring-shaped projections 6 around all the circumference of the two ring-shaped projections 6 or a part of the circumference of the two ring-shaped projections 6, but also from the outer space of the isolated interior of the mammal hollow organ, the outer space being isolated by the glued or adhered mucosal tissue within the isolated interior of the mammal hollow organ between the two ring-shaped projections 6 and the two isolating balloons 4 when the mucosal tissue of the inner wall of the isolated target section of the mammal hollow organ is closely glued or adhered to both the ring-shaped projections 6 around all the circumference of the two ring-shaped projections 6 or a part of the circumference of the two ring-shaped projections 6. Thus, in such embodiment of the present invention, the through passages or channels provided by the traverse grooves or cuts of the two ring-shaped projections 6 provided on the elongate catheter body of the catheter 100 allow the gaseous media, liquid media or fluid media (including the biological contents) to be aspirated or evacuated from all the isolated interior of the mammal hollow organ even when the mucosal tissue of the inner wall of the isolated target section of the mammal hollow organ is closely glued or adhered to both the ring-shaped projections 6 around all the circumference of the two ring-shaped projections 6 or a part of the circumference of the two ring-shaped projections 6. In such embodiment of the present invention, both the through passages or channels defined by the traverse grooves or cuts of the two ring-shaped projections 6 provided on the elongate catheter body of the catheter 100 allow the above-described inner space of the isolated interior of the mammal hollow organ and the above-described outer space of the isolated interior of the mammal hollow organ to be in fluid communication with each other, thereby allowing the gaseous media, liquid media or fluid media (including the biological contents) to pass, transfer or flow through said through passages or channels from the outer space to the inner space within the isolated interior of the mammal hollow organ when the negative pressure is produced in the isolated interior of the mammal hollow organ by using the second functional device. In turn, the aspiration or evacuation of the gaseous media, liquid media or fluid media, e.g. the biological contents, from both the inner space and the outer space of the isolated interior of the mammal hollow organ through the through passages or channels provided by the traverse grooves or cuts of the two ring-shaped projections 6 provided on the elongate catheter body of the catheter 100 results in collection of the biological contents related to all the isolated interior of the mammal hollow organ in the storage container of the aspirator, thereby increasing the accuracy and/or the reliability of the laboratory examinations to be performed on the collected biological contents. It is to note that both the traverse grooves or cuts provided in the two ring-shaped projections 6 provided on the elongate catheter body of the catheter 100 and used to enclose the functional opening 7 in such embodiment of the present invention are aligned with the functional opening 7 along the elongate catheter body of the catheter 100, thereby minimizing a length of a pathway from the traverse groove or cut provided in any of the two ring-shaped projections 6 to the functional opening 7, the pathway being required to be overcome by the gaseous media, liquid media or fluid media (including the biological contents) when producing the negative pressure in the isolated interior of the mammal hollow organ by means of the second functional device.

According to one embodiment of the present invention, each of the two ring-shaped projections 6 provided on the elongate catheter body of the catheter 100 is provided with one or more traverse grooves or cuts (e.g. one, two, three, four, five, six, seven, eight, nine, ten or more traverse grooves or cuts).

According to another embodiment of the present invention, each of the two ring-shaped projections 6 provided on the elongate catheter body of the catheter 100 is provided with two or more traverse grooves or cuts (e.g. two, three, four, five, six, seven, eight, nine, ten or more traverse grooves or cuts) equally spaced around the circumference of said ring-shaped projection 6 or circumferentially spaced at predetermined intervals from each other. In such another embodiment of the present invention, the four or more traverse grooves or cuts provided in the two ring-shaped projections 6 provided on the elongate catheter body of the catheter 100 define correspondently four or more through passages or channels which may be used for aspirating or evacuating the gaseous media, liquid media or fluid media (e.g. the biological contents) from the outer space of the isolated interior of the mammal hollow organ. In other words, in such another embodiment of the present invention, the increase of the through passages or channels defined by the traverse grooves or cuts in each of the two ring-shaped projections 6 provided on the elongate catheter body of the catheter 100 simplifies the aspiration or evacuation of the gaseous media, liquid media or fluid media (e.g. the biological contents) from the outer space of the isolated interior of the mammal hollow organ when the mucosal tissue of the inner wall of the isolated target section of the mammal hollow organ is closely glued or adhered to both the ring-shaped projections 6 around all the circumference of the two ring-shaped projections 6 or a part of the circumference of the two ring-shaped projections 6 due to the negative pressure produced by the second functional device in the interior of the isolated target section of the mammal hollow organ.

According to still another embodiment of the present invention, each of the two ring-shaped projections 6 provided on the elongate catheter body of the catheter 100 is provided with two traverse grooves or cuts which are positioned radially/circumferentially opposite to each other.

According to other embodiments of the present invention, each of the two ring-shaped projections 6 provided on the elongate catheter body of the catheter 100 is provided with one or more through-holes (e.g. one, two, three, four, five, six, seven, eight, nine, ten or more through-holes) in addition to the traverse grooves or cuts or instead of them. It is to note that each of the through-holes (not shown) as provided in a corresponding one of the two ring-shaped projections 6 provided on the elongate catheter body of the catheter 100 in such other embodiments of the present invention is designed such that said through-hole defines a further through passage or a further through channel which allows the gaseous media, liquid media or fluid media (including the biological contents) to be aspirated or evacuated through the defined through-holes from the outer space of the isolated interior of the mammal hollow organ when the mucosal tissue of the inner wall of the isolated target section of the mammal hollow organ is closely glued or adhered to both the ring-shaped projections 6 around all the circumference of the two ring-shaped projections 6 or a part of the circumference of the two ring-shaped projections 6. In other words, in such other embodiments of the present invention, the through passages or channels provided by the traverse grooves or cuts of the two ring-shaped projections 6 provided on the elongate catheter body of the catheter 100 and/or the further through passages or channels provided by the through-holes (not shown) of the two ring-shaped projections 6 provided on the elongate catheter body of the catheter 100 separately or in combination allow the gaseous media, liquid media or fluid media (including the biological contents) to be to aspirated or evacuated from both the inner space of the isolated interior of the mammal hollow organ and the outer space of the isolated interior of the mammal hollow organ, i.e. all the isolated interior of the mammal hollow organ, when the mucosal tissue of the inner wall of the isolated target section of the mammal hollow organ is closely glued or adhered to both the ring-shaped projections 6 around all the circumference of the ring-shaped projections 6 or a part of the circumference of the two ring-shaped projections 6. In such other embodiments of the present invention, the through passages or channels provided by the traverse grooves or cuts of the two ring-shaped projections 6 provided on the elongate catheter body of the catheter 100 and/or the further through passages or channels provided by the through-holes (not shown) of the two ring-shaped projections 6 provided on the elongate catheter body of the catheter 100 allow the above-described inner space of the isolated interior of the mammal hollow organ and the above-described outer space of the isolated interior of the mammal hollow organ to be in fluid communication with each other, thereby allowing the gaseous media, liquid media or fluid media (including the biological contents) to pass, transfer or flow through said through passages or channels of the two ring-shaped projections 6 and/or said further through passages or channels of the two ring-shaped projections 6 from the outer space to the inner space within the isolated interior of the mammal hollow organ when the mucosal tissue of the inner wall of the isolated target section of the mammal hollow organ is closely glued or adhered to both the ring-shaped projections 6 around all the circumference of the ring-shaped projections 6 or a part of the circumference of the two ring-shaped projections 6 due to the negative pressure produced in the isolated interior of the mammal hollow organ by means of the second functional device. In turn, in such other embodiments of the present invention, the aspiration or evacuation of the gaseous media, liquid media or fluid media, e.g. the biological contents, from both the inner space and the outer space of the isolated interior of the mammal hollow organ through the through passages or channels provided by the traverse grooves or cuts of the two ring-shaped projections 6 provided on the elongate catheter body of the catheter 100 and/or the further through passages or channels provided by the through-holes (not shown) of the two ring-shaped projections 6 provided on the elongate catheter body of the catheter 100 results in collection of the biological contents related to all the isolated interior of the mammal hollow organ in the storage container of the aspirator, thereby increasing the accuracy and/or the reliability of the laboratory examinations to be performed on the collected biological contents.

According to some embodiments of the present invention, each of the two ring-shaped projections 6 provided on the elongate catheter body of the catheter 100 is further provided with a through-hole (not shown), wherein both the through-holes provided in the two ring-shaped projections 6 enclosing the functional opening 7 are aligned with the functional opening 7 along the elongate catheter body of the catheter 100, thereby minimizing a length of a pathway from the through-hole provided in any of the two ring-shaped projections 6 to the functional opening 7, the pathway being required to be overcome by the gaseous media, liquid media or fluid media (including the biological contents) when producing the negative pressure in the isolated interior of the mammal hollow organ by means of the second functional device.

According to some other embodiments of the present invention, each of the two ring-shaped projections 6 provided on the elongate catheter body of the catheter 100 is further provided with one or more through-holes (e.g. one, two, three, four, five, six, seven, eight, nine, ten or more through-holes).

According to various embodiments of the present invention, each of the two ring-shaped projections 6 provided on the elongate catheter body of the catheter 100 is provided with two or more through-holes (e.g. two, three, four, five, six, seven, eight, nine, ten or more through-holes) equally spaced around the circumference of said ring-shaped projection 6 or circumferentially spaced at predetermined intervals from each other. In such various embodiments of the present invention, the four or more through-holes provided in the two ring-shaped projections 6 provided on the elongate catheter body of the catheter 100 define correspondently four or more through passages or channels which may be used for aspirating or evacuating the gaseous media, liquid media or fluid media (e.g. the biological contents) from the outer space of the isolated interior of the mammal hollow organ. In other words, in such various embodiments of the present invention, the increase of the through passages or channels defined by the through-holes in each of the two ring-shaped projections 6 provided on the elongate catheter body of the catheter 100 simplifies the aspiration or evacuation of the gaseous media, liquid media or fluid media (e.g. the biological contents) from the outer space of the isolated interior of the mammal hollow organ when the mucosal tissue of the inner wall of the isolated target section of the mammal hollow organ is closely glued or adhered to both the ring-shaped projections 6 around all the circumference of the ring-shaped projections 6 due to the negative pressure produced by the third functional device in the isolated interior of the mammal hollow organ.

According to still another embodiment of the present invention, each of the two ring-shaped projections 6 provided on the elongate catheter body of the catheter 100 is provided with two through-holes which are positioned radially/circumferentially opposite to each other.

Depending on a particular mammal hollow organ having the elongate catheter body of the catheter 100 inserted into the lumen of the particular mammal hollow organ, a length of the elongate catheter body of the catheter 100, a diameter of the elongate catheter body of the catheter 100, a wall thickness of the elongate catheter body of the catheter 100, locations of the isolating balloons 4 on the elongate catheter body of the catheter 100, diameters, sizes or volumes of the isolating balloons 4 in expanded/inflated states, a location of the functional opening 7 in the elongate catheter body of the catheter 100, a diameter of the functional opening 7, locations of the ring-shaped projections 6 on the elongate catheter body of the catheter 100, diameters of the ring-shaped projections 6, locations of the auxiliary holes 5-1, 5-2 in the elongate catheter body of the catheter 100 and/or diameters of the auxiliary holes 5-1, 5-2 may be preliminary optimized/modified/adjusted/adapted to particular anatomical parameters of the particular mammal hollow organ. Thus, any parameter of the catheter 100 can be easily optimized/modified/adjusted/adapted by one skilled in the art (e.g. the user) to particular parameters of the particular mammal hollow organ (e.g. particular dimensions of the particular mammal hollow organ).

The thickness of the main channel 1, the inflation channel 2 and the functional channel 3 provided in the elongate catheter body of the catheter 100 and the size of the isolating balloons 4 may be adjusted by one skilled in the art on the basis of information disclosed in the art, for example, U.S. Pat. Nos. 9,526,874, 6,692,465, 5,843,050, 5,919,163, international publication WO 2009/035581, U.S. Pat. Nos. 5,397,305, 8,398,589, 7,722,568, 6,712,798, 6,638,245, 1,009,865 and/or other prior art information sources.

The length of the elongate catheter body of the catheter 100, a distance between the isolating balloons 4 provided on the elongate catheter body, and a distance from the isolating balloons 4 to the distal end of the elongate catheter body may be preliminary matched by one skilled in the art on the basis of information disclosed in the art, for example, U.S. Pat. Nos. 5,314,409, 5,658,264, U.S. patent application No. 20150150572, U.S. Pat. Nos. 5,843,050, 5,397,305, 7,070,606, 6,712,798, 1,009,865 and/or other prior art information sources.

For example, in one of the preferable embodiments of the present invention, the catheter 100 may be implemented as a pancreatodigestive catheter having a length of 130 cm. In the present preferable embodiment of the present invention, the two isolating balloons 4 provided on an elongate catheter body of the pancreatodigestive catheter are correspondingly spaced at 15 cm and 25 cm from a distal end of the elongate catheter body of the pancreatodigestive catheter, the distal end being used for inserting the elongate catheter body of the pancreatodigestive catheter into a gastric cavity and a lumen of a mammal duodenum, so that the distance between the two isolating balloons 4 is equal to 10 cm. Thus, the expansion or inflation of the isolating balloons 4 allows an interior of the mammal duodenum, the interior corresponding to the distance between the two isolating balloons 4, to be isolated between the inflated isolating balloons 4. In the present preferred embodiment of the present invention, the functional opening 7 may be provided in the elongate catheter body of the pancreatodigestive catheter in the middle between the isolating balloons 4, i.e. at a distance of 5 cm from each of the isolating balloons 4 and at a distance of 20 cm from the distal end of the elongate catheter body of the pancreatodigestive catheter.

Use of the catheter 100 is illustratively described above in relation to the mammal duodenum. However the use of the catheter 100 is not limited by the mammal duodenum. Therefore, it is clear for one skilled in the art that the elongate catheter body of the catheter 100 according to any of the above-described embodiments can be similarly inserted into a lumen of any another mammal hollow (tubular) internal organ, for example into the esophagus, stomach, duodenum, small intestine, large intestine, respiratory tracts, urinary tracts (urogenital system tracts), veins, arteries, vagina, uterus, uterine (Fallopian) tubes, vertebral canal or any appropriate mammal internal tubular organ related to any functional system (an apparatus of organs) of the mammal organism, the functional system being selected from a group of mammal functional systems including the following mammal functional systems: digestive system, respiratory system, urinary and reproductive systems (combined into the genitourinary system or urogenital system), endocrine system, circulatory system and immune system, and skeletal system.

Therefore, the elongate catheter body of the catheter 100 according to the present invention may be inserted by the user into the lumen of the mammal duodenum under control of an endoscope (not shown) or under control of a radiographic equipment (for example, a fluoroscopy equipment) such that one of the two isolating balloons 4, the farthest from the distal end of the elongate catheter body of the catheter 100, is located in the bulb of the mammal duodenum, wherein the endoscope may be manipulated by the user or an endoscopist assisting the user. In particular, it is to note that the insertion process of the elongate catheter body of the catheter 100 into the lumen of the mammal duodenum, the aspiration process and the removal process of the elongate catheter body of the catheter 100 from the lumen of the mammal duodenum after sampling or taking a required amount the liquid media, gaseous media or fluid media from the isolated interior of the mammal duodenum are atraumatic due to the above-described modifications or improvements to the catheter 100 and do not depend on any anatomical features of both the mammal hollow organ and neoplasms. It is to further note that the two isolating balloons 4 are both in a deflated state when the elongate catheter body of the catheter 100 is inserted by the user into the lumen of the mammal duodenum.

According to one example, the elongate catheter body of the catheter 100 may be preliminary equipped with loop(s) or thread(s) designed to be gripped with endoscope biopsy forceps for capturing the elongate catheter body of the catheter 100. To insert the elongate catheter body of the catheter 100 into the lumen of the mammal duodenum to have a desired placement site in the mammal duodenum, the distal end of the elongate catheter body of the catheter 100, when preliminary well lubricated with a vaseline oil, is administered or inserted through the mammal nasal passage and advanced to the mammal gastric cavity; then the endoscope is further administered or advanced in parallel with the catheter 100 through the mammal oral cavity to the mammal gastric cavity so as to grip, by means of the endoscope biopsy forceps, the loop(s) or the thread(s) of the elongate catheter body of the catheter 100 for capturing the elongate catheter body of the catheter 100; the captured elongate catheter body is finally guided or advanced along with the endoscope to the mammal duodenum. Subsequently, one of the two isolating balloons 4 of the catheter 100, the farthest from the distal end of elongate catheter body of the catheter 100, is placed in the bulb of the mammal duodenum under control of the endoscope.

According to another example, a metal guidewire may be preliminary administered or inserted into the main channel 1 provided in the elongate catheter body of the catheter 100. To insert the elongate catheter body of the catheter 100 into the lumen of the mammal duodenum to have a desired placement site in the mammal duodenum, the distal end of the elongate catheter body of the catheter 100, when preliminary well lubricated with a vaseline oil, is administered or inserted through the mammal nasal passage and then advanced to the mammal gastric cavity by using the inserted metal guidewire; then an endoscope provided with biopsy forceps is inserted or advanced in parallel with the catheter 100 through the mammal oral cavity to the mammal gastric cavity to grip, by means of the endoscope biopsy forceps, a first ligature upon detection of the distal end of the elongate catheter body of the catheter 100 and to press the catheter 100 to the endoscope by pulling up the elongate catheter body of the catheter 100 by using the gripped first ligature. Subsequently, the endoscope and the catheter 100 pressed against the endoscope are guided through the mammal pylorus to the mammal duodenum, and one of the two isolating balloons 4, the farthest from the distal end of the elongate catheter body of the catheter 100, is placed within the bulb of the mammal duodenum under control of the endoscope.

Then, the user connects the above-described third functional device (not shown) to the side terminal 10 of the connector 12 for providing the fluid communication between the inner channel of the side terminal 10 and the inflation channel 2, and the connected third functional device is activated by the user for supplying the gaseous medium (e.g. air, another appropriate gas or another appropriate gas mixture) or the liquid medium (e.g. water, another appropriate liquid substance or another appropriate liquid solution) in a predetermined amount or a predetermined volume under a predetermined pressure to the inflation channel 2, in particular for supplying water in a volume of 40-100 ml, to allow both the isolating balloons 4 to be expanded and inflated with the supplied medium such that they both closely adjoin to the mucosal tissue of the inner wall of the mammal duodenum, thereby isolating a required interior of the mammal duodenum between the inflated isolating balloons 4, the isolated interior containing the major duodenal papilla and the minor duodenal papilla, and preventing gastric contents and/or duodenal contents with inactive digestive ferments from entering the isolated interior of the mammal duodenum. In particular, it is to note that results experimentally obtained by the inventors demonstrate that 40-100 ml of air is required to have both the isolating balloons 4 sufficiently inflated to a required size for providing the bilateral obturation or occlusion of the lumen of the mammal duodenum, the lumen having the elongate catheter body of the catheter 100 inserted thereinto, with the two inflated isolating balloons 4.

Then, the user initially connects the above-described second functional device (not shown) to the side terminal 8, while the inner channel of the side terminal 8 is in fluid communication with the functional channel 3, and the functional channel 3 is in fluid communication with the isolated interior of the mammal duodenum via the functional opening 7, wherein the second functional device connected to the side terminal 8 is implemented as the above-mentioned medical suction apparatus, the above-mentioned aspiration device or the above-mentioned aspirator preliminary configured to operate in a constant or variable operation mode for generating a required working pressure corresponding to a standard or natural pressure being specific to the mammal duodenum. The aspirator is then activated by the user to allow air in the isolated interior of the mammal duodenum, the isolated interior corresponding to a periampullary region of the mammal duodenum, to be evacuated or aspirated from the isolated interior under the generated working pressure via the functional opening 7, wherein the aspirated air is then transferred via the functional channel 3 to the inner channel of the side terminal 8 and finally transferred via the inner channel of the side terminal 8 to the atmosphere. Evacuation or aspiration of air from the isolated interior of the mammal duodenum allows a negative pressure, the negative pressure corresponding to a physiological negative pressure normally created by the peristalsis of the mammal duodenum (in particular, a negative pressure of 40-100 mmH2O or 9-14 kPa), to be produced in the isolated interior of the mammal duodenum. The negative pressure produced in the isolated interior of the mammal duodenum allows a biological liquid medium in the form of pancreatic juice or pancreas secretion in combination with bile to be evacuated or retrieved from the pancreas through the major duodenal papilla and the minor duodenal papilla, thereby sampling or taking said biological liquid from the isolated interior of the mammal duodenum via the functional opening 7 and via the functional channel 3 and subsequently collecting the taken biological liquid in the storage container (not shown) of the aspirator. In some examples, a mucosal secretion or the contents of the bile and pancreatic ducts, the mucosal secretion being one of varieties of the biological liquid being specific to the mammal duodenum, and/or some biological contents which could enter the bile and the pancreatic ducts as a result of reflux, and would contain mammal liquids produced by the mammal organism, the mammal liquids being specific to the mammal duodenum, may be further collected in the storage container of the aspirator. The biological liquid collected in the storage container of the aspirator (not shown) can be subsequently delivered to a laboratory for cytological examination, molecular genetic examination and/or other appropriate laboratory examinations to evaluate characteristics of a morphological structure of cellular elements of the pancreatic secretion, thereby allowing pathologic processes being specific to intraductal neoplasms, neuroendocrine tumors or pancreatic cancers to be detected or revealed, allowing neoplasms to be detected at an early development stage and allowing differential diagnosis between different types of tumors to be performed by determining expressions of specific markers (e.g. NKX2, S100P, CEA, EFR3A/B, MUC1, MUC2, MUC5, ANXA1, A2, KRT7, MMP7, MMP9, IGFBP3, PSCA, PRSS2, SHh, KRas, TP53, SMAD4, BRCA1, miRNA 21, and miRNA 155).

Furthermore, after disconnecting the aspirator from the side terminal 8, the above-mentioned medium supply device for supplying the liquid medium, the gaseous medium or the fluid medium (i.e. the combination of a liquid medium and a gaseous medium) under the pressure (for example, the injection syringe, the medical dropper or any other appropriate automated or semi-automated appliances or devices) may be connected by the user to the side terminal 8 instead of the disconnected aspirator, while the inner channel of the side terminal 8 is in fluid communication with the functional channel 3, and the functional channel 3 is in fluid communication with the isolated interior of the mammal duodenum via the functional opening 7. The medium supply device is then activated by the user to allow a required amount or volume of the liquid medium, gaseous medium or fluid medium to be delivered to the isolated interior of the mammal duodenum via the functional opening 7 (e.g. a liquid drug or a gaseous drug for treating the mucosal tissue of the inner wall of the mammal duodenum in the isolated interior of the mammal duodenum).

Alternatively, instead of using the above-described aspirator, the user may initially connect the above-mentioned medium supply device for supplying the liquid medium, the gaseous medium or the fluid medium (i.e. the combination of a liquid medium and a gaseous medium) under the pressure (for example, the injection syringe, the medical dropper or any other appropriate automated or semi-automated appliances or devices) to the side terminal 8, while the inner channel of the side terminal 8 is in fluid communication with the functional channel 3, and the functional channel 3 is in fluid communication with the isolated interior of the mammal duodenum via the functional opening 7. The medium supply device is then activated by the user to allow the supplied media to be delivered to the isolated interior of the mammal duodenum, the isolated interior corresponding to a periampullary region of the mammal duodenum, under a working pressure generated by the medium supply device (e.g. a liquid drug or a gaseous drug for treating the mucosal tissue of the inner wall of the mammal duodenum in the isolated interior of the mammal duodenum), wherein the supplied liquid medium, gaseous medium or fluid medium is initially supplied from the medium supply device to the inner channel of the side terminal 8, then transferred via the inner channel of the side terminal 8 to the functional channel 3 and finally transferred from the functional channel 3 via the functional opening 7 to the isolated interior. For example, a liquid drug or a gaseous drug may be supplied by the medium supply device to the isolated interior of the mammal duodenum for treating the mucosal tissue of the inner wall of the mammal duodenum in the isolated interior of the mammal duodenum.

Furthermore, after disconnecting the medium supply device from the side terminal 8, the aspirator may be connected by the user to the side terminal 8, thereby allowing the liquid medium, gaseous medium or fluid medium (e.g. the liquid drug or the gaseous drug) previously supplied by the medium supply device to the isolated interior of the mammal duodenum to be sampled or taken from the isolated interior and subsequently collected in the storage container (not shown) of the aspirator.

It is to note that the catheter 100 may remain inserted into the mammal duodenum for a sufficiently long period of time, for example up to seven days (i.e. up to 168 hours), to allow the pancreatic secretion in an appropriate amount to be collected by the catheter 100 for performing the reliable identification and verification of a pathologic tumor material, thereby generally improving the efficiency of diagnosis and subsequently treating the pancreatic cancer. Furthermore, the prolonged sampling increases the likelihood of detecting pathologic cells and other biomarkers not only for pancreatic cancers, but also for malignant conditions of bile ducts and liver, including cholangiocarcinoma and/or hepatocellular cancer.

It is to further note that artificial stimulation of the secretion of pancreatic juice and bile is not required when using the catheter 100, so that the biological contents collected by the catheter 100 has a true biochemical and physiological profile presenting a true functional state of pancreas, liver, bile ducts, contractility of the gallbladder.

If necessary, the excretory function of pancreas may be evaluated by examining a biochemical composition of the pancreatic secret collected together with the bile in the above-described manner in the storage container of the aspirator by using the catheter 100 according to the present invention.

Furthermore, a qualitative and quantitative analysis of the pancreatic juice and bile allows a true biochemical profile of both the biological materials and a volume of daily secretion to be evaluated, thereby diagnosing a functional condition of an organ.

The catheter 100 according to any one of the above-described embodiments of the present invention may be used for diagnosing or monitoring the development of at least one disease selected from a group comprising: gastritis, pancreatitis, pancreatic cancer, bile duct cancer, cholangiocarcinoma, hepatocellular cancer, cholangitis, cholelithiasis, wall defects of a hollow organ wall, autoimmune hepatitis, infectious hepatitis, aneurismal or diverticular protrusions of a hollow organ wall, bleeding to a hollow organ interior, strictures of mammal hollow organs and esophagus neuromuscular disorders, stomach, the duodenum, small intestine and large intestine, bile ducts, urinoexcretory ways and respiratory tracts.

Furthermore, the catheter 100 according to any one of the above-described embodiments of the present invention may be used for treating at least one disease selected from a group comprising: pancreatitis, cholangitis, gastrointestinal wall defects (e.g. fistulas), gastrointestinal wall injuries or traumas, vascular aneurisms, venous thrombs, ureter defects, respiratory tract defects, gastrointestinal hemorrhages, bleeding from the windpipe (trachea), bronchial bleeding, pulmonary hemorrhage, and uterine bleeding.

Furthermore, the catheter 100 according to any one of the above-described embodiments of the present invention may be used to isolate an interior of a mammal vessel when performing a surgical procedure or performing surgical manipulations, in particular when performing a surgical procedure or performing surgical manipulations in relation to tissues of an inner wall of the mammal vessel.

In particular, in an example, when the elongate catheter body of the catheter 100 is inserted into the lumen of the mammal duodenum, a treatment effect or a therapeutic effect may be achieved by restoring and facilitating the evacuation of bile or pancreatic juice from corresponding ducts into the mammal duodenum. The pathogenesis of acute inflammatory diseases, such as pancreatitis and cholangitis, is caused at least partly by the impairment or disruption of the outflow of pancreatic juice and bile, the outflow impairment being conditioned, in particular, by impaired motility (peristalsis) of the mammal duodenum, so that the isolated interior with a controllable negative pressure (for example, a negative pressure at level of 40-100 mmH2O) therein produced by the aspirator allows both the pancreatic juice and the bile to be sampled or taken from the isolated interior, thereby solving this medical problem and contributing to the treatment of the above acute diseases of the mammal duodenum.

In another example, when the elongate catheter body of the catheter 100 is inserted into the lumen of the mammal duodenum, a treatment or therapeutic effect may be achieved by evacuating biological contents related to an area of a wall defect of the mammal gastrointestinal tract, the area being within the isolated interior of the mammal duodenum, so that the isolated interior having a controllable negative pressure (for example, a negative pressure at level of 70-100 mmH2O) produced therein by the aspirator allows, for example, all the biological contents (e.g. biological liquids) related to the area of the wall defect to be sampled or taken from the isolated interior, thereby solving this medical problem and further contributing to the treatment of the wall defect.

It is clear for one skilled in the art that the catheter 100 may be made of any suitable materials on the basis of information disclosed in prior art documents, for example, in U.S. Pat. No. 7,722,568 or U.S. Pat. No. 6,638,245, wherein the catheter 100 may be preferably made of polymeric materials.

According to one embodiment of the present invention, each of the two ring-shaped projections 6 used to enclose the functional opening 7 and provided on the elongate catheter body of the catheter 100 may be further provided with a traverse groove, slot or cut (not shown) having a generally square form. In an alternative of the present embodiment of the present invention, the traverse groove, slot or cut (not shown) in the catheter 100 may have a generally rectangular form, a generally circle form, a generally triangle form, a generally oval form or any another appropriate geometrical form known in the art. In the present embodiment of the present invention, each of the two traverse grooves, slots or cuts of the two ring-shaped projections 6 provided on the elongate catheter body of the catheter 100 is designed such that said traverse groove, slot or cut extends through the entire width of a corresponding one of the two ring-shaped projections 6, wherein said width is defined between an inner side of the ring-shaped projection 6, the inner side being faced to the functional opening 7, and an outer side of the ring-shaped projection 6, the outer side being faced to a corresponding one of the two isolating balloons 4. Thus, in the present embodiment of the present invention, in case when the negative pressure is produced in the isolated interior of the mammal hollow organ by using the second functional device, the mucosal tissue of the inner wall of the isolated target section of the mammal hollow organ may be closely glued or adhered to both the ring-shaped projections 6 provided on the elongate catheter body of the catheter 100 around all the circumference of the two ring-shaped projections 6 or a part of the circumference of the two ring-shaped projections 6, thereby splitting or dividing the isolated interior of the mammal hollow organ into two separate spaces: (i) an inner space defined within the isolated interior of the mammal hollow organ by (i-a) the inner sides of the two ring-shaped projections 6, (i-b) the part of the elongate catheter body of the catheter 100 between the inner sides of the two ring-shaped projections 6 and (i-c) a portion of the mucosal tissue of the inner wall of the isolated target section of the mammal hollow organ, the portion of the mucosal tissue being closely glued or adhered to both the ring-shaped projections 6 around all the circumference of the two ring-shaped projections 6 or a part of the circumference of the two ring-shaped projections 6 and being positioned between the inner sides of the two ring-shaped projections 6, and (ii) an outer space defined within the isolated interior of the mammal hollow organ by (ii-a) the outer sides of the two ring-shaped projections 6, (ii-b) parts of the elongate catheter body of the catheter 100 between the outer sides of the two ring-shaped projections 6 and the two isolating balloons 4 and (ii-c) portions of the mucosal tissue of the inner wall of the isolated target section of the mammal hollow organ that are positioned between the outer sides of the two ring-shaped projections 6 and the two isolating balloons 4, wherein the inner space is generally surrounded by the outer space or within the outer space, and wherein both the inner space and the outer space in combination form the isolated interior of the mammal hollow organ. In other words, in the present embodiment of the present invention, when the negative pressure is produced in the isolated interior of the mammal hollow organ by using the second functional device, the inner space and the outer space surrounding the inner space may be defined within isolated interior of the mammal hollow organ and separated from each other by the mucosal tissue being closely glued or adhered to both the ring-shaped projections 6 provided on the elongate catheter body of the catheter 100 around all the circumference of the two ring-shaped projections 6 or a part of the circumference of the two ring-shaped projections 6. Also, in the present embodiment of the present invention, each of the two traverse grooves, slots or cuts (not shown), said traverse groove, slot or cut being provided in a corresponding one of the two ring-shaped projections 6 provided on the elongate catheter body of the catheter 100, is designed such that said traverse groove, slot or cut defines a through passage or through channel which allows the gaseous media, liquid media or fluid media (including the biological contents) to be aspirated or evacuated through the defined through passage/channel from the outer space of the isolated interior of the mammal hollow organ. In other words, in the present embodiment of the present invention, both the through passages or channels provided by the traverse grooves, slots or cuts of the two ring-shaped projections 6 provided on the elongate catheter body of the catheter 100 may separately or in combination allow the gaseous media, liquid media or fluid media (including the biological contents) to be to aspirated or evacuated not only from the inner space of the isolated interior of the mammal hollow organ, the inner space being generally isolated within the isolated interior of the mammal hollow organ between the two ring-shaped projections 6 when the mucosal tissue of the inner wall of the isolated target section of the mammal hollow organ is closely glued or adhered to both the ring-shaped projections 6 around all the circumference of the two ring-shaped projections 6 or a part of the circumference of the two ring-shaped projections 6, but also from the outer space of the isolated interior of the mammal hollow organ, the outer space being generally isolated within the isolated interior of the mammal hollow organ between the two ring-shaped projections 6 and the two isolating balloons 4 when the mucosal tissue of the inner wall of the isolated target section of the mammal hollow organ is closely glued or adhered to both the ring-shaped projections 6 around all the circumference of the two ring-shaped projections 6 or a part of the circumference of the two ring-shaped projections 6. Thus, in the present embodiment of the present invention, the through passages or channels provided by the traverse grooves, slots or cuts of the two ring-shaped projections 6 provided on the elongate catheter body of the catheter 100 allow the gaseous media, liquid media or fluid media (including the biological contents) to be aspirated or evacuated from all the isolated interior of the mammal hollow organ even when the mucosal tissue of the inner wall of the isolated target section of the mammal hollow organ is closely glued or adhered to both the ring-shaped projections 6 around all the circumference of the two ring-shaped projections 6 or a part of the circumference of the two ring-shaped projections 6. Also, in the present embodiment of the present invention, both the through passages or channels defined by the traverse grooves, slots or cuts 17 of the two ring-shaped projections 6 provided on the elongate catheter body of the catheter 100 allow the above-described inner space of the isolated interior of the mammal hollow organ and the above-described outer space of the isolated interior of the mammal hollow organ to be in fluid communication with each other, thereby allowing the gaseous media, liquid media or fluid media (including the biological contents) to pass, transfer or flow through said through passages or channels from the outer space to the inner space within the isolated interior of the mammal hollow organ when the negative pressure is produced in the isolated interior of the mammal hollow organ by using the second functional device. In turn, in the present embodiment of the present invention, the aspiration or evacuation of the gaseous media, liquid media or fluid media, e.g. the biological contents, from both the inner space and the outer space of the isolated interior of the mammal hollow organ through the through passages or channels provided by the traverse grooves, slots or cuts 17 of the two ring-shaped projections 6 provided on the elongate catheter body of the catheter 100 results in collection of the biological contents related to all the isolated interior of the mammal hollow organ in the storage container of the aspirator, thereby increasing the accuracy and/or the reliability of the laboratory examinations to be performed on the collected biological contents. Besides, in the present embodiment of the present invention, both the traverse grooves, slots or cuts (not shown) provided in the two ring-shaped projections 6 provided on the elongate catheter body of the catheter 100 and used to enclose the functional opening 7 are aligned with the functional opening 7 along the elongate catheter body of the catheter 100, thereby minimizing a length of a pathway from the traverse groove, slot or cut 17 provided in any of the two ring-shaped projections 6 to the functional opening 7, the pathway being required to be overcome by the gaseous media, liquid media or fluid media (including the biological contents) when producing the negative pressure in the isolated interior of the mammal hollow organ by means of the second functional device. In another alternative of the present embodiment of the present invention, each of the two ring-shaped projections 6 provided on the elongate catheter body of the catheter 100 may be provided with two or more traverse grooves, slots or cuts (e.g., two, three, four, five, six, seven, eight, nine, ten or more traverse grooves, slots or cuts) radially/circumferentially spaced from each other (e.g., equally spaced around the circumference of said ring-shaped projection 6 or circumferentially spaced around ring-shaped projection 6 at predetermined intervals from each other).

### EXAMPLES

Example No. 1. A patient No. 1 was admitted with girdle pain in the upper abdomen. Amylase in a blood was 1150 U/L. Results of ultrasound examination were as follows: pancreas was increased in size; the head was 35 mm; the catheter body was 32 mm; the tail was 21 mm; strongly heterogeneous; the contour was blurred; the parapancreatic tissue was edematous; liquids were not detected. The MSCT picture corresponds to acute hydropic pancreatitis. The patient started a therapy according to recommendations of the Russian Society of Surgeons, wherein the catheter 100 according to the present invention was inserted into the patient duodenum for 24 hours, and an active aspiration method was performed. After 24 hours, the level of amylase in a blood dropped to 230 U/L, and the pain syndrome was managed.

Example No. 2. A patient No. 2 was admitted with a clinical picture of obstructive jaundice. The examination revealed an increased level of leukocytes up to 14.1×109, wherein a total bilirubin was 145 mmol/L, and a direct bilirubin was 113.2 mmol/L. Amylase in a blood was 57 U/L, alanine aminotransferase (ALT) was 391 U/L, and aspartate aminotransferase (AST) was 90 U/L. Results of ultrasound were as follows: multiple calculi up to 1.3 cm in diameter were detected in a gallbladder, and bile ducts were dilated up to 11 mm. Results of magnetic resonance imaging (MRI) were as follows: a shadow corresponding to a calculus of 6 mm was detected in the distal section of the common bile duct. Results of endoscopic retrograde cholangiopancreatography (ERCP) were as follows: a single 8 mm calculus was identified, and then the identified calculus was endoscopically removed. To prevent the acute pancreatitis the catheter 100 according to the present invention was inserted into a patient duodenum for 24 hours, and an active aspiration method was performed. In the postoperative period, the level of amylase in a blood did not increase more than 87 U/L.

Example No. 3. A patient No. 3 was admitted routinely for examination and treatment. Previously performed MSCT allowed the formation of an anomalous body in pancreas to be detected. Endoscopic ultrasound allowed the formation of an anomalous body having the size of 2 cm by 3 cm to be detected, while a fine-needle biopsy was technically impossible. To selectively sample a pancreatic juice, the catheter 100 according to the present invention was inserted into a patient duodenum. Adenocarcinoma cells and mutations in driver genes, exosomes and protein markers were detected in the sampled pancreatic juice thanks to cytological and molecular examinations.

Example No. 4. A patient No. 4 was admitted with a clinical picture of obstructive jaundice. Results of MRI: a block at the level of confluence of the lobar right and left hepatic ducts was revealed, wherein the block corresponds to the Klatskin tumor of type IIA. The catheter 100 according to the present invention was inserted into the patient duodenum, and active aspiration was performed. Abnormal cells and markers of malignant neoplasms were detected in the obtained bile thanks to cytological and molecular examinations.

Example No. 5. Patient No. 5 was diagnosed with chronic pancreatitis. Despite the gastroenterologist's therapy and prescribed enzymatic drugs taken by the patient, body mass reduction, defecation disorders, and diarrhea were observed. To evaluate the functional state of pancreas the catheter 100 according to the present invention was inserted into the patient duodenum for 24 hours, and an active aspiration was performed for obtaining a pancreatic juice. As a result of a biochemical test performed on the obtained pancreatic juice, decreased levels of alpha amylase and lipase were revealed, and also a decrease in the daily excretion of pancreatic juice and bile was revealed. As a result, the dose of enzymatic drugs taken by the patient was increased, and ursodeoxycholic acid drugs and bile substitutes were additionally prescribed.

Example No. 6. A patient No. 6 with jaundice was admitted to an infectious diseases clinic in the city. Results of physical examination showed an enlarged liver, wherein results of a biochemical blood test showed the following: total bilirubin was 329 mmol/L, direct bilirubin was 141 mmol/L, ALT was 1040 U/L, AST was 804 U/L. Serological analysis of blood for HCV antigens, HBsAg was negative. The catheter 100 according to the present invention was inserted into the patient, wherein 100 ml of bile was obtained as a result of active aspiration, and a high copy number of the HCV virus was detected by PCR analysis of bile.

Example No. 7. A patient No. 7 was treated in an infectious diseases clinic for the verified viral hepatitis. After the course of interferons, a repeated PCR analysis of blood did not reveal an active viral process. The catheter 100 according to the present invention was inserted into the patient, wherein 200 ml of bile was obtained as a result of active aspiration, and the PCR analysis of the bile obtained showed an ongoing viral process, so that the scheme and duration of therapy were changed.

Example No. 8. A patient No. 8 was diagnosed with intestinal fistula and peritonitis. The operation, sanitation and drainage of the abdominal cavity were performed on an emergency basis. In order to isolate an intestinal wall defect, reduce its contact with intestinal contents, improve the healing process, and also to provide an additional modality for treating peritonitis by separating abdominal cavity and pathological substrate, the catheter 100 according to the present invention was inserted into an interior related to an intestinal injury such that the isolating balloons 4 were located above and below the injury. After inflating the isolating balloons 4, the isolated interior was produced, and another channel was connected to a suction apparatus to provide sanitization of the interior related to the intestinal fistula to facilitate defect healing. Enteral nutrition was also administered to the patient through the catheter 100.

Example No. 9. A patient No. 9 with girdle pain in the abdomen was admitted to the hospital as an emergency. Based on the results of examination, the patient was diagnosed with acute pancreatitis, of moderate severity, according to the Atlanta classification. At the time of admission, the amylase level was 1320 U/L. The catheter 100 according to the present invention was inserted into the patient, wherein the functional channel 3 was connected to a pump for producing a controllable negative pressure, while enteral nutrition and medical products were administered to the patients through the same catheter 100. As a result, the dynamics of biochemical parameters became positive in accordance with the BISAP scale within 24 hours, and the patient recovered after 7 days.

Example No. 10. A patient No. 10 was admitted to the hospital as an emergency with pain in the right hypochondrium, hectic form of fever and periodic yellowing of the skin. The anamnesis showed that 3 months before the current hospitalization the patient underwent endoscopic manipulation in relation to the bile ducts in order to remove calculus from them. Ultrasound, MSCT showed no changes in the liver, ducts or the presence of abscesses. In order to diagnose cholangitis, the catheter 100 according to the present invention was inserted into the patient, and 50.0 ml of bile was sampled and subjected to bacteriological examination. As a result, the abundant growth of Klebsiella spp. was revealed, and its sensitivity to antibacterial drugs was determined. After the selection of pathognomonic antibiotic therapy, the cholangitis symptoms disappeared, and the patient was discharged for an outpatient treatment.

Example No. 11. Patient No. 11 underwent sigmoid colon resection due to the presence of a mass lesion. It was revealed during intestine mobilization that a formation invaded all the layers of intestine, wherein there was regional lymphadenopathy with involvement of retroperitoneal tissue. When mobilizing the intestine with a trumor, communication was established with the injured ureter of the left kidney. Urologists were called in the operating room and treated surgically the defect of the ureter. To prevent complications, an adjusted in size catheter 100 according to the present invention was administered retrogradely along a guide wire, and one of the isolating balloons 4 was inflated in the pelvis of the left kidney, and the other isolating balloon 4 (the lower isolating balloon) was inflated in the bladder, wherein the functional channel 3 was connected to a suction apparatus for producing a controllable negative pressure. The catheter 100 was removed in 14 days, wherein the excretory function of the left kidney was not impaired. The patient was discharged for outpatient treatment.

Example No. 12. A patient No. 12 with a pulmonary hemorrhage was admitted. Bronchoscopy revealed a putrescent bleeding tumor of the right main bronchus. For the purpose of hemostasis and preservation of bronchial patency, and also for the prevention of lung atelectasis, catheter 100 according to the present invention was administered into the right bronchus. The catheter 100 according to the present invention was inserted such that the trumor was between the isolating balloons 4, wherein inflation of the isolating balloons 4 stopped the blood flowing to other parts of the bronchus. To stop the bleeding a hemostatic agent was administered through the functional channel 3 of the catheter 100. As a result, there was no relapse of the bleeding for three (3) days of observation. The catheter 100 according to the present invention was subsequently removed. The patient was discharged for outpatient treatment ten (10) days later.

Example No. 13. A patient No. 13 with an instrumentally confirmed clinic of mediasthenitis was admitted on an emergency basis. The examination revealed an esophagus defect due to necrosis caused by a piece of meat. For treating the esophagus, the catheter 100 for isolating an interior of a mammal hollow organ according to the present invention was inserted. The isolating balloons 4 of the catheter 100 were inflated to isolate the defect, and the functional channel 3 was connected to a suction apparatus to provide a controllable negative pressure. As a result, the patient was discharged for outpatient treatment after 1.5 months.

Example No. 14. A patient No. 14 with headaches and general cerebral symptoms was admitted on an emergency basis. MSCT with intravenous contrast enhancement revealed an aneurysm in the basin of the right middle cerebral artery with signs of extrusion. The patient was taken to the angiographic operating room, wherein the catheter 100 according to the present invention was administered through the femoral approach. Positioning of the catheter 100 was performed under control of fluoroscopy such that an interior of the aneurysm was isolated by the isolating balloons 4, wherein the functional channel 3 was opposite the aneurysm. A filling solution filling the aneurysm cavity was administered through the functional channel 3. After the necessary exposure, the catheter 100 allowed to maintain a circumferential blood flow. Subsequently the catheter 100 was removed. The patient was discharged from a hospital in 21 days.

Example No. 15. A patient No. 15 was admitted routinely for surgical treatment of pancreatic cancer. An examination showed a head trumor invading the inferior vena cava. To reduce blood loss the catheter 100 according to the present invention was inserted into the vein, and the isolating balloons 4 were inflated above and below the pancreas head tumor, thereby shunting blood and returning blood to the heart. During the resection of the gland, a section of the inferior vena cava was resected with autoplasty of the saphenous vein of the lower extremity. The blood loss was 500 ml. Therefore, optimal temporary and physiological conditions were created both for the patient and surgeons.

Example No. 16. A patient No. 16 was admitted for surgical treatment of sigmoid colon cancer. In a postoperative period, the patient developed colo-rectoanastomosis failure for ⅓ of the circumference. To treat the colon the catheter 100 according to the present invention was retrogradely inserted into the rectum such that one of the isolating balloons 4 of the catheter 100 was positioned most proximally in relation to the defect, and the other isolating balloon 4 of the catheter 100 was positioned most distally in relation to the defect. When the isolating balloons 4 of the catheter 100 were inflated, an interior related to the defect was isolated from other sections of the large intestine. Due to the main channel, proximal sections of the bowel were cleaned with water. As a result, the defect was closed in 3 weeks.

Example No. 17. A patient No. 17 was admitted with suspected liver trumor. To perform a liquid biopsy the catheter 100 according to the present invention was inserted; an interior related to the major and minor duodenal papillas was isolated by the isolating balloons 4 of the catheter 100, and aspiration of bile was started. When the catheter 100 was removed, a bile aspirate was delivered for genetic and cytological examination, wherein no pathologic markers and cells were detected. When the bile aspirate is taken, the grid enclosure 16 covering the isolating balloons 4 of the catheter 100 was removed and washed with a buffer solution, whereupon the water was collected, and a cell sediment was obtained after centrifugation. Cytological examination of the obtained cell sediment allowed the presence of hepatocellular cancer cells to be revealed.

Example No. 18. A patient No. 18 was admitted on an emergency basis with a uterine bleeding. A colposcopy allowed a putrescent cervical cancer to be revealed. To stop the bleeding, the catheter 100 was inserted into a vaginal and uterine cavity, and an interior related to a tumor area was isolated by the isolating balloons 4 of the catheter 100. The instillation with a hemostatic drug was performed for the trumor through the functional channel of the catheter 100. The bleeding was stopped, and the patient left a hospital.

Therefore, the above-described catheter 100 allows functional investigations of any mammal hollow organ to be conducted or performed, and allows appropriate treatments to be performed on the mammal hollow organ. Furthermore, the examination and/or the laboratory analysis of the biological contents collected by the catheter 100 allows inflammatory formations, benign formations and malignant growth, and infectious diseases to be differentially diagnosed with a high accuracy. The catheter 100 allows inflammatory diseases, the bleeding and wall defects of the mammal hollow organ to be treated by sampling or taking a specific biological contents (e.g. a biological liquid) from the isolated interior of the mammal hollow organ. The isolated interior providing a bypass function during a surgical manipulation allows a hemostasis to be controlled, and also allows surgical manipulations to be performed, in particular allows the resection of a mammal vessel site to be performed and also allows a plastic surgery of the resected mammal vessel site to be performed. It is to further note that the catheter 100 or the above-described system comprising the catheter 100 allows a high volume of the biological contents having a normal physiological and biochemical state to be collected by the catheter 100 without any additional stimulation of the excretory function of the mammal hollow organ, in particular due to the production of the negative pressure corresponding to a physiological negative pressure of the mammal hollow organ, the physiological negative pressure being normally created, for example, due to the peristalsis of the intestine in the isolated interior of the mammal hollow organ having the catheter 100 inserted into the lumen thereof.

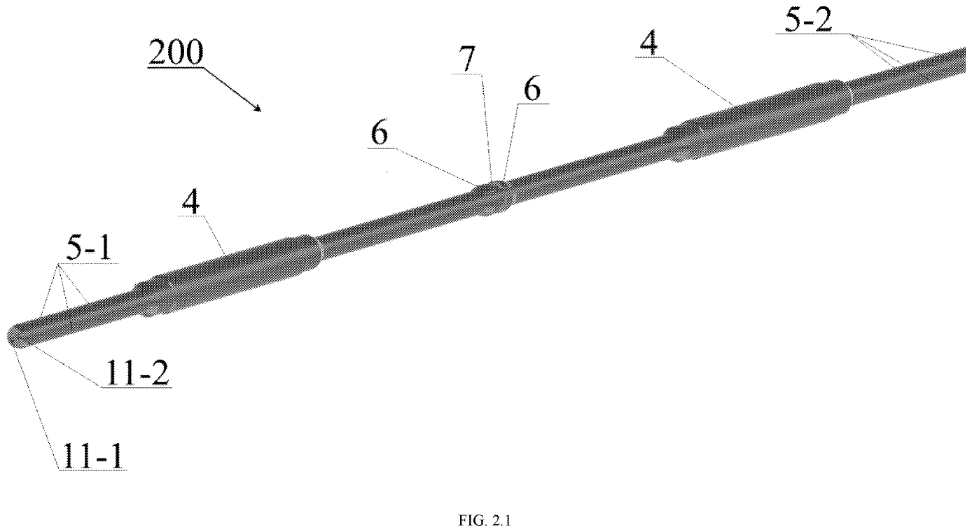

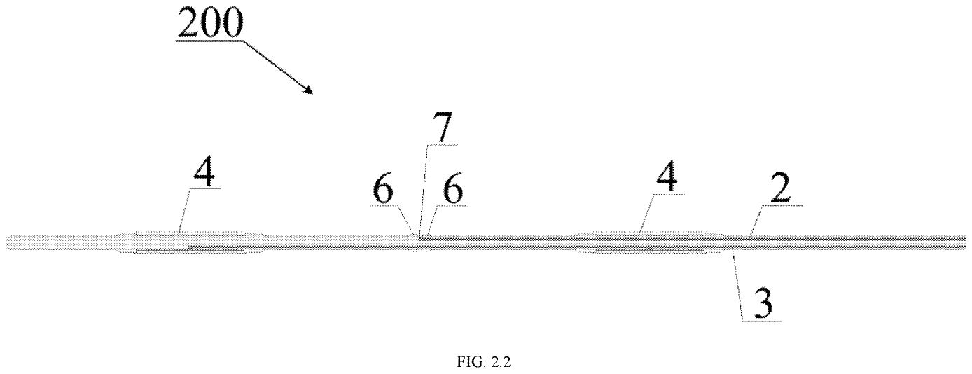

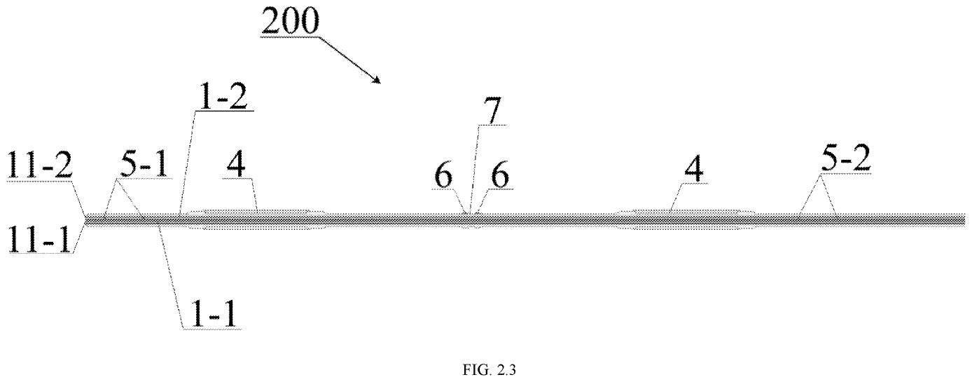

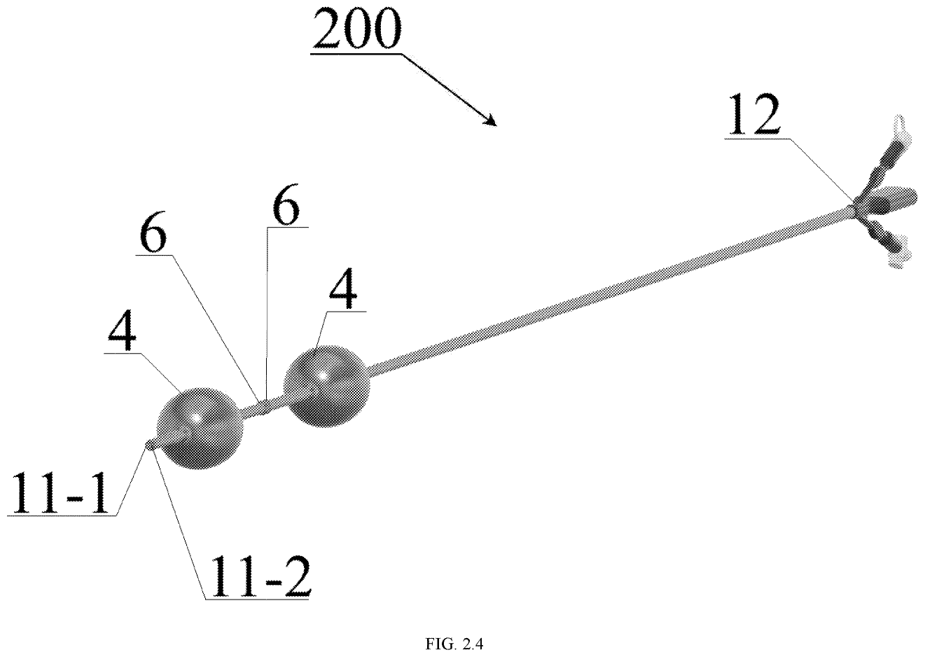

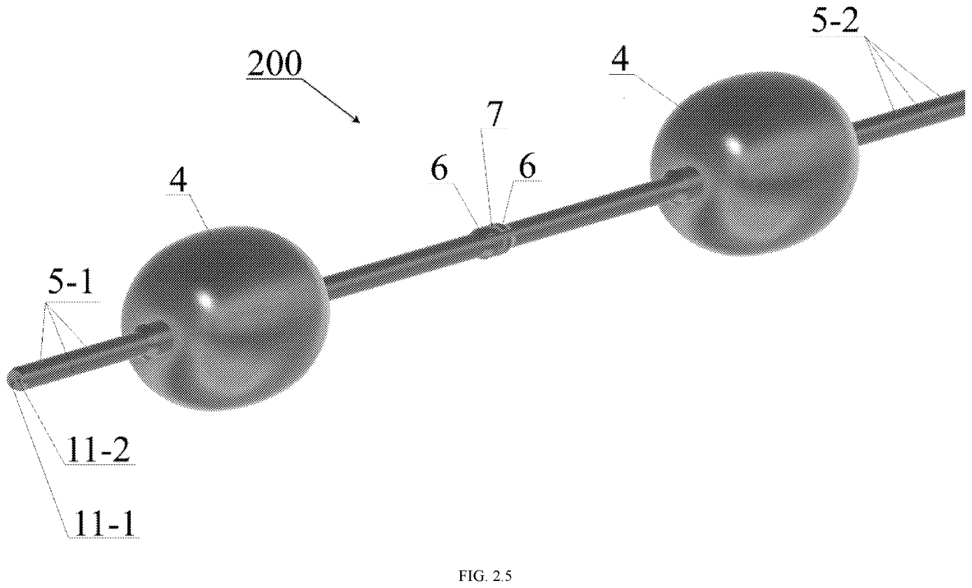

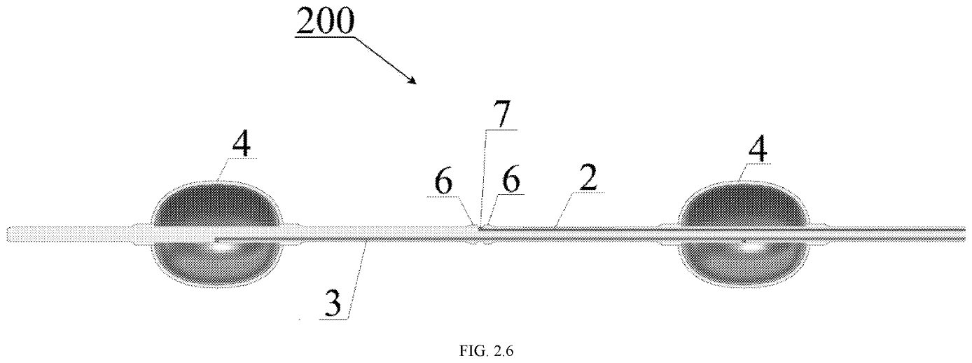

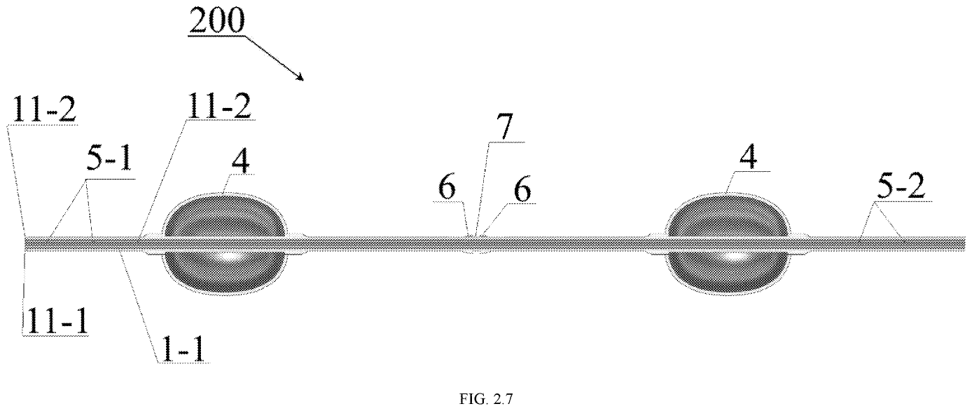

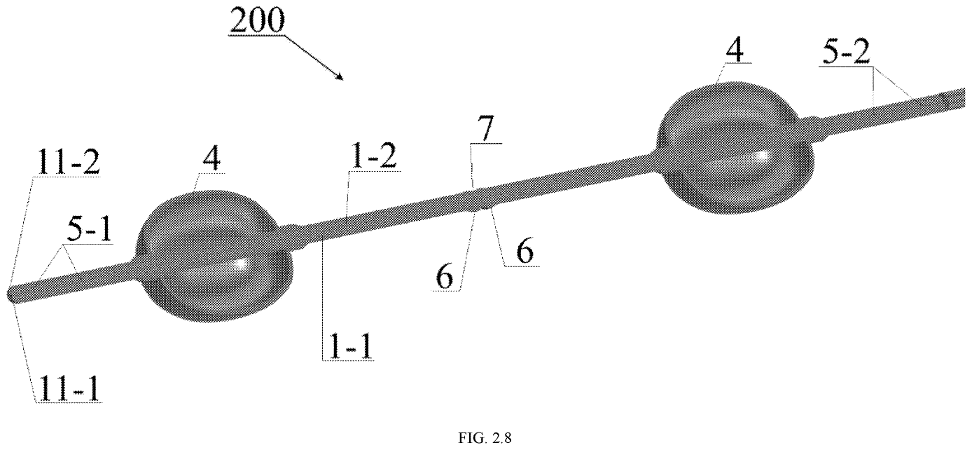

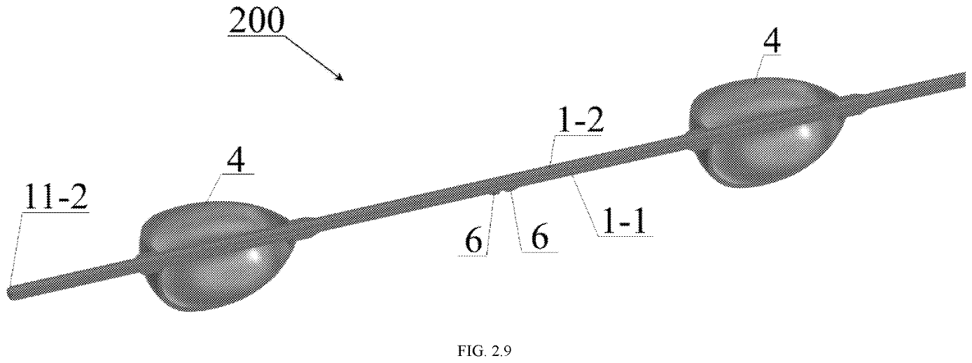

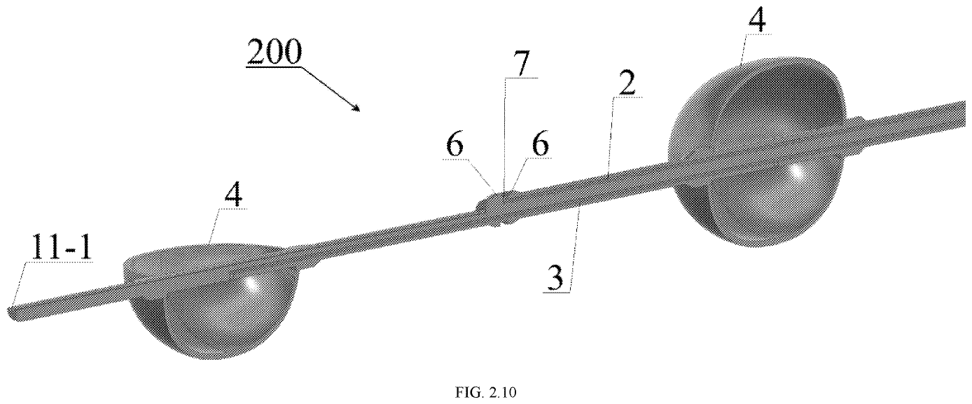

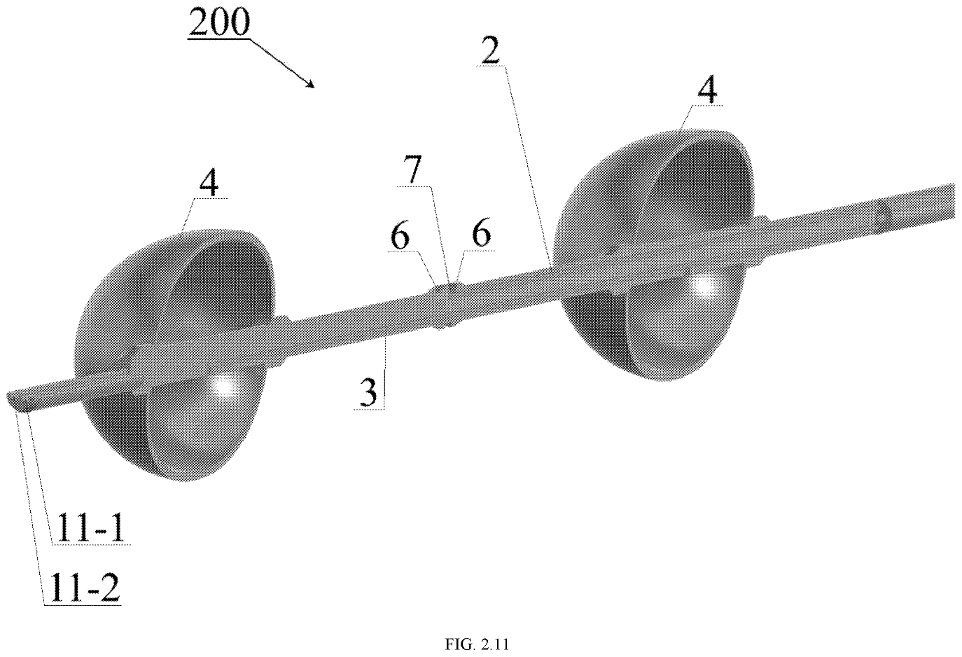

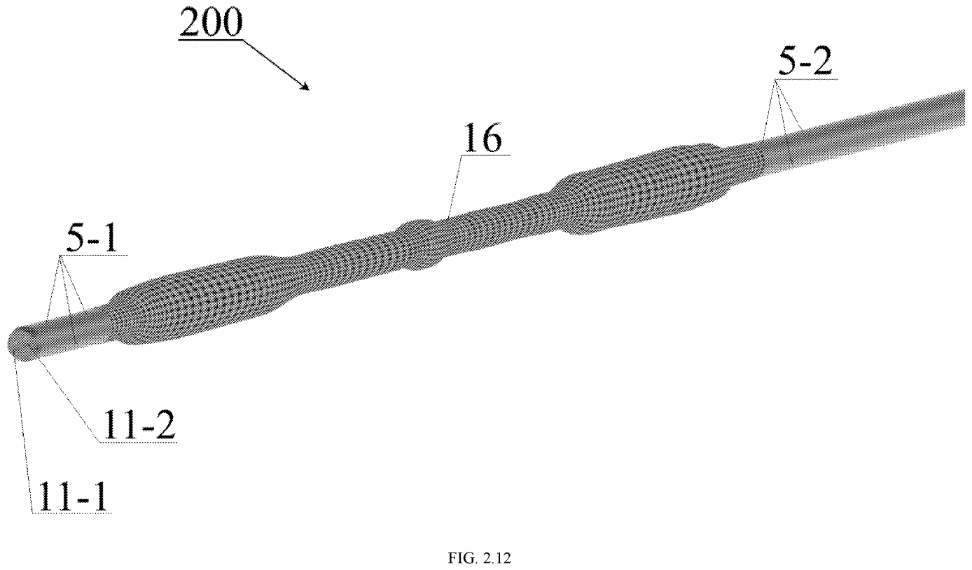

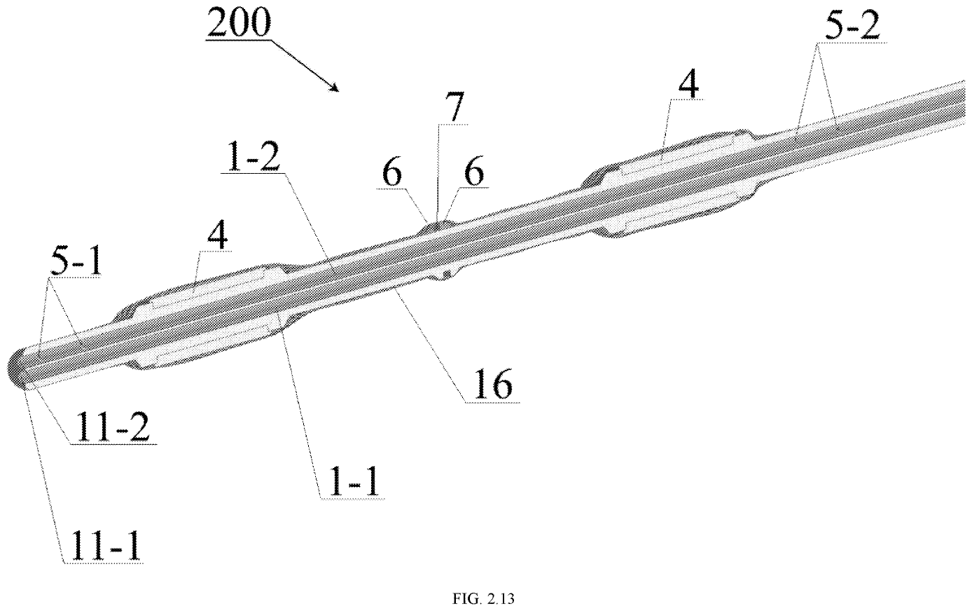

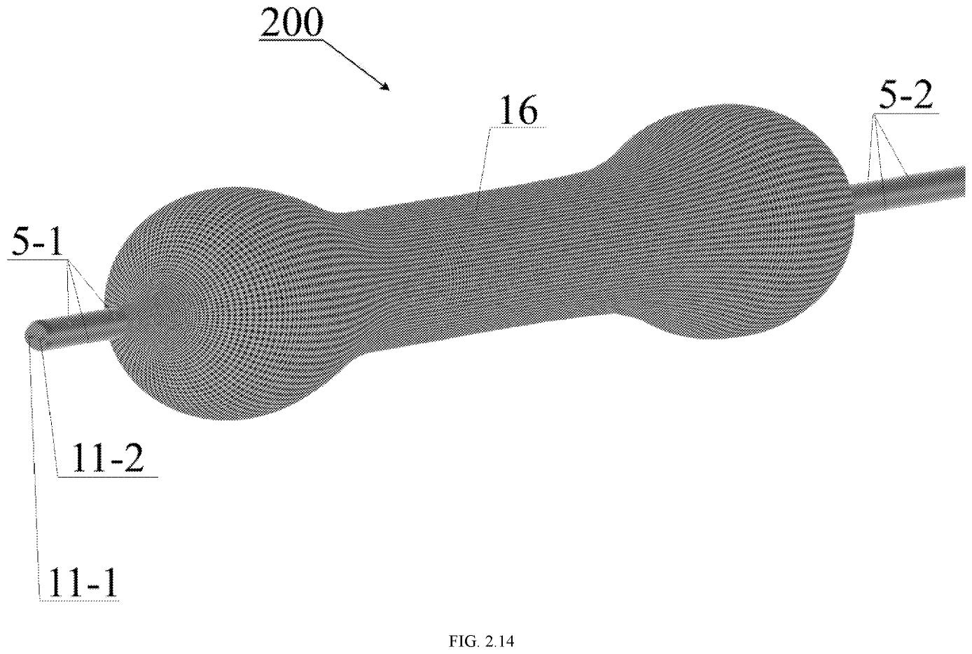

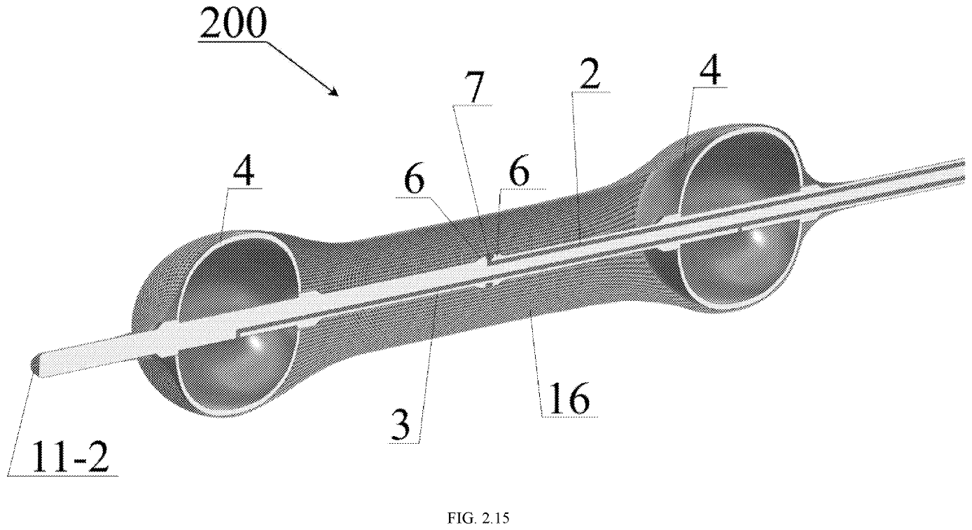

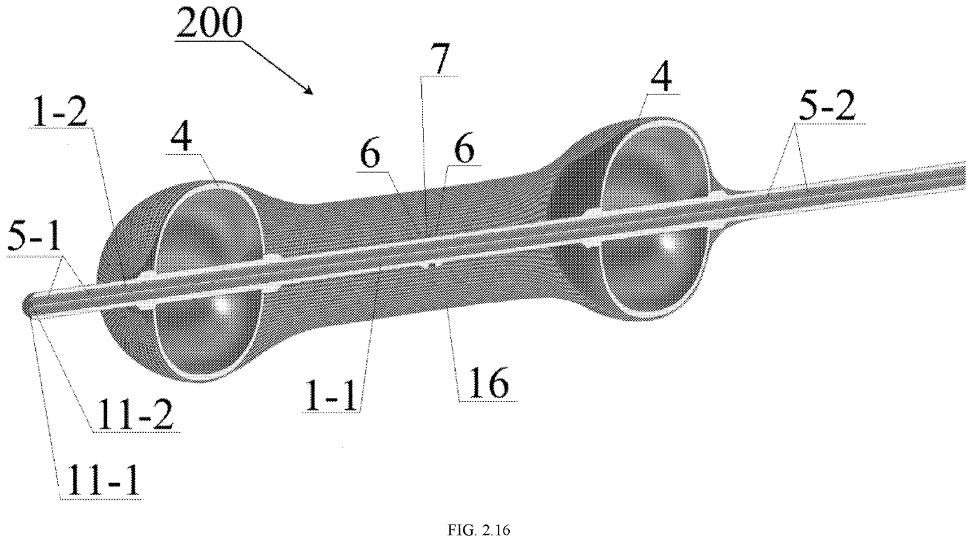

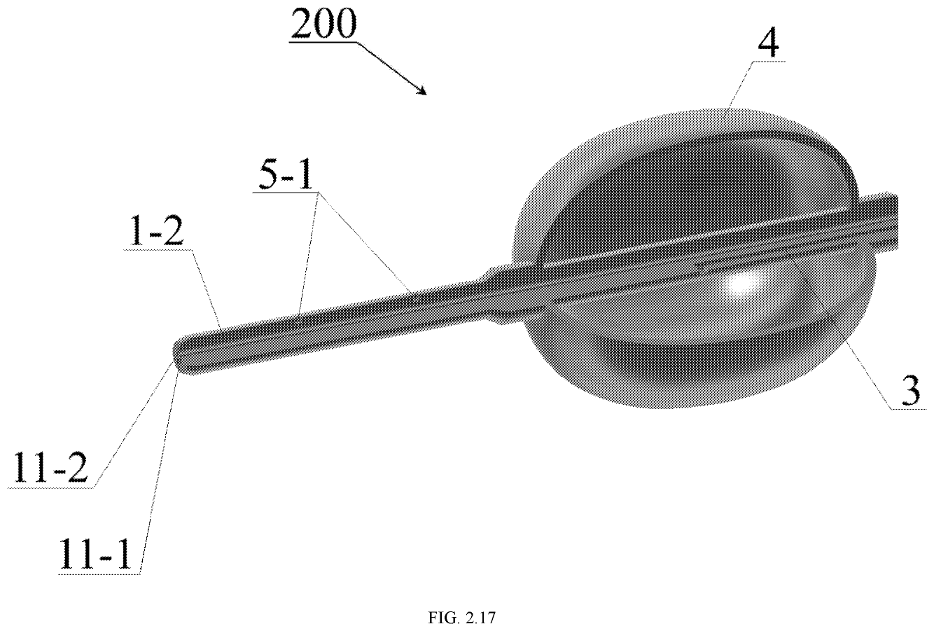

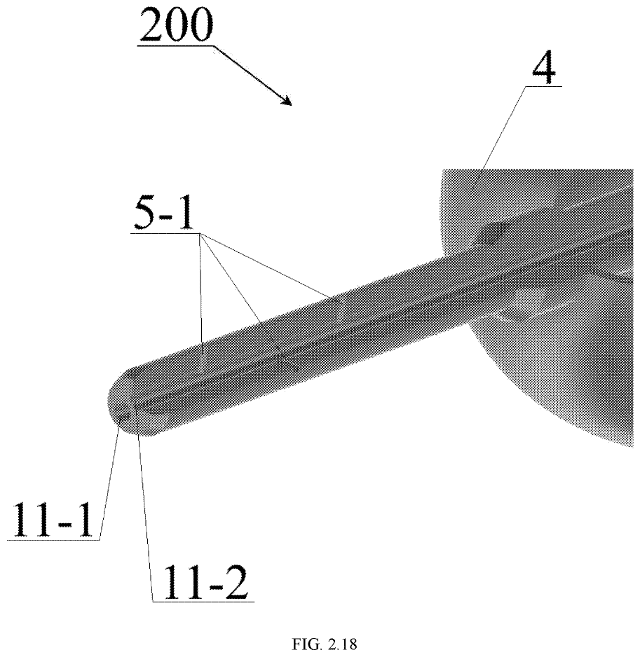

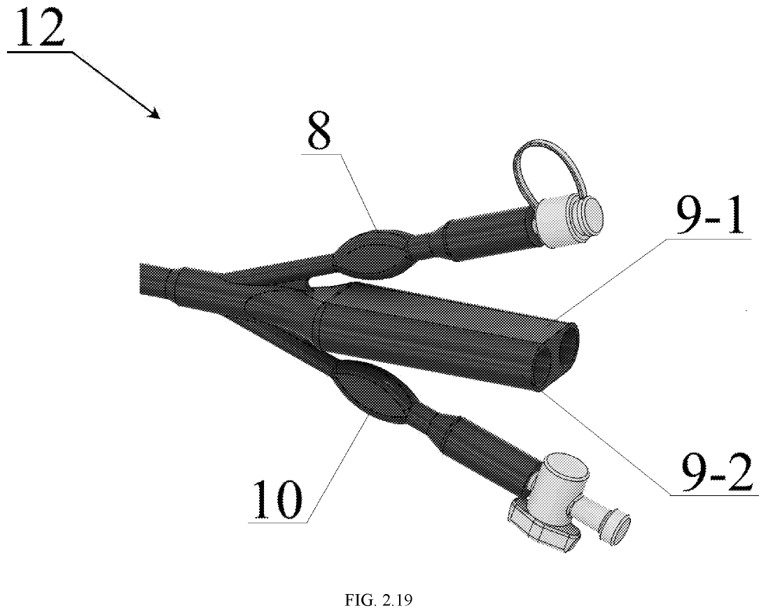

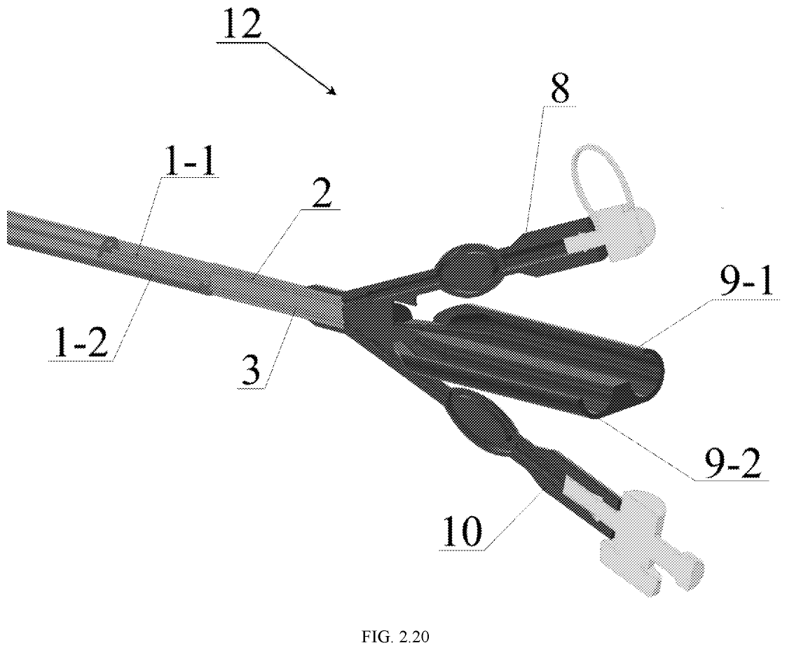

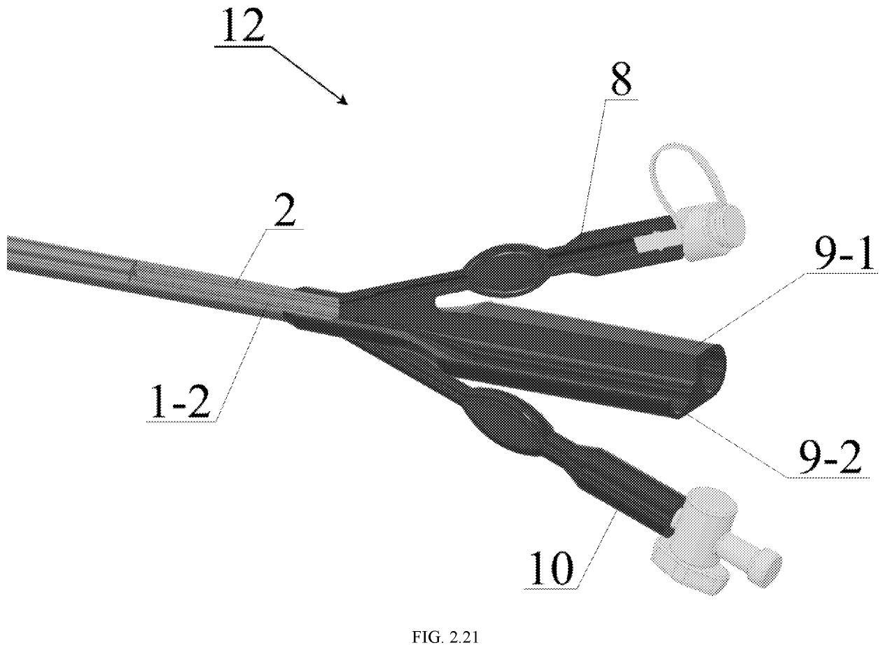

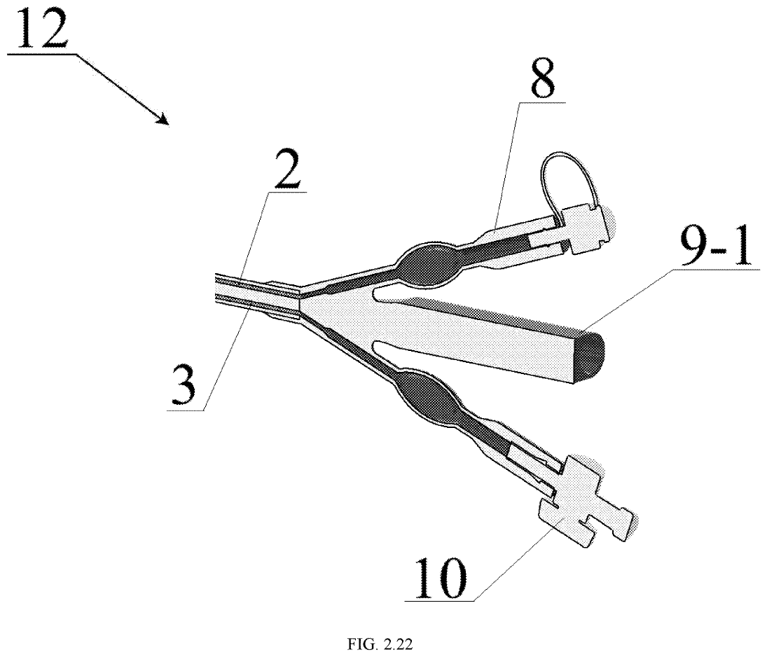

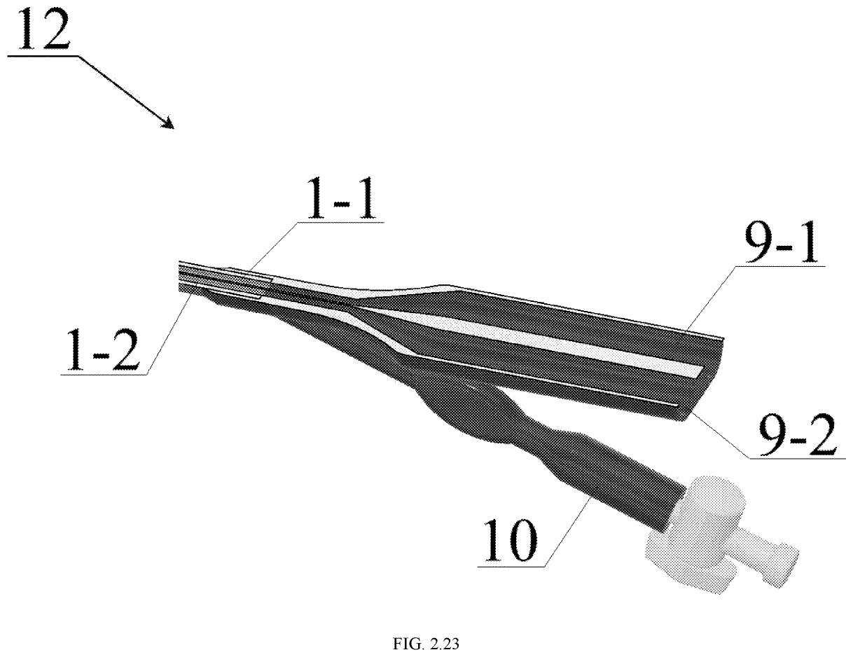

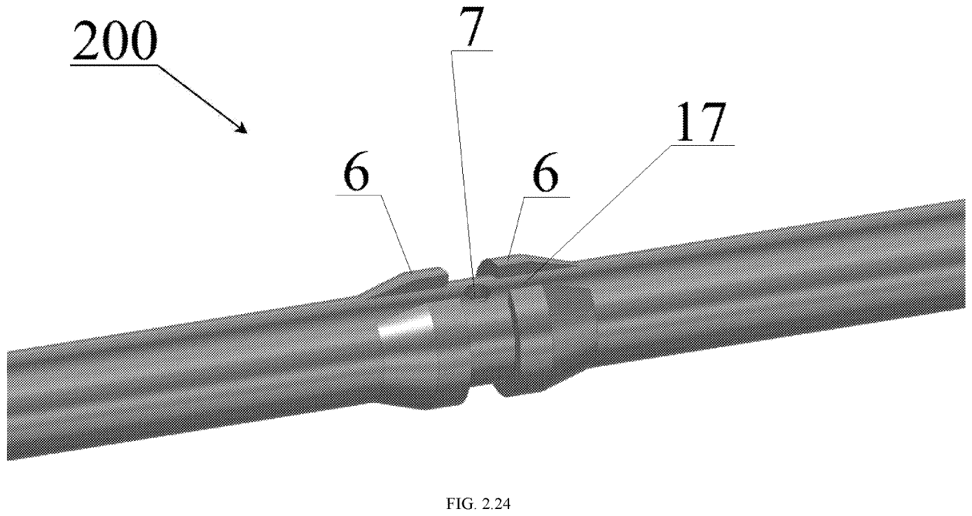

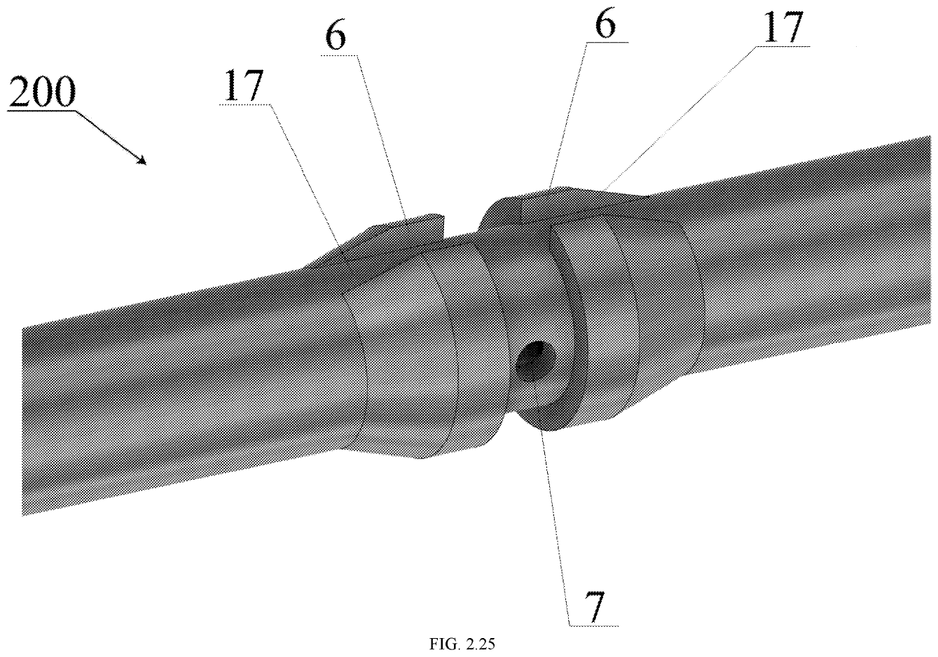

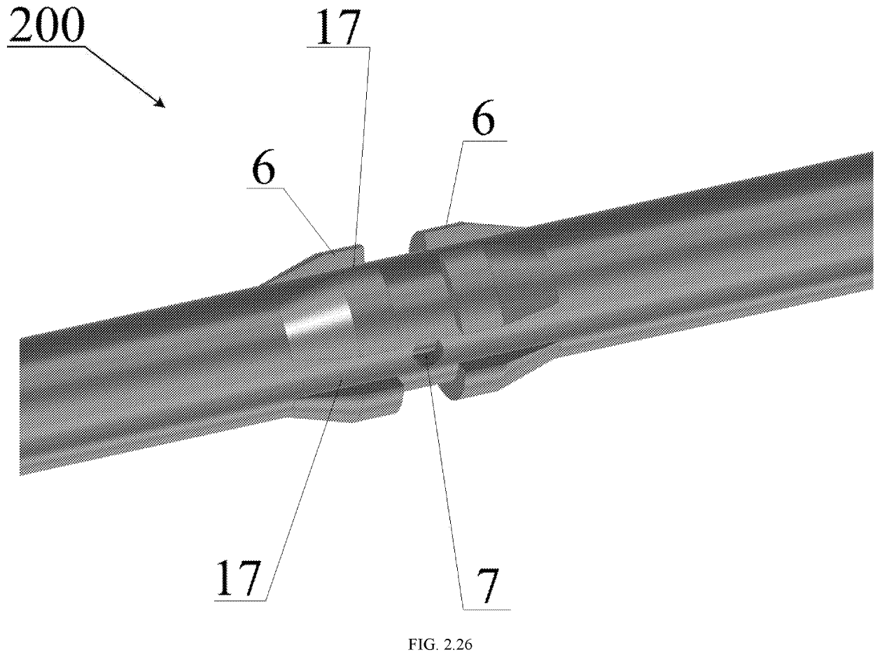

FIGS. 2.1-2.18 schematically illustrate a catheter 200 for isolating an interior of a mammal hollow organ according to the present invention, wherein the catheter 200 is generally structurally and functionally similar to the above-described catheter 100. In particular, similar to the catheter 100, the catheter 200 comprises (i) an elongate catheter body designed to be inserted into a lumen of the mammal hollow organ; (ii) two isolating balloons 4, individually and separately disposed along the elongate catheter body of the catheter 200 and designed to be inflated via an inflation channel 2 by using the above-described third functional device connected to a side terminal 10 of a connector 12 hermetically connected to a proximal end of the elongate catheter body of the catheter 200 to isolate the interior of the mammal hollow organ therebetween; and (iii) a functional channel 3 extending in the elongate catheter body of the catheter 200 and comprising a functional opening 7 provided in the elongate catheter body of the catheter 200 between the two isolating balloons 4, wherein the functional channel 3 is designed to allow (a) a negative pressure to be produced in the isolated interior to sample or take a gaseous medium, a liquid medium or a fluid medium (i.e. a gas, a liquid or a fluid as any appropriate combination of a liquid and a gas) therefrom via the functional opening 7 by using the above-described second functional device (particularly, the above-described aspirator) connected to a side terminal 8 of the connector 12 or (b) gaseous medium, a liquid medium or a fluid medium (i.e. a combination of a liquid medium and a gaseous medium) to be supplied into the isolated interior via the functional opening 7 by using the above-described second functional device (particularly, the above-described medium supply device) connected to the side terminal 8. As shown in FIGS. 2.12-2.16, similar to the catheter 100, the catheter 200 comprises (iv) the net or enclosure 16 being permeable to a gaseous medium, a liquid medium and/or a fluid medium (i.e. a gas, a liquid or a fluid), wherein the net or enclosure 16 is externally provided on the elongate catheter body of the catheter 200 and at least partly encloses the catheter body part defined by the isolating balloons 4 so as to cover the functional opening 7, and wherein the net or enclosure 16 is attached to both the isolating balloons 4 or encloses them such that the net or enclosure 16 becomes strained when the isolating balloons 4 are inflated by the above-described third functional device. Also, similar to the catheter 100, the catheter 200 comprises (v) two ring-shaped projections 6 enclosing the functional opening 7, wherein the two ring-shaped projections are provided on the elongate catheter body between the isolating balloons 4 such that the functional opening 7 is between the two ring-shaped projections 6, and wherein the two ring-shaped projections 6 are designed such that they allow a distance between a mucosal tissue of the mammal hollow organ, the mucosal tissue being between the inflated isolating balloons 4, and the functional opening 7 to be provided when the negative pressure is produced by the second functional device. In other words, the two isolating balloons 4, the functional opening 7, the functional channel 3, the inflation channel 2, the two ring-shaped projections 6 and the net or enclosure 16 in the catheter 200 are completely structurally and functionally equal to that of the catheter 100, so that the above description related to the above-mentioned components of the catheter 100, including their particular embodiments described above in relation to the catheter 100, are completely applicable to the catheter 200. Moreover, as shown in FIGS. 2.19-2.23, the side terminals 8, 10 in the connector 12 hermetically connected to the proximal end of the elongate catheter body of the catheter 200 are completely structurally and functionally equal to that of the above-described connector 12 hermetically connected to the proximal end of the elongate catheter body of the catheter 100, so that the above description related to the above-mentioned terminals of the connector 12 used for the catheter 100, including their particular embodiments described above in relation to the catheter 100, are completely applicable to the connector 12 used for the catheter 200.

However, contrary to the catheter 100, the catheter 200 comprises a medium supply channel 1-1 extending within the elongate catheter body of the catheter 200 and generally corresponding to the above described main channel 1 and comprises a medium drainage channel 1-2 extending within the elongate catheter body of the catheter 200 in parallel to the medium supply channel 1-1, wherein the medium drainage channel 1-2 is hermetically isolated from the medium supply channel 1-1, and wherein three auxiliary holes 5-1 and three auxiliary holes 5-2 provided in the elongate catheter body of the catheter 200 outside of the catheter body part defined by the isolating balloons 4 are all in fluid communication with the medium drainage channel 1-2, The three auxiliary holes 5-1 are all provided at the distal end of elongate catheter body of the catheter 200 at a predetermined distance from a distal isolating balloon 4 spaced closer to the distal end of elongate catheter body of the catheter 200, and the three auxiliary holes 5-2 are all provided at the proximal end of elongate catheter body of the catheter 200 at a predetermined distance from a proximal isolating balloon 4 spaced closer to the proximal end of elongate catheter body of the catheter 200. Thus, the elongate catheter body of the catheter 200 is provided with four separate channels: (i) the medium supply channel 1-1, (ii) the medium drainage channel 1-2, (iii) the inflation channel 2, and (iv) the functional channel 3, wherein all the channels extending within the elongate catheter body of the catheter 200 are hermetically isolated from each other. It is to note that the three auxiliary holes 5-1 and the three auxiliary holes 5-2 provided in the elongate catheter body of the catheter 200 are generally structurally and functionally equal to that provided in the elongate catheter body of the catheter 100, so that the above description related to the above-mentioned auxiliary holes provided in the elongate catheter body of the catheter 100, including their particular embodiments described above in relation to the catheter 100, are substantially applicable to the auxiliary holes 5-1 provided in the elongate catheter body of the catheter 200.

In other words, in the catheter 200 as compared to the catheter 100, the above-described main channel 1 provided in the elongate catheter body of the catheter 100 is substantially replaced with two separate parallel channels provided in the elongate catheter body of the catheter 200 and hermetically isolated from each other, in particular the medium supply channel 1-1 and the medium drainage channel 1-2, wherein only the medium drainage channel 1-2 is in fluid communication with the three auxiliary holes 5-1 and with the three auxiliary holes 5-2.

In an embodiment of the present invention, the medium drainage channel 1-2 provided within the elongate catheter body of the catheter 200 comprises one or more auxiliary holes 5-1 (e.g. one, two, three, four, five, six, seven, eight, nine, ten or more auxiliary holes 5-1) located all at the distal end of elongate catheter body of the catheter 200 at the predetermined distance from the distal isolating balloon 4 spaced closer to the distal end of elongate catheter body of the catheter 200 and one or more auxiliary holes 5-2 (e.g. one, two, three, four, five, six, seven, eight, nine, ten or more auxiliary holes 5-2) located all at the proximal end of elongate catheter body of the catheter 200 at the predetermined distance from a proximal isolating balloon 4 spaced closer to the proximal end of elongate catheter body of the catheter 200, wherein the auxiliary holes 5-1 and the auxiliary holes 5-2 are provided in the elongate catheter body of the catheter 200 outside of the catheter body part defined by the two isolating balloons 4 (i.e. the distal isolating balloon 4 and the proximal isolating balloon 4), and wherein the auxiliary holes 5-1 and the auxiliary holes 5-2 are in fluid communication with each other via the medium drainage channel 1-2.

In another embodiment of the present invention, the medium drainage channel 1-2 provided within the elongate catheter body of the catheter 200 comprises only one or more auxiliary holes 5-2 (e.g. one, two, three, four, five, six, seven, eight, nine, ten or more auxiliary holes 5-2), while the above-described auxiliary holes 5-1 are not provided in the elongate catheter body of the catheter 200, wherein the auxiliary holes 5-2 are all located at the proximal end of elongate catheter body of the catheter 200 at the predetermined distance from the proximal isolating balloon 4 spaced closer to the proximal end of elongate catheter body of the catheter 200 and provided in the elongate catheter body of the catheter 200 outside of the catheter body part defined by the two isolating balloons 4, and wherein the auxiliary holes 5-2 are all in fluid communication with the medium drainage channel 1-2.

As shown in FIGS. 2.4-2.5 and 2.8-2.11, the distal end of the elongate catheter body of the catheter 200, the distal end being used for inserting the elongate catheter body of the catheter 200 into the lumen of the mammal hollow organ and being opposite to the proximal end of the elongate catheter body of the catheter 200, is provided with an end-face opening 11-1 generally corresponding to the above-described axial opening 11 provided in the elongate catheter body of the catheter 100 and further provided with an end-face opening 11-2.

The medium supply channel 1-1 is in fluid communication with the end-face opening 11-1 provided at the distal end of the elongate catheter body of the catheter 200, and the medium drainage channel 1-2 is in fluid communication with the end-face opening 11-2 provided at the distal end of the elongate catheter body of the catheter 200.

It is to note that when the elongate catheter body of the catheter 200 is inserted into the lumen of the mammal hollow organ, both the end-face openings 11-1, 11-2 are opened to an interior of a distal section of the mammal hollow organ and in fluid communication with the interior of the distal section of the mammal hollow organ, so that the end-face opening 11-1 provides the fluid communication between the interior of the distal section of the mammal hollow organ and the medium supply channel 1-1, and the end-face opening 11-2 provides the fluid communication between the interior of the distal section of the mammal hollow organ and the medium drainage channel 1-2.

In an embodiment of the present invention, the elongate catheter body of the catheter 200 is provided with one or more auxiliary holes 5-1 (e.g. one, two, three, four, five, six, seven, eight, nine, ten or more auxiliary holes 5-1) located all at the distal end of elongate catheter body of the catheter 200 at a predetermined distance from a distal isolating balloon 4 spaced closer to the distal end of elongate catheter body of the catheter 200 and provided with one or more auxiliary holes 5-2 (e.g. one, two, three, four, five, six, seven, eight, nine, ten or more auxiliary holes 5-2) located all at the proximal end of elongate catheter body of the catheter 200 at a predetermined distance from a proximal isolating balloon 4 spaced closer to the proximal end of elongate catheter body of the catheter 200, while the above-described end-face opening 11-2 is not provided at the distal end of the elongate catheter body of the catheter 200. In other words, in the present embodiment of the present invention, the medium drainage channel 1-2 in the elongate catheter body of the catheter 200 comprises only one or more auxiliary holes 5-1 and one or more auxiliary holes 5-2 and does not comprise the above-described end-face opening 11-2, wherein the auxiliary holes 5-1 and the auxiliary holes 5-2 are in fluid communication with each other via the medium drainage channel 1-2.

In another embodiment of the present invention, the elongate catheter body of the catheter 200 is provided with the end-face opening 11-2 and provided with one or more auxiliary holes 5-2 (e.g., one, two, three, four, five, six, seven, eight, nine, ten or more auxiliary holes 5-2) located all at the proximal end of elongate catheter body of the catheter 200 at a predetermined distance from a proximal isolating balloon 4 spaced closer to the proximal end of elongate catheter body of the catheter 200, while the above-described auxiliary holes 5-1 are not provided at the distal end of elongate catheter body of the catheter 200. In other words, in the present another embodiment of the present invention, the medium drainage channel 1-2 comprises only the end-face opening 11-2 and one or more auxiliary holes 5-2 and does not comprise the above-described auxiliary holes 5-1, wherein the end-face opening 11-2 and the auxiliary holes 5-2 are in fluid communication with each other via the medium drainage channel 1-2.

Also, contrary to the catheter 100, the connector 12 shown in FIGS. 2.19-2.23 hermetically connected to the proximal end of the elongate catheter body of the catheter 200 is provided with four (4) branches or terminals: (i) a medium supply terminal 9-1 generally corresponding to the above-described central terminal 9, (ii) a medium drainage terminal 9-2 hermetically isolated from the medium supply terminal 9-1, (iii) the side terminal 8 identical to that of the above-described connector 12 hermetically connected to the proximal end of the elongate catheter body of the catheter 100, and (iv) the side terminal 10 identical to that of the above-described connector 12 hermetically connected to the proximal end of the elongate catheter body of the catheter 100, wherein each of the side terminals 8, 10 are hermetically isolated from both the medium supply terminal 9-1 and the medium drainage terminal 9-2. In other words, the connector 12 hermetically connected to the proximal end of the elongate catheter body of the catheter 200 is a four-way connector.

Similar to the above-described side terminals 8, 10, each of the medium supply terminal 9-1 and the medium drainage terminal 9-2 has an opening provided at a free end of said terminal and an inner channel provided inside said terminal, wherein said opening is in fluid communication with said inner channel. In other words, the opening provided at the free end of the medium supply terminal 9-1 is in fluid communication with the inner channel of the medium supply terminal 9-1, and the opening provided at the free end of the medium drainage terminal 9-2 is in fluid communication with the inner channel of the medium drainage terminal 9-2.

It is to note that all the terminals 8, 9-1, 9-2, 10 in the connector 12 provided on the proximal end of elongate catheter body of the catheter 200 are located outside the mammal body when the elongate catheter body of the catheter 200 is inserted into the lumen of the mammal hollow organ of the mammal body and, therefore, communicate with the ambient atmosphere or environment via corresponding openings provided at free ends of said terminals 8, 9-1, 9-2, 10 in the connector 12.

Similar to the catheter 100, the terminals 8, 9-1, 9-2, 10 in the connector 12 provided on the proximal end of elongate catheter body of the catheter 200 are each designed to have an appropriate functional appliance or functional device (not shown) operatively releasably connected thereto.

In particular, as shown in FIGS. 2.19-2.23, the medium supply terminal 9-1 and the medium drainage terminal 9-2 are both positioned between the side terminals 8, 10 in the connector 12 of the catheter 200, wherein the medium supply terminal 9-1 is designed to connect the above-described first functional device thereto, and the medium drainage terminal 9-2 is generally free (i.e. no any functional device needs to be generally connected to the medium drainage terminal 9-2).

In an embodiment of the present invention, the medium drainage terminal 9-2 is further designed to connect a fourth functional device (not shown) thereto, wherein the fourth functional device designed to aspirate or evacuate an air medium or an air under a pressure, wherein the pressure generated by the fourth functional device for aspirating or evacuating the air medium or the air may be set/controlled/regulated/adjusted automatically or manually by a skilled person (e.g. a user). In the present embodiment, the fourth functional device may be implemented as a medical suction apparatus, an aspiration device or an aspirator (not shown), each comprising (i) a storage reservoir or a storage container (not shown) for collecting a gaseous medium, a liquid medium or a fluid medium (any combination of a gaseous medium and a liquid medium) specific to the mammal hollow organ and (ii) an air compressor (not shown) for aspirating or evacuating the air under the pressure, wherein the storage container of the fourth functional device may be in fluid communication with the opening of the medium drainage terminal 9-2 via the fourth functional device.

The medium supply channel 1-1 provided in the elongate catheter body of the catheter 200 is in fluid communication with the medium supply terminal 9-1 of the connector 12 and extends substantially along all the length of the elongate catheter body of the catheter 200, wherein the medium supply channel 1-1 is further designed to be in fluid communication with the interior of the mammal hollow organ via the end-face opening 11-1 provided at the distal end of the elongate catheter body of the catheter 200. In particular, the medium supply channel 1-1 has an inlet hole or an inlet at the proximal end of the elongate catheter body, the inlet being opened to the inner channel of the medium supply terminal 9-1 to allow the medium supply channel 1-1 to be in fluid communication with the inner channel of the medium supply terminal 9-1, and also has an outlet hole or an outlet corresponding to the end-face opening 11-1 of the elongate catheter body to allow the medium supply channel 1-1 to be in fluid communication with the interior of the mammal hollow organ.

When the elongate catheter body of the catheter 200, the elongate catheter body having the connector 12 hermetically connected to the proximal end of the elongate catheter body, is initially inserted into the lumen of the mammal hollow organ, and when the isolating balloons 4 are subsequently inflated by using the above-described third functional device, the inlet of the medium supply channel 1-1 communicates with an ambient atmosphere or environment via the inner channel of the medium supply terminal 9-1 and via the opening of the medium supply terminal 9-1, and the end-face opening 11-1 of the medium supply channel 1-1 is in fluid communication with the interior of the distal section of the mammal hollow organ (e,g, esophagus, stomach and duodenum, small intestine or large intestine, and also respiratory tracts, biliary tract, urinary tracts, vessels, etc.).

When the above-described first functional device (not shown) is then connected to the medium supply terminal 9-1 of the connector 12, the gaseous medium, the liquid medium or the fluid medium (i.e. a combination of a liquid medium and a gaseous medium) may be firstly supplied under a pressure from the first functional device to the inner channel of the medium supply terminal 9-1 via the opening of the medium supply terminal 9-1, then supplied from the inner channel of the medium supply terminal 9-1 to the medium supply channel 1-1 via the inlet of the medium supply channel 1-1, and then finally supplied from the medium supply channel 1-1 to the interior of the distal section of the mammal hollow organ via the end-face opening 11-1. It is to note that the pressure generated by the first functional device to supply the gaseous medium, the liquid medium or the fluid medium into the medium supply terminal 9-1 may be set/controlled/regulated/adjusted automatically or manually by a skilled person (e.g. a user).

In other words, the medium supply channel 1-1 extending in the elongate catheter body of the catheter 200 and comprising the end-face opening 11-1 is used in the catheter 200 to connect the above-described first functional device thereto via the inner channel of the medium supply terminal 9-1 when the elongate catheter body of the catheter 200 is inserted into the lumen of the mammal hollow organ.

For example, the above-described first functional device, when connected to the medium supply terminal 9-1 of the connector 12, may be used or actuated for supplying medical products, in particular the enteral or parenteral nutrition mixture, under a pressure to the interior of the distal section of the mammal hollow organ via the inner channel of the medium supply terminal 9-1 and the medium supply channel 1-1. Thus, the medium supply channel 1-1 having the first functional device connected thereto allows the enteral or parenteral nutrition mixture to be supplied via the end-face opening 11-1 to the interior of the distal section of the mammal hollow organ so as to provide the mammal organism with liquids, minerals, basic nutrients, energy, vitamins, macronutrients, micronutrients and/or etc., while (i) sampling or taking the gaseous medium, the liquid medium or the fluid medium from the isolated interior of the mammal hollow organ via the functional opening 7 by using the above-described second functional device (particularly, the above-described aspirator) connected to the side terminal 8 of the connector 12 or (ii) supplying the gaseous medium, the liquid medium or the fluid medium to the isolated interior of the mammal hollow organ via the functional opening 7 by using the above-described second functional device (particularly, the above-described medium supply device) connected to the side terminal 8 of the connector 12.

The medium drainage channel 1-2 provided in the elongate catheter body of the catheter 200 is in fluid communication with the medium drainage terminal 9-2 of the connector 12 and extends substantially along all the length of the elongate catheter body of the catheter 200, wherein the medium drainage channel 1-2 is further designed to be in fluid communication with the interior of the mammal hollow organ via the end-face opening 11-2. In particular, the medium drainage channel 1-2 comprises a hole at the proximal end of the elongate catheter body of the catheter 200, the hole being opened to the inner channel of the medium drainage terminal 9-2 to allow the medium drainage channel 1-2 to be in fluid communication with the inner channel of the medium drainage terminal 9-2. The medium drainage channel 1-2 further comprises the three auxiliary holes 5-1, the three auxiliary holes 5-2 and the end-face opening 11-2 to allow the medium drainage channel 1-2 to be in fluid communication with the interior of the mammal hollow organ.

When the elongate catheter body of the catheter 200, the elongate catheter body having the connector 12 hermetically connected to the proximal end of the elongate catheter body, is initially inserted into the lumen of the mammal hollow organ (meanwhile both the isolating balloons 4 are not inflated), the medium drainage channel 1-2 still communicates with the ambient atmosphere or environment via the hole of the medium drainage channel 1-2, via the inner channel of the medium drainage terminal 9-2 and via the opening of the medium drainage terminal 9-2, while the end-face opening 11-2 of the medium drainage channel 1-2, the three auxiliary holes 5-1 and the three auxiliary holes 5-2 are each in fluid communication with the interior of the mammal hollow organ (e.g., esophagus, stomach and duodenum, small intestine or large intestine, and also respiratory tracts, biliary tract, urinary tracts, vessels, etc.).

When both the isolating balloons 4 are then inflated by using the above-described third functional device, the medium drainage channel 1-2 remains to be in communication with the ambient atmosphere or environment via the hole of the medium drainage channel 1-2, via the inner channel of the medium drainage terminal 9-2 and via the opening of the medium drainage terminal 9-2, while the end-face opening 11-2 of the medium drainage channel 1-2 and the three auxiliary holes 5-1 are each in fluid communication only with the interior of the distal section of the mammal hollow organ, and the three auxiliary holes 5-2 are each in fluid communication only with the interior of the proximal section of the mammal hollow organ.

The medium drainage channel 1-2 extending in the elongate catheter body of the catheter 200 and comprising the three auxiliary holes 5-1, the three auxiliary holes 5-2 and the end-face opening 11-2 allows a flow comprised of biological liquid media, biological gaseous media or biological fluid media (i.e. any combination of a biological liquid media and a biological gaseous media), the flow being specific to the mammal hollow organ (i.e. a flow of biological contents being specific to the mammal hollow organ), to be passed bidirectionally through the medium drainage channel 1-2 provided in the elongate catheter body of the catheter 200 under a natural pressure produced by the mammal hollow organ, i.e. passed through the medium drainage channel 1-2 provided in the elongate catheter body of the catheter 200 from the proximal section of the mammal hollow organ to the distal section of the mammal hollow organ (wherein the flow enters the medium drainage channel 1-2 though the three auxiliary holes 5-2 operating as inlets, and said flow exits/leaves the medium drainage channel 1-2 through both the three auxiliary holes 5-1 and the end-face opening 11-2 that operate together as outlets) or vice versa (wherein the flow enters the medium drainage channel 1-2 though both the three auxiliary holes 5-1 and the end-face opening 11-2 that operate together as inlets, and said flow exits/leaves the medium drainage channel 1-2 through the three auxiliary holes 5-2), so that the mammal hollow organ may not be blocked or occluded by excessively accumulated biological liquid media, biological gaseous media or biological fluid media (i.e. excessively accumulated biological contents being specific to the mammal hollow organ), thereby preventing the mammal hollow organ from being damaged (for example, due to an initiated clotting and thrombosis conditioned by the accumulated biological liquid media, biological gaseous media or biological fluid media). Therefore, in the catheter 200, the formation of congestive processes and/or inflammatory processes in the mammal hollow organ due to the biological liquid media, biological gaseous media or biological fluid media (i.e. the biological contents) accumulated outside the interior isolated between the inflated balloons 4 is also prevented or excluded due to the provided bidirectional passage through the medium drainage channel 1-2 provided in the elongate catheter body of the catheter 200, so that said biological liquid media, biological gaseous media or biological fluid media (i.e. the biological contents), when accumulated in the proximal section of the mammal hollow organ, may enter the medium drainage channel 1-2 through the three auxiliary holes 5-2 and may exit/leave the medium drainage channel 1-2 through both the three auxiliary holes 5-1 and the end-face opening 11-2 or vice versa (i.e. said biological liquid media, biological gaseous media or biological fluid media, when accumulated in the distal section of the mammal hollow organ, may enter the medium drainage channel 1-2 through both the three auxiliary holes 5-1 and the end-face opening 11-2 and may exit/leave the medium drainage channel 1-2 through the three auxiliary holes 5-2). Therefore, the medium drainage channel 1-2 provided in the elongate catheter body of the catheter 200 allows the flow comprised of biological liquid media, biological gaseous media or biological fluid media (i.e. any combination of a biological liquid media and a biological gaseous media) to be bidirectionally transferred therethrough, thereby maintaining the normal functioning of the mammal hollow organ having the catheter 200 inserted into the lumen of the mammal hollow organ and further improving the safe use of the catheter 200.

It is to note that use of the three auxiliary holes 5-2 in combination with the end-face opening 11-2 at the distal end of the elongate catheter body of the catheter 200 significantly increases a volume of the biological contents that may enter the medium drainage channel 1-2 from the distal section of the mammal hollow organ or may be supplied/delivered to the distal section of the mammal hollow organ when exiting/leaving the medium drainage channel 1-2.

It is to further note that the hermetic isolation/separation of the medium supply channel 1-1 and the medium drainage channel 1-2 from each other prevents the biological contents transferred through the medium drainage channel 1-2 from being mixed with the medical products (e.g. the enteral or parenteral nutrition mixture) supplied to the medium supply channel 1-1 via the medium supply terminal 9-1 of the connector 12.

In particular, when the flow of biological contents is naturally provided within the mammal hollow organ in a direction from the proximal section of the mammal hollow organ to the distal section of the mammal hollow organ, the medium drainage channel 1-2 allows the biological contents accumulated in the proximal section of the mammal hollow organ, said proximal section being defined by the inflated isolating balloon 4 positioned farther or farthest from the distal end of the elongate catheter body of the catheter 200, to be drained from the proximal section of the mammal hollow organ to the distal section of the mammal hollow organ through the three auxiliary holes 5-2, without entering the isolated target section of the mammal hollow organ, so that the drainage of the accumulated biological contents through the three auxiliary holes 5-2 decompress said biological contents in the proximal section of the mammal hollow organ and eliminates any congestive processes in the proximal section of the mammal hollow organ that would otherwise caused by the accumulated biological contents. Notwithstanding the fact that the flow of biological contents is naturally provided from the proximal section of the mammal hollow organ to the distal section of the mammal hollow organ, an excessively increased pressure within the mammal hollow organ may cause the passing of the flow of biological contents in a backwards direction (i.e. from the distal section of the mammal hollow organ back to the proximal section of the mammal hollow organ), so that the flow of biological contents under the excessively increased pressure may enter the medium drainage channel 1-2 through both the three auxiliary holes 5-1 and the end-face opening 11-2 and may be then drained from the medium drainage channel 1-2 through the three auxiliary holes 5-2, so that the drainage of the biological contents from the medium drainage channel 1-2 through the three auxiliary holes 5-2 prevents the biological contents within the medium drainage channel 1-2 from being supplied/delivered to the medium drainage terminal 9-2 and, subsequently, sprayed to the user.

In various embodiments of the present invention, in the catheter 200, the three auxiliary holes 5-2 of the medium drainage channel 1-2 may be cone-shaped holes, i.e. may have each a geometric shape tapering towards the interior of the medium drainage channel 1-2. In other words, in such various embodiments of the present invention, the three auxiliary holes 5-2 of the medium drainage channel 1-2 may be tapered holes or conical holes tapering towards the interior of the medium drainage channel 1-2, thereby eliminating or reducing leakage of the biological contents being specific to the mammal hollow organ from the inside of the medium drainage channel 1-2 into the proximal section of the mammal hollow organ through the auxiliary holes 5-2 when the excessively increased pressure within the mammal hollow organ is provided. In particular, in such various embodiments of the present invention, the excessively increased pressure provided within the mammal hollow organ causes the passing of the biological contents being specific to the mammal hollow organ in a backwards direction (i.e. from the distal section of the mammal hollow organ back to the proximal section of the mammal hollow organ), so that the biological contents under the excessively increased pressure enter the medium drainage channel 1-2 through both the three auxiliary holes 5-1 and the end-face opening 11-2 and then transferred via the medium drainage channel 1-2 towards the proximal end of the elongate catheter body of the catheter 200, while the conical shapes of the auxiliary holes 5-2 prevent the transferred biological contents from entering the proximal section of the mammal hollow organ via the auxiliary holes 5-2 and, therefore, allow the transferred biological contents to be substantially completely drained via the opening of the medium drainage terminal 9-2.

The above description, including the description of particular embodiments, is analogously applied to the inverse situation: when the flow of biological contents is naturally provided within the mammal hollow organ in a direction from the distal section of the mammal hollow organ to the proximal section of the mammal hollow organ, the medium drainage channel 1-2 allows the biological contents accumulated in the distal section of the mammal hollow organ, said distal section being defined by the inflated isolating balloon 4 positioned closer or closest to the distal end of the elongate catheter body of the catheter 200, to be drained from the distal section of the mammal hollow organ to the proximal section of the mammal hollow organ through both the three auxiliary holes 5-1 and the end-face opening 11-2, without entering the isolated target section of the mammal hollow organ, so that the drainage of the accumulated biological contents through both the three auxiliary holes 5-1 and the end-face opening 11-2 decompress said biological contents in the distal section of the mammal hollow organ and eliminates any congestive processes in the distal section of the mammal hollow organ that would otherwise caused by the accumulated biological contents. Notwithstanding the fact that the flow of biological contents is naturally provided from the distal section of the mammal hollow organ to the proximal section of the mammal hollow organ, an excessively increased pressure within the mammal hollow organ may cause the passing of the flow of biological contents in a backwards direction (i.e. from the proximal section of the mammal hollow organ back to the distal section of the mammal hollow organ), so that the flow of biological contents under the excessively increased pressure may enter the medium drainage channel 1-2 through the three auxiliary holes 5-2 and may be then drained from the medium drainage channel 1-2 through both the three auxiliary holes 5-1 and the end-face opening 11-2, so that the drainage of the biological contents from the medium drainage channel 1-2 through both the three auxiliary holes 5-1 and the end-face opening 11-2 prevents the medium drainage channel 1-2 from being blocked or occluded by the accumulated biological contents.

In the embodiment of the present invention, when the fourth functional device (not shown) is connected to the medium drainage terminal 9-2 of the connector 12 and actuated by the user, air medium or air available in the inner channel of the medium drainage terminal 9-2 and/or air medium or air available in the medium drainage channel 1-2 are removed from the medium drainage terminal 9-2 and the medium drainage channel 1-2 under the pressure generated by the air compressor of the fourth functional device via the opening of the central terminal 9 so as to generate or produce a negative pressure (also referred to in the art as a technical vacuum) in the medium drainage terminal 9-2 and the medium drainage channel 1-2, thereby promoting the aspiration or the evacuation of the biological contents being specific to the mammal hollow organ from distal and proximal sections of the mammal hollow organ. In particular, the biological contents available in the medium drainage channel 1-2 is firstly aspirated/evacuated therefrom to the storage container of the fourth functional device via the opening of the side terminal 8 so as to collect the evacuated biological contents in the storage container. Secondly, the negative pressure produced in the medium drainage channel 1-2 allows the biological contents available outside the medium drainage channel 1-2 in the distal section of the mammal hollow organ to be sucked into the medium drainage channel 1-2 through both the three auxiliary holes 5-1 and the end-face opening 11-2 and allows the biological contents available outside the medium drainage channel 1-2 in the proximal section of the mammal hollow organ to be sucked into the medium drainage channel 1-2 through the three auxiliary holes 5-2, wherein the sucked biological contents is subsequently aspirated/evacuated from the medium drainage channel 1-2 to the storage container of the fourth functional device via the opening of the side terminal 8 so as to collect such a further evacuated biological contents in the storage container.

Therefore, the medium drainage channel 1-2, when connected to the fourth functional device via the medium drainage terminal 9-2, further allows any excessive biological contents being specific to the mammal hollow organ to be partly or completely deflected or vented from the distal and proximal sections of the mammal hollow organ without taking any target biological contents available in the isolated interior of the mammal hollow organ. Furthermore, the removal of the excessive biological contents being specific to the mammal hollow organ from the mammal hollow organ via the medium drainage channel 1-2 having the fourth functional device connected thereto via the medium drainage terminal 9-2 also allows an excessive pressure provided by the excessive biological contents within the distal and proximal sections of the mammal hollow organ to be reduced.

It is to note that the medium drainage channel 1-2 comprising the three auxiliary holes 5-1, the end-face opening 11-2 and the three auxiliary holes 5-2 allows the flow of biological contents being specific to the mammal hollow organ to be passed bidirectionally through the medium drainage channel 1-2, while (i) sampling or taking the gaseous medium, the liquid medium or the fluid medium from the isolated interior of the mammal hollow organ via the functional opening 7 by using the above-described second functional device (particularly, the above-described aspirator) connected to the side terminal 8 of the connector 12 or (ii) supplying the gaseous medium, the liquid medium or the fluid medium to the isolated interior of the mammal hollow organ via the functional opening 7 by using the above-described second functional device (particularly, the above-described medium supply device) connected to the side terminal 8 of the connector 12.

It is to further note that the medium drainage channel 1-2 comprising the three auxiliary holes 5-1, the end-face opening 11-2 and the three auxiliary holes 5-2 also allows any excessive biological contents being specific to the mammal hollow organ to be removed from the distal and proximal sections of the mammal hollow organ, while (i) sampling or taking the gaseous medium, the liquid medium or the fluid medium from the isolated interior of the mammal hollow organ via the functional opening 7 by using the above-described second functional device (particularly, the above-described aspirator) connected to the side terminal 8 of the connector 12 or (ii) supplying the gaseous medium, the liquid medium or the fluid medium to the isolated interior of the mammal hollow organ via the functional opening 7 by using the above-described second functional device (particularly, the above-described medium supply device) connected to the side terminal 8 of the connector 12.

Also, it is to note that the medium drainage channel 1-2 comprising the three auxiliary holes 5-1, the end-face opening 11-2 and the three auxiliary holes 5-2 allows the flow of biological contents being specific to the mammal hollow organ to be passed bidirectionally through the medium drainage channel 1-2, while supplying medical products, in particular the enteral or parenteral nutrition mixture, under the pressure generated by the first functional device connected to the medium supply terminal 9-1.

In some embodiments of the present invention, in the catheter 200, a proximal terminus of the medium drainage channel 1-2 (in particular, a section of the medium drainage channel 1-2 that is between the three auxiliary holes 5-2 and the inner channel of the medium drainage terminal 9-2) may be further provided with a shut-off valve (not shown) installed inside the proximal terminus of the medium drainage channel 1-2, wherein the shut-off valve is designed to be operated by the user or automatically operated to prevent the backflow of the biological contents available in the medium drainage channel 1-2 (especially, under an excessively increased pressure such as an excessively increased intra-abdominal pressure) from the medium drainage channel 1-2 to the medium drainage terminal 9-2. Thus, the shut-off valve installed within the proximal terminus of the medium drainage channel 1-2 prevents any biological contents (e.g. bile) from being uncontrollably passed from the medium drainage channel 1-2 to the medium drainage terminal 9-2 and, therefore, prevents the passed biological contents from being sprayed to the user via the opening of the medium drainage terminal 9-2. In other words, the shut-off valve installed inside the proximal terminus of the medium drainage channel 1-2 is designed to block the leakage of the biological contents available in the medium drainage channel 1-2 under the excessively increased pressure from the medium drainage channel 1-2 in the direction to the opening of the medium drainage terminal 9-2. It is to note that the shut-off valve installed within the proximal terminus of the medium drainage channel 1-2 allows the fourth functional device, when connected to the medium drainage terminal 9-2 and actuated by the user, to provide the controlled discharge of the biological contents from the medium drainage channel 1-2 and from the distal and proximal sections of the mammal hollow organ (i.e. sections of the mammal hollow organ that are outside the isolated interior of the mammal hollow organ) via the opening of the medium drainage terminal 9-2.

In other embodiments of the present invention, in the catheter 200, the medium drainage terminal 9-2 may be further provided with a shut-off valve (not shown) installed inside the inner channel of the medium drainage terminal 9-2, wherein the shut-off valve is designed to be operated by the user or automatically operated to prevent the backflow of the biological contents available in the medium drainage channel 1-2 (especially, under an excessively increased pressure such as an excessively increased intra-abdominal pressure) from the medium drainage channel 1-2 to the opening of the medium drainage terminal 9-2. Thus, the shut-off valve installed within the inner channel of the medium drainage terminal 9-2 prevents any biological contents (e.g. bile) from being uncontrollably passed from the medium drainage channel 1-2 to the opening of the medium drainage terminal 9-2 and, therefore, prevents the passed biological contents from being sprayed to the user via the opening of the medium drainage terminal 9-2. In other words, the shut-off valve installed within the inner channel of the medium drainage terminal 9-2 is designed to block the leakage of the biological contents available in the medium drainage channel 1-2 under the excessively increased pressure from the medium drainage channel 1-2 in the direction to the opening of the medium drainage terminal 9-2. It is to note that the shut-off valve installed within inner channel of the medium drainage terminal 9-2 allows the fourth functional device, when connected to the medium drainage terminal 9-2 and actuated by the user, to provide the controlled discharge of the biological contents from the medium drainage channel 1-2 and from the distal and proximal sections of the mammal hollow organ (i.e. sections of the mammal hollow organ that are outside the isolated interior of the mammal hollow organ) via the opening of the medium drainage terminal 9-2.

In some embodiments of the present invention, in the catheter 200, a proximal terminus of the medium supply channel 1-1 may be further provided with a shut-off valve (not shown) installed inside the proximal terminus of the medium supply channel 1-1, wherein the shut-off valve is designed to be operated by the user or automatically operated to prevent the backflow of the biological contents available in the medium supply channel 1-1 (especially, under an excessively increased pressure such as an excessively increased intra-abdominal pressure) from the medium supply channel 1-1 to the medium supply terminal 9-1. Thus, the shut-off valve installed within the proximal terminus of the medium supply channel 1-1 prevents any biological contents (e.g. bile) from being uncontrollably passed from the medium supply channel 1-1 to the medium supply terminal 9-1 and, therefore, prevents the passed biological contents from being sprayed to the user via the opening of the medium supply terminal 9-1. In other words, the shut-off valve installed inside the proximal terminus of the medium supply channel 1-1 is designed to block the leakage of the biological contents available in the medium supply channel 1-1 under the excessively increased pressure from the medium supply channel 1-1 in the direction to the opening of the medium supply terminal 9-1. It is to note that the shut-off valve installed within the proximal terminus of the medium supply channel 1-1 allows a functional device similar to the fourth functional device, when connected to the medium supply terminal 9-1 and actuated by the user, to provide the controlled discharge of the biological contents from the medium supply channel 1-1 and from the distal and proximal sections of the mammal hollow organ (i.e. sections of the mammal hollow organ that are outside the isolated interior of the mammal hollow organ) via the opening of the medium supply terminal 9-1.

In other embodiments of the present invention, in the catheter 200, the medium supply terminal 9-1 may be further provided with a shut-off valve (not shown) installed inside the inner channel of the medium supply terminal 9-1, wherein the shut-off valve is designed to be operated by the user or automatically operated to prevent the backflow of the biological contents available in the medium supply channel 1-1 (especially, under an excessively increased pressure such as an excessively increased intra-abdominal pressure) from the medium supply channel 1-1 to the opening of the medium supply terminal 9-1. Thus, the shut-off valve installed within the inner channel of the medium supply terminal 9-1 prevents any biological contents (e.g. bile) from being uncontrollably passed from the medium supply channel 1-1 to the opening of the medium supply terminal 9-1 and, therefore, prevents the passed biological contents from being sprayed to the user via the opening of the medium supply terminal 9-1. In other words, the shut-off valve installed within the inner channel of the medium supply terminal 9-1 is designed to block the leakage of the biological contents available in the medium supply channel 1-1 under the excessively increased pressure from the medium supply channel 1-1 in the direction to the opening of the medium supply terminal 9-1. It is to note that the shut-off valve installed within inner channel of the medium supply terminal 9-1 allows a functional device similar to the fourth functional device, when connected to the medium supply terminal 9-1 and actuated by the user, to provide the controlled discharge of the biological contents from the medium supply channel 1-1 and from the distal and proximal sections of the mammal hollow organ (i.e. sections of the mammal hollow organ that are outside the isolated interior of the mammal hollow organ) via the opening of the medium supply terminal 9-1.

In some embodiments of the present invention, the elongate catheter body of the catheter 200 is provided only with the inflation channel 2 and the functional channel 3, wherein the medium supply channel 1-1 and/or the medium drainage channel 1-2 are not provided within the elongate catheter body of the catheter 200.

As shown in FIG. 2.24, each of the two ring-shaped projections 6 used to enclose the functional opening 7 and provided on the elongate catheter body of the catheter 200 is further provided with a traverse groove, slot or cut 17 having a generally square form. Alternatively, in the catheter 200, the traverse groove, slot or cut 17 may have a generally rectangular form, a generally circle form, a generally triangle form, a generally oval form or any another appropriate geometrical form known in the art. It is to note that the each of the two traverse grooves, slots or cuts 17 of the two ring-shaped projections 6 provided on the elongate catheter body of the catheter 200 is designed such that said traverse groove, slot or cut 17 extends through the entire width of a corresponding one of the two ring-shaped projections 6, wherein said width is defined between an inner side of the ring-shaped projection 6, the inner side being faced to the functional opening 7, and an outer side of the ring-shaped projection 6, the outer side being faced to a corresponding one of the two isolating balloons 4.

In case when the negative pressure is produced in the isolated interior of the mammal hollow organ by using the second functional device, the mucosal tissue of the inner wall of the isolated target section of the mammal hollow organ may be closely glued or adhered to both the ring-shaped projections 6 provided on the elongate catheter body of the catheter 200 around all the circumference of the two ring-shaped projections 6 or a part of the circumference of the two ring-shaped projections 6, thereby splitting or dividing the isolated interior of the mammal hollow organ into two separate spaces: (i) an inner space defined within the isolated interior of the mammal hollow organ by (i-a) the inner sides of the two ring-shaped projections 6, (i-b) the part of the elongate catheter body of the catheter 200 between the inner sides of the two ring-shaped projections 6 and (i-c) a portion of the mucosal tissue of the inner wall of the isolated target section of the mammal hollow organ, the portion of the mucosal tissue being closely glued or adhered to both the ring-shaped projections 6 around all the circumference of the two ring-shaped projections 6 or a part of the circumference of the two ring-shaped projections 6 and being positioned between the inner sides of the two ring-shaped projections 6, and (ii) an outer space defined within the isolated interior of the mammal hollow organ by (ii-a) the outer sides of the two ring-shaped projections 6, (ii-b) parts of the elongate catheter body of the catheter 200 between the outer sides of the two ring-shaped projections 6 and the two isolating balloons 4 and (ii-c) portions of the mucosal tissue of the inner wall of the isolated target section of the mammal hollow organ that are positioned between the outer sides of the two ring-shaped projections 6 and the two isolating balloons 4, wherein the inner space is generally surrounded by the outer space or within the outer space, and wherein both the inner space and the outer space in combination form the isolated interior of the mammal hollow organ. In other words, when the negative pressure is produced in the isolated interior of the mammal hollow organ by using the second functional device, the inner space and the outer space surrounding the inner space may be defined within isolated interior of the mammal hollow organ and separated from each other by the mucosal tissue being closely glued or adhered to both the ring-shaped projections 6 provided on the elongate catheter body of the catheter 200 around all the circumference of the two ring-shaped projections 6 or a part of the circumference of the two ring-shaped projections 6.

Meanwhile, as shown in FIG. 2.24, each of the two traverse grooves, slots or cuts 17, said traverse groove, slot or cut 17 being provided in a corresponding one of the two ring-shaped projections 6 provided on the elongate catheter body of the catheter 200, is designed such that said traverse groove, slot or cut 17 defines a through channel which extends/runs through said ring-shaped projection 6 and which allows the gaseous media, liquid media or fluid media (including the biological contents) to be aspirated or evacuated through the defined through channel from the outer space of the isolated interior of the mammal hollow organ to the inner space within the isolated interior of the mammal hollow organ when the mucosal tissue of the inner wall of the isolated target section of the mammal hollow organ is closely glued or adhered to both the ring-shaped projections 6 around all the circumference of the two ring-shaped projections 6 or around a part of the circumference of the two ring-shaped projections 6 due to the negative pressure produced by the second functional device in the interior of the isolated target section of the mammal hollow organ.

In other words, both the through channels provided by the traverse grooves, slots or cuts 17 of the two ring-shaped projections 6 provided on the elongate catheter body of the catheter 200 separately or in combination allow the gaseous media, liquid media or fluid media (including the biological contents) to be to aspirated or evacuated not only from the inner space of the isolated interior of the mammal hollow organ, the inner space being generally isolated within the isolated interior of the mammal hollow organ between the two ring-shaped projections 6 when the mucosal tissue of the inner wall of the isolated target section of the mammal hollow organ is closely glued or adhered to both the ring-shaped projections 6 around all the circumference of the two ring-shaped projections 6 or a part of the circumference of the two ring-shaped projections 6, but also from the outer space of the isolated interior of the mammal hollow organ, the outer space being generally isolated within the isolated interior of the mammal hollow organ between the two ring-shaped projections 6 and the two isolating balloons 4 when the mucosal tissue of the inner wall of the isolated target section of the mammal hollow organ is closely glued or adhered to both the ring-shaped projections 6 around all the circumference of the two ring-shaped projections 6 or a part of the circumference of the two ring-shaped projections 6. Thus, the through channels provided by the traverse grooves, slots or cuts 17 of the two ring-shaped projections 6 provided on the elongate catheter body of the catheter 200 allow the gaseous media, liquid media or fluid media (including the biological contents) to be aspirated or evacuated from all the isolated interior of the mammal hollow organ even when the mucosal tissue of the inner wall of the isolated target section of the mammal hollow organ is closely glued or adhered to both the ring-shaped projections 6 around all the circumference of the two ring-shaped projections 6 or a part of the circumference of the two ring-shaped projections 6.

Both the through channels defined by the traverse grooves, slots or cuts 17 of the two ring-shaped projections 6 provided on the elongate catheter body of the catheter 200 allow the above-described inner space of the isolated interior of the mammal hollow organ and the above-described outer space of the isolated interior of the mammal hollow organ to be in fluid communication with each other, thereby allowing the gaseous media, liquid media or fluid media (including the biological contents) to pass, transfer or flow through said through channels from the outer space to the inner space within the isolated interior of the mammal hollow organ when the mucosal tissue of the inner wall of the isolated target section of the mammal hollow organ is closely glued or adhered to both the ring-shaped projections 6 around all the circumference of the two ring-shaped projections 6 or a part of the circumference of the two ring-shaped projections 6 due to the negative pressure produced by the second functional device in the interior of the isolated target section of the mammal hollow organ. In turn, the aspiration or evacuation of the gaseous media, liquid media or fluid media, e.g. the biological contents, from both the inner space and the outer space of the isolated interior of the mammal hollow organ through the through channels provided by the traverse grooves, slots or cuts 17 of the two ring-shaped projections 6 provided on the elongate catheter body of the catheter 200 results in collection of the biological contents related to all the isolated interior of the mammal hollow organ in the storage container of the aspirator, thereby increasing the accuracy and/or the reliability of the laboratory examinations to be performed on the collected biological contents.

As shown in FIG. 2.24, both the traverse grooves, slots or cuts 17 provided in the two ring-shaped projections 6 provided on the elongate catheter body of the catheter 200 are aligned with the functional opening 7 along the elongate catheter body of the catheter 200, thereby minimizing a length of a pathway from the traverse groove, slot or cut 17 provided in any of the two ring-shaped projections 6 to the functional opening 7, the pathway being required to be overcome by the gaseous media, liquid media or fluid media (including the biological contents) before entering the functional opening 7 when producing the negative pressure in the isolated interior of the mammal hollow organ by means of the second functional device.

As shown in FIG. 2.24, both the traverse grooves, slots or cuts 17 are provided in the two ring-shaped projections 6 provided on the elongate catheter body of the catheter 200 such that they are located/positioned in front of each other or aligned with each other along the length of the elongate catheter body of the catheter 200, thereby forming a straightforward through passage which extends/runs through both the ring-shaped projections 6 and which allows the gaseous media, liquid media or fluid media (including the biological contents) to be aspirated or evacuated through the formed through passage from the outer space of the isolated interior of the mammal hollow organ to the inner space within the isolated interior of the mammal hollow organ when the mucosal tissue of the inner wall of the isolated target section of the mammal hollow organ is closely glued or adhered to both the ring-shaped projections 6 around all the circumference of the two ring-shaped projections 6 or around a part of the circumference of the two ring-shaped projections 6 due to the negative pressure produced by the second functional device in the interior of the isolated target section of the mammal hollow organ, wherein the functional opening 7 is located/positioned in the formed straightforward through passage. In other words, the straightforward through passage extending/running through both the ring-shaped projections 6 comprises the functional opening 7 located/positioned between the two traverse grooves, slots or cuts 17 and aligned with the two traverse grooves, slots or cuts 17.

It is to further note that the functional opening 7 provided in the elongate catheter body of the catheter 200 between the isolating balloons 4 is located/positioned in a generally circular channel formed between inner sides of the two ring-shaped projections 6, wherein the circular channel defined by the inner sides of the two ring-shaped projections 6 and both the through channels formed in the two ring-shaped projections 6 are in fluid communication with each other.

In an embodiment of the present invention, the two traverse grooves, slots or cuts 17 provided in the two ring-shaped projections 6 provided on the elongate catheter body of the catheter 200 may be radially/circumferentially displaced from each other (e.g., spaced at a predetermined interval from each other around the circumference of the elongate catheter body of the catheter 200), so that the through passage formed in the two ring-shaped projections 6 by the two through channels of the two ring-shaped projections 6 may be curved, wherein the functional opening 7 is located/positioned in the formed curved through passage and enclosed by sections of the two ring-shaped projections 6, thereby increasing a length of a pathway from any of the two traverse grooves, slots or cuts 17 provided in the two ring-shaped projections 6 to the functional opening 7, the pathway being required to be overcome by the gaseous media, liquid media or fluid media (including the biological contents) before entering the functional opening 7 when producing the negative pressure in the isolated interior of the mammal hollow organ by means of the second functional device.

In an embodiment of the present invention, each of the two ring-shaped projections 6 provided on the elongate catheter body of the catheter 200 is provided with two or more traverse grooves, slots or cuts 17 (e.g., two, three, four, five, six, seven, eight, nine, ten or more traverse grooves, slots or cuts 17) radially/circumferentially spaced from each other (e.g., equally spaced around the circumference of said ring-shaped projection 6 or circumferentially spaced around said ring-shaped projection 6 at predetermined intervals from each other), thereby forming two or more through channels, each extending/running through a corresponding one of the two ring-shaped projections 6 and each allowing the gaseous media, liquid media or fluid media (including the biological contents) to be aspirated or evacuated through said formed through channel from the above-described outer space of the isolated interior of the mammal hollow organ to the inner space within the isolated interior of the mammal hollow organ when the mucosal tissue of the inner wall of the isolated target section of the mammal hollow organ is closely glued or adhered to both the ring-shaped projections 6 around all the circumference of the two ring-shaped projections 6 or around a part of the circumference of the two ring-shaped projections 6 due to the negative pressure produced by the second functional device in the interior of the isolated target section of the mammal hollow organ. In a first variant of the present embodiment of the present invention, the two or more traverse grooves, slots or cuts 17 provided in one of the two ring-shaped projections 6 and the two or more traverse grooves, slots or cuts 17 provided in the other of the two ring-shaped projections 6 form two or more pairs of longitudinally aligned traverse grooves, slots or cuts and, therefore, form two or more pairs of longitudinally aligned through channels, thereby forming two or more straightforward through passages in the two ring-shaped projection 6, each straightforward through passage comprising a corresponding one of the formed pairs of longitudinally aligned through channels, wherein each of the formed straightforward through passages extends/runs through the two ring-shaped projections 6 and allows the gaseous media, liquid media or fluid media (including the biological contents) to be aspirated or evacuated through said straightforward through passage from the above-described outer space of the isolated interior of the mammal hollow organ to the inner space within the isolated interior of the mammal hollow organ when the mucosal tissue of the inner wall of the isolated target section of the mammal hollow organ is closely glued or adhered to both the ring-shaped projections 6 around all the circumference of the two ring-shaped projections 6 or around a part of the circumference of the two ring-shaped projections 6 due to the negative pressure produced by the second functional device in the interior of the isolated target section of the mammal hollow organ. In the first variant of the present embodiment of the present invention, the functional opening 7 is located/positioned within one of the formed straightforward through passages. In a second variant of the present embodiment of the present invention, the two or more traverse grooves, slots or cuts 17 provided in one of the two ring-shaped projections 6 and the two or more traverse grooves, slots or cuts 17 provided in the other of the two ring-shaped projections 6 are radially/circumferentially displaced from each other and, therefore, through channels defined by said traverse grooves, slots or cuts 17 are radially/circumferentially displaced from each other, thereby forming two or more curved through passages in the two ring-shaped projections 6 that extend/run each through the two ring-shaped projections 6 and that allow each the gaseous media, liquid media or fluid media (including the biological contents) to be aspirated or evacuated through each of the formed curved through passages from the above-described outer space of the isolated interior of the mammal hollow organ to the inner space within the isolated interior of the mammal hollow organ when the mucosal tissue of the inner wall of the isolated target section of the mammal hollow organ is closely glued or adhered to both the ring-shaped projections 6 around all the circumference of the two ring-shaped projections 6 or around a part of the circumference of the two ring-shaped projections 6 due to the negative pressure produced by the second functional device in the interior of the isolated target section of the mammal hollow organ, wherein the functional opening 7 is located/positioned within one of the formed curved through passages. In a third variant of the present embodiment of the present invention, two or more functional openings 7 (e.g., two, three, four, five, six, seven, eight, nine, ten or more functional openings 7) are provided in the elongate catheter body of the catheter 200 between the two ring-shaped projections 6, wherein the functional openings 7 are radially/circumferentially displaced from each other (e.g., equally spaced around the circumference of the elongate catheter body of the catheter 200 or circumferentially spaced around the elongate catheter body of the catheter 200 at predetermined intervals from each other) (alternatively, aligned with each other such that the functional openings 7 are positioned in line with each other along the length of the catheter body part defined by the isolating balloons 4 and spaced from each other at predetermined or equal intervals along the length of the catheter body part defined by the isolating balloons 4) and enclosed by the two ring-shaped projections 6, wherein each of the functional openings 7 is located in a corresponding one of the straightforward through passages mentioned above for the first variant of the present embodiment of the present invention or a corresponding one of the curved through passages mentioned above for the second variant of the present embodiment of the present invention. In the third variant of the present embodiment of the present invention, the increase of number of the functional openings 7 and corresponding through passages, each comprising one of the functional openings, further improves the aspiration or evacuation of the gaseous media, liquid media or fluid media (including the biological contents) from the above-described outer space of the isolated interior of the mammal hollow organ to the inner space within the isolated interior of the mammal hollow organ when the mucosal tissue of the inner wall of the isolated target section of the mammal hollow organ is closely glued or adhered to both the ring-shaped projections 6 around all the circumference of the two ring-shaped projections 6 or around a part of the circumference of the two ring-shaped projections 6 due to the negative pressure produced by the second functional device in the interior of the isolated target section of the mammal hollow organ.

FIG. 2.25 illustrates an alternative embodiment of the present invention where the two traverse grooves, slots or cuts 17 provided in the two ring-shaped projections 6 provided on the elongate catheter body of the catheter 200 are not aligned with the functional opening 7. In particular, as shown in FIG. 2.25, both the traverse grooves, slots or cuts 17 are aligned with each other such that the two through channels defined by the two traverse grooves, slots or cuts 17 form a straightforward through passage extending/running through the two ring-shaped projections 6, while the functional opening 7 is provided in the elongate catheter body of the catheter 200 between the two ring-shaped projections 6 such that the functional opening 7 is enclosed by the two ring-shaped projections 6 and radially/circumferentially displaced from both the traverse grooves, slots or cuts 17 and, therefore, radially/circumferentially displaced from the formed straightforward through passage (i.e. the functional opening 7 is not located/positioned within the formed straightforward through passage), wherein the functional opening 7 is located/positioned in a generally circular channel formed between the inner sides of the two ring-shaped projections 6, and wherein the circular channel defined by the inner sides of the two ring-shaped projections 6 and both the through channels formed in the two ring-shaped projections 6 are in fluid communication with each other, thereby further increasing a length of a pathway from any of the two traverse grooves, slots or cuts 17 provided in the two ring-shaped projections 6 to the functional opening 7, the pathway being required to be overcome by the gaseous media, liquid media or fluid media (including the biological contents) before entering the functional opening 7 when producing the negative pressure in the interior of the isolated target section of the mammal hollow organ by means of the second functional device.

FIG. 2.26 illustrates another alternative embodiment of the present invention where each of the two ring-shaped projections 6 provided on the elongate catheter body of the catheter 200 is provided with four traverse grooves, slots or cuts 17 radially/circumferentially displaced from each other (e.g., equally spaced around the circumference of said ring-shaped projection 6 or circumferentially spaced around said ring-shaped projection 6 at predetermined intervals from each other). As shown in FIG. 2.26, the eight traverse grooves, slots or cuts 17 provided in the two ring-shaped projections 6 provided on the elongate catheter body of the catheter 200 define correspondently eight through channels, each through channel extending/running through a corresponding one of the two ring-shaped projections 6, wherein all the through channels provided in the two ring-shaped projections 6 may be used each for aspirating or evacuating the gaseous media, liquid media or fluid media (e.g. the biological contents) from the outer space of the isolated interior of the mammal hollow organ to the inner space within the isolated interior of the mammal hollow organ when the mucosal tissue of the inner wall of the isolated target section of the mammal hollow organ is closely glued or adhered to both the ring-shaped projections 6 around all the circumference of the two ring-shaped projections 6 or around a part of the circumference of the two ring-shaped projections 6 due to the negative pressure produced by the second functional device in the interior of the isolated target section of the mammal hollow organ. In other words, in such alternative embodiment of the present invention as shown in FIG. 2.26, the increased number of the through channels defined by the traverse grooves, slots or cuts 17 in each of the two ring-shaped projections 6 provided on the elongate catheter body of the catheter 200 simplifies the aspiration or evacuation of the gaseous media, liquid media or fluid media (e.g. the biological contents) from the outer space of the isolated interior of the mammal hollow organ to the inner space within the isolated interior of the mammal hollow organ when the mucosal tissue of the inner wall of the isolated target section of the mammal hollow organ is closely glued or adhered to both the ring-shaped projections 6 around all the circumference of the two ring-shaped projections 6 or around a part of the circumference of the two ring-shaped projections 6 due to the negative pressure produced by the second functional device in the interior of the isolated target section of the mammal hollow organ. Besides, as shown in FIG. 2.26, the four traverse grooves, slots or cuts 17 provided in one of the two ring-shaped projections 6 and the four traverse grooves, slots or cuts 17 provided in the other of the two ring-shaped projections 6 form four pairs of longitudinally aligned traverse grooves, slots or cuts and, therefore, form four pairs of longitudinally aligned through channels, thereby forming four straightforward through passages in the two ring-shaped projection 6, each straightforward through passage comprising a corresponding one of the formed four pairs of longitudinally aligned through channels, wherein each of the four formed straightforward through passages extends/runs through both the ring-shaped projections 6 and allows the gaseous media, liquid media or fluid media (including the biological contents) to be aspirated or evacuated through said straightforward through passage from the above-described outer space of the isolated interior of the mammal hollow organ to the inner space within the isolated interior of the mammal hollow organ when the mucosal tissue of the inner wall of the isolated target section of the mammal hollow organ is closely glued or adhered to both the ring-shaped projections 6 around all the circumference of the two ring-shaped projections 6 or around a part of the circumference of the two ring-shaped projections 6 due to the negative pressure produced by the second functional device in the interior of the isolated target section of the mammal hollow organ. Besides, as shown in FIG. 2.26, the functional opening 7 is located/positioned within one of the four straightforward through passages formed in the two ring-shaped projections 6 and located/positioned in the generally circular channel formed between the inner sides of the two ring-shaped projections 6, the generally circular channel being in fluid communication with said straightforward through passage, while the four straightforward through passages generally run/extend in parallel to each other and are radially/circumferentially displaced from each other (e.g., equally spaced around the circumference of the elongate catheter body of the catheter 200 or circumferentially spaced around the elongate catheter body of the catheter 200 at predetermined intervals from each other).

In still another embodiment of the present invention, each of the two ring-shaped projections 6 provided on the elongate catheter body of the catheter 200 is provided with two traverse grooves, slots or cuts 17 which are positioned radially/circumferentially opposite to each other, thereby forming two radially/circumferentially opposite straightforward or curved through passages in the two ring-shaped projections 6.

In other embodiments of the present invention, each of the two ring-shaped projections 6 provided on the elongate catheter body of the catheter 200 is provided with one or more through-holes (e.g. one, two, three, four, five, six, seven, eight, nine, ten or more through-holes) in addition to the traverse grooves, slots or cuts 17 or instead of them. It is to note that each of the through-holes (not shown) as provided in a corresponding one of the two ring-shaped projections 6 provided on the elongate catheter body of the catheter 200 in such other embodiments of the present invention is designed such that said through-hole defines a further through channel which allows the gaseous media, liquid media or fluid media (including the biological contents) to be aspirated or evacuated through the defined through-holes from the outer space of the isolated interior of the mammal hollow organ to the inner space within the isolated interior of the mammal hollow organ when the mucosal tissue of the inner wall of the isolated target section of the mammal hollow organ is closely glued or adhered to both the ring-shaped projections 6 around all the circumference of the two ring-shaped projections 6 or around a part of the circumference of the two ring-shaped projections 6. In other words, in such other embodiments of the present invention, the through channels provided by the traverse grooves, slots or cuts 17 of the two ring-shaped projections 6 provided on the elongate catheter body of the catheter 200 and/or the further through channels provided by the through-holes (not shown) of the two ring-shaped projections 6 provided on the elongate catheter body of the catheter 200 separately or in combination allow the gaseous media, liquid media or fluid media (including the biological contents) to be to aspirated or evacuated from both the inner space of the isolated interior of the mammal hollow organ and the outer space of the isolated interior of the mammal hollow organ, i.e. all the isolated interior of the mammal hollow organ, when the mucosal tissue of the inner wall of the isolated target section of the mammal hollow organ is closely glued or adhered to both the ring-shaped projections 6 around all the circumference of the ring-shaped projections 6 or a part of the circumference of the two ring-shaped projections 6. In such other embodiments of the present invention, the through channels provided by the traverse grooves, slots or cuts 17 of the two ring-shaped projections 6 provided on the elongate catheter body of the catheter 200 and/or the further through channels provided by the through-holes (not shown) of the two ring-shaped projections 6 provided on the elongate catheter body of the catheter 200 allow the above-described inner space of the isolated interior of the mammal hollow organ and the above-described outer space of the isolated interior of the mammal hollow organ to be in fluid communication with each other, thereby allowing the gaseous media, liquid media or fluid media (including the biological contents) to pass, transfer or flow through said through channels of the two ring-shaped projections 6 and/or said further through channels of the two ring-shaped projections 6 from the outer space to the inner space within the isolated interior of the mammal hollow organ when the mucosal tissue of the inner wall of the isolated target section of the mammal hollow organ is closely glued or adhered to both the ring-shaped projections 6 around all the circumference of the ring-shaped projections 6 or around a part of the circumference of the two ring-shaped projections 6 due to the negative pressure produced in the isolated interior of the mammal hollow organ by means of the second functional device. In turn, in such other embodiments of the present invention, the aspiration or evacuation of the gaseous media, liquid media or fluid media, e.g. the biological contents, from both the inner space and the outer space of the isolated interior of the mammal hollow organ through the through channels provided by the traverse grooves, slots or cuts 17 of the two ring-shaped projections 6 provided on the elongate catheter body of the catheter 200 and/or the further through channels provided by the through-holes (not shown) of the two ring-shaped projections 6 provided on the elongate catheter body of the catheter 200 results in collection of the biological contents related to all the isolated interior of the mammal hollow organ in the storage container of the aspirator, thereby increasing the accuracy and/or the reliability of the laboratory examinations to be performed on the collected biological contents.

In some embodiments of the present invention, each of the two ring-shaped projections 6 provided on the elongate catheter body of the catheter 200 is further provided with a through-hole (not shown), wherein both the through-holes provided in the two ring-shaped projections 6 enclosing the functional opening 7 are aligned with the functional opening 7 along the elongate catheter body of the catheter 200, thereby minimizing a length of a pathway from the through-hole provided in any of the two ring-shaped projections 6 to the functional opening 7, the pathway being required to be overcome by the gaseous media, liquid media or fluid media (including the biological contents) when producing the negative pressure in the isolated interior of the mammal hollow organ by means of the second functional device.

In some other embodiments of the present invention, each of the two ring-shaped projections 6 provided on the elongate catheter body of the catheter 200 is further provided with one or more through-holes (e.g. one, two, three, four, five, six, seven, eight, nine, ten or more through-holes).

In various embodiments of the present invention, each of the two ring-shaped projections 6 provided on the elongate catheter body of the catheter 200 is provided with two or more through-holes (e.g. two, three, four, five, six, seven, eight, nine, ten or more through-holes) equally spaced around the circumference of said ring-shaped projection 6 or circumferentially spaced at predetermined intervals from each other. In such various embodiments of the present invention, the four or more through-holes provided in the two ring-shaped projections 6 provided on the elongate catheter body of the catheter 200 define correspondently four or more through channels which may be used for aspirating or evacuating the gaseous media, liquid media or fluid media (e.g. the biological contents) from the outer space of the isolated interior of the mammal hollow organ to the inner space within the isolated interior of the mammal hollow organ when the mucosal tissue of the inner wall of the isolated target section of the mammal hollow organ is closely glued or adhered to both the ring-shaped projections 6 around all the circumference of the two ring-shaped projections 6 or around a part of the circumference of the two ring-shaped projections 6 due to the negative pressure produced by the second functional device in the interior of the isolated target section of the mammal hollow organ. In other words, in such various embodiments of the present invention, the increase of the through channels defined by the through-holes in each of the two ring-shaped projections 6 provided on the elongate catheter body of the catheter 200 simplifies the aspiration or evacuation of the gaseous media, liquid media or fluid media (e.g. the biological contents) from the outer space of the isolated interior of the mammal hollow organ to the inner space within the isolated interior of the mammal hollow organ when the mucosal tissue of the inner wall of the isolated target section of the mammal hollow organ is closely glued or adhered to both the ring-shaped projections 6 around all the circumference of the two ring-shaped projections 6 or around a part of the circumference of the two ring-shaped projections due to the negative pressure produced by the second functional device in the interior of the isolated target section of the mammal hollow organ.

In still another embodiment of the present invention, each of the two ring-shaped projections 6 provided on the elongate catheter body of the catheter 200 is provided with two through-holes which are positioned radially/circumferentially opposite to each other.

According to one embodiment of the present invention, the elongate catheter body of the catheter 200 may be preliminary provided or equipped with at least one loop or thread (for example, one, two, three, four, five, six, seven, eight, nine, ten or more loops or threads) designed to be gripped with endoscope biopsy forceps for capturing the elongate catheter body of the catheter 200. To insert the elongate catheter body of the catheter 200 into the lumen of the mammal duodenum to have a desired placement site in the mammal duodenum, the distal end of the elongate catheter body of the catheter 200, when preliminary well lubricated with a vaseline oil, is administered or inserted through the mammal nasal passage and advanced to the mammal gastric cavity; then the endoscope is further administered or advanced in parallel with the catheter 200 through the mammal oral cavity to the mammal gastric cavity so as to grip, by means of the endoscope biopsy forceps, the loop or the thread of the elongate catheter body of the catheter 200 for capturing the elongate catheter body of the catheter 200; the captured elongate catheter body is finally guided or advanced along with the endoscope to the mammal duodenum. Subsequently, one of the two isolating balloons 4 of the catheter 200, the farthest from the distal end of elongate catheter body of the catheter 200, is placed in the bulb of the mammal duodenum under control of the endoscope.

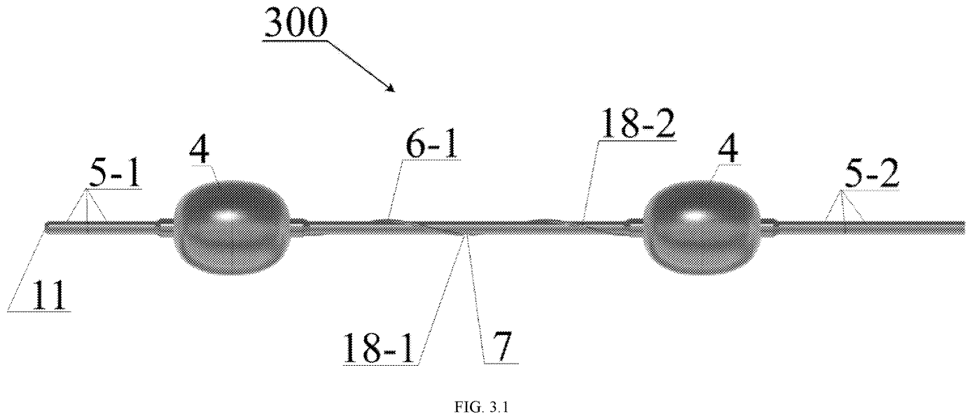

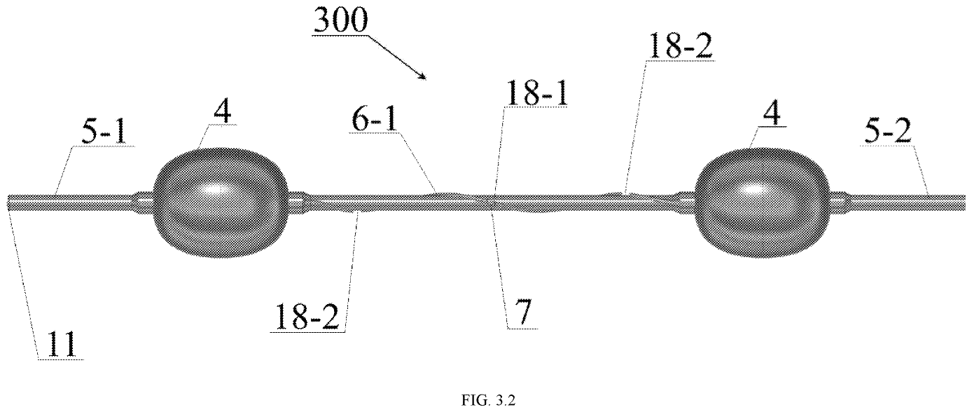

FIGS. 3.1-3.3 schematically illustrate a catheter 300 for isolating an interior of a mammal hollow organ according to the present invention, wherein the catheter 300 is generally structurally and functionally similar to the above-described catheter 100 or the above-described catheter 200, so that the above description of the same structural/functional components of the catheter 100 or 200, including particular embodiments thereof, is applicable to that of the catheter 300.

Contrary to the catheter 100 or the catheter 200, the elongate catheter body of the catheter 300 is not provided with the above-described ring-shaped projections 6 provided on the elongate catheter body of the catheter 100 or the above-described ring-shaped projections 6 provided on the elongate catheter body of the catheter 200. However, the elongate catheter body of the catheter 300 is externally provided with a helical or spiral-shaped projection 6-1, wherein the spiral-shaped projection 6-1 provided on the elongate catheter body of the catheter 300 extends or runs around the catheter body part of the catheter 300 as defined by the isolating balloons 4, and wherein the spiral-shaped projection 6-1 is integral with said catheter body part. It is to note that the spiral-shaped projection 6-1 extends or runs along all the length of the catheter body part of the catheter 300 as defined by the isolating balloons 4. In an embodiment of the present invention, the spiral-shaped projection 6-1 extends or runs along a part or portion of the length of the catheter body part of the catheter 300 as defined by the isolating balloons 4. Thus, except the spiral-shaped projection 6-1, the catheter 300 is structurally and functionally designed as the above-described catheter 100 according to any embodiments thereof or the above-described catheter 200 according to any embodiments thereof.

As shown in FIGS. 3.1-3.3, the spiral-shaped projection 6-1 is designed such that the spiral-shaped projection 6-1 provided on the catheter body part defined by the isolating balloons 4 provided on the elongate catheter body of the catheter 300 has a semicircle-shaped cross-section. Thus, as shown in FIGS. 3.1-3.3, the spiral-shaped projection 6-1 provided on the catheter body part defined by the isolating balloons 4 provided on the elongate catheter body of the catheter 300 is rounded or has a rounded apex or top (i.e. rounded at the top or apex), thereby preventing the mucosal tissue of the inner wall of the isolated target section of the mammal hollow organ from being damaged when said mucosal tissue is closely glued or adhered to the spiral-shaped projection 6-1 around all the circumference of the curved subsections or the spiral turns of the spiral-shaped projection 6-1 or around a part of the circumference of the curved subsections or the spiral turns of the spiral-shaped projection 6-1 due to the negative pressure produced by the second functional device in the interior of the isolated target section of the mammal hollow organ.

In another embodiment of the present invention, the spiral-shaped projection 6-1 externally provided on the catheter body part defined by the isolating balloons 4 provided on the elongate catheter body of the catheter 300 may be designed such that the spiral-shaped projection 6-1 has a rectangle-shaped cross-section. Thus, in the present another embodiment of the present invention, the spiral-shaped projection 6-1 externally provided on the catheter body part defined by the isolating balloons 4 provided on the elongate catheter body of the catheter 300 is flattened (e.g. flattened at the top or apex) or has a flattened apex or top (i.e. the apex or top of the spiral-shaped projection 6-1 is flat and regular), thereby preventing the mucosal tissue of the inner wall of the isolated target section of the mammal hollow organ from being damaged when said mucosal tissue is closely glued or adhered to the spiral-shaped projection 6-1 around all the circumference of the curved subsections or the spiral turns of the spiral-shaped projection 6-1 or around a part of the circumference of the curved subsections or the spiral turns of the spiral-shaped projection 6-1 due to the negative pressure produced by the second functional device in the interior of the isolated target section of the mammal hollow organ. In one variant of the present another embodiment of the present invention, the spiral-shaped projection 6-1 externally provided on the catheter body part of the catheter 300 has a square-shaped cross-section, trapezoid-shaped cross-section or any another suitable cross-section shape known in the art.

As shown in FIGS. 3.1-3.3, the spiral-shaped projection 6-1 is divided by a gap 18-1 into two separate sections having a curved form, the gap 18-1 comprising the functional opening 7 positioned between the two sections of the spiral-shaped projection 6-1. In other words, the two sections of the spiral-shaped projection 6-1 enclose the functional opening 7 provided in the elongate catheter body between the two sections of the spiral-shaped projection 6-1. The functional opening 7 is adjacent to both the sections of the spiral-shaped projection 6-1, wherein the two sections of the spiral-shaped projection 6-1 are equally spaced from the functional opening 7 along the length of the catheter body part of the catheter 300 as defined between the isolating balloons 4 (i.e. a distance between one of the two sections of the spiral-shaped projection 6-1 and the functional opening 7 is equal to a distance between the other section of the spiral-shaped projection 6-1 and the functional opening 7). In other words, the two sections of the spiral-shaped projection 6-1 are equally positioned on different sides of the functional opening 7 along the length of the elongate catheter body of the catheter 300 and each located next or close to the functional opening 7. Therefore, the two sections of the spiral-shaped projection 6-1 that enclose the functional opening 7 are provided on the elongate catheter body of the catheter 300 between the isolating balloons 4 such that the functional opening 7 is between the two sections of the spiral-shaped projection 6-1, wherein the functional opening 7 enclosed by the two sections of the spiral-shaped projection 6-1 is within the gap 18-1 provided in the spiral-shaped projection 6-1.

It is to note that the spiral-shaped projection 6-1 externally provided on the catheter body part of the catheter 300 as defined between the isolating balloons 4 comprises two spiral turns, wherein the gap 18-1 comprising the functional opening 7 is generally provided in a junction of the two spiral turns, the junction being used for connecting the two spiral turns with each other.

In another embodiment of the present invention, the spiral-shaped projection 6-1 comprises one or more spiral turns (in particular, one, two, three, four, five, six, seven, eight, nine, ten or more spiral turns). In still another embodiment of the present invention, the gap 18-1 comprising the functional opening 7 is provided in any of the two spiral turns of the spiral-shaped projection 6-1.

It is to further note that both the spiral turns of the spiral-shaped projection 6-1 have identical sizes (e.g. an inner radius/diameter, an outer radius/diameter, a thickness, a pitch, an inclination/an angle of ascent, curvature, etc.) and have a predetermined axial distance therebetween.

As shown in FIGS. 3.1-3.3, each of the two spiral turns of the spiral-shaped projection 6-1 is further provided with an additional gap 18-2, wherein said spiral turn of the spiral-shaped projection 6-1 is divided by the additional gap 18-2 into two separate sections. In other words, the spiral-shaped projection 6-1 is provided with the two additional gaps 18-2, wherein the gap 18-1 comprising the functional opening 7 is generally positioned between the two additional gaps 18-2.

It is to note that the gap 18-1 and the two additional gaps 18-2 provided in the spiral-shaped projection 6-1 provided on the elongate catheter body of the catheter 300 are radially/circumferentially displaced from each other (e.g., spaced at predetermined or equal intervals from each other around the circumference of the elongate catheter body of the catheter 300 or circumferentially displaced in relation to each other).

It is to further note that the gap 18-1 and the two additional gaps 18-2 provided in the spiral-shaped projection 6-1 provided on the elongate catheter body of the catheter 300 are spaced or distanced from each other along the catheter body part defined by the two isolating balloons 4 (i.e. placed at predetermined or equal intervals from each other along the length of the catheter body part defined by the two isolating balloons 4).

In an embodiment of the present invention, the gap 18-1 and the two additional gaps 18-2 provided in the spiral-shaped projection 6-1 provided on the elongate catheter body of the catheter 300 may be aligned with each other along the length of the catheter body part defined by the two isolating balloons 4.

In another embodiment of the present invention, the spiral-shaped projection 6-1 provided on the elongate catheter body of the catheter 300 is divided by two or more gaps (for example, one, two, three, four, five, six, seven, eight, nine, ten or more gaps), each being designed as the above-described gap 18-1 or the above-described gap 18-2, into three or more separate curved subsections, wherein the functional opening 7 is provided within one of said gaps of the spiral-shaped projection 6-1. In a variant of the present embodiment of the present invention, at least one or each of the curved subsections of the spiral-shaped projection 6-1 may be formed as spiral turns.

Thus, the spiral-shaped projection 6-1 provided on the elongate catheter body of the catheter 300 is divided by the gap 18-1 and the two additional gaps 18-2 into four separate curved subsections, wherein two central curved subsections, each being defined by the gap 18-1 and a corresponding one of the two additional gaps 18-2, are generally identically sized and shaped. It is to note that the two additional gaps 18-2 are provided in the spiral-shaped projection 6-1 such that they are generally equally spaced from the gap 18-1 and positioned at opposite sides of the catheter body part of the catheter 300 as defined by the isolating balloons 4, wherein the gap 18-1 and the two additional gaps 18-2 are circumferentially displaced in relation to each other, i.e. the gap 18-1 and the two additional gaps 18-2 are displaced in relation to each other around the circumference of the catheter body part of the catheter 300 as defined by the isolating balloons 4. Such displaced positions of the gap 18-1 and the two additional gaps 18-2 around the circumference of the catheter body part of the catheter 300 facilitates the gaseous media, liquid media or fluid media (including the biological contents) from the outer space of the isolated interior of the mammal hollow organ to pass, transfer or flow into the inner space within the isolated interior of the mammal hollow organ when the mucosal tissue of the inner wall of the isolated target section of the mammal hollow organ is closely glued or adhered to the spiral-shaped projection 6-1 provided on the elongate catheter body of the catheter 300 around all the circumference of the spiral turns of the spiral-shaped projection 6-1 or around a part of the circumference of the spiral turns of the spiral-shaped projection 6-1 due to the negative pressure produced by the second functional device in the interior of the isolated target section of the mammal hollow organ.

It is to note that the spiral-shaped projection 6-1 provided on the elongate catheter body of the catheter 300 is designed such that the two sections of the spiral-shaped projection 6-1 allow a distance between the functional opening 7, the functional opening 7 being located in the gap 18-1 between the two sections of the spiral-shaped projection 6-1 and being provided in catheter body part of the catheter 300 as defined between the isolating balloons 4, and a mucosal tissue of an inner wall of the mammal hollow organ, the mucosal tissue corresponding to the isolated target section of the mammal hollow organ and being between the inflated isolating balloons 4, to be provided when the negative pressure is produced by the second functional device in the interior of the isolated target section of the mammal hollow organ. In other words, in the catheter 300, the two sections of the spiral-shaped projection 6-1 that enclose the functional opening 7 are intended to prevent the mucosal tissue of the inner wall of the mammal hollow organ, the mucosal tissue being between the inflated isolating balloons 4 and corresponding to the isolated target section of the mammal hollow organ, from contacting with the functional opening 7 or from approaching the functional opening 7 for a distance appropriate for suction of the mucosal tissue into the functional opening 7 when the negative pressure is produced by the second functional device in the isolated target section of the mammal hollow organ, thereby preventing the mucosal tissue of the inner wall of the isolated target section of the mammal hollow organ from being damaged and preventing the functional opening 7 from being blocked or occluded by the sucked mucosal tissue of the inner wall of the isolated target section of the mammal hollow organ. In other words, the two sections of the spiral-shaped projection 6-1 provided on the elongate catheter body of the catheter 300 prevent the suction of the mucosal tissue of the inner wall of the isolated target section of the mammal hollow organ into the functional opening 7, while allowing the gaseous medium, the liquid medium or the fluid medium available in the interior of the isolated target section of the mammal hollow organ to be aspirated or evacuated by the second functional device (not shown) from all the interior of the isolated target section of the mammal hollow organ. Therefore, the two sections of the spiral-shaped projection 6-1 provided on the elongate catheter body of the catheter 300 improve a safety, a reliability and an efficiency of the catheter 300.

Similar to the above described traverse grooves, slots or cuts 17, each of the two additional gaps 18-2 provided in the spiral-shaped projection 6-1, the spiral-shaped projection 6-1 being provided on the elongate catheter body of the catheter 300, allows a positive effect to be provided in case when the mucosal tissue of the inner wall of the isolated target section of the mammal hollow organ is closely glued or adhered to the spiral-shaped projection 6-1 provided on the elongate catheter body of the catheter 300 around all the circumference of the spiral turns of the spiral-shaped projection 6-1 or a part of the circumference of the spiral turns of the spiral-shaped projection 6-1 due to the negative pressure produced in the isolated interior of the mammal hollow organ by means of the second functional device, thereby splitting or dividing the isolated interior of the mammal hollow organ into an inner space of the isolated interior of the mammal hollow organ, the inner space being generally isolated between the glued or adhered mucosal tissue and the spiral turns of the spiral-shaped projection 6-1, and an outer space of the isolated interior of the mammal hollow organ, the outer space being isolated by the glued or adhered mucosal tissue generally between the spiral turns of the spiral-shaped projection 6-1 and the two isolating balloons 4. In other words, when the negative pressure is produced in the isolated interior of the mammal hollow organ by using the second functional device, the inner space and the outer space surrounding the inner space may be defined within isolated interior of the mammal hollow organ and separated from each other by the mucosal tissue being closely glued or adhered to the spiral turns of the spiral-shaped projection 6-1 provided on the elongate catheter body of the catheter 300 around all the circumference of the spiral turns of the spiral-shaped projection 6-1 or a part of the circumference of the spiral turns of the spiral-shaped projection 6-1.

It is to note that the helical or spiral shape of the spiral-shaped projection 6-1 provided on the elongate catheter body of the catheter 300 minimizes the possibility of complete isolation of the inner space of the isolated interior of the mammal hollow organ from the outer space of the isolated interior of the mammal hollow organ, thereby allowing a higher volume of the gaseous media, liquid media or fluid media (including the biological contents) from said outer space to pass, transfer or flow into said inner space when the negative pressure is produced by the second functional device in the interior of the isolated target section of the mammal hollow organ. It is to note that a curved passage is substantially formed by the mucosal tissue of the inner wall of the isolated target section of the mammal hollow organ, said mucosal tissue being closely glued or adhered to the spiral-shaped projection 6-1 provided on the elongate catheter body of the catheter 300, between the spiral-shaped projection 6-1 and the external surface of the catheter body part of the catheter 300, thereby facilitating the gaseous media, liquid media or fluid media (including the biological contents) from said outer space to pass, transfer or flow into said inner space through the formed curved passage.

Meanwhile, each of the two additional gaps 18-2 as provided in the spiral-shaped projection 6-1 provided on the elongate catheter body of the catheter 300 is designed such that said additional gap 18-2 defines a through channel which allows the gaseous media, liquid media or fluid media (including the biological contents) to be aspirated or evacuated through the defined through channel from the outer space of the isolated interior of the mammal hollow organ to the inner space within the isolated interior of the mammal hollow organ when the mucosal tissue of the inner wall of the isolated target section of the mammal hollow organ is closely glued or adhered to the spiral-shaped projection 6-1 provided on the elongate catheter body of the catheter 300 around all the circumference of the spiral turns of the spiral-shaped projection 6-1 or around a part of the circumference of the spiral turns of the spiral-shaped projection 6-1 due to the negative pressure produced by the second functional device in the interior of the isolated target section of the mammal hollow organ.

In other words, both the through channels provided by the two additional gaps 18-2 of the spiral-shaped projection 6-1 provided on the elongate catheter body of the catheter 300 separately or in combination allow the gaseous media, liquid media or fluid media (including the biological contents) to be to aspirated or evacuated not only from the inner space of the isolated interior of the mammal hollow organ via the functional opening 7, but also from the outer space of the isolated interior of the mammal hollow organ to the inner space provided within the isolated interior of the mammal hollow organ, when the mucosal tissue of the inner wall of the isolated target section of the mammal hollow organ is closely glued or adhered to the spiral-shaped projection 6-1 provided on the elongate catheter body of the catheter 300 around all the circumference of the spiral turns of the spiral-shaped projection 6-1 or around a part of the circumference of the spiral turns of the spiral-shaped projection 6-1. Thus, the through channels provided by the two additional gaps 18-2 of the spiral-shaped projection 6-1 provided on the elongate catheter body of the catheter 300 allow the gaseous media, liquid media or fluid media (including the biological contents) to be aspirated or evacuated from all the isolated interior of the mammal hollow organ even when the mucosal tissue of the inner wall of the isolated target section of the mammal hollow organ is closely glued or adhered to the spiral-shaped projection 6-1 provided on the elongate catheter body of the catheter 300 around all the circumference of the spiral turns of the spiral-shaped projection 6-1 or a part of the circumference of the spiral turns of the spiral-shaped projection 6-1.

Both the through channels defined by the two additional gaps 18-2 of the spiral-shaped projection 6-1 provided on the elongate catheter body of the catheter 300 allow the above-described inner space of the isolated interior of the mammal hollow organ and the above-described outer space of the isolated interior of the mammal hollow organ to be in fluid communication with each other, thereby allowing the gaseous media, liquid media or fluid media (including the biological contents) to pass, transfer or flow through said through channels from the outer space to the inner space within the isolated interior of the mammal hollow organ when the mucosal tissue of the inner wall of the isolated target section of the mammal hollow organ is closely glued or adhered to the spiral-shaped projection 6-1 provided on the elongate catheter body of the catheter 300 around all the circumference of the spiral turns of the spiral-shaped projection 6-1 or around a part of the circumference of the spiral turns of the spiral-shaped projection 6-1 due to the negative pressure produced by the second functional device in the interior of the isolated target section of the mammal hollow organ. In turn, the aspiration or evacuation of the gaseous media, liquid media or fluid media, e.g. the biological contents, from both the inner space and the outer space of the isolated interior of the mammal hollow organ through the through channels provided by the two additional gaps 18-2 of the spiral-shaped projection 6-1 provided on the elongate catheter body of the catheter 300 results in collection of the biological contents related to all the isolated interior of the mammal hollow organ in the storage container of the aspirator, thereby increasing the accuracy and/or the reliability of the laboratory examinations to be performed on the collected biological contents.

Therefore, the two additional gaps 18-2 of the spiral-shaped projection 6-1 provided on the elongate catheter body of the catheter 300 further improve a safety, a reliability and an efficiency of the catheter 300.

It is obvious for those skilled in the art that the gap 18-1 and the two additional gaps 18-2 provided in the spiral-shaped projection 6-1 provided on the elongate catheter body of the catheter 300 may be each alternatively named as a slot or an interruption.

In other embodiments of the present invention, the spiral-shaped projection 6-1 provided on the elongate catheter body of the catheter 300 is provided with one or more additional gaps 18-2 (e.g. one, two, three, four, five, six, seven, eight, nine, ten or more additional gaps 18-2).

In some other embodiments of the present invention, the gap 18-1 and the additional gaps 18-2 are aligned with each other along the catheter body part of the catheter 300 as defined by the isolating balloons 4.

In various embodiments of the present invention, at least one or each turn of the spiral-shaped projection 6-1 provided on the elongate catheter body of the catheter 300 is provided with two opposite additional gaps 18-2.

In different embodiments of the present invention, the spiral-shaped projection 6-1 provided on the elongate catheter body of the catheter 300 is only provided with the gap 18-1 comprising the functional opening 7, while the additional gaps 18-2 are not provided in the spiral-shaped projection 6-1.

According to an embodiment of the present invention, two or more functional openings 7 (e.g., two, three, four, five, six, seven, eight, nine, ten or more functional openings 7) are provided in the elongate catheter body of the catheter 300, wherein the spiral-shaped projection 6-1 provided on the elongate catheter body of the catheter 300 is divided by two or more gaps (for example, one, two, three, four, five, six, seven, eight, nine, ten or more gaps), each being designed as the above-described gap 18-1 or the above-described additional gap 18-2, into three or more separate curved subsections, wherein each of the functional openings 7 is provided within one of said gaps of the spiral-shaped projection 6-1. In other words, in the present embodiment of the present invention, the functional openings 7 provided in the elongate catheter body of the catheter 300 are radially/circumferentially displaced from each other (e.g., equally spaced around the circumference of the elongate catheter body of the catheter 300 or circumferentially spaced around the elongate catheter body of the catheter 300 at predetermined intervals from each other), wherein functional openings 7 provided in the elongate catheter body of the catheter 300 are alternatively aligned with each other such that the functional openings 7 are positioned in line with each other along the length of the catheter body part defined by the isolating balloons 4 and spaced from each other at predetermined or equal intervals along the length of the catheter body part defined by the isolating balloons 4). In the present embodiment of the present invention, the increase of number of the functional openings 7, each functional opening 7 being provided in the elongate catheter body of the catheter 300 in a corresponding one of the gaps of the spiral-shaped projection 6-1, further improves the aspiration or evacuation of the gaseous media, liquid media or fluid media (including the biological contents) from the above-described outer space of the isolated interior of the mammal hollow organ to the inner space within the isolated interior of the mammal hollow organ when the mucosal tissue of the inner wall of the isolated target section of the mammal hollow organ is closely glued or adhered to the spiral-shaped projection 6-1 around all the circumference of the spiral-shaped projection 6-1 or around a part of the circumference of the spiral-shaped projection 6-1 due to the negative pressure produced by the second functional device in the interior of the isolated target section of the mammal hollow organ.

According to one embodiment of the present invention, the elongate catheter body of the catheter 300 may be preliminary provided or equipped with at least one loop or thread (for example, one, two, three, four, five, six, seven, eight, nine, ten or more loops or threads) designed to be gripped with endoscope biopsy forceps for capturing the elongate catheter body of the catheter 300. To insert the elongate catheter body of the catheter 300 into the lumen of the mammal duodenum to have a desired placement site in the mammal duodenum, the distal end of the elongate catheter body of the catheter 300, when preliminary well lubricated with a vaseline oil, is administered or inserted through the mammal nasal passage and advanced to the mammal gastric cavity; then the endoscope is further administered or advanced in parallel with the catheter 300 through the mammal oral cavity to the mammal gastric cavity so as to grip, by means of the endoscope biopsy forceps, the loop or the thread of the elongate catheter body of the catheter 300 for capturing the elongate catheter body of the catheter 300; the captured elongate catheter body is finally guided or advanced along with the endoscope to the mammal duodenum. Subsequently, one of the two isolating balloons 4 of the catheter 300, the farthest from the distal end of elongate catheter body of the catheter 300, is placed in the bulb of the mammal duodenum under control of the endoscope.

FIGS. 4.1-4.2 schematically illustrate a catheter 400 for isolating an interior of a mammal hollow organ according to the present invention, wherein the catheter 400 is generally structurally and functionally similar to the above-described catheter 100, so that the above description of the same structural/functional components of the catheter 100, including particular embodiments thereof, is applicable to that of the catheter 400. In an alternative embodiment of the present invention, the catheter 400 may be structurally and functionally similar to the above-described catheter 200, so that the above description of the same structural/functional components of the catheter 200, including particular embodiments thereof, may be also applicable to that of the catheter 400.

Contrary to the catheter 100 or the catheter 200, the elongate catheter body of the catheter 400 is not provided with the above-described ring-shaped projections 6. However, the elongate catheter body of the catheter 400 is externally provided with two helical or spiral-shaped projections 6-2, wherein the two spiral-shaped projections 6-2 provided on the elongate catheter body of the catheter 400 extend or run generally in parallel to each other around the catheter body part of the catheter 400 as defined by the isolating balloons 4, and wherein the two spiral-shaped projections 6-2 are each integral with said catheter body part. It is to note that the two spiral-shaped projections 6-2 both extend or run along all the length of the catheter body part of the catheter 400 as defined by the isolating balloons 4. It is clear for those skilled in art that each of the two spiral-shaped projections 6-2 provided on the catheter body part of the catheter 400 are each generally identical to the above-described spiral-shaped projection 6-1 provided on the catheter body part of the catheter 300. Thus, except the two spiral-shaped projections 6-2, the catheter 400 is structurally and functionally designed as the above-described catheter 100 according to any embodiments thereof or the above-described catheter 200 according to any embodiments thereof.

In an embodiment of the present invention, the two spiral-shaped projections 6-2 both extend or run along a part or portion of the length of the catheter body part of the catheter 400 as defined by the isolating balloons 4.

As shown in FIGS. 4.1-4.2, each of the two spiral-shaped projections 6-2 is designed such that said spiral-shaped projection 6-2 provided on the catheter body part defined by the isolating balloons 4 provided on the elongate catheter body of the catheter 400 has a semicircle-shaped cross-section. Thus, as shown in FIGS. 4.1-4.2, each of the two spiral-shaped projections 6-2 provided on the catheter body part defined by the isolating balloons 4 provided on the elongate catheter body of the catheter 400 is rounded or has a rounded apex or top (i.e. rounded at the top or apex), thereby preventing the mucosal tissue of the inner wall of the isolated target section of the mammal hollow organ from being damaged when said mucosal tissue is closely glued or adhered to both the spiral-shaped projections 6-2 around all the circumference of the curved subsections or the spiral turns of the spiral-shaped projections 6-2 or around a part of the circumference of the curved subsections or the spiral turns of the spiral-shaped projections 6-2 due to the negative pressure produced by the second functional device in the interior of the isolated target section of the mammal hollow organ.

In another embodiment of the present invention, each of the two spiral-shaped projections 6-2 externally provided on the catheter body part defined by the isolating balloons 4 provided on the elongate catheter body of the catheter 400 may be designed such that said spiral-shaped projection 6-2 has a rectangle-shaped cross-section. Thus, in the present another embodiment of the present invention, each of the two spiral-shaped projections 6-2 externally provided on the catheter body part defined by the isolating balloons 4 provided on the elongate catheter body of the catheter 400 is flattened (e.g. flattened at the top or apex) or has a flattened apex or top (i.e. the apex or top of said spiral-shaped projections 6-2 is flat and regular), thereby preventing the mucosal tissue of the inner wall of the isolated target section of the mammal hollow organ from being damaged when said mucosal tissue is closely glued or adhered to both the spiral-shaped projections 6-2 around all the circumference of the curved subsections or the spiral turns of the spiral-shaped projections 6-2 or around a part of the circumference of the curved subsections or the spiral turns of the spiral-shaped projections 6-2 due to the negative pressure produced by the second functional device in the interior of the isolated target section of the mammal hollow organ. In one variant of the present another embodiment of the present invention, each of the two spiral-shaped projections 6-2 externally provided on the catheter body part of the catheter 400 has a square-shaped cross-section, trapezoid-shaped cross-section or any another suitable cross-section shape known in the art.

As also shown in FIGS. 4.1-4.2, the two spiral-shaped projections 6-2 provided on the catheter body part of the catheter 400 are both divided by a gap 18-3 into two separate pairs of parallel sections each having a curved form, wherein the gap 18-3 comprises the functional opening 7 positioned between the two separate pairs of parallel sections of the two spiral-shaped projections 6-2. In other words, the two separate pairs of parallel sections of the two spiral-shaped projections 6-2 enclose the functional opening 7 provided in the elongate catheter body between the two separate pairs of parallel sections of the two spiral-shaped projections 6-2. The functional opening 7 is adjacent to both the two pairs of parallel sections of the two spiral-shaped projections 6-2, wherein the two pairs of parallel sections of the two spiral-shaped projections 6-2 are equally spaced from the functional opening 7 along the length of the catheter body part of the catheter 400 as defined between the isolating balloons 4 (i.e. a distance between one of the two pairs of parallel sections of the two spiral-shaped projections 6-2 and the functional opening 7 is equal to a distance between the other couple of parallel sections of the two spiral-shaped projections 6-2 and the functional opening 7). In other words, the two pairs of parallel sections of the two spiral-shaped projections 6-2 are equally positioned on different sides of the functional opening 7 along the length of the elongate catheter body of the catheter 400 and each located next or close to the functional opening 7. Therefore, the two separate pairs of parallel sections of the two spiral-shaped projections 6-2 that enclose the functional opening 7 are provided on the elongate catheter body of the catheter 400 between the isolating balloons 4 such that the functional opening 7 is between the two separate pairs of parallel sections of the two spiral-shaped projections 6-2, wherein the functional opening 7 enclosed by the two separate pairs of parallel sections of the two spiral-shaped projections 6-2 is within the gap 18-3 provided in the two spiral-shaped projections 6-2.

As also shown in FIGS. 4-1-4-2, the two spiral-shaped projections 6-2 run in parallel at a predetermined distance from each other, thereby forming a curved/spiral-shaped conduct or channel 19 between the two spiral-shaped projections 6-2, wherein the spiral-shaped channel 19 has an interruption corresponding to the gap 18-3. Thus, the two spiral-shaped projections 6-2 are designed such that they define the spiral-shaped channel 19 therebetween, wherein the functional opening 7 is provided in the catheter body part of the catheter 400 in the interruption of the spiral-shaped channel 19 and positioned in the spiral-shaped channel 19 between the two spiral-shaped projections 6-2. In other words, the functional opening 7 provided in the catheter body part of the catheter 400 is positioned between the two spiral-shaped projections 6-2 in a region corresponding to the gap 18-3.

In an embodiment of the present invention, the functional opening 7 provided in the catheter body part of the catheter 400 may be provided in the spiral-shaped channel 19 and may be enclosed by the two spiral-shaped projections 6-2, while the interruption corresponding to the gap 18-3 may be radially/circumferentially displaced from the functional opening 7, thereby increasing a length of a pathway from the interruption provided in the two spiral-shaped projections 6-2 to the functional opening 7, the pathway being required to be overcome by the gaseous media, liquid media or fluid media (including the biological contents) before entering the functional opening 7, when the mucosal tissue of the inner wall of the isolated target section of the mammal hollow organ is closely glued or adhered to both the spiral-shaped projections 6-2 provided on the elongate catheter body of the catheter 400 around all the circumference of the spiral turns of the two spiral-shaped projections 6-2 or around a part of the circumference of the spiral turns of the two spiral-shaped projections 6-2 due to the negative pressure produced by the second functional device in the interior of the isolated target section of the mammal hollow organ.

It is to note that each of the two spiral-shaped projections 6-2 externally provided on the catheter body part of the catheter 400 as defined between the isolating balloons 4 comprises two spiral turns, wherein the four spiral turns of the two spiral-shaped projections 6-2 form two pairs of spinal turns, wherein the two spinal turns related to one of the two pairs of spinal turns are separated by the gap 18-3 from corresponding two spinal turns related to the other pair of spinal turns, and wherein the two spinal turns in each of the two pairs of spinal turns are both are designed such that they are generally spaced from each other and run in parallel with each other, and wherein the gap 18-3 comprising the functional opening 7 is generally provided in two junctions, each being used for connecting the two spiral turns with each other in a corresponding one of the two spiral-shaped projections 6-2.

In another embodiment of the present invention, each of the two spiral-shaped projections 6-2 comprises one or more spiral turns (in particular, one, two, three, four, five, six, seven, eight, nine, ten or more spiral turns), so that one or more pairs of spinal turns are formed by the spiral turns of the two spiral-shaped projections 6-2, wherein the two spinal turns in any of the pairs of spinal turns are both are designed such that they are generally spaced from each other and run in parallel with each other. In still another embodiment of the present invention, the gap 18-3 comprising the functional opening 7 is provided in the two spinal turns in any of the pairs of spiral turns of the two spiral-shaped projections 6-2.

It is to further note that both the spiral turns in each of the two spiral-shaped projections 6-2 have identical sizes (e.g. an inner radius/diameter, an outer radius/diameter, a thickness, a pitch, an inclination/an angle of ascent, curvature, etc.) and have a predetermined axial distance therebetween.

It is to further note that both the spiral turns in each of the two pairs of spinal turns of the two spiral-shaped projections have identical sizes (e.g. an inner radius/diameter, an outer radius/diameter, a thickness, a pitch, an inclination/an angle of ascent, curvature, etc.) and are circumferentially displaced at a predetermined distance from each other.

As shown in FIGS. 4.1-4.2, each of the two pairs of spiral turns of the two spiral-shaped projections 6-2 is further provided with an additional gap 18-4, wherein said spiral turn in said pair of spiral turns is divided by the additional gap 18-4 into two separate sections. In other words, the two spiral-shaped projections 6-2 are provided with the two additional gaps 18-4, wherein the gap 18-3 comprising the functional opening 7 is generally positioned between the two additional gaps 18-4.

It is to note that the gap 18-3 and the two additional gaps 18-4 provided in the two spiral-shaped projections 6-2 provided on the elongate catheter body of the catheter 400 are radially/circumferentially displaced from each other (e.g., spaced at predetermined or equal intervals from each other around the circumference of the elongate catheter body of the catheter 400).

In an embodiment of the present invention, the two spiral-shaped projections 6-2 provided on the elongate catheter body of the catheter 400 are divided by two or more gaps (for example, one, two, three, four, five, six, seven, eight, nine, ten or more gaps), each being designed as the above-described gap 18-3 or the above-described gap 18-4, into three or more pairs of curved subsections or into three or more pairs of spiral turns, wherein the functional opening 7 is provided within one of said gaps of the spiral-shaped projection 6-1 and also located/positioned within the spiral-shaped channel 19 defined by the two spiral-shaped projections 6-2.

Thus, the two spiral-shaped projections 6-2 provided on the elongate catheter body of the catheter 400 are divided by the gap 18-3 and the two additional gaps 18-4 into four separate pairs of curved subsections, wherein two central pairs of curved subsections, each being defined by the gap 18-3 and a corresponding one of the two additional gaps 18-4, are generally identically sized and shaped. It is to note that the two additional gaps 18-4 are provided in the two spiral-shaped projections 6-2 such that they are generally equally spaced from the gap 18-3 and positioned at opposite sides of the catheter body part of the catheter 400 as defined by the isolating balloons 4, wherein the gap 18-3 and the two additional gaps 18-4 are circumferentially displaced in relation to each other, i.e. the gap 18-3 and the two additional gaps 18-4 are displaced in relation to each other around the circumference of the catheter body part of the catheter 400 as defined by the isolating balloons 4. Such displaced positions of the gap 18-3 and the two additional gaps 18-4 around the circumference of the catheter body part of the catheter 400 facilitates the flow of the gaseous media, liquid media or fluid media (including the biological contents) from the outer space of the isolated interior of the mammal hollow organ to pass, transfer or flow to the inner space provided within the isolated interior of the mammal hollow organ when the mucosal tissue of the inner wall of the isolated target section of the mammal hollow organ is closely glued or adhered to both the spiral-shaped projections 6-2 provided on the elongate catheter body of the catheter 400 around all the circumference of the spiral turns of the two spiral-shaped projections 6-2 or around a part of the circumference of the spiral turns of the two spiral-shaped projections 6-2 due to the negative pressure produced by the second functional device in the interior of the isolated target section of the mammal hollow organ.

It is to note that the two spiral-shaped projections 6-2 provided on the elongate catheter body of the catheter 400 are both designed such that the two pairs of parallel sections of the two spiral-shaped projections 6-2 allow a distance between the functional opening 7, the functional opening 7 being located in the gap 18-3 between the two pairs of parallel sections of the two spiral-shaped projections 6-2 and being provided in catheter body part of the catheter 400 as defined between the isolating balloons 4, and the mucosal tissue of the inner wall of the mammal hollow organ, the mucosal tissue corresponding to the isolated target section of the mammal hollow organ and being between the inflated isolating balloons 4, to be provided when the negative pressure is produced by the second functional device in the interior of the isolated target section of the mammal hollow organ. In other words, in the catheter 400, the two pairs of parallel sections of the two spiral-shaped projections 6-2 that enclose the functional opening 7 are intended to prevent the mucosal tissue of the inner wall of the mammal hollow organ, the mucosal tissue being between the inflated isolating balloons 4 and corresponding to the isolated target section of the mammal hollow organ, from contacting with the functional opening 7 or from approaching the functional opening 7 for a distance appropriate for suction of the mucosal tissue into the functional opening 7 when the negative pressure is produced by the second functional device in the isolated target section of the mammal hollow organ, thereby preventing the mucosal tissue of the inner wall of the isolated target section of the mammal hollow organ from being damaged and preventing the functional opening 7 from being blocked or occluded by the sucked mucosal tissue of the inner wall of the isolated target section of the mammal hollow organ. In other words, the two pairs of parallel sections of the two spiral-shaped projections 6-2 provided on the elongate catheter body of the catheter 400 prevent the suction of the mucosal tissue of the inner wall of the isolated target section of the mammal hollow organ into the functional opening 7, while allowing the gaseous medium, the liquid medium or the fluid medium available in the interior of the isolated target section of the mammal hollow organ to be aspirated or evacuated by the second functional device (not shown) from all the interior of the isolated target section of the mammal hollow organ. Therefore, the two pairs of parallel sections of the two spiral-shaped projections 6-2 provided on the elongate catheter body of the catheter 400 improve a safety, a reliability and an efficiency of the catheter 400.

Similar to the above described traverse grooves, slots or cuts 17, each of the two additional gaps 18-4 provided in the two spiral-shaped projections 6-2, the two spiral-shaped projections 6-2 being provided on the elongate catheter body of the catheter 400, allows a positive effect to be provided in case when the mucosal tissue of the inner wall of the isolated target section of the mammal hollow organ is closely glued or adhered to the two spiral-shaped projections 6-2 provided on the elongate catheter body of the catheter 400 around all the circumference of the spiral turns of the two spiral-shaped projections 6-2 or a part of the circumference of the spiral turns of the two spiral-shaped projections 6-2 due to the negative pressure produced in the isolated interior of the mammal hollow organ by means of the second functional device, thereby splitting or dividing the isolated interior of the mammal hollow organ into an inner space of the isolated interior of the mammal hollow organ, the inner space being generally isolated between the glued or adhered mucosal tissue and the spiral turns of the two spiral-shaped projections 6-2, and an outer space of the isolated interior of the mammal hollow organ, the outer space being isolated by the glued or adhered mucosal tissue generally between the spiral turns of the two spiral-shaped projections 6-2 and the two isolating balloons 4. In other words, when the negative pressure is produced in the isolated interior of the mammal hollow organ by using the second functional device, the inner space and the outer space surrounding the inner space may be defined within isolated interior of the mammal hollow organ and separated from each other by the mucosal tissue being closely glued or adhered to the spiral turns of the two spiral-shaped projections 6-2 provided on the elongate catheter body of the catheter 400 around all the circumference of the spiral turns of the two spiral-shaped projections 6-2 or a part of the circumference of the spiral turns of the two spiral-shaped projections 6-2.

It is to note that the helical or spiral shape of each of the two spiral-shaped projections 6-2 provided on the elongate catheter body of the catheter 400 minimizes the possibility of complete isolation of the inner space of the isolated interior of the mammal hollow organ from the outer space of the isolated interior of the mammal hollow organ, thereby allowing a higher volume of the gaseous media, liquid media or fluid media (including the biological contents) from said outer space to pass, transfer or flow to said inner space when the mucosal tissue of the inner wall of the isolated target section of the mammal hollow organ is closely glued or adhered to both the spiral-shaped projections 6-2 provided on the elongate catheter body of the catheter 400 around all the circumference of the spiral turns of the two spiral-shaped projections 6-2 or around a part of the circumference of the spiral turns of the two spiral-shaped projections 6-2 due to the negative pressure produced by the second functional device in the interior of the isolated target section of the mammal hollow organ due to the negative pressure produced by the second functional device in the interior of the isolated target section of the mammal hollow organ.

Meanwhile, each of the two additional gaps 18-4 as provided in the two spiral-shaped projections 6-2 provided on the elongate catheter body of the catheter 400 is designed such that said additional gap 18-4 defines a through channel which allows the gaseous media, liquid media or fluid media (including the biological contents) to be aspirated or evacuated through the defined through channel from the outer space of the isolated interior of the mammal hollow organ to the inner space provided within the isolated interior of the mammal hollow organ when the mucosal tissue of the inner wall of the isolated target section of the mammal hollow organ is closely glued or adhered to both the spiral-shaped projections 6-2 provided on the elongate catheter body of the catheter 400 around all the circumference of the spiral turns of the two spiral-shaped projections 6-2 or around a part of the circumference of the spiral turns of the two spiral-shaped projections 6-2 due to the negative pressure produced by the second functional device in the interior of the isolated target section of the mammal hollow organ.

In other words, both the through channels provided by the two additional gaps 18-4 of the two spiral-shaped projections 6-2 provided on the elongate catheter body of the catheter 400 separately or in combination allow the gaseous media, liquid media or fluid media (including the biological contents) to be to aspirated or evacuated not only from the inner space provided within the isolated interior of the mammal hollow organ, but also from the outer space of the isolated interior of the mammal hollow organ to the inner space provided within the isolated interior of the mammal hollow organ, when the mucosal tissue of the inner wall of the isolated target section of the mammal hollow organ is closely glued or adhered to both the spiral-shaped projections 6-2 provided on the elongate catheter body of the catheter 400 around all the circumference of the spiral turns of the two spiral-shaped projections 6-2 or around a part of the circumference of the spiral turns of the two spiral-shaped projections 6-2 due to the negative pressure produced by the second functional device in the interior of the isolated target section of the mammal hollow organ. Thus, the through channels provided by the two additional gaps 18-4 of the two spiral-shaped projections 6-2 provided on the elongate catheter body of the catheter 400 allow the gaseous media, liquid media or fluid media (including the biological contents) to be aspirated or evacuated from all the isolated interior of the mammal hollow organ even when the mucosal tissue of the inner wall of the isolated target section of the mammal hollow organ is closely glued or adhered to the two spiral-shaped projections 6-2 provided on the elongate catheter body of the catheter 400 around all the circumference of the spiral turns of the two spiral-shaped projections 6-2 or a part of the circumference of the spiral turns of the two spiral-shaped projections 6-2.

Both the through channels defined by the two additional gaps 18-4 of the two spiral-shaped projections 6-2 provided on the elongate catheter body of the catheter 400 allow the above-described inner space of the isolated interior of the mammal hollow organ and the above-described outer space of the isolated interior of the mammal hollow organ to be in fluid communication with each other, thereby allowing the gaseous media, liquid media or fluid media (including the biological contents) to pass, transfer or flow through said through channels from the outer space to the inner space provided within the isolated interior of the mammal hollow organ when the mucosal tissue of the inner wall of the isolated target section of the mammal hollow organ is closely glued or adhered to both the spiral-shaped projections 6-2 provided on the elongate catheter body of the catheter 400 around all the circumference of the spiral turns of the two spiral-shaped projections 6-2 or around a part of the circumference of the spiral turns of the two spiral-shaped projections 6-2 due to the negative pressure produced by the second functional device in the interior of the isolated target section of the mammal hollow organ due to the negative pressure produced by the second functional device in the interior of the isolated target section of the mammal hollow organ by using the second functional device. In turn, the aspiration or evacuation of the gaseous media, liquid media or fluid media, e.g. the biological contents, from both the inner space and the outer space of the isolated interior of the mammal hollow organ through the through channels provided by the two additional gaps 18-4 of the two spiral-shaped projections 6-2 provided on the elongate catheter body of the catheter 400 results in collection of the biological contents related to all the isolated interior of the mammal hollow organ in the storage container of the aspirator, thereby increasing the accuracy and/or the reliability of the laboratory examinations to be performed on the collected biological contents.

Moreover, in the catheter 400, each of the gap 18-3 and two additional gaps 18-4 is in fluid communication with the spiral-shaped channel 19 extending between the two spiral-shaped projections 6-2 provided on the elongate catheter body of the catheter 400, thereby further allowing the gaseous media, liquid media or fluid media (including the biological contents) to pass, transfer or flow from the outer space of the isolated interior of the mammal hollow organ to the inner space provided within the isolated interior of the mammal hollow organ when the mucosal tissue of the inner wall of the isolated target section of the mammal hollow organ is closely glued or adhered to both the spiral-shaped projections 6-2 provided on the elongate catheter body of the catheter 400 around all the circumference of the spiral turns of the two spiral-shaped projections 6-2 or around a part of the circumference of the spiral turns of the two spiral-shaped projections 6-2 due to the negative pressure produced by the second functional device in the interior of the isolated target section of the mammal hollow organ.

Therefore, the two additional gaps 18-4 of the two spiral-shaped projections 6-2 provided on the elongate catheter body of the catheter 400 further improve a safety, a reliability and an efficiency of the catheter 400.

It is obvious for those skilled in the art that the gap 18-3 and the two additional gaps 18-4 provided in the two spiral-shaped projections 6-2 provided on the elongate catheter body of the catheter 400 may be each alternatively named as a slot or an interruption.

In other embodiments of the present invention, the two spiral-shaped projections 6-2 provided on the elongate catheter body of the catheter 400 is provided with one or more additional gaps 18-4 (e.g. one, two, three, four, five, six, seven, eight, nine, ten or more additional gaps 18-4).

In some other embodiments of the present invention, the gap 18-3 and the additional gaps 18-4 are aligned with each other along the catheter body part of the catheter 400 as defined by the isolating balloons 4.

In various embodiments of the present invention, at least one or each turn in each of the two spiral-shaped projections 6-2 provided on the elongate catheter body of the catheter 400 is provided with two opposite additional gaps 18-4.

In different embodiments of the present invention, the two spiral-shaped projections 6-2 provided on the elongate catheter body of the catheter 400 is only provided with the gap 18-3 comprising the functional opening 7, while the additional gaps 18-4 are not provided in the two spiral-shaped projections 6-2.

According to an embodiment of the present invention, two or more functional openings 7 (e.g., two, three, four, five, six, seven, eight, nine, ten or more functional openings 7) are provided in the elongate catheter body of the catheter 400, wherein the two spiral-shaped projections 6-2 provided on the elongate catheter body of the catheter 400 are divided by two or more gaps (for example, one, two, three, four, five, six, seven, eight, nine, ten or more gaps), each gap being designed as the above-described gap 18-3 or the above-described additional gap 18-4, into three or more pairs of curved subsections or into three or more pairs of spiral turns, wherein each of the functional openings 7 is provided within one of said gaps of the two spiral-shaped projections 6-2 and also located/positioned within the spiral-shaped channel 19 defined by the two spiral-shaped projections 6-2. In other words, in the present embodiment of the present invention, the functional openings 7 provided in the elongate catheter body of the catheter 400 are radially/circumferentially displaced from each other (e.g., equally spaced around the circumference of the elongate catheter body of the catheter 400 or circumferentially spaced around the elongate catheter body of the catheter 400 at predetermined intervals from each other), wherein the functional openings 7 provided in the elongate catheter body of the catheter 400 are alternatively aligned with each other such that the functional openings 7 are in line with each other along the length of the catheter body part defined by the isolating balloons 4 and spaced from each other at predetermined or equal intervals along the length of the catheter body part defined by the isolating balloons 4. In the present embodiment of the present invention, the increase of number of the functional openings 7, each functional opening 7 being provided in the elongate catheter body of the catheter 400 in a corresponding one of the gaps of the two spiral-shaped projections 6-2, further improves the aspiration or evacuation of the gaseous media, liquid media or fluid media (including the biological contents) from the above-described outer space of the isolated interior of the mammal hollow organ to the inner space within the isolated interior of the mammal hollow organ when the mucosal tissue of the inner wall of the isolated target section of the mammal hollow organ is closely glued or adhered to both the spiral-shaped projections 6-2 around all the circumference of the curved subsections or the spiral turns of the spiral-shaped projections 6-2 or around a part of the circumference of the curved subsections or the spiral turns of the spiral-shaped projections 6-2 due to the negative pressure produced by the second functional device in the interior of the isolated target section of the mammal hollow organ.

According to another embodiment of the present invention, the elongate catheter body of the catheter 400 may be preliminary provided or equipped with at least one loop or thread (for example, one, two, three, four, five, six, seven, eight, nine, ten or more loops or threads) designed to be gripped with endoscope biopsy forceps for capturing the elongate catheter body of the catheter 400. To insert the elongate catheter body of the catheter 400 into the lumen of the mammal duodenum to have a desired placement site in the mammal duodenum, the distal end of the elongate catheter body of the catheter 400, when preliminary well lubricated with a vaseline oil, is administered or inserted through the mammal nasal passage and advanced to the mammal gastric cavity; then the endoscope is further administered or advanced in parallel with the catheter 400 through the mammal oral cavity to the mammal gastric cavity so as to grip, by means of the endoscope biopsy forceps, the loop or the thread of the elongate catheter body of the catheter 400 for capturing the elongate catheter body of the catheter 400; the captured elongate catheter body is finally guided or advanced along with the endoscope to the mammal duodenum. Subsequently, one of the two isolating balloons 4 of the catheter 400, the farthest from the distal end of elongate catheter body of the catheter 400, is placed in the bulb of the mammal duodenum under control of the endoscope.

FIGS. 4.3-4.4 illustrates an alternative embodiment of the present invention where gap 18-3 is not provided in the two spiral-shaped projections 6-2 provided on the elongate catheter body of the catheter 400, and two gaps 18-4 are only provided in the two spiral-shaped projections 6-2, wherein each of the two spiral-shaped projections 6-2 is designed such that said spiral-shaped projection 6-2 has a semicircle-shaped cross-section. Thus, as shown in FIGS. 4.3-4.4, the each of the two spiral-shaped projections 6-2 is rounded or has a rounded apex or top (i.e. rounded at the top or apex), thereby preventing the mucosal tissue of the inner wall of the isolated target section of the mammal hollow organ from being damaged when said mucosal tissue is closely glued or adhered to both the spiral-shaped projections 6-2 around all the circumference of the curved subsections or the spiral turns of the spiral-shaped projections 6-2 or around a part of the circumference of the curved subsections or the spiral turns of the spiral-shaped projections 6-2 due to the negative pressure produced by the second functional device in the interior of the isolated target section of the mammal hollow organ. As shown in FIGS. 4.3-4.4, the functional opening 7 is within the spiral-shaped channel 19 defined by the two spiral-shaped projections 6-2, i.e. the functional opening 7 is not further located/positioned within the gap 18-3, wherein the spiral-shaped channel 19 is in fluid communication with both the gaps 18-4, thereby providing the fluid communication between the inner space and the outer space of the isolated interior of the mammal hollow organ when the mucosal tissue of the inner wall of the isolated target section of the mammal hollow organ is closely glued or adhered to both the spiral-shaped projections 6-2 around all the circumference of the curved subsections or the spiral turns of the spiral-shaped projections 6-2 or around a part of the circumference of the curved subsections or the spiral turns of the spiral-shaped projections 6-2 due to the negative pressure produced by the second functional device in the interior of the isolated target section of the mammal hollow organ. In the present alternative embodiment of the present invention, that fact that the functional opening 7 located/positioned within the spiral-shaped channel 19 is spaced from both the gaps 18-4 provided in the spiral-shaped projections 6-2 along the length of the elongate catheter body of the catheter 400 and radially/circumferentially displaced from both the gaps 18-4 provided in spiral-shaped projections 6-2 increases a length of a pathway from any the two interruptions provided in the two spiral-shaped projections 6-2 (wherein each of the two interruptions corresponds to a particular one of the two gaps 18-4) to the functional opening 7, the pathway being required to be overcome by the gaseous media, liquid media or fluid media (including the biological contents) before entering the functional opening 7, when the mucosal tissue of the inner wall of the isolated target section of the mammal hollow organ is closely glued or adhered to both the spiral-shaped projections 6-2 provided on the elongate catheter body of the catheter 400 around all the circumference of the spiral turns of the two spiral-shaped projections 6-2 or around a part of the circumference of the spiral turns of the two spiral-shaped projections 6-2 due to the negative pressure produced by the second functional device in the interior of the isolated target section of the mammal hollow organ. In a first variant of the present alternative embodiment of the present invention, two or more functional openings 7 (e.g., two, three, four, five, six, seven, eight, nine, ten or more functional openings 7) are within the spiral-shaped channel 19 and spaced from each other along the length of the elongate catheter body of the catheter 400 (wherein all the functional openings 7 or a part of them are further radially/circumferentially displaced from each other), wherein all the functional openings 7 are located/positioned between the gaps 18-4 provided in the two spiral-shaped projections 6-2 along the length of the elongate catheter body of the catheter 400. In a second variant of the present alternative embodiment of the present invention, three functional openings 7 may be provided in the elongate catheter body of the catheter 400 within the spiral-shaped channel 19, and three gaps, each gap corresponding to the above-described gap 18-3 or the above-described gap 18-4, are provided in the two spiral-shaped projections 6-2, wherein the three functional openings 7 are equally spaced along the length of the elongate catheter body of the catheter 400 and radially/circumferentially displaced in relation to each other (alternatively, aligned with each other such that the functional openings 7 are in line with each other along the length of the catheter body part defined by the isolating balloons 4 and spaced from each other at predetermined or equal intervals along the length of the catheter body part defined by the isolating balloons 4), and wherein the three gaps provided in the two spiral-shaped projections 6-2 are equally spaced along the length of the elongate catheter body of the catheter 400 and radially/circumferentially displaced in relation to each other. In the second variant of the present alternative embodiment of the present invention, each of the three functional openings 7 is within a corresponding one of the three gaps provided in the two spiral-shaped projections 6-2.

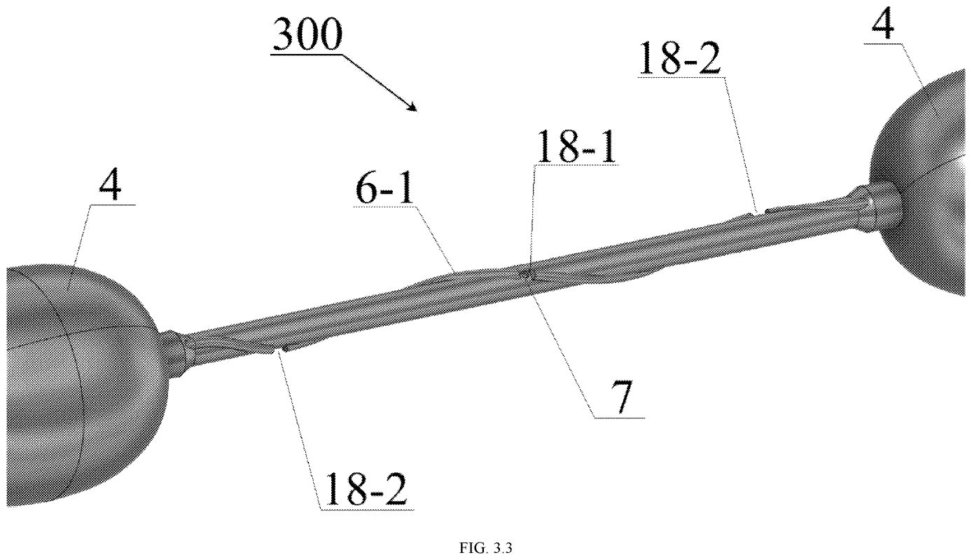

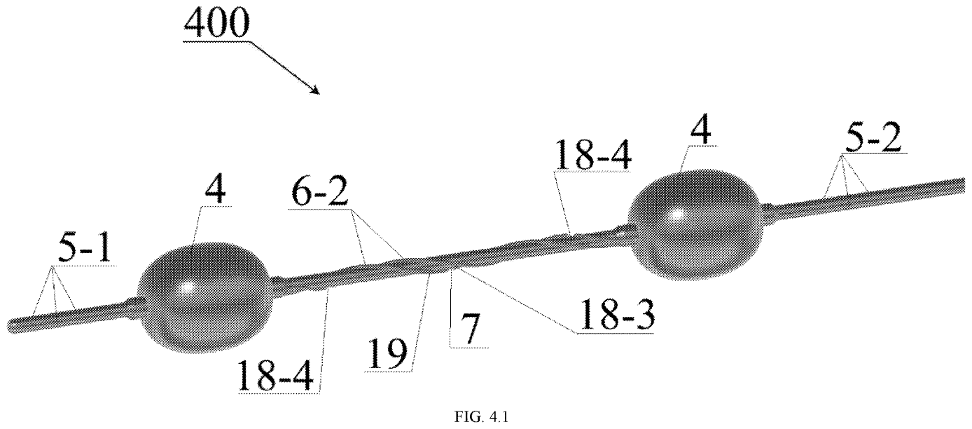

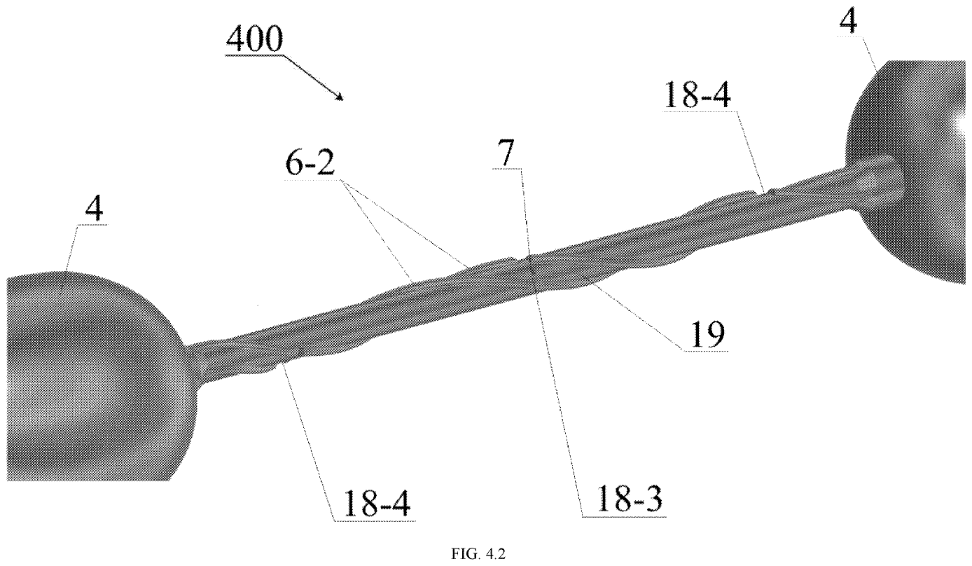

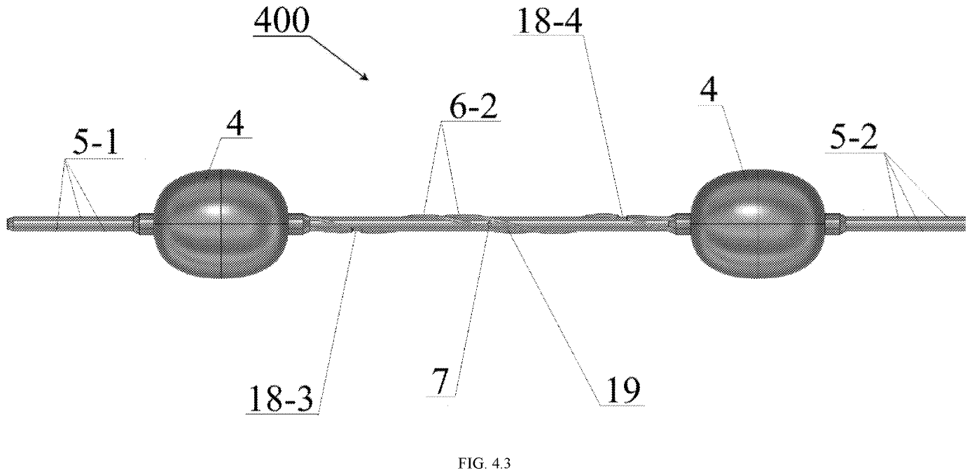

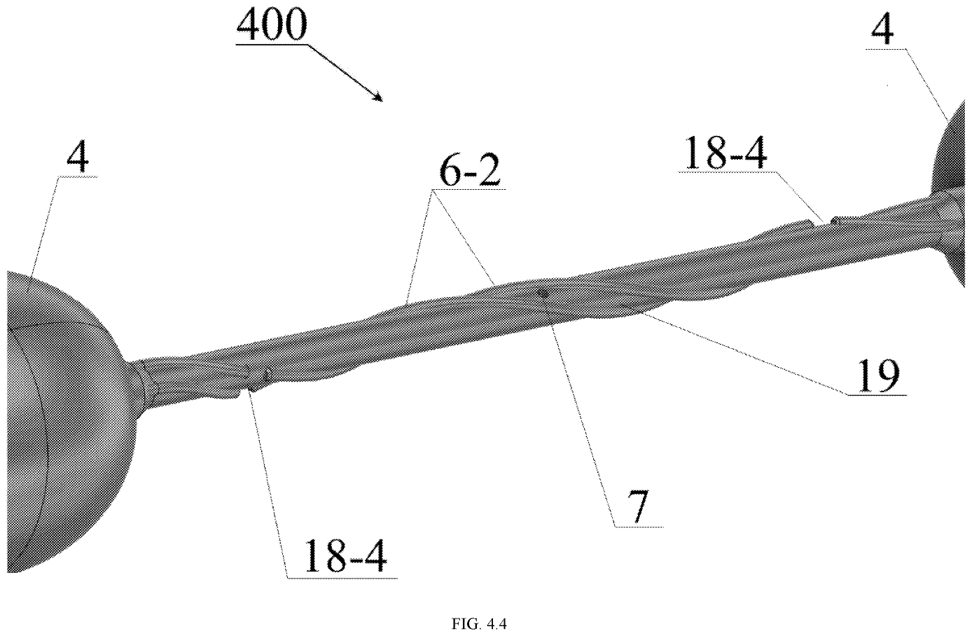

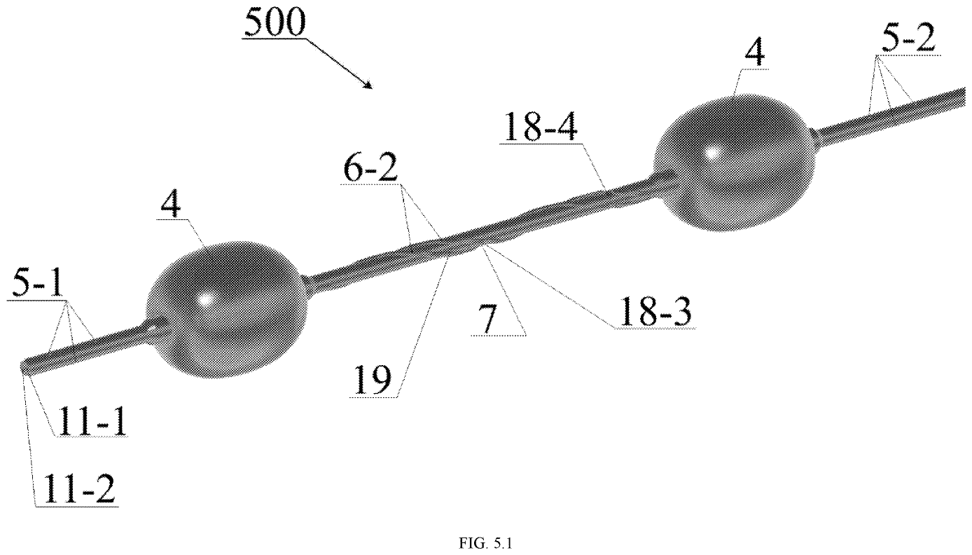

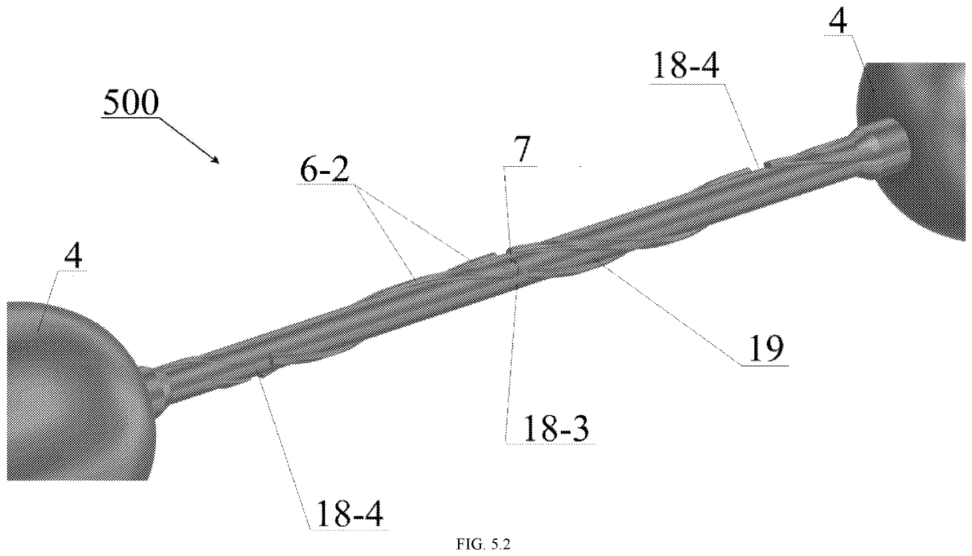

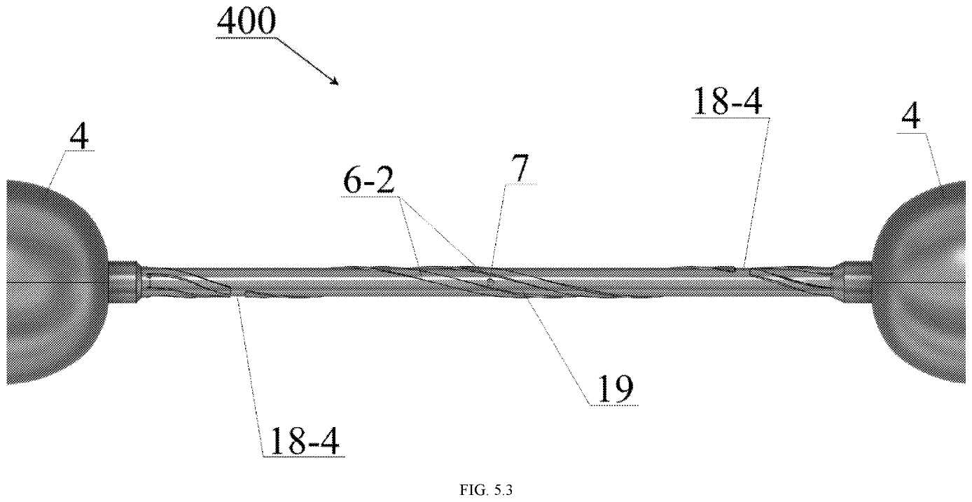

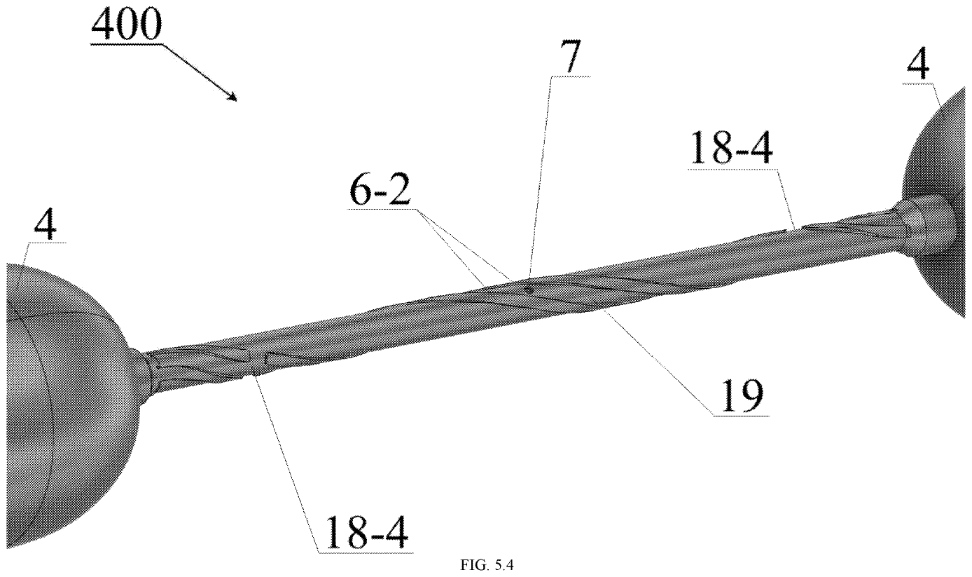

FIGS. 5.1-5.2 schematically illustrate a catheter 500 for isolating an interior of a mammal hollow organ according to the present invention, wherein the catheter 500 is generally structurally and functionally similar to the above-described catheter 200, so that the above description of the same structural/functional components of the catheter 200, including particular embodiments thereof, is applicable to that of the catheter 500. In an alternative embodiment of the present invention, the catheter 500 may be structurally and functionally similar to the above-described catheter 100, so that the above description of the same structural/functional components of the catheter 100, including particular embodiments thereof, may be also applicable to that of the catheter 500.

Contrary to the catheter 100 or the catheter 200, the elongate catheter body of the catheter 500 shown in FIGS. 5.1-5.2 is not provided with the above-described ring-shaped projections 6. However, the elongate catheter body of the catheter 500 shown in FIGS. 5.1-5.2 is externally provided with two helical or spiral-shaped projections 6-2, wherein the two spiral-shaped projections 6-2 provided on the elongate catheter body of the catheter 500 extend or run generally in parallel to each other around the catheter body part of the catheter 500 as defined by the isolating balloons 4, and wherein the two spiral-shaped projections 6-2 are each integral with said catheter body part. It is to note that the two spiral-shaped projections 6-2 both extend or run along all the length of the catheter body part of the catheter 500 shown in FIGS. 5.1-5.2, the catheter body part being defined by the isolating balloons 4. It is clear for those skilled in art that each of the two spiral-shaped projections 6-2 provided on the catheter body part of the catheter 500 shown in FIGS. 5.1-5.2 are each generally identical to the above-described spiral-shaped projection 6-1 provided on the catheter body part of the catheter 300. Thus, except the two spiral-shaped projections 6-2, the catheter 500 shown in FIGS. 5.1-5.2 is structurally and functionally designed as the above-described catheter 100 according to any embodiments thereof or the above-described catheter 200 according to any embodiments thereof.

As shown in FIGS. 5.1-5.2, each of the two spiral-shaped projections 6-2 is designed such that said spiral-shaped projection 6-2 has a rectangle-shaped cross-section. Thus, as shown in FIGS. 5.1-5.2, the each of the two spiral-shaped projections 6-2 is flattened (e.g. flattened at the top or apex) or has a flattened apex or top (i.e. the apex or top of said spiral-shaped projections 6-2 is flat and regular), thereby preventing the mucosal tissue of the inner wall of the isolated target section of the mammal hollow organ from being damaged when said mucosal tissue is closely glued or adhered to both the spiral-shaped projections 6-2 around all the circumference of the curved subsections or the spiral turns of the spiral-shaped projections 6-2 or around a part of the circumference of the curved subsections or the spiral turns of the spiral-shaped projections 6-2 due to the negative pressure produced by the second functional device in the interior of the isolated target section of the mammal hollow organ. In one variant of the present alternative embodiment of the present invention, each of the two spiral-shaped projections 6-2 externally provided on the catheter body part of the catheter 500 shown in FIGS. 5.1-5.2 has a square-shaped cross-section, trapezoid-shaped cross-section or any another suitable cross-section shape known in the art.

In an embodiment of the present invention, the two spiral-shaped projections 6-2 both extend or run along a part or portion of the length of the catheter body part of the catheter 500 FIGS. 5.1-5.2, the catheter body part being defined by the isolating balloons 4.

As shown in FIGS. 5.1-5.2, the two spiral-shaped projections 6-2 provided on the catheter body part of the catheter 500 are both divided by a gap 18-3 into two separate pairs of parallel sections each having a curved form, wherein the gap 18-3 comprises the functional opening 7 positioned between the two separate pairs of parallel sections of the two spiral-shaped projections 6-2. In other words, the two separate pairs of parallel sections of the two spiral-shaped projections 6-2 enclose the functional opening 7 provided in the elongate catheter body between the two separate pairs of parallel sections of the two spiral-shaped projections 6-2 externally provided on the catheter body part of the catheter 500 shown in FIGS. 5.1-5.2. The functional opening 7 is adjacent to both the two pairs of parallel sections of the two spiral-shaped projections 6-2, wherein the two pairs of parallel sections of the two spiral-shaped projections 6-2 are equally spaced from the functional opening 7 along the length of the catheter body part of the catheter 500 shown in FIGS. 5.1-5.2 as defined between the isolating balloons 4 (i.e. a distance between one of the two pairs of parallel sections of the two spiral-shaped projections 6-2 and the functional opening 7 is equal to a distance between the other couple of parallel sections of the two spiral-shaped projections 6-2 and the functional opening 7). In other words, the two pairs of parallel sections of the two spiral-shaped projections 6-2 are equally positioned on different sides of the functional opening 7 along the length of the elongate catheter body of the catheter 500 shown in FIGS. 5.1-5.2 and each located next or close to the functional opening 7. Therefore, the two separate pairs of parallel sections of the two spiral-shaped projections 6-2 that enclose the functional opening 7 are provided on the elongate catheter body of the catheter 500 shown in FIGS. 5.1-5.2 between the isolating balloons 4 such that the functional opening 7 is between the two separate pairs of parallel sections of the two spiral-shaped projections 6-2, wherein the functional opening 7 enclosed by the two separate pairs of parallel sections of the two spiral-shaped projections 6-2 is within the gap 18-3 provided in the two spiral-shaped projections 6-2.

As also shown in FIGS. 5.1-5.2, the two spiral-shaped projections 6-2 run in parallel at a predetermined distance from each other, thereby forming a curved/spiral-shaped conduct or channel 19 between the two spiral-shaped projections 6-2, wherein the spiral-shaped channel 19 has an interruption corresponding to the gap 18-3. Thus, the two spiral-shaped projections 6-2 are designed such that they define the spiral-shaped channel 19 therebetween, wherein the functional opening 7 is provided in the catheter body part of the catheter 500 shown in FIGS. 5.1-5.2 in the interruption of the spiral-shaped channel 19 and positioned in the spiral-shaped channel 19 between the two spiral-shaped projections 6-2. In other words, the functional opening 7 provided in the catheter body part of the catheter 500 is positioned between the two spiral-shaped projections 6-2 in a region corresponding to the gap 18-3.

In an embodiment of the present invention, the functional opening 7 provided in the catheter body part of the catheter 500 shown in FIGS. 5.1-5.2 may be provided in the spiral-shaped channel 19 and may be enclosed by the two spiral-shaped projections 6-2, while the interruption corresponding to the gap 18-3 may be radially/circumferentially displaced from the functional opening 7, thereby increasing a length of a pathway from the interruption provided in the two spiral-shaped projections 6-2 to the functional opening 7, the pathway being required to be overcome by the gaseous media, liquid media or fluid media (including the biological contents) before entering the functional opening 7, when the mucosal tissue of the inner wall of the isolated target section of the mammal hollow organ is closely glued or adhered to both the spiral-shaped projections 6-2 provided on the elongate catheter body of the catheter 500 around all the circumference of the spiral turns of the two spiral-shaped projections 6-2 or around a part of the circumference of the spiral turns of the two spiral-shaped projections 6-2 due to the negative pressure produced by the second functional device in the interior of the isolated target section of the mammal hollow organ.

It is to note that each of the two spiral-shaped projections 6-2 externally provided on the catheter body part of the catheter 500 shown in FIGS. 5.1-5.2, the catheter body part being defined between the isolating balloons 4, comprises two spiral turns, wherein the four spiral turns of the two spiral-shaped projections 6-2 form two pairs of spinal turns, wherein the two spinal turns related to one of the two pairs of spinal turns are separated by the gap 18-3 from corresponding two spinal turns related to the other pair of spinal turns, and wherein the two spinal turns in each of the two pairs of spinal turns are both are designed such that they are generally spaced from each other and run in parallel with each other, and wherein the gap 18-3 comprising the functional opening 7 is generally provided in two junctions, each being used for connecting the two spiral turns with each other in a corresponding one of the two spiral-shaped projections 6-2.

In another embodiment of the present invention, each of the two spiral-shaped projections 6-2 provided on the elongate catheter body of the catheter 500 shown in FIGS. 5.1-5.2 comprises one or more spiral turns (in particular, one, two, three, four, five, six, seven, eight, nine, ten or more spiral turns), so that one or more pairs of spinal turns are formed by the spiral turns of the two spiral-shaped projections 6-2, wherein the two spinal turns in any of the pairs of spinal turns are both are designed such that they are generally spaced from each other and run in parallel with each other. In still another embodiment of the present invention, the gap 18-3 comprising the functional opening 7 provided in the elongate catheter body of the catheter 500 shown in FIGS. 5.1-5.2 is provided in the two spinal turns in any of the pairs of spiral turns of the two spiral-shaped projections 6-2.

It is to further note that both the spiral turns in each of the two spiral-shaped projections 6-2 provided on the elongate catheter body of the catheter 500 shown in FIGS. 5.1-5.2 have identical sizes (e.g. an inner radius/diameter, an outer radius/diameter, a thickness, a pitch, an inclination/an angle of ascent, curvature, etc.) and have a predetermined axial distance therebetween.

It is to further note that both the spiral turns in each of the two pairs of spinal turns of the two spiral-shaped projections 6-2 provided on the elongate catheter body of the catheter 500 shown in FIGS. 5.1-5.2 have identical sizes (e.g. an inner radius/diameter, an outer radius/diameter, a thickness, a pitch, an inclination/an angle of ascent, curvature, etc.) and are circumferentially displaced at a predetermined distance from each other.

As shown in FIGS. 5.1-5.2, each of the two pairs of spiral turns of the two spiral-shaped projections 6-2 is further provided with an additional gap 18-4, wherein said spiral turn in said pair of spiral turns is divided by the additional gap 18-4 into two separate sections. In other words, the two spiral-shaped projections 6-2 are provided with the two additional gaps 18-4, wherein the gap 18-3 comprising the functional opening 7 is generally positioned between the two additional gaps 18-4.

It is to note that the gap 18-3 and the two additional gaps 18-4 provided in the two spiral-shaped projections 6-2 provided on the elongate catheter body of the catheter 500 shown in FIGS. 5.1-5.2 are radially/circumferentially displaced from each other (e.g., spaced at predetermined or equal intervals from each other around the circumference of the elongate catheter body of the catheter 500).

In an embodiment of the present invention, the two spiral-shaped projections 6-2 provided on the elongate catheter body of the catheter 500 shown in FIGS. 5.1-5.2 are divided by two or more gaps (for example, one, two, three, four, five, six, seven, eight, nine, ten or more gaps), each being designed as the above-described gap 18-3 or the above-described gap 18-4, into three or more pairs of curved subsections or into three or more pairs of spiral turns, wherein the functional opening 7 is provided within one of said gaps of the spiral-shaped projection 6-1 and also located/positioned within the spiral-shaped channel 19 defined by the two spiral-shaped projections 6-2.

Thus, the two spiral-shaped projections 6-2 provided on the elongate catheter body of the catheter 500 shown in FIGS. 5.1-5.2 are divided by the gap 18-3 and the two additional gaps 18-4 into four separate pairs of curved subsections, wherein two central pairs of curved subsections, each being defined by the gap 18-3 and a corresponding one of the two additional gaps 18-4, are generally identically sized and shaped. It is to note that the two additional gaps 18-4 are provided in the two spiral-shaped projections 6-2 such that they are generally equally spaced from the gap 18-3 and positioned at opposite sides of the catheter body part of the catheter 500 as defined by the isolating balloons 4, wherein the gap 18-3 and the two additional gaps 18-4 are circumferentially displaced in relation to each other, i.e. the gap 18-3 and the two additional gaps 18-4 are displaced in relation to each other around the circumference of the catheter body part of the catheter 500 as defined by the isolating balloons 4. Such displaced positions of the gap 18-3 and the two additional gaps 18-4 around the circumference of the catheter body part of the catheter 500 shown in FIGS. 5.1-5.2 facilitates the flow of the gaseous media, liquid media or fluid media (including the biological contents) from the outer space of the isolated interior of the mammal hollow organ to pass, transfer or flow to the inner space provided within the isolated interior of the mammal hollow organ when the mucosal tissue of the inner wall of the isolated target section of the mammal hollow organ is closely glued or adhered to both the spiral-shaped projections 6-2 provided on the elongate catheter body of the catheter 500 around all the circumference of the spiral turns of the two spiral-shaped projections 6-2 or around a part of the circumference of the spiral turns of the two spiral-shaped projections 6-2 due to the negative pressure produced by the second functional device in the interior of the isolated target section of the mammal hollow organ.

It is to note that the two spiral-shaped projections 6-2 provided on the elongate catheter body of the catheter 500 shown in FIGS. 5.1-5.2 are both designed such that the two pairs of parallel sections of the two spiral-shaped projections 6-2 allow a distance between the functional opening 7, the functional opening 7 being located in the gap 18-3 between the two pairs of parallel sections of the two spiral-shaped projections 6-2 and being provided in catheter body part of the catheter 500 as defined between the isolating balloons 4, and the mucosal tissue of the inner wall of the mammal hollow organ, the mucosal tissue corresponding to the isolated target section of the mammal hollow organ and being between the inflated isolating balloons 4, to be provided when the negative pressure is produced by the second functional device in the interior of the isolated target section of the mammal hollow organ. In other words, in the catheter 500 shown in FIGS. 5.1-5.2, the two pairs of parallel sections of the two spiral-shaped projections 6-2 that enclose the functional opening 7 are intended to prevent the mucosal tissue of the inner wall of the mammal hollow organ, the mucosal tissue being between the inflated isolating balloons 4 and corresponding to the isolated target section of the mammal hollow organ, from contacting with the functional opening 7 or from approaching the functional opening 7 for a distance appropriate for suction of the mucosal tissue into the functional opening 7 when the negative pressure is produced by the second functional device in the isolated target section of the mammal hollow organ, thereby preventing the mucosal tissue of the inner wall of the isolated target section of the mammal hollow organ from being damaged and preventing the functional opening 7 from being blocked or occluded by the sucked mucosal tissue of the inner wall of the isolated target section of the mammal hollow organ. In other words, the two pairs of parallel sections of the two spiral-shaped projections 6-2 provided on the elongate catheter body of the catheter 500 shown in FIGS. 5.1-5.2 prevent the suction of the mucosal tissue of the inner wall of the isolated target section of the mammal hollow organ into the functional opening 7, while allowing the gaseous medium, the liquid medium or the fluid medium available in the interior of the isolated target section of the mammal hollow organ to be aspirated or evacuated by the second functional device (not shown) from all the interior of the isolated target section of the mammal hollow organ. Therefore, the two pairs of parallel sections of the two spiral-shaped projections 6-2 provided on the elongate catheter body of the catheter 500 shown in FIGS. 5.1-5.2 improve a safety, a reliability and an efficiency of the catheter 500.

Similar to the above described traverse grooves, slots or cuts 17, each of the two additional gaps 18-4 provided in the two spiral-shaped projections 6-2, the two spiral-shaped projections 6-2 being provided on the elongate catheter body of the catheter 500 shown in FIGS. 5.1-5.2, allows a positive effect to be provided in case when the mucosal tissue of the inner wall of the isolated target section of the mammal hollow organ is closely glued or adhered to the two spiral-shaped projections 6-2 provided on the elongate catheter body of the catheter 500 around all the circumference of the spiral turns of the two spiral-shaped projections 6-2 or a part of the circumference of the spiral turns of the two spiral-shaped projections 6-2 due to the negative pressure produced in the isolated interior of the mammal hollow organ by means of the second functional device, thereby splitting or dividing the isolated interior of the mammal hollow organ into an inner space of the isolated interior of the mammal hollow organ, the inner space being generally isolated between the glued or adhered mucosal tissue and the spiral turns of the two spiral-shaped projections 6-2, and an outer space of the isolated interior of the mammal hollow organ, the outer space being isolated by the glued or adhered mucosal tissue generally between the spiral turns of the two spiral-shaped projections 6-2 and the two isolating balloons 4. In other words, when the negative pressure is produced in the isolated interior of the mammal hollow organ by using the second functional device, the inner space and the outer space surrounding the inner space may be defined within isolated interior of the mammal hollow organ and separated from each other by the mucosal tissue being closely glued or adhered to the spiral turns of the two spiral-shaped projections 6-2 provided on the elongate catheter body of the catheter 500 shown in FIGS. 5.1-5.2 around all the circumference of the spiral turns of the two spiral-shaped projections 6-2 or a part of the circumference of the spiral turns of the two spiral-shaped projections 6-2.

It is to note that the helical or spiral shape of each of the two spiral-shaped projections 6-2 provided on the elongate catheter body of the catheter 500 shown in FIGS. 5.1-5.2 minimizes the possibility of complete isolation of the inner space of the isolated interior of the mammal hollow organ from the outer space of the isolated interior of the mammal hollow organ, thereby allowing a higher volume of the gaseous media, liquid media or fluid media (including the biological contents) from said outer space to pass, transfer or flow to said inner space when the mucosal tissue of the inner wall of the isolated target section of the mammal hollow organ is closely glued or adhered to both the spiral-shaped projections 6-2 provided on the elongate catheter body of the catheter 500 around all the circumference of the spiral turns of the two spiral-shaped projections 6-2 or around a part of the circumference of the spiral turns of the two spiral-shaped projections 6-2 due to the negative pressure produced by the second functional device in the interior of the isolated target section of the mammal hollow organ due to the negative pressure produced by the second functional device in the interior of the isolated target section of the mammal hollow organ.

Meanwhile, each of the two additional gaps 18-4 as provided in the two spiral-shaped projections 6-2 provided on the elongate catheter body of the catheter 500 shown in FIGS. 5.1-5.2 is designed such that said additional gap 18-4 defines a through channel which allows the gaseous media, liquid media or fluid media (including the biological contents) to be aspirated or evacuated through the defined through channel from the outer space of the isolated interior of the mammal hollow organ to the inner space provided within the isolated interior of the mammal hollow organ when the mucosal tissue of the inner wall of the isolated target section of the mammal hollow organ is closely glued or adhered to both the spiral-shaped projections 6-2 provided on the elongate catheter body of the catheter 500 around all the circumference of the spiral turns of the two spiral-shaped projections 6-2 or around a part of the circumference of the spiral turns of the two spiral-shaped projections 6-2 due to the negative pressure produced by the second functional device in the interior of the isolated target section of the mammal hollow organ.

In other words, both the through channels provided by the two additional gaps 18-4 of the two spiral-shaped projections 6-2 provided on the elongate catheter body of the catheter 500 shown in FIGS. 5.1-5.2 separately or in combination allow the gaseous media, liquid media or fluid media (including the biological contents) to be to aspirated or evacuated not only from the inner space provided within the isolated interior of the mammal hollow organ, but also from the outer space of the isolated interior of the mammal hollow organ to the inner space provided within the isolated interior of the mammal hollow organ, when the mucosal tissue of the inner wall of the isolated target section of the mammal hollow organ is closely glued or adhered to both the spiral-shaped projections 6-2 provided on the elongate catheter body of the catheter 500 around all the circumference of the spiral turns of the two spiral-shaped projections 6-2 or around a part of the circumference of the spiral turns of the two spiral-shaped projections 6-2 due to the negative pressure produced by the second functional device in the interior of the isolated target section of the mammal hollow organ. Thus, the through channels provided by the two additional gaps 18-4 of the two spiral-shaped projections 6-2 provided on the elongate catheter body of the catheter 500 shown in FIGS. 5.1-5.2 allow the gaseous media, liquid media or fluid media (including the biological contents) to be aspirated or evacuated from all the isolated interior of the mammal hollow organ even when the mucosal tissue of the inner wall of the isolated target section of the mammal hollow organ is closely glued or adhered to the two spiral-shaped projections 6-2 provided on the elongate catheter body of the catheter 500 around all the circumference of the spiral turns of the two spiral-shaped projections 6-2 or a part of the circumference of the spiral turns of the two spiral-shaped projections 6-2.

Both the through channels defined by the two additional gaps 18-4 of the two spiral-shaped projections 6-2 provided on the elongate catheter body of the catheter 500 shown in FIGS. 5.1-5.2 allow the above-described inner space of the isolated interior of the mammal hollow organ and the above-described outer space of the isolated interior of the mammal hollow organ to be in fluid communication with each other, thereby allowing the gaseous media, liquid media or fluid media (including the biological contents) to pass, transfer or flow through said through channels from the outer space to the inner space provided within the isolated interior of the mammal hollow organ when the mucosal tissue of the inner wall of the isolated target section of the mammal hollow organ is closely glued or adhered to both the spiral-shaped projections 6-2 provided on the elongate catheter body of the catheter 500 around all the circumference of the spiral turns of the two spiral-shaped projections 6-2 or around a part of the circumference of the spiral turns of the two spiral-shaped projections 6-2 due to the negative pressure produced by the second functional device in the interior of the isolated target section of the mammal hollow organ due to the negative pressure produced by the second functional device in the interior of the isolated target section of the mammal hollow organ by using the second functional device. In turn, the aspiration or evacuation of the gaseous media, liquid media or fluid media, e.g. the biological contents, from both the inner space and the outer space of the isolated interior of the mammal hollow organ through the through channels provided by the two additional gaps 18-4 of the two spiral-shaped projections 6-2 provided on the elongate catheter body of the catheter 500 shown in FIGS. 5.1-5.2 results in collection of the biological contents related to all the isolated interior of the mammal hollow organ in the storage container of the aspirator, thereby increasing the accuracy and/or the reliability of the laboratory examinations to be performed on the collected biological contents.

Moreover, in the catheter 500 shown in FIGS. 5.1-5.2, each of the gap 18-3 and two additional gaps 18-4 is in fluid communication with the spiral-shaped channel 19 extending between the two spiral-shaped projections 6-2 provided on the elongate catheter body of the catheter 500, thereby further allowing the gaseous media, liquid media or fluid media (including the biological contents) to pass, transfer or flow from the outer space of the isolated interior of the mammal hollow organ to the inner space provided within the isolated interior of the mammal hollow organ when the mucosal tissue of the inner wall of the isolated target section of the mammal hollow organ is closely glued or adhered to both the spiral-shaped projections 6-2 provided on the elongate catheter body of the catheter 500 around all the circumference of the spiral turns of the two spiral-shaped projections 6-2 or around a part of the circumference of the spiral turns of the two spiral-shaped projections 6-2 due to the negative pressure produced by the second functional device in the interior of the isolated target section of the mammal hollow organ.

Therefore, the two additional gaps 18-4 of the two spiral-shaped projections 6-2 provided on the elongate catheter body of the catheter 500 shown in FIGS. 5.1-5.2 further improve a safety, a reliability and an efficiency of the catheter 500.

It is obvious for those skilled in the art that the gap 18-3 and the two additional gaps 18-4 provided in the two spiral-shaped projections 6-2 provided on the elongate catheter body of the catheter 500 shown in FIGS. 5.1-5.2 may be each alternatively named as a slot or an interruption.

In other embodiments of the present invention, the two spiral-shaped projections 6-2 provided on the elongate catheter body of the catheter 500 shown in FIGS. 5.1-5.2 is provided with one or more additional gaps 18-4 (e.g. one, two, three, four, five, six, seven, eight, nine, ten or more additional gaps 18-4).

In some other embodiments of the present invention, the gap 18-3 and the additional gaps 18-4 are aligned with each other along the catheter body part of the catheter 500 shown in FIGS. 5.1-5.2, the catheter body part being defined by the isolating balloons 4.

In various embodiments of the present invention, at least one or each turn in each of the two spiral-shaped projections 6-2 provided on the elongate catheter body of the catheter 500 shown in FIGS. 5.1-5.2 is provided with two opposite additional gaps 18-4.

In different embodiments of the present invention, the two spiral-shaped projections 6-2 provided on the elongate catheter body of the catheter 500 shown in FIGS. 5.1-5.2 is only provided with the gap 18-3 comprising the functional opening 7, while the additional gaps 18-4 are not provided in the two spiral-shaped projections 6-2.

According to an embodiment of the present invention, two or more functional openings 7 (e.g., two, three, four, five, six, seven, eight, nine, ten or more functional openings 7) are provided in the elongate catheter body of the catheter 500 shown in FIGS. 5.1-5.2, wherein the two spiral-shaped projections 6-2 provided on the elongate catheter body of the catheter 500 are divided by two or more gaps (for example, one, two, three, four, five, six, seven, eight, nine, ten or more gaps), each gap being designed as the above-described gap 18-3 or the above-described additional gap 18-4, into three or more pairs of curved subsections or into three or more pairs of spiral turns, wherein each of the functional openings 7 is provided within one of said gaps of the two spiral-shaped projections 6-2 and also located/positioned within the spiral-shaped channel 19 defined by the two spiral-shaped projections 6-2. In other words, in the present embodiment of the present invention, the functional openings 7 provided in the elongate catheter body of the catheter 500 shown in FIGS. 5.1-5.2 are radially/circumferentially displaced from each other (e.g., equally spaced around the circumference of the elongate catheter body of the catheter 500 or circumferentially spaced around the elongate catheter body of the catheter 500 at predetermined intervals from each other), wherein the functional openings 7 provided in the elongate catheter body of the catheter 500 are alternatively aligned with each other such that the functional openings 7 are in line with each other along the length of the catheter body part defined by the isolating balloons 4 and spaced from each other at predetermined or equal intervals along the length of the catheter body part defined by the isolating balloons 4. In the present embodiment of the present invention, the increase of number of the functional openings 7, each functional opening 7 being provided in the elongate catheter body of the catheter 500 shown in FIGS. 5.1-5.2 in a corresponding one of the gaps of the two spiral-shaped projections 6-2, further improves the aspiration or evacuation of the gaseous media, liquid media or fluid media (including the biological contents) from the above-described outer space of the isolated interior of the mammal hollow organ to the inner space within the isolated interior of the mammal hollow organ when the mucosal tissue of the inner wall of the isolated target section of the mammal hollow organ is closely glued or adhered to both the spiral-shaped projections 6-2 around all the circumference of the curved subsections or the spiral turns of the spiral-shaped projections 6-2 or around a part of the circumference of the curved subsections or the spiral turns of the spiral-shaped projections 6-2 due to the negative pressure produced by the second functional device in the interior of the isolated target section of the mammal hollow organ.

According to another embodiment of the present invention, the elongate catheter body of the catheter 500 shown in FIGS. 5.1-5.2 may be preliminary provided or equipped with at least one loop or thread (for example, one, two, three, four, five, six, seven, eight, nine, ten or more loops or threads) designed to be gripped with endoscope biopsy forceps for capturing the elongate catheter body of the catheter 500. To insert the elongate catheter body of the catheter 500 shown in FIGS. 5.1-5.2 into the lumen of the mammal duodenum to have a desired placement site in the mammal duodenum, the distal end of the elongate catheter body of the catheter 500, when preliminary well lubricated with a vaseline oil, is administered or inserted through the mammal nasal passage and advanced to the mammal gastric cavity; then the endoscope is further administered or advanced in parallel with the catheter 500 through the mammal oral cavity to the mammal gastric cavity so as to grip, by means of the endoscope biopsy forceps, the loop or the thread of the elongate catheter body of the catheter 500 for capturing the elongate catheter body of the catheter 500; the captured elongate catheter body is finally guided or advanced along with the endoscope to the mammal duodenum. Subsequently, one of the two isolating balloons 4 of the catheter 500 shown in FIGS. 5.1-5.2, the farthest from the distal end of elongate catheter body of the catheter 500, is placed in the bulb of the mammal duodenum under control of the endoscope.

FIGS. 5.3-5.4 illustrates another alternative embodiment of the present invention where gap 18-3 is not provided in the two spiral-shaped projections 6-2 provided on the elongate catheter body of the catheter 500, and two gaps 18-4 are only provided in the two spiral-shaped projections 6-2, wherein each of the two spiral-shaped projections 6-2 is designed such that said spiral-shaped projection 6-2 has a rectangle-shaped cross-section. Thus, as shown in FIGS. 5.3-5.4, the each of the two spiral-shaped projections 6-2 is flattened (e.g. flattened at the top or apex) or has a flattened apex or top (i.e. the apex or top of said spiral-shaped projections 6-2 is flat and regular), thereby preventing the mucosal tissue of the inner wall of the isolated target section of the mammal hollow organ from being damaged when said mucosal tissue is closely glued or adhered to both the spiral-shaped projections 6-2 around all the circumference of the curved subsections or the spiral turns of the spiral-shaped projections 6-2 or around a part of the circumference of the curved subsections or the spiral turns of the spiral-shaped projections 6-2 due to the negative pressure produced by the second functional device in the interior of the isolated target section of the mammal hollow organ. In one variant of the present alternative embodiment of the present invention, each of the two spiral-shaped projections 6-2 has a square-shaped cross-section, trapezoid-shaped cross-section or any another suitable cross-section shape known in the art. As shown in FIGS. 5.3-5.4, the functional opening 7 is within the spiral-shaped channel 19 defined by the two spiral-shaped projections 6-2, i.e. the functional opening 7 is not further located/positioned within the gap 18-3, wherein the spiral-shaped channel 19 is in fluid communication with both the gaps 18-4, thereby providing the fluid communication between the inner space and the outer space of the isolated interior of the mammal hollow organ when the mucosal tissue of the inner wall of the isolated target section of the mammal hollow organ is closely glued or adhered to both the spiral-shaped projections 6-2 around all the circumference of the curved subsections or the spiral turns of the spiral-shaped projections 6-2 or around a part of the circumference of the curved subsections or the spiral turns of the spiral-shaped projections 6-2 due to the negative pressure produced by the second functional device in the interior of the isolated target section of the mammal hollow organ. In the present alternative embodiment of the present invention, that fact that the functional opening 7 located/positioned within the spiral-shaped channel 19 is spaced from both the gaps 18-4 provided in the spiral-shaped projections 6-2 along the length of the elongate catheter body of the catheter 500 shown in FIGS. 5.3-5.4 and radially/circumferentially displaced from both the gaps 18-4 provided in spiral-shaped projections 6-2 increases a length of a pathway from any the two interruptions provided in the two spiral-shaped projections 6-2 (wherein each of the two interruptions corresponds to a particular one of the two gaps 18-4) to the functional opening 7, the pathway being required to be overcome by the gaseous media, liquid media or fluid media (including the biological contents) before entering the functional opening 7, when the mucosal tissue of the inner wall of the isolated target section of the mammal hollow organ is closely glued or adhered to both the spiral-shaped projections 6-2 provided on the elongate catheter body of the catheter 500 around all the circumference of the spiral turns of the two spiral-shaped projections 6-2 or around a part of the circumference of the spiral turns of the two spiral-shaped projections 6-2 due to the negative pressure produced by the second functional device in the interior of the isolated target section of the mammal hollow organ. In a first variant of the present alternative embodiment of the present invention, two or more functional openings 7 (e.g., two, three, four, five, six, seven, eight, nine, ten or more functional openings 7) are within the spiral-shaped channel 19 and spaced from each other along the length of the elongate catheter body of the catheter 500 shown in FIGS. 5.3-5.4 (wherein all the functional openings 7 or a part of them are further radially/circumferentially displaced from each other or alternatively aligned with each other), wherein all the functional openings 7 are located/positioned between the gaps 18-4 provided in the two spiral-shaped projections 6-2 along the length of the elongate catheter body of the catheter 500. In a second variant of the present alternative embodiment of the present invention, three functional openings 7 may be provided in the elongate catheter body of the catheter 500 shown in FIGS. 5.3-5.4 within the spiral-shaped channel 19, and three gaps, each gap corresponding to the above-described gap 18-3 or the above-described gap 18-4, are provided in the two spiral-shaped projections 6-2, wherein the three functional openings 7 are equally spaced along the length of the elongate catheter body of the catheter 500 and radially/circumferentially displaced in relation to each other (alternatively, aligned with each other such that the functional openings 7 are positioned in line with each other along the length of the catheter body part defined by the isolating balloons 4 and spaced from each other at predetermined or equal intervals along the length of the catheter body part defined by the isolating balloons 4), and wherein the three gaps provided in the two spiral-shaped projections 6-2 are equally spaced along the length of the elongate catheter body of the catheter 500 and radially/circumferentially displaced in relation to each other. In the second variant of the present alternative embodiment of the present invention, each of the functional openings 7 is within a corresponding one of the three gaps provided in the two spiral-shaped projections 6-2 externally provided on the catheter body part of the catheter 500 shown in FIGS. 5.3-5.4. or aligned with each other along the length of the catheter body part defined by the isolating balloons 4

Therefore, each of the above-described catheter 200, the above-described catheter 300, the above-described catheter 400 and the above-described catheter 500 also allows functional investigations of any mammal hollow organ to be conducted or performed, and allows appropriate treatments to be performed on the mammal hollow organ. Furthermore, the examination and/or the laboratory analysis of the biological contents collected by the catheter 200, the catheter 300, the catheter 400 or the catheter 500 allows inflammatory formations, benign formations and malignant growth, and infectious diseases to be differentially diagnosed with a high accuracy. The catheter 200, the catheter 300, the catheter 400 or the catheter 500 allows inflammatory and other diseases, the bleeding and wall defects or injury of the mammal hollow organ to be treated by sampling or taking a specific biological contents (e.g. a biological liquid) from the isolated interior of the mammal hollow organ. The isolated interior providing a bypass function during a surgical manipulation allows a hemostasis to be controlled, and also allows surgical manipulations to be performed, in particular allows the resection of a mammal vessel site to be performed and also allows a plastic surgery of the resected mammal vessel site to be performed. It is to further note that the catheter 200, the catheter 300, the catheter 400 or the catheter 500 or the above-described system comprising the catheter 200, the catheter 300, the catheter 400 or the catheter 500 allows a high volume of the biological contents having a normal physiological and biochemical state to be collected by the catheter 200, the catheter 300, the catheter 400 or the catheter 500 without any additional artificial stimulation of the excretory function of the mammal hollow organ, in particular due to the production of the negative pressure corresponding to a physiological negative pressure of the mammal hollow organ, the physiological negative pressure being normally created, for example, due to the peristalsis of the intestine in the isolated interior of the mammal hollow organ having the catheter 200, the catheter 300, the catheter 400 or the catheter 500 inserted into the lumen thereof.

We claim:

1. A catheter for isolating an interior of a mammal hollow organ, the catheter comprising:
an elongate catheter body designed to be inserted into a lumen of the mammal hollow organ;
two balloons (4), individually and separately disposed along the elongate catheter body and designed to be inflated to isolate the interior of the mammal hollow organ therebetween; and
a functional channel (3) extending in the elongate catheter body and comprising a functional opening (7) provided in the elongate catheter body between the balloons (4),
wherein the functional channel (3) is designed to allow a negative pressure to be produced in the isolated interior to take a gaseous medium, a liquid medium or a fluid medium therefrom via the functional opening (7), and wherein
a spiral-shaped projection (6-1) divided by a gap (18-1) into two curved sections is further provided on a catheter body part defined by the two balloons (4) such that the gap (18-1) comprises the functional opening (7), and the two curved sections of the spiral-shaped projection (6-1) enclose the functional opening (7),
wherein the spiral-shaped projection (6-1) is designed such that the spiral-shaped projection (6-1) allows a distance between a mucosal tissue of the mammal hollow organ, the mucosal tissue being between the inflated balloons (4), and the functional opening (7).

2. The catheter of claim 1, wherein the spiral-shaped projection (6-1) is further provided with two additional gaps (18-2), wherein the two additional gaps (18-2) are spaced from the gap (18-1) such that the gap (18-1) is positioned between the two additional gaps (18-2).

3. The catheter of claim 2, wherein the gap (18-1) and the two additional gaps (18-2) are further circumferentially displaced in relation to each other or aligned with each other along the catheter body part.

4. The catheter of claim 1, further comprising an inflation channel (2) extending in the elongate catheter body to deliver a liquid or gaseous medium to the balloons (4) to provide inflation thereof, wherein the functional channel (3) and the inflation channel (2) are hermetically isolated from each other.

5. The catheter of claim 4, further comprising a main channel (1) extending in the elongate catheter body, wherein the main channel (1) is provided at opposite ends thereof with an inlet (5-2) and an outlet (5-1) provided both in the elongate catheter body outside the catheter body part defined by the two balloons (4), and wherein the main channel (1) is hermetically isolated from the functional channel (3) and the inflation channel (2), and wherein the main channel is further designed to supply a gaseous medium, a liquid medium or a fluid medium thereinto and comprises an additional outlet provided at a distal end of the elongate catheter body, the distal end being used for inserting the elongate catheter body into the lumen of the mammal hollow organ.

6. The catheter of claim 5, further comprising a shut-off valve installed in a proximal terminus of the main channel (1).

7. The catheter of claim 4, further comprising a medium supply channel (1-1) extending in the elongate catheter body, wherein the medium supply channel (1-1) is hermetically isolated from the functional channel (3) and the inflation channel (2), and wherein the medium supply channel (1-1) is further designed to supply a gaseous medium, a liquid medium or a fluid medium thereinto and comprises an end-face opening (11-1) provided at a distal end of the elongate catheter body, the distal end being used for inserting the elongate catheter body into the lumen of the mammal hollow organ.

8. The catheter of claim 7, further comprising a shut-off valve installed in a proximal terminus of the medium supply channel (1-1).

9. The catheter of claim 7, further comprising a medium drainage channel (1-2) extending in the elongate catheter body, wherein the medium drainage channel (1-2) is provided at opposite ends thereof with an inlet (5-2) and an outlet (5-1) provided both in the elongate catheter body outside the catheter body part defined by the two balloons (4), and wherein the medium drainage channel (1-2) is hermetically isolated from the functional channel (3), the inflation channel (2) and the medium supply channel (1-1).

10. The catheter of claim 9, wherein the medium drainage channel (1-2) further comprises an end-face opening (11-2) provided at a distal end of the elongate catheter body, the distal end being used for inserting the elongate catheter body into the lumen of the mammal hollow organ, wherein the end-face opening (11-2) is in fluid communication with the inlet (5-2) and the outlet (5-1).

11. The catheter of claim 9, further comprising a shut-off valve installed in a proximal terminus of the medium drainage channel (1-2).

12. The catheter of claim 1, wherein the elongate catheter body is further provided with a loop or a thread designed to be gripped with an endoscope for guiding the gripped catheter body within the mammal hollow organ.

13. The catheter of claim 1, wherein the elongate catheter body is further provided with a net or enclosure (16) being permeable to a liquid medium and/or a gaseous medium, wherein the net or enclosure (16) at least partly encloses a catheter body part defined by the balloons (4) so as to cover the functional opening (7), and wherein the net or enclosure (16) is attached to the balloons (4) or encloses them such that the net or enclosure (16) becomes strained when the balloons (4) are inflated.

14. A catheter for isolating an interior of a mammal hollow organ, the catheter comprising:
an elongate catheter body designed to be inserted into a lumen of the mammal hollow organ;
two balloons (4), individually and separately disposed along the elongate catheter body and designed to be inflated to isolate the interior of the mammal hollow organ therebetween; and
a functional channel (3) extending in the elongate catheter body and comprising a functional opening (7) provided in the elongate catheter body between the balloons (4),
wherein the functional channel (3) is designed to allow a negative pressure to be produced in the isolated interior to take a gaseous medium, a liquid medium or a fluid medium therefrom via the functional opening (7), and wherein
two spiral-shaped projections (6-2) divided by a gap (18-3) into two pairs of curved sections are further provided on a catheter body part defined by the two balloons (4) such that the gap (18-3) comprises the functional opening (7), and the two pairs of curved sections enclose the functional opening (7),
wherein the two spiral-shaped projections (6-2) are designed such that both the spiral-shaped projections (6-2) allow a distance between a mucosal tissue of the mammal hollow organ, the mucosal tissue being between the inflated balloons (4), and the functional opening (7).

15. The catheter of claim 14, wherein the two spiral-shaped projections (6-2) are further provided with two additional gaps (18-4), wherein the two additional gaps (18-4) are spaced from the gap (18-3) such that the gap (18-3) is positioned between the two additional gaps (18-4).

16. The catheter of claim 15, wherein the gap (18-3) and the two additional gaps (18-4) are further circumferentially displaced in relation to each other or aligned with each other along the catheter body part.

17. The catheter of claim 14, wherein a spiral-shaped channel (19) is further defined between the two spiral-shaped projections (6-2), and the functional opening (7) is within the spiral-shaped channel (19).

18. The catheter of claim 14, further comprising an inflation channel (2) extending in the elongate catheter body to deliver a liquid or gaseous medium to the balloons (4) to provide inflation thereof, wherein the functional channel (3) and the inflation channel (2) are hermetically isolated from each other.

19. The catheter of claim 18, further comprising a main channel (1) extending in the elongate catheter body, wherein the main channel (1) is provided at opposite ends thereof with an inlet (5-2) and an outlet (5-1) provided both in the elongate catheter body outside the catheter body part defined by the two balloons (4), and wherein the main channel (1) is hermetically isolated from the functional channel (3) and the inflation channel (2), and wherein the main channel is further designed to supply a gaseous medium, a liquid medium or a fluid medium thereinto and comprises an additional outlet provided at a distal end of the elongate catheter body, the distal end being used for inserting the elongate catheter body into the lumen of the mammal hollow organ.

20. The catheter of claim 19, further comprising a shut-off valve installed in a proximal terminus of the main channel (1).

21. The catheter of claim 18, further comprising a medium supply channel (1-1) extending in the elongate catheter body, wherein the medium supply channel (1-1) is hermetically isolated from the functional channel (3) and the inflation channel (2), and wherein the medium supply channel (1-1) is further designed to supply a gaseous medium, a liquid medium or a fluid medium thereinto and comprises an end-face opening (11-1) provided at a distal end of the elongate catheter body, the distal end being used for inserting the elongate catheter body into the lumen of the mammal hollow organ.

22. The catheter of claim 21, further comprising a shut-off valve installed in a proximal terminus of the medium supply channel (1-1).

23. The catheter of claim 21, further comprising a medium drainage channel (1-2) extending in the elongate catheter body, wherein the medium drainage channel (1-2) is provided at opposite ends thereof with an inlet (5-2) and an outlet (5-1) provided both in the elongate catheter body outside the catheter body part defined by the two balloons (4), and wherein the medium drainage channel (1-2) is hermetically isolated from the functional channel (3), the inflation channel (2) and the medium supply channel (1-1).

24. The catheter of claim 23, wherein the medium drainage channel (1-2) further comprises an end-face opening (11-2) provided at a distal end of the elongate catheter body, the distal end being used for inserting the elongate catheter body into the mammal hollow organ, wherein the end-face opening (11-2) is in fluid communication with the inlet (5-2) and the outlet (5-1).

25. The catheter of claim 23, further comprising a shut-off valve installed in a proximal terminus of the medium drainage channel (1-2).

26. The catheter of claim 14, wherein the elongate catheter body is further provided with a net or enclosure (16) being permeable to a liquid medium and/or a gaseous medium, wherein the net or enclosure (16) at least partly encloses a catheter body part defined by the balloons (4) so as to cover the functional opening (7), and wherein the net or enclosure (16) is attached to the balloons (4) or encloses them such that the net or enclosure (16) becomes strained when the balloons (4) are inflated.

27. The catheter of claim 14, wherein the elongate catheter body is further provided with a loop or a thread designed to be gripped with an endoscope for guiding the gripped catheter body within the mammal hollow organ.

28. A catheter for isolating an interior of a mammal hollow organ, the catheter comprising:
an elongate catheter body designed to be inserted into a lumen of the mammal hollow organ;
two balloons (4), individually and separately disposed along the elongate catheter body and designed to be inflated to isolate the interior of the mammal hollow organ therebetween; and
a functional channel (3) extending in the elongate catheter body and comprising a functional opening (7) provided in the elongate catheter body between the balloons (4),
wherein the functional channel (3) is designed to allow a negative pressure to be produced in the isolated interior to take a gaseous medium, a liquid medium or a fluid medium therefrom via the functional opening (7), and wherein
two ring-shaped projections (6) enclosing the functional opening (7) are further provided on the elongate catheter body between the balloons (4) such that the functional opening (7) is between the two ring-shaped projections (6), and
wherein the two ring-shaped projections (6) are designed such that they allow a distance between a mucosal tissue of the mammal hollow organ, the mucosal tissue being between the inflated balloons (4), and the functional opening (7), and
wherein each of the two ring-shaped projections (6) is provided with a traverse groove or cut (17), and
wherein the traverse groove or cut (17) is designed such that the traverse groove or cut (17) allows the gaseous medium, the liquid medium or the fluid medium to be taken via the functional opening (7) to pass therethrough.

29. The catheter of claim 28, wherein each of the two ring-shaped projections (6) is further provided with one or more though-holes, each being designed to allow the gaseous medium, the liquid medium or the fluid medium to be taken via the functional opening (7) to pass therethrough.

30. The catheter of claim 28, further comprising an inflation channel (2) extending in the elongate catheter body to deliver a liquid or gaseous medium to the balloons (4) to provide inflation thereof, wherein the functional channel (3) and the inflation channel (2) are hermetically isolated from each other.

31. The catheter of claim 30, further comprising a main channel (1) extending in the elongate catheter body, wherein the main channel (1) is provided at opposite ends thereof with an inlet (5-2) and an outlet (5-1) provided both in the elongate catheter body outside the catheter body part defined by the two balloons (4), and wherein the main channel (1) is hermetically isolated from the functional channel (3) and the inflation channel (2), and wherein the main channel is further designed to supply a gaseous medium, a liquid medium or a fluid medium thereinto and comprises an additional outlet provided at a distal end of the elongate catheter body, the distal end being used for inserting the elongate catheter body into the lumen of the mammal hollow organ.

32. The catheter of claim 31, wherein at least one or each of the inlet (5-2) and the outlet (5-1) of the main channel (1) is a cone-shaped hole tapering towards an interior of the main channel (1).

33. The catheter of claim 31, further comprising a shut-off valve installed in a proximal terminus of the main channel (1).

34. The catheter of claim 30, further comprising a medium supply channel (1-1) extending in the elongate catheter body, wherein the medium supply channel (1-1) is hermetically isolated from the functional channel (3) and the inflation channel (2), and wherein the medium supply channel (1-1) is further designed to supply a gaseous medium, a liquid medium or a fluid medium thereinto and comprises an end-face opening (11-1) provided at a distal end of the elongate catheter body, the distal end being used for inserting the elongate catheter body into the lumen of the mammal hollow organ.

35. The catheter of claim 34, further comprising a shut-off valve installed in a proximal terminus of the medium supply channel (1-1).

36. The catheter of claim 34, further comprising a medium drainage channel (1-2) extending in the elongate catheter body, wherein the medium drainage channel (1-2) is provided at opposite ends thereof with an inlet (5-2) and an outlet (5-1) provided both in the elongate catheter body outside the catheter body part defined by the two balloons (4), and wherein the medium drainage channel (1-2) is hermetically isolated from the functional channel (3), the inflation channel (2) and the medium supply channel (1-1).

37. The catheter of claim 36, wherein the medium drainage channel (1-2) further comprises an end-face opening (11-2) provided at a distal end of the elongate catheter body, the distal end being used for inserting the elongate catheter body into the mammal hollow organ, wherein the end-face opening (11-2) is in fluid communication with the inlet (5-2) and the outlet (5-1).

38. The catheter of claim 36, further comprising a shut-off valve installed in a proximal terminus of the medium drainage channel (1-2).

39. The catheter of claim 37, wherein at least one or each of the inlet (5-2) and the outlet (5-1) of the medium drainage channel (1-2) is a cone-shaped hole tapering towards an interior of the medium drainage channel (1-2).

40. The catheter of claim 28, wherein the elongate catheter body is further provided with a net or enclosure (16) being permeable to a liquid medium and/or a gaseous medium, wherein the net or enclosure (16) at least partly encloses a catheter body part defined by the balloons (4) so as to cover the functional opening (7), and wherein the net or enclosure (16) is attached to the balloons (4) or encloses them such that the net or enclosure (16) becomes strained when the balloons (4) are inflated.

41. The catheter of claim 28, wherein the elongate catheter body is further provided with a loop or a thread designed to be gripped with an endoscope for guiding the gripped catheter body within the mammal hollow organ.
